(19) United States
(12) Patent Application Publication
(10) Pub. No.: US 2011/0318738 A1
(43) Pub. Date: Dec. 29, 2011

(54) IDENTIFICATION AND REGULATION OF A NOVEL DNA DEMETHYLASE SYSTEM

(75) Inventors: David Jones, Salt Lake City, UT (US); Bradley Cairns, Salt Lake City, UT (US); Kunal Rai, Salt Lake City, UT (US)

(73) Assignee: University of Utah Research Foundation, Salt Lake City, UT (US)

(21) Appl. No.: 13/141,432

(22) PCT Filed: Dec. 4, 2009
(86) PCT No.: PCT/US09/66710

§ 371 Date: Sep. 12, 2011

Related U.S. Application Data
(60) Provisional application No. 61/203,586, filed on Dec. 23, 2008.

(51) Int. Cl. C12Q 1/68

(52) U.S. Cl. 435/6.11; 435/6.12

(57) ABSTRACT

Disclosed herein are methods and systems directed at detecting, evaluating, ameliorating, preventing and treating an oncogenic event. The disclosed methods and systems can comprise one or more Demethylase System Components or other compositions that can be used alone or in combination to detect, evaluate, treat, ameliorate, or prevent an oncogenic event.

### STATEMENT REGARDING FEDERALLY FUNDED RESEARCH

Portions of the research and inventions disclosed herein may have been made with U.S. Government support under the National Institutes of Health Grants Nos. R01 CA116468-02 and No. R01 HD058506-01. The U.S. government has certain rights in this invention.

### BACKGROUND

DNA methylation is associated with gene silencing and also plays several important roles in mammalian development and genomic imprinting (Reik, 2007). Misregulation of DNA methylation also contributes to oncogenic events by causing genomic instability and inappropriate silencing of tumor suppressor genes (Esteller, 2008). Although genome-wide hypomethylation is a hallmark of many oncogenic events, including but not limited to, the development of a variety of cancers including colorectal cancer, the roles of active DNA demethylation during these oncogenic events are unknown.

To date the mechanisms and enzymes involved in active DNA demethylation in vertebrates remain unclear. Proposed mechanisms include (1) direct removal of the methyl group, regenerating cytosine, (2) direct removal of the base (via glycosylase/lyase base excision activity, as in plants), followed by repair/replacement with cytosine, (3) conversion of the base to thymine (via deamination), followed by removal and subsequent repair, and (4) excision of one or more nucleotides surrounding 5-meC, followed by repair. Although the DNA methyltransferase (DNMT) enzymes that generate 5-methylcytosine (5-meC) in vertebrates have been studied (Goll and Bestor, 2005), the evidence for a vertebrate enzyme exhibiting reproducible DNA demethylation either in vitro or in vivo is still lacking.

Accordingly, there exists a need in the art for elucidating the mechanisms, systems, and compositions that participate in DNA methylation and demethylation, thereby contributing to oncogenic events.

A description of and the advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention. These are non-limiting examples.

FIG. 1 shows the qRT-PCR determinations from embryos injected with M-DNA (A), and at different fragment concentrations (B). FIG. 1 also shows the methylation status of M-DNA assessed by HpaII digestion and Southern blotting (C), or LC-MS quantitation of total 5-MeC (D) in total genomic DNA isolated from embryos at 13 hpf, injected at the single-cell stage with M-DNA (200 pg) and morpholinos as indicated. Lanes 1, 7, and 13 correspond to wild-type sample. AAAmm refers to a set of three control morpholinos against AID (4 pg), Apobec2a (4 pg), and Apobec2b (2 pg) (AAA), which each contain five mismatched (mm) bases (of 25 total to prevent binding) relative to the efficacious morpholino (same amount as controls). For HpaII/MspI susceptibility, one representative of at least three biological repeats is shown. LC-MS measurements; two biological replicates. For this figure and all others, asterisks (*) depict statistical significance (p<0.05) and the error bars equal +/_ one standard deviation.

FIG. 2 shows the methylation status assessed by HpaII digestion of total genomic DNA (A) with LC-MS quantitation (B—upper panel). FIG. 2 also shows the HpaII digestion of M-DNA (Southern analysis) (B—lower panel) and bisulphite sequencing of M-DNA (C). Lanes 1, 7, and 13 in (A) and lane 1 in (B) correspond to wild-type sample. For (B), M-DNA was injected at 5 pg, below the threshold level for eliciting demethylation on its own. For (C), twenty clones were subjected to bisulphite sequencing, and the methylation status of each HpaII/MspI (CCGG) site reported as a percentage of total sites tested. For each experiment, one representative of at least three biological repeats is shown except in LC-MS measurement where graph is prepared from values of two biological replicates.

FIG. 3A shows a schematic of the PCR reaction for thymine (CmeCGG>CTGG) detection at M-DNA HpaII/MspI sites using an A-tailed primer (only 3 of the ˜22 bases shown) with an adenosine at the 3′ end. FIG. 3B shows the detection of a G:T mismatch on M-DNA by PCR. M-DNA, AID mRNA, and RNA encoding either wild-type or catalytically inactive hMbd4 (D560A) was injected at the single-cell stage and assessed at 13 hpf.

FIG. 4 shows that Gadd45 family members are upregulated by M-DNA, assessed by RT-PCR.

FIG. 5 shows the enrichment of AID, MBD4, and Gadd45α on pCMV-Luc, which contains both methylated (Me) and unmethylated (U) regions. ChIP experiments with extracts from embryos (12 hpf) injected at the single-cell stage with V5-tagged AID, HA-tagged hMbd4, His-tagged Gadd45α and in vitro-methylated (by HpaII methylase) pCMV-Luc (Me-P). Y-axis values represent the ratio of enrichment on a DNA segment containing in vitro methylated CmeCGG sites to enrichment on a site (also on pCMV-Luc) containing no CCGG elements. Me-P and U-P on axis depict methylated and unmethylated plasmid, respectively. The graph shows one representative experiment of three biological repeats.

FIG. 6A shows a schematic of the neurod2 promoter and start site region. R1 and R2 show regions of bisulfite sequencing (Results shown for only R1; R2 remains unmethylated and unaffected). FIG. 6B shows the enrichment of AID and hMbd4 at neurod2 (P1 versus P2). ChIP experiments with extracts from embryos at 80% epiboly, which were initially injected at the single-cell stage with VS-tagged AID and HA-tagged hMbd4. The graph shows one representative biological experiment (two biological repeats), with the average of three technical replicates shown.

FIG. 7 shows a model for 5-meC Demethylation wherein demethylation can occur through a two-step coupled enzymatic process, promoted by Gadd45. The first enzymatic step can involve deamination of 5-meC by AID (amine group removed —NH₂) generating a thymine product and a G:T mismatch. The second step can involve thymine base removal by Mdb4, generating an abasic site. As the transient G:T intermediate is not detected in cells with active Mbd4, but is with catalytically inactive Mbd4, the thymine is likely rapidly removed, indicating a coupling between deaminase and glycosylase activity. Gadd45 may promote functional or physical interactions between AID and Mbd4 at the site of demethylation. Mbd4 can couple with a lyase to help promote base replacement through base excision repair. Targeting of AID/Mbd4 can be promoted by recognition of the 5-meCpG (methyl group in red), or through other mechanisms.

FIG. 8 shows the relative expression of AID (A), zMbd4 (B) and TDG (C) as determined by semiquantitative RT-PCR in cDNAs made from embryos the stages of development shown. Values indicated are normalized to 28S levels.

FIG. 9 shows the identification of gene targets of AID and MBD4 by methylated DNA immunoprecipitation (Me-DIP). Genomic DNA prepared from wild type embryos or those injected (at 80% epiboly) with AID scr mo (sequence scrambled control morpholino; 2 pg), AID morpholino (2 pg), MBD4 scr mo (sequence scrambled control morpholino; 2 pg), or MBD4 morpholino (2 pg) were subjected to immunoprecipitation using an antibody directed against 5-methylcytosine. PCR analysis was subsequently performed for multiple genes including (A) neurod2 (˜200 bp upstream of TSS), (B) sox1a (˜450 bp downstream of TSS), (C) hoxb2a (˜3700 bp upstream of TSS), (D) atoh1a (˜350 bp upstream of TSS), (E) pyruvate carboxylase (˜4800 bp upstream of TSS), (F) nucleoside phosphorylase (˜300 bp upstream of TSS), (G) noggin2 (˜500 bp downstream of TSS), (H) foxd3 (˜10 bp upstream of TSS), (I) sox2 (˜3350 bp upstream of TSS), (J) lin-28 (˜400 bp upstream of TSS), and (K) carbonic anhydrase 7 (˜50 bp upstream of TSS). Y-axis shows enrichment at these loci relative to a control neurod2 locus (see R2, FIG. 6A). Of the loci tested neurod2, sox1a, hoxb2a, atoh1a, pyruvate carboxylase, nucleoside phosphorylase, and noggin2 promoters/genes showed selective enrichment, whereas foxd3, sox2, lin-28 and carbonic anhydrase 7 did not. Primer information for the Me-DIP PCR is provided in Table 2. The graph shows one representative biological experiment (three biological repeats), with the average of two technical replicates shown.

FIG. 10A shows MeDIP-qPCR for several genes that were selected from genome-wide MeDIP-ChIP microarray analysis in apcmcr and apcwt (72 hpf), which are either uninjected or injected with aaa Mo (combination of aid, apobec2a and apobec2b morpholinos; 0.5 ng each) of mbd4 and tdg morpholinos together (1 ng each), or V5-Dnmt1 expressing plasmid (1 pg). FIG. 10B shows the MeDIP-qPCR for several genes selected from genome-wide MeDIP-ChIP microarray analysis in human adenomas and matching uninvolved tissues from FAP patients. P1-P10 refers to ten different patients. In both E and F, the Y-axis shows values for each promoter region normalized to a negative control region lacking CpGs, and then normalized to the values from wild type or uninvolved, valued at 1.

FIG. 11 shows quantitative RT-PCR for aid, mbd4 and gadd45α in apcmcr and apcwt treated with DMSO or all-trans retinoic acid (ATRA) (A) or in different RA deficiency models in zebrafish (B). Expression of genes are first normalized to 28S, and then to the control embryo mRNA/28S ratio, valued as 1. FIG. 11 also shows quantitative RT-PCR for AID, MBD4 and GADD45α in two RA responsive human colon carcinoma cell lines (HT29 and DLD1) treated with either DMSO (vehicle) or ATRA (C). In panels A-C, expression of genes are normalized first to 28S (A-B) or 18S (C) and then to the control sample values as 1. FIG. 11D shows the MeDIP-qPCR for various genes in apc mutants (72 hpf) which are either untreated or treated with ATRA.

FIG. 12A shows RT-PCR for Pou5f1 (Oct4) and Cebpβ in apcmcr and apcwt treated with DMSO or ATRA (1 μM). The Y-axis shows fold induction normalized to 28S and wild type DMSO treated sample. FIG. 12B shows a graph illustrating the fold enrichment near the aid or gadd45α TSS (a region which contains overlapping Oct and Cebp binding sites) for Cebpβ and Pou5f1 in embryos injected with V5-Cebpβ (along with Pou5f1 mo, 80 pg) or V5-Pou5f1 expressing plasmids. ChIP was performed with antibodies against the tags. Normalization control primers are located 3 kb upstream (a region without Cebpβ sites) of TSS of Gadd45α gene.

FIG. 13 shows Lef1 enrichment on the rdh1l promoter in apcmcr zebrafish was greater than ˜7 fold compared to their wild type siblings. Y-axis shows fold enrichment on a region containing Lef1 sites compared to an internal control region (without Lef1 binding site, P2) on the rdh1l promoter. Values obtained from Lef1 antibody were normalized to ones obtained using a non specific antibody and then expressed as fold enrichment compared to apcwt.

FIG. 14 shows quantitative PCR measuring DHRS9 (RDHL) expression in DLD1, SW480, and HT29 cells which were transfected with either a Scrambled (Scr) siRNA or a specific siRNA against LEF1 or TLE3 (A) or siRNAs against LSD1 or CoREST (B) or treated with pargyline (3 mM). Y-axis values represent fold change in DHRS9 expression. Normalization for DHRS9 absolute values was done first to 18S rRNA values and then to DHRS9/18S ratio from Scr siRNA.

FIG. 15A shows RT-PCR for rdh1l levels were performed and compared to 28S levels in apcwt and apcmcr embryos injected with control/lsd1/corest morpholinos or treated with pargyline (to inhibit Lsd1 activity). FIG. 15B shows ChIP for H3K4me2 marks on the rdh1l promoter were performed in apcwt and apcmcr zebrafish embryos injected with either control or lsd1 morpholino. The graph shows levels of H3K4me2 compared to total H3 levels on a region containing Lef1 sites and normalized to a region lacking Lef1 sites. Knockdown of Lsd1 increases H3K4me2 in apcmcr zebrafish, but knockdown in apcwt does not show a significant change. FIGS. 15C-D show a model of APC regulation of intestinal fating via retinoic acid and demethylase. APC can promote RA production by directly negatively regulating CtBP1 levels in a proteasome-dependent fashion. APC can also inhibit the transcription of LSD1, CoREST, LEF1 and TLE3. LEF1 binds to the RDH promoter and recruits TLE3 (Groucho2)/CtBP1/LSD1 repressors which can silence RDH expression. Retinoic acid negatively regulates demethylase components by inhibiting Pou5f1 and Cebpβ. Furthermore, regulation of demethylase components by APC is independent of β-catenin. Demethylase promotes the demethylation of key fate regulators (like aldh1a2, hoxa13a, evx1) and proliferative genes (like cyclind1 and pitx2). Fate regulators like aldh1a2 can help in maintaining a progenitor cell population.

FIG. 16A shows RT-PCR measuring dnmt1 levels in apc mutants (apc^{mcr}) and siblings (apc^{wt}). Y-axis shows fold change normalized to 28S levels first and then to dnmt1/28S ratio from apcwt, valued as 1. FIG. 16B show RT-PCR showing expression of AID, Gadd45α, and Mbd4 in adenoma tissues isolated from FAP patients, who are patients bearing mutations in APC gene. P1 through P1− refers to patient sample ID. Y-axis shows fold changes in expression of indicated genes normalized first to 28S levels and then to dnmt1/28S ratio from matching uninvolved tissue, valued as 1.

FIG. 17 shows RT-PCR showing aldh1a2 and fabp2 expression in apc mutants (apcmcr) and siblings (apcwt) injected with control Mo/aaa Mo/mbd4+tdg Mo.

FIG. 18 shows quantitative RT-PCR showing fold upregulation of lef1, groucho2, lsd1 and corest in apcmcr zebrafish compared to wild type siblings. Transcript levels normalized to 28S rRNA transcripts and then to wild type values.

FIG. 19 shows quantitative RT-PCR analyses for (A) LEF1 and (B) TLE3 in adenomas from FAP patients show increased levels in adenomas compared to their matched normal tissue. X-axis numbers refer to patients sample number. Y-axis values are fold changes in expression of indicated genes in adenomas normalized first to 28s levels and then mRNA/28S ratio from matching uninvolved tissue, valued at 1. FIG. 19(C) shows quantitative RT-PCR performed with primers specific for LEF1 using total RNA from the indicated cells following transfection with LEF1 siRNA or a control siRNA. Fold induction was calculated as total LEF1 transcripts normalized 18S rRNA and then to LEF1/18S values obtained for control siRNA transfected cells. FIG. 19(D) shows quantitative RT-PCR analyses for LSD1 in adenomas from FAP patients show increased levels in adenomas compared to their matched normal tissue. X-axis numbers refer to patients sample number. Y-axis values are fold changes in expression of indicated genes in polyps normalized first to 28S levels and then to LSD1/28S ratio from matching uninvolved tissue, valued at 1.

FIG. 20 shows ALDH1 positive colon cancer cells express high levels of the demethylase components.

### DETAILED DESCRIPTION OF THE INVENTION

The present invention comprises methods and systems directed at detecting, evaluating, ameliorating, preventing and treating an oncogenic event. The disclosed methods and systems can comprise one or more Demethylase System Components or other compositions that can be used alone or in combination detect, evaluate, treat, ameliorate, or prevent an oncogenic event. Disclosed herein are methods comprising one or more Demethylase System Components or other compositions that can be used alone or in combination that can be used to determine the efficacy of an active agent against an oncogenic event. Also disclosed herein are methods of affecting cell differentiation.

All patents, patent applications and publications cited herein, whether supra or infra, are hereby incorporated by reference in their entireties into this application in order to more fully describe the state of the art as known to those skilled therein as of the date of the invention described and claimed herein.

It is to be understood that this invention is not limited to specific synthetic methods, or to specific recombinant biotechnology methods unless otherwise specified, or to particular reagents unless otherwise specified, to specific pharmaceutical carriers, or to particular pharmaceutical formulations or administration regimens, as such may, of course, vary.

### DEFINITIONS AND NOMENCLATURE

The terminology used herein is for the purpose of describing particular embodiments only and is not intended to be limiting.

As used in the specification and the appended claims, the singular forms “a,” “an” and “the” can include plural referents unless the context clearly dictates otherwise. Thus, for example, reference to “a compound” includes mixtures of compounds, reference to “a pharmaceutical carrier” includes mixtures of two or more such carriers, and the like. Reference to “a component” can include a single or multiple components or a mixtures of components unless the context clearly dictates otherwise.

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The word “or” as used herein means any one member of a particular list and also includes any combination of members of that list.

By “sample” is meant an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

By “modulate” or “alter” is meant to alter, by increasing or decreasing.

By “normal subject” is meant an individual who does not have an oncogenic condition or who is not undergoing an oncogenic event.

“Peptide” as used herein refers to any peptide, oligopeptide, polypeptide, gene product, expression product, or protein. For example, a peptide can be an enzyme. A polypeptide is comprised of consecutive amino acids. The term “polypeptide” encompasses naturally occurring or synthetic molecules.

In addition, as used herein, the term “peptide” or “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

As used herein, the term “amino acid sequence” refers to a list of abbreviations, letters, characters or words representing amino acid residues.

The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

By “increased susceptibility to develop an oncogenic condition or oncogenic event” is meant a subject who has a greater than normal chance of developing an oncogenic condition or event compared to the general population. Such subjects could include, for example, subjects whose expression levels of one or more Demethylase System Components in a subject are higher than levels from a normal subject.

By an “effective amount” of a compound as provided herein is meant a sufficient amount of the compound to provide the desired effect. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

By “treat” is meant to administer a compound or molecule to a subject, such as a human or other mammal (for example, an animal model), that has an oncogenic condition/event or an increased susceptibility for developing an oncogenic condition/event, in order to prevent or delay a worsening of the effects of the condition or event, or to partially or fully reverse the effects of the condition or event. To “treat” can also refer to non-pharmacological methods of preventing or delaying a worsening of the effects of the condition or event, or to partially or fully reversing the effects of the condition or event. For example, “treat” is meant to mean a course of action to prevent or delay a worsening of the effects of the condition or the event or to partially or fully reverse the effects of the condition or event other than by administering a compound.

By “prevent” is meant to minimize the chance that a subject who has a susceptibility for developing an oncogenic condition or event will develop an oncogenic condition or event or one or more symptoms associated with an oncogenic condition or event.

By “specifically binds” is meant that the composition recognizes and physically interacts with its cognate target (for example, its antigen0 (for example, a Demethylase System Component) and does not significantly recognize and interact with other targets.

By “probe,” “primer,” or oligonucleotide is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for c-Met nucleic acids (for example, genes and/or mRNAs) have at least 80%-90% sequence complementarity, preferably at least 91%-95% sequence complementarity, more preferably at least 96%-99% sequence complementarity, and most preferably 100% sequence complementarity to the region of the target to which they hybridize. Probes, primers, and oligonucleotides may be detectably-labeled, either radioactively, or non-radioactively, by methods well-known to those skilled in the art. Probes, primers, and oligonucleotides are used for methods involving nucleic acid hybridization, such as: nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

By “specifically hybridizes” is meant that a probe, primer, or oligonucleotide recognizes and physically interacts (that is, base-pairs) with a substantially complementary nucleic acid (for example, a Demethylase System Component as a nucleic acid) under high stringency conditions, and does not substantially base pair with other nucleic acids.

By “high stringency conditions” is meant conditions that allow hybridization comparable with that resulting from the use of a DNA probe of at least 40 nucleotides in length, in a buffer containing 0.5 M NaHPO4, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. (See, for example, F. Ausubel et al., Current Protocols in Molecular Biology, John Wiley & Sons, New York, N.Y., 1998).

By “normal subject” is meant an individual who does not have an oncogenic event. By “normal subject” can also refer to an individual who does not have an oncogenic disorder. By “normal subject” can also refer to an individual who does not have an oncogenic condition.

By “normal sample” is meant a sample without an oncogenic event. By “normal sample” can also refer to a sample without an oncogenic disorder. By “normal sample” can also refer to a sample without an oncogenic condition.

### Compositions

### A. Demethylase System

The term “Demethylase System” refers to a system of components that can function to regulate or alter the methylation of nucleic acids, including, but not limited to, various genes, promoters, and other nucleic acid elements. The Demethylase System can comprise one or more Demethylase System Components, or homologs thereof. The Demethylase System can also comprises genetic or protein components that can interact with Demethylase System cofactors such as PCNA, p21, Cdc2/CyclinB1, MEKK4, and p38 kinase.

### B. Demethylase System Components

Demethylase System Components can include, but are not limited to, Demethylase System cytidine deaminases, Demethylase System thymine glycosylases, and Demethylase System cofactors. Demethylase System Components, can exist as natural or synthetic genetic or protein material. For example, Demethylase System Components can be a nucleic acid a peptide, a peptide fragment, a peptide complex, a peptide fragment complex, a protein, or a protein complex.

Demethylase System cytidine deaminases are capable of deaminating 5-meC in single stranded DNA. Demethylase System cytidine deaminases can also be capable of generating thymine and yielding a G:T mismatch. For example, Demethylase System cytidine deaminases can insert mutations in both DNA and RNA via deamination of cytidine to uridine, and can share a characteristic zinc-coordination motif. Cytidine deaminases can deaminate cytidines located within hotspot motifs. Such hotspot motifs include, but are not limited to (1) WRCY motifs, wherein W is adenosine or thymidine, R is purine, C is cytidine, and Y is pyrimidine, and (2) RGYW, wherein R is purine, G is guanidine, Y is pyrimidine, and W is adenosine or thymidine. Some Demethylase System cytidine deaminases also deaminate 5-meC. Demethylase System cytidine deaminases include, but are not limited to, activation induced deaminase (AID), apolipoprotein B RNA-editing catalytic component (Apobec-1 or Apobec-2a/b), DNA methyltransferase (Dnmt), and homologs thereof.

Demethylase System thymine glycosylases can remove thymine moieties from G:T mismatches by hydrolyzing the carbon-nitrogen bond between the sugar-phosphate backbone of the DNA and the mispaired thymine Demethylase System thymine glycosylases include, but are not limited to, methyl-binding domain protein 4 (Mbd4), thymidine DNA glycosylase (TDG), and homologs thereof. Demethylase System thymine glycosylases can remove thymine from C/T and T/T mispairings, and can remove uracil and 5-bromouracil from mispairings with guanine. The Demethylase System can also comprises proteins that can interact with a Demethylase System thymine glycosylase, including MeCP2, Mbd1, Mbd2, and Mbd3.

The term “G:T intermediate” or “G:T mismatch” refers to a mutagenic intermediate product that can be recognized and repaired, for example, by the Demethylase System thymine glycosylase Mbd4.

Demethylase System cofactors can interact with one or more Demethylase System thymine glycosylases or Demethylase System cytidine deaminases. For example, Demethylase System cofactors can couple a Demethylase System thymine glycosylase or Demethylase System cytidine deaminase. Demethylase System cofactors can promote functional interactions, physical interactions, or both functional and physical interactions between Demethylase System thymine glycosylases and Demethylase System cytidine deaminases. Examples of Demethylase System cofactors include, but are not limited to, non-enzymatic factors such as growth arrest and DNA-damage-induce gene 45 (GADD45), Gadd45α, Gadd45β, and Gadd45γ.

Disclosed herein are also genes and peptides that can be used as biomarkers for an oncogenic event or oncogenic condition. For example, one or more Demethylase System Components can be used as biomarkers for oncogenic events or oncogenic conditions.

### C. Oncogenic Events and Conditions

The term “oncogenic event” can refer to any one of a series of genetic, epigenetic, and cellular events that can reprogram a cell to undergo uncontrolled cell division. An oncogenic event can therefore be uncontrolled cell division or the formation of a malignant mass. Generally, a malignant mass can be characterized by one or more of the following: (a) acquisition of self-sufficiency in growth signals, which can lead to unchecked growth; (b) loss of sensitivity to anti-growth signals, which can lead to unchecked growth, (c) loss of capacity for apoptosis, which can allow growth despite genetic errors and external anti-growth signals, (d) loss of capacity for senescence, which can lead to limitless replicative potential, (e) acquisition of sustained angiogenesis, which can allow the mass to grow beyond the limitations of passive nutrient diffusion, (f) acquisition of ability to invade neighboring tissues, which can be a defining property of invasive carcinoma, (g) acquisition of ability to build metastases at distant sites, which can be a classical property of malignant tumors, and (h) loss of capacity to repair genetic errors, which can lead to an increased mutation rate or genomic instability. Oncogenic events can also include but are not limited to events that contribute to, sustain, or precipitate aberrant DNA methylation, unregulated cell growth, failure to differentiate, tumorigenesis, cancer, metastasis.

The term “oncogenic condition” or “oncogenic disorder” can refer to the occurrence of at least one oncogenic event.

An oncogenic event can also be an epigenetic event(s), that can include, but is not limited to DNA methylation, or the methylation or acetylation of histone proteins bound to chromosomal DNA. The term “epigenetic” as used herein refers to factors other than the primary sequence of the genome that affect the development or function of an organism, they can affect the phenotype of an organism without changing the genotype. Epigenetic factors can include, but are not limited to modifications in gene expression that are controlled by heritable but potentially reversible changes in DNA methylation and chromatin structure.

An oncogenic event or oncogenic condition can be a cancer. The cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers, testicular cancer, colon and rectal cancers, prostatic cancer, and pancreatic cancer.

The term “aberrant DNA methylation” can refer to atypical, unusual, abnormal, or inappropriate changes in DNA methylation patterns. For example, aberrant DNA methylation includes but is not limited to (a) hypermethylation of tumor suppressor genes, (b) aberrant expression of DNA (cytosine-5-)-methyltransferase 1 (DNMT1) or other DNMTs that can methylate genomic DNA involved in the process of gene inactivation, chromatin organization, X chromosome inactivation, and genomic imprinting, and (c) hypomethylation of unique genes and repetitive sequences.

The term “hypomethylation” can refer to a decrease in the epigenetic methylation of cytosine and adenosine residues.

The term “hypermethylation” can refer to an increase in the epigenetic methylation of cytosine and adenosine residues.

### Kits

Disclosed herein are kits that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

It is understood that the disclosed kits can be used for numerous applications including but not limited to immunoblot detection, immunofluorescence detection, and tissue array. Thus, the disclosed kits can be modified to be more suitable for each given application. It is further understood that there are numerous means to detect the presence of monoclonal antibody binding. Such methods can include direct detection through the use of a labeled monoclonal antibody or through detection of a secondary antibody which is labeled and which secondary antibody binds to the monoclonal antibody. Examples can include monoclonal antibodies to one or more of the Demethylase System Components. Thus, disclosed herein are kits further comprising a secondary antibody that can bind to the monoclonal antibody. Alternatively detection mechanisms include visualization reagents such as horseradish peroxidase. It is further contemplated that said kits can include buffers, blocking reagents, substrates, and retrieval solutions. It is understood that there are many known methods of detection known to those of skill in the art. Specifically contemplated are kits comprising any detection mechanism now known.

### Methods

The present invention comprises methods and systems directed to detecting an oncogenic event. The disclosed methods and systems can comprise one or more Demethylase System Components that can be used to detect, ameliorate, treat or prevent an oncogenic event. Disclosed herein are methods comprising one or more Demethylase System Components that can be used to determine the efficacy of an active agent against an oncogenic event. Also disclosed herein are methods of affecting cell differentiation comprising modulating modulating one of more Demethylase System Components.

The term “expression level” refers to a quantifiable amount of a genetic or protein material. For example, the methods described herein can be used to detect or measure the amount of Demethylase System Components present in a sample. The methods described herein can be used to determine the expression level of one or more Demethylase System Components. For example, the methods described herein can be used to detect the level of one or more Demethylase System Components nucleic acids, such as DNA or RNA or DNA-RNA hybrid, single-stranded or double-stranded, sense or antisense. The methods described herein can also be used to determine the expression level of one or more Demethylase System Components. For example, the methods described herein can be used to detect the level of one or more Demethylase System Components peptide, oligopeptide, polypeptide, gene product, expression product, or protein.

Genetic or protein material can be a nucleic acid, a peptide, a peptide fragment, a peptide complex, a peptide fragment complex, a protein, or a protein complex. The disclosed methods can detect an increase in an expression level, wherein the expression level of one or more Demethylase System Components is increased by 10%, 15%, 20%, 25%, 30%, 35%, 40%, 45%, 50%, 55%, 60%, 65%, 70%, 75%, 80%, 85%, 90%, 95%, or 100% when compared to the expression level of a “normal” subject. The disclosed methods can detect an decrease in expression levels, wherein the expression level of one or more Demethylase System Components is decreased by 10%, 15%, 20%, 25%, 30%, 35%, 40%, 45%, 50%, 55%, 60%, 65%, 70%, 75%, 80%, 85%, 90%, 95%, or 100% when compared to the expression level of a “normal” subject.

By “sample” is meant an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide or nucleic acid), which is assayed as described herein. A sample may also be any body fluid or excretion (for example, but not limited to, blood, urine, stool, saliva, tears, bile) that contains cells or cell components.

Sample can also refer to transgenic non-human animals which express a heterologous Demethylase System Component gene, or which have had one or more genomic Demethylase System Component gene(s) disrupted in at least one of the tissue or cell-types of the animal. For instance, transgenic mice that are disrupted at their Demethylase System Component gene locus can be generated. Disclosed herein are animal models for an Demethylase System-associated oncogenic event or oncogenic condition, which has a mis-expressed or non-expressed Demethylase System Component allele. For example, a animal can be bred which has a Demethylase System allele deleted, or in which all or part of one or more Demethylase System exons are deleted. Such an animal can then be used to study disorders arising from mis-expression of a Demethylase System gene. The Demethylase System transgene can encode the wild-type form of the protein, or can encode homologs thereof, including both agonists and antagonists, as well as antisense constructs. The Demethylase System transgene can include a Demethylase System nucleotide sequence or fragments thereof. The expression of the transgene is restricted to specific subsets of cells, tissues or developmental stages utilizing, for example, cis-acting sequences that control expression in the desired pattern.

Genetic techniques which allow for the expression of transgenes can be regulated via site-specific genetic manipulation in vivo are known to those skilled in the art. For instance, genetic systems are available which allow for the regulated expression of a recombinase that catalyzes the genetic recombination a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of the Demethylase System Component polypeptides. For example, excision of a target sequence which interferes with the expression of a recombinant Demethylase System gene can be designed to activate expression of that gene. This interference with expression of the protein can result from a variety of mechanisms, such as spatial separation of the Demethylase System gene from the promoter element or an internal stop codon. Moreover, the transgene can be made wherein the coding sequence of the gene is flanked recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

For example, either the cre/loxP recombinase system of bacteriophage PI (Lakso et al., (1992) Proc. Natl. Acad. Sci. USA 89:6232-6236; Orban et al., (1992) Proc. Natl. Acad. Sci. USA 89:6861-6865) or the FLP recombinase system of Saccharomyces cerevisiae (O'Gorman et al., (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al., (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

As used herein, “screening” refers to methods for detecting or identifying test compounds, compositives, treatments, agents, or therapies, which modulate the expression level of one or more Demethylase System Components, or modulate the methylation of one or more Demethylase System Components. In many drug screening programs which test libraries of compounds and natural extracts, high throughput assays are desirable in order to survey in a given period of time the maximize the number of active agents, which can be a metal, a chemical, a pharmaceutical agent, or combinations thereof, which can be administered to a subject to treat an oncogenic event or oncogenic conditions.

As used herein, “detection” can refer to discovering the presence or absence of one or more Demethylase System Components or related components. Detection can also refer to determining the expression level of one or more Demethylase System Components or related components. Information regarding the expression level, and optionally the quantitative level of the expression of the Demethylase System Components, may then be used to draw inferences about the nature of the biological sample and, if the biological sample was obtained from a subject, the health state of the subject. For example, detection can refer to quantitation of one or more Demethylase System Component or related components on an absolute basis or on a relative basis by comparing the expression level to one or more constitutively expressed standards. The Examples below provide specific examples of methods that can be used to determine the expression level of one or more of the Demethylase System Components or related components described herein.

Samples should generally be prepared in a manner that is consistent with the detection system to be employed. For example, a sample to be used in a protein detection system should generally be prepared in the absence of proteases. Likewise, a sample to be used in a nucleic acid detection system should generally be prepared in the absence of nucleases. In many instances, a sample for use in an antibody-based detection system will not be subjected to substantial preparatory steps. For example, urine may be used directly, as may saliva and blood, although blood will, in certain preferred embodiments, be separated into fractions such as plasma and serum.

A variety of assay formats can be used and, in light of the present disclosure, those not expressly described herein will nevertheless considered to be within the purview of ordinary skill in the art. Assay formats can approximate such conditions as the expression level of one more Demethylase System Components, the methylation status of DNA, tumor suppressing activity, transcriptional activating activity, and can be generated in many different forms. In many embodiments, the invention provides assays including both cell-free systems and cell-based assays which utilize intact cells.

In view of this specification, one of skill in the art will recognize a wide range of techniques that may be employed to detect and optionally quantitate the presence of nucleic acids for one or more Demethylase System Components or other compositions. For example, quantitation may be on an absolute basis, or may be relative to a constitutively expressed standard.

Nucleic acid detection systems generally involve preparing a purified nucleic acid fraction of a sample, and subjecting the sample to a direct detection assay or an amplification process followed by a detection assay. Amplification may be achieved, for example, by polymerase chain reaction (PCR), reverse transcriptase (RT) and coupled RT-PCR. Detection of a nucleic acid is generally accomplished by probing the purified nucleic acid fraction with a probe that hybridizes to the nucleic acid of interest, and in many instances detection involves an amplification as well.

Nucleic acid probes that bind specifically to a Demethylase System Component can be labeled with, for example, a fluorescent moiety, a radionuclide, an enzyme or an affinity tag such as a biotin moiety. For example, the TaqMan™ system employs nucleic acid probes that are labeled in such a way that the fluorescent signal is quenched when the probe is free in solution and bright when the probe is incorporated into a larger nucleic acid.

Northern blots, dot blots, microarrays, quantitative PCR, and quantitative RT-PCR are all well known methods for detecting a nucleic acid in a sample. PCR can refer to traditional PCR techniques allele-specific PCR, assembly PCR or polymerase cycling assembly (PCA), asymmetric or symmetric PCR assays, helicase-dependent amplification, hot-start PCR, intersequence-specific PCR (ISSR), inverse PCR, ligation-mediated PCR, methylation-specific PCR (MSP), miniprimer PCR, multiplex ligation-dependent probe amplification (MLPA), multiplex-PCR, nested PCR, overlap-extension PCR, quantitative PCR (Q-PCR), RT-PCR, real-time PCR, solid phase PCR, TAIL-PCR, touchdown PCR, PAN-AC, and universal fast walking, all of which can be used to determine expression levels.

Immunoscintigraphy using monoclonal antibodies directed at one or more Demethylase System Components can be used to detect the expression level of the one or more Demethylase System Components. For example, monoclonal antibodies against the Demethylase System Component marker labeled with ⁹⁹Technetium, ¹¹¹Indium, ¹²⁵Iodine—can be effectively used for such imaging. As will be evident to the skilled artisan, the amount of radioisotope to be administered is dependent upon the radioisotope. Those having ordinary skill in the art can readily formulate the amount of the imaging agent to be administered based upon the specific activity and energy of a given radionuclide used as the active moiety. Typically 0.1-100 millicuries per dose of imaging agent, preferably 1-10 millicuries, most often 2-5 millicuries are administered. Thus, compositions according to the present invention useful as imaging agents comprising a targeting moiety conjugated to a radioactive moiety comprise 0.1-100 millicuries, in some embodiments preferably 1-10 millicuries, in some embodiments preferably 2-5 millicuries, in some embodiments more preferably 1-5 millicuries.

In view of this specification, one of skill in the art will recognize a wide range of techniques that may be employed to detect and optionally quantitate the presence of proteins or peptides for one or more Demethylase System Components or other compositions. For example, quantitation may be on an absolute basis, or may be relative to a constitutively expressed standard.

For example, the one or more Demethylase System Component proteins or peptides can be detected with an antibody, which can involve bringing the sample and the antibody into contact so that the antibody has an opportunity to bind to proteins having the corresponding epitope. An antibody-based detection assay can also typically involve a system for detecting the presence of antibody-epitope complexes, thereby achieving a detection of the presence of the proteins having the corresponding epitope. Antibodies can also be used in a variety of other detection techniques, including enzyme-linked immunosorbent assays (ELISAs), immunoprecipitations, Western blots. Antibody-independent techniques for identifying a protein may also be employed. For example, mass spectroscopy, particularly coupled with liquid chromatography, permits detection and quantification of large numbers of proteins in a sample. Two-dimensional gel electrophoresis may also be used to identify proteins, and may be coupled with mass spectroscopy or other detection techniques, such as N-terminal protein sequencing. RNA aptamers with specific binding for the protein of interest may also be generated and used as a detection reagent.

A variety of methods can be used to determine if a Demethylase System Component has been produced in a reaction assay. One way to determine if a Demethylase System Component product has been produced in the reaction is to analyze a portion of the reaction by agarose gel electrophoresis. For example, a horizontal agarose gel of from 0.6 to 2.0% agarose is made and a portion of the Demethylase System Component reaction mixture is electrophoresed through the agarose gel. After electrophoresis, the agarose gel is stained with ethidium bromide. Demethylase System Components are visible when the gel is viewed during illumination with ultraviolet light. By comparison to standardized size markers, it is determined if the Demethylase System Component is of the correct expected size.

Methods for detecting methylation can cover any assay for detecting DNA methylation. Another example method for detecting methylation of DNA is by using “methylation-sensitive” restriction endonucleases. Such methods comprise treating the genomic DNA isolated from a subject with an methylation-sensitive restriction endonuclease and then using the restriction endonuclease-treated DNA as a template in a PCR reaction. Herein, methylation-sensitive restriction endonucleases recognize and cleave a specific sequence within the DNA if C bases within the recognition sequence are not methylated. If C bases within the recognition sequence of the restriction endonuclease are methylated, the DNA will not be cleaved. Examples of such methylation-sensitive restriction endonucleases include, but are not limited to HpaII, SmaI, SacII, EagI, MspI, BstUI, and BssHII. In this technique, a recognition sequence for a methylation-sensitive restriction endonuclease is located within the template DNA, at a position between the forward and reverse primers used for the PCR reaction. In the case that a C base within the methylation-sensitive restriction endonuclease recognition sequence is not methylated, the endonuclease will cleave the DNA template and a PCR product will not be formed when the DNA is used as a template in the PCR reaction. In the case that a C base within the methylation-sensitive restriction endonuclease recognition sequence is methylated, the endonuclease will not cleave the DNA template and a PCR product will be formed when the DNA is used as a template in the PCR reaction. Therefore, methylation of C bases can be determined by the absence or presence of a PCR product (Kane, et al., 1997, Cancer Res, 57:808-11). No sodium bisulfite is used in this technique.

Some exemplary methods for detecting methylation of DNA are modified methylation-sensitive polymerase chain reaction (MSP), the MS-SnuPE method, and combined bisulfite restriction analysis (COBRA) (see, e.g., Gonzalgo, et al., 1997, Nucleic Acids Res., 25:2529-31; Xiong, et al., 1997, Nucleic Acids Res, 25:2532-4).

Disclosed herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event. The one or more Demethylase System Components can include at least one Demethylase System cytidine deaminase. In addition, the one or more Demethylase System Components can include at least one Demethylase System thymine glycosylase. Also, the one or more Demethylase System Components can include at least one Demethylase System cofactor.

Disclosed herein are methods of detecting an oncogenic event in a sample comprising determining the expression level of one or more Demethylase System cytidine deaminases and one or more Demethylase System thymine glycosylases in a sample and comparing those expression levels to the expression levels of a normal sample, wherein an increase in the expression level of one or more of the Demethylase System cytidine deaminases and an increase in the expression level one or more of the Demethylase System thymine glycosylases compared to the expression levels of a normal sample can indicate an oncogenic event. The disclosed methods can further comprise determining the expression level of one or more Demethylase System cofactors, wherein an increase in the expression level of the one or more Demethylase System cofactor can indicate an oncogenic event. In the disclosed methods, the one or more Demethylase System cofactors can be GADD45, gadd45α, gadd45β, or gadd45γ.

Also disclosed herein are methods of detecting an oncogenic event in a sample comprising determining the expression level of one or more Demethylase System cytidine deaminases and one or more Demethylase System cofactors in a sample and comparing those expression levels to the expression levels of a normal sample, wherein an increase in the expression level of one or more of the Demethylase System cytidine deaminases and an increase in the expression level one or more of the Demethylase System cofactors compared to the expression levels of a normal sample can indicate an oncogenic event. In the disclosed methods, the one or more Demethylase System cofactors can be GADD45, gadd45α, gadd45β, or gadd45γ. The disclosed methods can further comprise determining the expression level of one or more Demethylase System thymine glycosylases, wherein an increase in the expression level of the one or more Demethylase System thymine glycosylase compared to the expression levels of a normal sample can indicate an oncogenic event.

The disclosed methods can further comprise determining the level of methylated DNA in the sample. The level of methylated DNA can be determined, for example, by assessing susceptibility to the restriction enzyme HpaII, which is methylation-inhibited. In the disclosed methods, a decrease in the level of methylated DNA indicates hypomethylation. Hypomethylation, in turn, can indicate an oncogenic event.

Also disclosed are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the level of DNA methylation of one or more of the promoters selected from the group consisting of aldh1a2, hox13a, evx1, pitx2, cyclind1, hoxd13a, junb1, frizzled8a, cdx4, sox9b, cyclinb2, sox4, Fabp2, Raldh2, pcna, and cyclinD1. In the disclosed methods, a decrease in the level of DNA methylation of the one or more promoters indicates an oncogenic event.

Also disclosed are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the presence of a G:T intermediate. The presence of the G:T intermediate can be determined, for example, using PCR. PCR can be conducted using a forward primer with a 3′-terminal adenosine that is complementary to the thymine base derived from the deamination of 5-meC, and using a reverse primer that is complementary to a downstream region of the target. In the disclosed methods, the presence of a G:T intermediate can indicate an oncogenic event.

In addition, disclosed herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the expression level of retinoic acid in the sample. In the disclosed methods, retinoic acid can antagonize and suppress the expression level of Cebpβ and Pou5f1. By antagonizing and suppressing the expression level of Cebpβ and Pou5f1 in the disclosed methods, retinoic acid can directly repress or downregulate other Demethylase System Components, thereby promoting DNA methylation of key genes and helping progenitors commit to differentiation. In the disclosed methods, a decrease in the expression level of retinoic acid can indicate an oncogenic event.

Also disclosed are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the expression level of Cebpβ or Pou5f1. In the disclosed methods, Cebpβ and Pou5f1 can directly activate Demethylase System Components. Thus, Cebpβ and Pou5f1 can be positive regulators or activators of various Demethylase System Components. For example, some Demethylase System Components, such as Gadd45α and aid, contain Cebpβ and Pou5f1 sites in their promoters. In the disclosed methods, an increase in the expression level of Cebpβ or Pou5f1 can indicate an oncogenic event.

Also disclosed herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the presence or absence of a mutation in the adenomatous polyposis coli (APC) tumor suppressor gene. In the disclosed methods, the loss or mutation of APC can be a key initiating event in a series of genetic and epigenetic events that can indicate an oncogenic event. The disclosed methods can further comprise determining the expression level of a retinol dehydrogenase or a alcohol dehydrogenase. Alcohol dehydrogenases (ADH) catalyzes the conversion of retinol into retinal and retinol dehydrogenase catalyzes the conversion of dietary retinol into retinaldehyde. In the disclosed methods, a mutation in the APC gene and a decrease in the expression level of the retinol dehydrogenase or the alcohol dehydrogenase can indicate an oncogenic event. The disclosed methods can further comprise determining the expression level of ALDH1. ALDH is a commonly used marker of stem cells and cancer stems including those derived from human colon and colon carcinoma. In the disclosed method, an increase in the expression level of ALDH1 and the presence of a mutation in APC can indicate an oncogenic event. The methods can further comprise determining the expression level of a retinol dehydrogenase or a alcohol dehydrogenase, wherein a decrease in the expression level of the retinol dehydrogenase or the alcohol dehydrogenase, an increase in the expression level of ALDH1, and the presence of a mutation in APC can indicate an oncogenic event.

Also described herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the expression level of a retinol dehydrogenase or a alcohol dehydrogenase and the expression level of retinol. Production of retinoic acid first requires converting dietary retinol (vitamin A) into retinoic acid, a process that occurs via two enzymatic step: the conversion of retinol into retinal by alcohol dehydrogenases (ADH) and short chain dehydrogenases (SDR), followed by the conversion of retinal into retinoic acid via aldehyde dehydrogenases (ALDH). In the disclosed methods, a decrease in the expression level of the retinol dehydrogenase or the alcohol dehydrogenase and an increase in the expression level of retinol i can indicate an oncogenic event. In the disclosed methods, an increase in the expression level of retinol can also indicate a defect in the absorption process.

Also disclosed herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample can indicate an oncogenic event, wherein the methods can further comprise determining the expression level of DNA methyltransferase (dnmt1). Dnmt1 is an enzyme that generates 5-methylcytosine (5-meC) in vertebrates. The inability of DNA methyltransferase to maintain normal patterns in highly proliferative cells is implicated in the genome-wide hypomethylation that occurs during tumorigenesis. A loss of dnmt1 can occur in parallel with upregulation of various Demethylase System Components in apc mutants. In the disclosed methods, a decrease in the expression level of dnmt1 can indicate an oncogenic event.

Also disclosed are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample can indicate an oncogenic event, wherein the methods can further comprise determining the expression level of APC. In the disclosed methods, a decrease in the expression level of APC can indicate an oncogenic event.

Disclosed herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample can indicate an oncogenic event, wherein the methods can further comprise determining the expression level of LEF1 and Groucho2/TLE3. Lef1, Groucho2/TLE3, CtBP1, LSD1 and Corest can work together to repress the production of retinoic acid by direct binding to, and repression of, the rdh1 promoter. In the disclosed methods, an increase in the expression levels of LEF1 and Groucho2/TLE3 can indicate an oncogenic event.

Also disclosed herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample can indicate an oncogenic event, wherein the methods can further comprise determining the expression level of LSD1, Corest or CrBP1. Lef1, Groucho2/TLE3, CtBP1, LSD1 and Corest can work together to repress the production of retinoic acid by direct binding to, and repression of, the rdh1 promoter. In the disclosed methods, an increase in the expression level of LSD1, Corest, or CrBP1 can indicate an oncogenic event.

Disclosed herein are methods of detecting an oncogenic event in a sample comprising determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylases, wherein the one or more Demethylase System Components includes at least one thymine Demethylase System cofactor, and wherein a decrease in the expression level of the one or more of the Demethylase System thymine glycosylases and an increase in the expression level of the one or more Demethylase System cytidine deaminases and an increase in the expression level of the one or more Demethylase System cofactors compared to the expression levels of a normal sample can indicate an oncogenic event.

Also disclosed are methods of detecting an oncogenic event in a sample comprising determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylases, wherein the one or more Demethylase System Components includes at least one Demethylase System cofactor, and wherein a decrease in the expression level of the one or more of the Demethylase System thymine glycosylases and an increase in the expression level of the one or more Demethylase System cytidine deaminases compared to the expression levels of a normal sample and the detection of the one or more Demethylase System cofactors can indicate an oncogenic event.

Also disclosed are methods of detecting an oncogenic event in a sample comprising determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylases, wherein the one or more Demethylase System Components includes at least one Demethylase System cofactor, and wherein a decrease in the expression level of the one or more of the Demethylase System thymine glycosylases and an increase in the expression level of the one or more Demethylase System cofactors compared to the expression levels of a normal sample and the detection of the one or more Demethylase System cytidine deaminases can indicate an oncogenic event.

Also disclosed are methods of detecting an oncogenic event in a sample comprising determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylases, wherein the one or more Demethylase System Components includes at least one Demethylase System cofactor, and wherein a decrease in the expression level of the one or more of the Demethylase System thymine glycosylases compared to the expression levels of a normal sample and the detection of the one or more Demethylase System cytidine deaminases and the detection of the one or more Demethylase System cofactors can indicate an oncogenic event.

Also disclosed are methods of detecting an oncogenic event in a sample comprising determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylases, wherein the one or more Demethylase System Components includes at least one Demethylase System cofactor, wherein a decrease in the expression level of the one or more of the Demethylase System thymine glycosylases compared to the expression levels of a normal sample can indicate an oncogenic event.

Also disclosed are methods of detecting an oncogenic event in a sample comprising determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylases, wherein the one or more Demethylase System Components includes at least one Demethylase System cofactor, wherein a decrease in the expression level of the one or more of the Demethylase System thymine glycosylases and an increase in the expression level of the one or more of the Demethylase System cytidine deaminases compared to the expression levels of a normal sample can indicate an oncogenic event.

Also disclosed are methods of detecting an oncogenic event in a sample comprising determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylase, wherein the one or more Demethylase System Components includes at least one Demethylase System cofactor, wherein a decrease in the expression level of the one or more of the Demethylase System thymine glycosylases compared to the expression levels of a normal sample and the detection of the expression of one or more Demethylase System cytidine deaminases in the sample can indicate an oncogenic event.

Also disclosed are methods of detecting an oncogenic event in a sample comprising determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylases, wherein the one or more Demethylase System Components includes at least one Demethylase System cofactor, wherein a decrease in the expression level of the one or more of the Demethylase System thymine glycosylases and an increase in the expression of the one or more of the Demethylase System cofactors compared to the expression levels of the normal sample can indicate an oncogenic event.

Also disclosed are methods of detecting an oncogenic event in a sample comprising determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylase, wherein the one or more Demethylase System Components includes at least one Demethylase System cofactor, wherein a decrease in the expression level of the one or more of the Demethylase System thymine glycosylases compared to the expression levels of a normal sample and the detection of the expression of one or more Demethylase System cofactors in the sample can indicate e an oncogenic event.

The disclosed methods can further comprise determining the level of methylated DNA in the sample. The level of methylated DNA can be determined, for example, by assessing susceptibility to the restriction enzyme HpaII, which is methylation-inhibited. In the disclosed methods, a decrease in the level of methylated DNA indicates hypomethylation. Hypomethylation, in turn, can indicate an oncogenic event.

Also disclosed are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the level of DNA methylation of one or more of the promoters selected from the group consisting of aldh1a2, hox13a, evx1, pitx2, cyclind1, hoxd13a, junb1, frizzled8a, cdx4, sox9b, cyclinb2, sox4, Fabp2, Raldh2, pcna, and cyclinD1. In the disclosed methods, a decrease in the level of DNA methylation of the one or more promoters indicates an oncogenic event.

Also disclosed are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the presence of a G:T intermediate. The presence of the G:T intermediate can be determined, for example, using PCR. PCR can be conducted using a forward primer with a 3′-terminal adenosine that is complementary to the thymine base derived from the deamination of 5-meC, and using a reverse primer that is complementary to a downstream region of the target. In the disclosed methods, the presence of a G:T intermediate can indicate an oncogenic event.

In addition, disclosed herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the expression level of retinoic acid in the sample. In the disclosed methods, retinoic acid can antagonize and suppress the expression level of Cebpβ and Pou5f1. By antagonizing and suppressing the expression level of Cebpβ and Pou5f1 in the disclosed methods, retinoic acid can directly repress or downregulate other Demethylase System Components, thereby promoting DNA methylation of key genes and helping progenitors commit to differentiation. In the disclosed methods, a decrease in the expression level of retinoic acid can indicate an oncogenic event.

Also disclosed are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the expression level of Cebpβ or Pou5f1. In the disclosed methods, Cebpβ and Pou5f1 can directly activate Demethylase System Components. Thus, Cebpβ and Pou5f1 can be positive regulators or activators of various Demethylase System Components. For example, some Demethylase System Components, such as Gadd45α and aid, contain Cebpβ and Pou5f1 sites in their promoters. In the disclosed methods, an increase in the expression level of Cebpβ or Pou5f1 can indicate an oncogenic event.

Also disclosed herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the presence or absence of a mutation in the adenomatous polyposis coli (APC) tumor suppressor gene. In the disclosed methods, the loss or mutation of APC can be a key initiating event in a series of genetic and epigenetic events that can indicate an oncogenic event. The disclosed methods can further comprise determining the expression level of a retinol dehydrogenase or a alcohol dehydrogenase. Alcohol dehydrogenases (ADH) catalyzes the conversion of retinol into retinal and retinol dehydrogenase catalyzes the conversion of dietary retinol into retinaldehyde. In the disclosed methods, a mutation in the APC gene and a decrease in the expression level of the retinol dehydrogenase or the alcohol dehydrogenase can indicate an oncogenic event. The disclosed methods can further comprise determining the expression level of ALDH1. ALDH is a commonly used marker of stem cells and cancer stems including those derived from human colon and colon carcinoma. In the disclosed method, an increase in the expression level of ALDH1 and the presence of a mutation in APC can indicate an oncogenic event. The methods can further comprise determining the expression level of a retinol dehydrogenase or a alcohol dehydrogenase, wherein a decrease in the expression level of the retinol dehydrogenase or the alcohol dehydrogenase, an increase in the expression level of ALDH1, and the presence of a mutation in APC can indicate an oncogenic event.

Also described herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the expression level of a retinol dehydrogenase or a alcohol dehydrogenase and the expression level of retinol. Production of retinoic acid first requires converting dietary retinol (vitamin A) into retinoic acid, a process that occurs via two enzymatic step: the conversion of retinol into retinal by alcohol dehydrogenases (ADH) and short chain dehydrogenases (SDR), followed by the conversion of retinal into retinoic acid via aldehyde dehydrogenases (ALDH). In the disclosed methods, a decrease in the expression level of the retinol dehydrogenase or the alcohol dehydrogenase and an increase in the expression level of retinol i can indicate an oncogenic event. In the disclosed methods, an increase in the expression level of retinol can also indicate a defect in the absorption process.

Also disclosed herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample can indicate an oncogenic event, wherein the methods can further comprise determining the expression level of DNA methyltransferase (dnmt1). Dnmt1 is an enzyme that generates 5-methylcytosine (5-meC) in vertebrates. The inability of DNA methyltransferase to maintain normal patterns in highly proliferative cells is implicated in the genome-wide hypomethylation that occurs during tumorigenesis. A loss of dnmt1 can occur in parallel with upregulation of various Demethylase System Components in apc mutants. In the disclosed methods, a decrease in the expression level of dnmt1 can indicate an oncogenic event.

Also disclosed are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample can indicate an oncogenic event, wherein the methods can further comprise determining the expression level of APC. In the disclosed methods, a decrease in the expression level of APC can indicate an oncogenic event.

Disclosed herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample can indicate an oncogenic event, wherein the methods can further comprise determining the expression level of LEF1 and Groucho2/TLE3. Lef1, Groucho2/TLE3, CtBP1, LSD1 and Corest can work together to repress the production of retinoic acid by direct binding to, and repression of, the rdh1 promoter. In the disclosed methods, an increase in the expression levels of LEF1 and Groucho2/TLE3 can indicate an oncogenic event.

Also disclosed herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample can indicate an oncogenic event, wherein the methods can further comprise determining the expression level of LSD1, Corest or CrBP1. Lef1, Groucho2/TLE3, CtBP1, LSD1 and Corest can work together to repress the production of retinoic acid by direct binding to, and repression of, the rdh1 promoter. In the disclosed methods, an increase in the expression level of LSD1, Corest, or CrBP1 can indicate an oncogenic event.

Disclosed herein are methods of detecting an oncogenic event in a sample comprising determining the presence or absence of a mutation in the adenomatous polyposis coli (APC) tumor suppressor gene in a sample, wherein a mutation in the adenomatous polyposis coli (APC) tumor suppressor gene can indicate s an oncogenic event.

Disclosed herein are methods of detecting an oncogenic event in a sample comprising determining the presence or absence of a mutation in the adenomatous polyposis coli (APC) tumor suppressor gene in a sample, wherein a mutation in the adenomatous polyposis coli (APC) tumor suppressor gene can indicate an oncogenic event, and determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylase, wherein the one or more Demethylase System Components includes at least one thymine Demethylase System cofactor, and wherein a decrease in the expression level of the one or more of the Demethylase System thymine glycosylases and an increase in the expression level of expression of the one or more Demethylase System cytidine deaminases and an increase in the expression level of the one or more Demethylase System cofactors compared to the expression levels of a normal sample can indicate an oncogenic event.

Disclosed herein are methods of detecting an oncogenic event in a sample comprising determining the presence or absence of a mutation in the adenomatous polyposis coli (APC) tumor suppressor gene in a sample, wherein a mutation in the adenomatous polyposis coli (APC) tumor suppressor gene can indicate an oncogenic event, and determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylase, wherein the one or more Demethylase System Components includes at least one Demethylase System cofactor, and wherein a decrease in the expression level of the one or more of the Demethylase System thymine glycosylases and an increase in the expression level of the one or more Demethylase System cytidine deaminases compared to the expression levels of a normal sample and the detection of the one or more Demethylase System cofactors can indicate an oncogenic event.

Disclosed herein are methods of detecting an oncogenic event in a sample comprising determining the presence or absence of a mutation in the adenomatous polyposis coli (APC) tumor suppressor gene in a sample, wherein a mutation in the adenomatous polyposis coli (APC) tumor suppressor gene can indicate s an oncogenic event, and determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylase, wherein the one or more Demethylase System Components includes at least one Demethylase System cofactor, and wherein a decrease in the expression level of the one or more of the Demethylase System thymine glycosylases and an increase in the expression level of the one or more Demethylase System cofactors compared to the expression levels of a normal sample and the detection of the one or more Demethylase System cytidine deaminases can indicate an oncogenic event.

Disclosed herein are methods of detecting an oncogenic event in a sample comprising determining the presence or absence of a mutation in the adenomatous polyposis coli (APC) tumor suppressor gene in a sample, wherein a mutation in the adenomatous polyposis coli (APC) tumor suppressor gene can indicate an oncogenic event, and determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylase, wherein the one or more Demethylase System Components includes at least one Demethylase System cofactor, and wherein a decrease in the expression level of the one or more of the Demethylase System thymine glycosylases compared to the expression levels of a normal sample and the detection of the one or more Demethylase System cytidine deaminases and the detection of the one or more Demethylase System cofactors can indicate an oncogenic event.

Disclosed herein are methods of detecting an oncogenic event in a sample comprising determining the presence or absence of a mutation in the adenomatous polyposis coli (APC) tumor suppressor gene in a sample, wherein a mutation in the adenomatous polyposis coli (APC) tumor suppressor gene can indicate an oncogenic event, and determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylase, wherein the one or more Demethylase System Components includes at least one Demethylase System cofactor, wherein a decrease in the expression level of the one or more of the Demethylase System thymine glycosylases compared to the expression levels of a normal sample can indicate an oncogenic event.

Disclosed herein are methods of detecting an oncogenic event in a sample comprising determining the presence or absence of a mutation in the adenomatous polyposis coli (APC) tumor suppressor gene in a sample, wherein a mutation in the adenomatous polyposis coli (APC) tumor suppressor gene indicates an oncogenic event, and determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylase, wherein the one or more Demethylase System Components includes at least one Demethylase System cofactor, wherein a decrease in the expression level of the one or more of the Demethylase System thymine glycosylases and an increase in the expression level of the one or more of the Demethylase System cytidine deaminases compared to the expression levels of a normal sample can indicate an oncogenic event.

Disclosed herein are methods of detecting an oncogenic event in a sample comprising determining the presence or absence of a mutation in the adenomatous polyposis coli (APC) tumor suppressor gene in a sample, wherein a mutation in the adenomatous polyposis coli (APC) tumor suppressor gene can indicate an oncogenic event, and determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylase, wherein the one or more Demethylase System Components includes at least one Demethylase System cofactor, wherein a decrease in the expression level of the one or more of the Demethylase System thymine glycosylases compared to the expression levels of a normal sample and the detection of the expression of one or more Demethylase System cytidine deaminases in the sample can indicate an oncogenic event.

Disclosed herein are methods of detecting an oncogenic event in a sample comprising determining the presence or absence of a mutation in the adenomatous polyposis coli (APC) tumor suppressor gene in a sample, wherein a mutation in the adenomatous polyposis coli (APC) tumor suppressor gene can indicate an oncogenic event, and determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylase, wherein the one or more Demethylase System Components includes at least one Demethylase System cofactor, wherein a decrease in the expression level of the one or more of the Demethylase System thymine glycosylases and an increase in the expression of the one or more of the Demethylase System cofactors compared to the expression levels of the normal sample can indicate an oncogenic event.

Also disclosed are methods of detecting an oncogenic event in a sample comprising determining the presence or absence of a mutation in the adenomatous polyposis coli (APC) tumor suppressor gene in a sample, wherein a mutation in the adenomatous polyposis coli (APC) tumor suppressor gene can indicate an oncogenic event, and determining the expression level of one or more Demethylase System Components in a sample and comparing those expression levels to the expression levels of a normal sample, wherein the one or more Demethylase System Components includes at least one Demethylase System cytidine deaminase, wherein the one or more Demethylase System Components includes at least one Demethylase System thymine glycosylase, wherein the one or more Demethylase System Components includes at least one Demethylase System cofactor, wherein a decrease in the expression level of the one or more of the Demethylase System thymine glycosylases compared to the expression levels of a normal sample and the detection of the expression of one or more Demethylase System cofactors in the sample can indicate an oncogenic event.

The disclosed methods can further comprise determining the level of methylated DNA in the sample. The level of methylated DNA can be determined, for example, by assessing susceptibility to the restriction enzyme HpaII, which is methylation-inhibited. In the disclosed methods, a decrease in the level of methylated DNA indicates hypomethylation. Hypomethylation, in turn, can indicate an oncogenic event.

Also disclosed are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the level of DNA methylation of one or more of the promoters selected from the group consisting of aldh1a2, hox13a, evx1, pitx2, cyclind1, hoxd13a, junb1, frizzled8a, cdx4, sox9b, cyclinb2, sox4, Fabp2, Raldh2, pcna, and cyclinD1. In the disclosed methods, a decrease in the level of DNA methylation of the one or more promoters indicates an oncogenic event.

Also disclosed are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the presence of a G:T intermediate. The presence of the G:T intermediate can be determined, for example, using PCR. PCR can be conducted using a forward primer with a 3′-terminal adenosine that is complementary to the thymine base derived from the deamination of 5-meC, and using a reverse primer that is complementary to a downstream region of the target. In the disclosed methods, the presence of a G:T intermediate can indicate an oncogenic event.

In addition, disclosed herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the expression level of retinoic acid in the sample. In the disclosed methods, retinoic acid can antagonize and suppress the expression level of Cebpβ and Pou5f1. By antagonizing and suppressing the expression level of Cebpβ and Pou5f1 in the disclosed methods, retinoic acid can directly repress or downregulate other Demethylase System Components, thereby promoting DNA methylation of key genes and helping progenitors commit to differentiation. In the disclosed methods, a decrease in the expression level of retinoic acid can indicate an oncogenic event.

Also disclosed are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the expression level of Cebpβ or Pou5f1. In the disclosed methods, Cebpβ and Pou5f1 can directly activate Demethylase System Components. Thus, Cebpβ and Pou5f1 can be positive regulators or activators of various Demethylase System Components. For example, some Demethylase System Components, such as Gadd45α and aid, contain Cebpβ and Pou5f1 sites in their promoters. In the disclosed methods, an increase in the expression level of Cebpβ or Pou5f1 can indicate an oncogenic event.

The disclosed methods can further comprise determining the expression level of a retinol dehydrogenase or a alcohol dehydrogenase. Alcohol dehydrogenases (ADH) catalyzes the conversion of retinol into retinal and retinol dehydrogenase catalyzes the conversion of dietary retinol into retinaldehyde. In the disclosed methods, a mutation in the APC gene and a decrease in the expression level of the retinol dehydrogenase or the alcohol dehydrogenase can indicate an oncogenic event. The disclosed methods can further comprise determining the expression level of ALDH1. ALDH is a commonly used marker of stem cells and cancer stems including those derived from human colon and colon carcinoma. In the disclosed method, an increase in the expression level of ALDH1 and the presence of a mutation in APC can indicate an oncogenic event. The methods can further comprise determining the expression level of a retinol dehydrogenase or a alcohol dehydrogenase, wherein a decrease in the expression level of the retinol dehydrogenase or the alcohol dehydrogenase, an increase in the expression level of ALDH1, and the presence of a mutation in APC can indicate an oncogenic event.

Also described herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample indicates an oncogenic event, wherein the methods can further comprise determining the expression level of a retinol dehydrogenase or a alcohol dehydrogenase and the expression level of retinol. Production of retinoic acid first requires converting dietary retinol (vitamin A) into retinoic acid, a process that occurs via two enzymatic step: the conversion of retinol into retinal by alcohol dehydrogenases (ADH) and short chain dehydrogenases (SDR), followed by the conversion of retinal into retinoic acid via aldehyde dehydrogenases (ALDH). In the disclosed methods, a decrease in the expression level of the retinol dehydrogenase or the alcohol dehydrogenase and an increase in the expression level of retinol i can indicate an oncogenic event. In the disclosed methods, an increase in the expression level of retinol can also indicate a defect in the absorption process.

Also disclosed herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample can indicate an oncogenic event, wherein the methods can further comprise determining the expression level of DNA methyltransferase (dnmt1). Dnmt1 is an enzyme that generates 5-methylcytosine (5-meC) in vertebrates. The inability of DNA methyltransferase to maintain normal patterns in highly proliferative cells is implicated in the genome-wide hypomethylation that occurs during tumorigenesis. A loss of dnmt1 can occur in parallel with upregulation of various Demethylase System Components in apc mutants. In the disclosed methods, a decrease in the expression level of dnmt1 can indicate an oncogenic event.

Also disclosed are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample can indicate an oncogenic event, wherein the methods can further comprise determining the expression level of APC. In the disclosed methods, a decrease in the expression level of APC can indicate an oncogenic event.

Disclosed herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample can indicate an oncogenic event, wherein the methods can further comprise determining the expression level of LEF1 and Groucho2/TLE3. Lef1, Groucho2/TLE3, CtBP1, LSD1 and Corest can work together to repress the production of retinoic acid by direct binding to, and repression of, the rdh1 promoter. In the disclosed methods, an increase in the expression levels of LEF1 and Groucho2/TLE3 can indicate an oncogenic event.

Also disclosed herein are methods of detecting an oncogenic event in a sample comprising determining the expression level of one or more Demethylase System Components in a sample and comparing those expression levels to the expression levels of a normal sample, wherein an increase in the expression levels of the one or more Demethylase System Components compared to the expression levels of a normal sample can indicate an oncogenic event, wherein the methods can further comprise determining the expression level of LSD1, Corest or CrBP1. Lef1, Groucho2/TLE3, CtBP1, LSD1 and Corest can work together to repress the production of retinoic acid by direct binding to, and repression of, the rdh1 promoter. In the disclosed methods, an increase in the expression level of LSD1, Corest, or CrBP1 can indicate an oncogenic event.

Disclosed herein are solid supports comprising one or more primers, probes, polypeptides, or antibodies capable of hybridizing or binding to one or more of the Demethylase System Components genes or peptides described herein attached to the solid support. For example, an aspect of the present invention comprises solid supports comprising one or more primers, probes, polypeptides, or antibodies capable of hybridizing or binding to one or more Demethylase System Components described herein attached to the solid support.

Solid supports are solid-state substrates or supports with which molecules, such as analytes and analyte binding molecules, can be associated. Analytes, such as calcifying nano-particles and proteins, can be associated with solid supports directly or indirectly. For example, analytes can be directly immobilized on solid supports. Analyte capture agents, such a capture compounds, can also be immobilized on solid supports. For example, disclosed herein are antigen binding agents capable of specifically binding to Demethylase System Components. A form of solid support is an array. Another form of solid support is an array detector. An array detector is a solid support to which multiple different capture compounds or detection compounds have been coupled in an array, grid, or other organized pattern.

Solid-state substrates for use in solid supports can include any solid material to which molecules can be coupled. This includes materials such as acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumarate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A form for a solid-state substrate is a microtiter dish, such as a standard 96-well type. In preferred embodiments, a multiwell glass slide can be employed that normally contain one array per well. This feature allows for greater control of assay reproducibility, increased throughput and sample handling, and ease of automation.

Different compounds can be used together as a set. The set can be used as a mixture of all or subsets of the compounds used separately in separate reactions, or immobilized in an array. Compounds used separately or as mixtures can be physically separable through, for example, association with or immobilization on a solid support. An array can include a plurality of compounds immobilized at identified or predefined locations on the array. Each predefined location on the array generally can have one type of component (that is, all the components at that location are the same). Each location will have multiple copies of the component. The spatial separation of different components in the array allows separate detection and identification of the polynucleotides or polypeptides of one or more of the Demethylase System Components disclosed herein.

It is not required that a given array be a single unit or structure. The set of compounds may be distributed over any number of solid supports. For example, at one extreme, each compound may be immobilized in a separate reaction tube or container, or on separate beads or microparticles. Different modes of the disclosed method can be performed with different components (for example, different compounds specific for different proteins) immobilized on a solid support. Some solid supports can have capture compounds, such as antibodies, attached to a solid-state substrate.

Methods for immobilizing antibodies (and other proteins) to solid-state substrates are well established Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. A preferred attachment agent is the heterobifunctional cross-linker N-[γ-Maleimidobutyryloxy]succinimide ester (GMBS). These and other attachment agents, as well as methods for their use in attachment, are described in Protein immobilization: fundamentals and applications, Richard F. Taylor, ed. (M. Dekker, New York, 1991); Johnstone and Thorpe, Immunochemistry In Practice (Blackwell Scientific Publications, Oxford, England, 1987) pages 209-216 and 241-242, and Immobilized Affinity Ligands; Craig T. Hermanson et al., eds. (Academic Press, New York, 1992) which are incorporated by reference in their entirety for methods of attaching antibodies to a solid-state substrate. Antibodies can be attached to a substrate by chemically cross-linking a free amino group on the antibody to reactive side groups present within the solid-state substrate. For example, antibodies may be chemically cross-linked to a substrate that contains free amino, carboxyl, or sulfur groups using glutaraldehyde, carbodiimides, or GMBS, respectively, as cross-linker agents. In this method, aqueous solutions containing free antibodies are incubated with the solid-state substrate in the presence of glutaraldehyde or carbodiimide.

A method for attaching antibodies or other proteins to a solid-state substrate is to functionalize the substrate with an amino- or thiol-silane, and then to activate the functionalized substrate with a homobifunctional cross-linker agent such as (Bis-sulfo-succinimidyl suberate (BS³) or a heterobifunctional cross-linker agent such as GMBS. For cross-linking with GMBS, glass substrates are chemically functionalized by immersing in a solution of mercaptopropyltrimethoxysilane (1% vol/vol in 95% ethanol pH 5.5) for 1 hour, rinsing in 95% ethanol and heating at 120° C. for 4 hrs. Thiol-derivatized slides are activated by immersing in a 0.5 mg/mL solution of GMBS in 1% dimethylformamide, 99% ethanol for 1 hour at room temperature. Antibodies or proteins are added directly to the activated substrate, which are then blocked with solutions containing agents such as 2% bovine serum albumin, and air-dried. Other standard immobilization chemistries are known by those of skill in the art.

Each of the components (compounds, for example) immobilized on the solid support preferably is located in a different predefined region of the solid support. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components preferably are immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

Optionally, at least one address on the solid support can be a sequence that encodes one or more Demethylase System Components disclosed herein. Disclosed are solid supports where at least one address is the sequences or portion of sequences set forth in any of the peptide sequences disclosed herein. Solid supports can also contain at least one address is a variant of the sequences or part of the sequences set forth in any of the nucleic acid sequences disclosed herein. Solid supports can also contain at least one address is a variant of the sequences or portion of sequences set forth in any of the peptide sequences disclosed herein.

Disclosed are antigen microarrays for multiplex characterization of antibody responses. For example, disclosed are antigen arrays and miniaturized antigen arrays to perform large-scale multiplex characterization of antibody responses directed against the polypeptides, polynucleotides and antibodies described herein, using submicroliter quantities of biological samples as described in Robinson et al., Autoantigen microarrays for multiplex characterization of autoantibody responses, Nat. Med., 8(3):295-301 (2002), which in herein incorporated by reference in its entirety for its teaching of constructing and using antigen arrays to perform large-scale multiplex characterization of antibody responses directed against structurally diverse antigens, using submicroliter quantities of biological samples.

As noted herein, to improve sensitivity, multiple mutations may be assayed within a given sample. Binding agents specific for different proteins, antibodies, nucleic acids thereto provided herein may be combined within a single assay. Further, multiple primers or probes may be used concurrently. The selection of receptors may be based on routine experiments to determine combinations that results in optimal sensitivity. To assist with such assays, specific biomarkers can assist in the specificity of such tests. As such, disclosed herein is a biomarker, wherein the biomarker is capable of binding to or hybridizing with one or more Demethylase System Component gene or peptide.

The more Demethylase System Components or entities that bind or hybridize to the same can be used as biomarkers. The biomarkers described herein can be in any form that provides information regarding presence or absence of an oncogenic event or oncogenic condition or symptoms thereof. For example, the disclosed biomarkers can be, but is not limited to a nucleic acid molecule, a polypeptide, or an antibody.

The present invention also provides a computer system comprising a) a database including records comprising a plurality of biomarkers and associated diagnosis and therapy data; and b) a user interface capable of receiving a selection of one or more test results for use in determining expression levels of the one or more Demethylase System Components described herein and displaying the records associated with the expression levels.

It will be appreciated by those skilled in the art that the nucleic acids provided herein as well as the nucleic acid sequences identified from subjects can be stored, recorded, and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate a list of sequences or expression data comprising one or more of the nucleic acids or peptides of the one or more Demethylase System Components disclosed herein of the invention. Another aspect of the present invention is a computer readable medium having recorded thereon at least 2, 5, 10, 15, 20, 25, 30, 50, 100, 200, 250, 300, 400, 500, 1000, 2000, 3000, 4000 or 5000 nucleic acids of the invention or nucleic acid sequences identified from subjects. Computer readable media include magnetically readable media, optically readable media, electronically readable media and magnetic/optical media. For example, the computer readable media may be a hard disc, a floppy disc, a magnetic tape, CD-ROM, DVD, RAM, or ROM as well as other types of other media known to those skilled in the art.

Embodiments of the present invention include systems, particularly computer systems which contain the sequence or expression information described herein. As used herein, “a computer system” refers to the hardware components, software components, and data storage components used to store and/or analyze the nucleotide sequences of the present invention or other sequences. The computer system preferably includes the computer readable media described above, and a processor for accessing and manipulating the sequence data. Preferably, the computer is a general purpose system that comprises a central processing unit (CPU), one or more data storage components for storing data, and one or more data retrieving devices for retrieving the data stored on the data storage components. A skilled artisan can readily appreciate that any one of the currently available computer systems are suitable.

In one particular embodiment, the computer system includes a processor connected to a bus which is connected to a main memory, preferably implemented as RAM, and one or more data storage devices, such as a hard drive and/or other computer readable media having data recorded thereon. In some embodiments, the computer system further includes one or more data retrieving devices for reading the data stored on the data storage components. The data retrieving device may represent, for example, a floppy disk drive, a compact disk drive, a magnetic tape drive, a hard disk drive, a CD-ROM drive, a DVD drive, etc. In some embodiments, the data storage component is a removable computer readable medium such as a floppy disk, a compact disk, a magnetic tape, etc. containing control logic and/or data recorded thereon. The computer system may advantageously include or be programmed by appropriate software for reading the control logic and/or the data from the data storage component once inserted in the data retrieving device. Software for accessing and processing the expression data described herein (such as search tools, compare tools, modeling tools, etc.) may reside in main memory during execution. In some embodiments, the computer system may further comprise a sequence or expression comparer for comparing the nucleic acid sequences or expression levels stored on a computer readable medium to another test sequence stored on a computer readable medium. A “sequence comparer” refers to one or more programs which are implemented on the computer system to compare a nucleotide sequence with other nucleotide sequences or to compare the expression level of one sample or subject with the expression level of another sample or subject.

Disclosed herein are methods of determining responsiveness of an oncogenic condition in a subject to treatment with an active agent comprising administering the active agent to the subject, determining the expression level of one or more Demethylase System Components in a sample, and comparing those expression levels to the expression levels of the subject prior to administering the active agent, wherein a decrease in the expression level of the one or more Demethylase System Components in the subject after administering the active agent to the subject can indicate responsiveness to the active agent.

Disclosed herein are methods of determining responsiveness of an oncogenic condition in a subject to therapy comprising before, during, or after therapy, determining the expression level of one or more Demethylase System Components in a sample and comparing those expression levels to the expression levels of the subject prior to administering the active agent, wherein a decrease in the expression level of one or more of Demethylase System Components in the subject after therapy can indicate responsiveness to the therapy. The therapy can include administering one or more active agents or any other known therapy known to treat an oncogenic condition.

Disclosed herein are methods of screening the efficacy of an active agent for the ability to treat an oncogenic condition comprising determining the expression level of one or more Demethylase System Components in a subject, wherein the active agent was administered to the subject, wherein a decrease in the expression level of one or more Demethylase System Components in the subject after treatment can indicate efficacy of the active agent.

Disclosed herein are methods of determining responsiveness of an oncogenic condition in a sample comprising, contacting the sample with the active agent, determining the expression level of one or more Demethylase System Components in the sample and comparing those expression levels to the expression levels of the one or more Demethylase System Components of the sample prior to administering the active agent, wherein a decrease in the expression level of the one or more Demethylase System Components in the sample after administering the active agent to the sample can indicate responsiveness to the active agent.

Disclosed herein are methods of determining the efficacy of an oncogenic therapy, comprising determining the expression level of one or more Demethylase System Components before and after providing one or more oncogenic therapies to a subject, wherein a decrease in the expression level of one or more Demethylase System Components after oncogenic therapy can indicate the efficacy of the oncogenic therapy. The oncogenic therapy can include administering one or more active agents or any other known therapy known to treat an oncogenic condition.

The one or more Demethylase System Components can include at least one Demethylase System cytidine deaminase. In addition, the one or more Demethylase System Components can include at least one Demethylase System thymine glycosylase. Also, the one or more Demethylase System Components can include at least one Demethylase System cofactor.

The disclosed methods can further comprise determining the level of methylated DNA in the sample. The level of methylated DNA can be determined, for example, by assessing susceptibility to the restriction enzyme HpaII, which is methylation-inhibited. In the disclosed method, an increase in the level of methylated DNA indicates responsiveness to the active agent.

Disclosed herein are methods of determining responsiveness of an oncogenic condition in a subject to treatment with an active agent comprising administering the active agent to the subject, determining the expression level of one or more Demethylase System Components in a sample, and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the level of DNA methylation of one or more of the promoters selected from the group consisting of aldh1a2, hox13a, evx1, pitx2, cyclind1, hoxd13a, junb1, frizzled8a, cdx4, sox9b, cyclinb2, sox4, Fabp2, Raldh2, pcna, and cyclinD1. In the disclosed methods, an increase in the level of DNA methylation of the one or more of the promoters selected from the group consisting of aldh1a2, hox13a, evx1, pitx2, cyclind1, hoxd13a, junb1, frizzled8a, cdx4, sox9b, cyclinb2, sox4, Fabp2, Raldh2, pcna, or cyclinD1 can indicate responsiveness to the active agent.

Disclosed herein are methods of determining responsiveness of an oncogenic condition in a subject to treatment with an active agent comprising administering the active agent to the subject, determining the expression level of one or more Demethylase System Components in a sample, and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the presence of a G:T intermediate. The presence of the G:T intermediate can be determined, for example, using PCR. PCR can be conducted using a forward primer with a 3′-terminal adenosine that is complementary to the thymine base derived from the deamination of 5-meC and using a reverse primer that is complementary to a downstream region of the target. In the disclosed methods, the absence or reduction of a G:T intermediate can indicate responsiveness to an active agent.

Disclosed herein are methods of determining responsiveness of an oncogenic condition in a subject to treatment with an active agent comprising administering the active agent to the subject, determining the expression level of one or more Demethylase System Components in a sample and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the expression level of retinoic acid in the sample. In the disclosed method, an increase in the expression level of retinoic acid in the sample can indicate responsiveness to the active agent.

Disclosed herein are methods of determining responsiveness of an oncogenic condition in a subject to treatment with an active agent comprising administering the active agent to the subject, determining the expression level of one or more Demethylase System Components in a sample and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the expression level of Cebpβ or Pou5f1. In the disclosed methods, a decrease in the expression level of Cebpβ or Pou5f1 can indicate responsiveness to the active agent.

Disclosed herein are methods of determining responsiveness of an oncogenic condition in a subject to treatment with an active agent comprising administering the active agent to the subject, determining the expression level of one or more Demethylase System Components in a sample and comparing those expression levels to the expression levels of the subject prior to administering the active agent, wherein a decrease in the expression level of the one or more Demethylase System Components in the subject after administering the active agent to the subject indicates responsiveness to the active agent, wherein the method can further comprises determining expression level of a retinol dehydrogenase or alcohol dehydrogenase. In the disclosed method, an increase in the expression level of the retinol dehydrogenase or alcohol dehydrogenase can indicate responsiveness to the active agent. The disclosed methods can further comprise determining the expression level of ALDH1, wherein a decrease in the expression level of ALDH1 can indicate responsiveness to the active agent.

Disclosed herein are methods of determining responsiveness of an oncogenic condition in a subject to treatment with an active agent comprising administering the active agent to the subject, determining the expression level of one or more Demethylase System Components in a sample and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining expression levels of a retinol dehydrogenase or alcohol dehydrogenase and the expression level of retinol. In the disclosed methods, an increase in the expression level of retinol can indicate a defect in the absorption process. In the disclosed methods, an increase in the expression level of the retinol dehydrogenase or the alcohol dehydrogenase and a decrease in the expression level of retinol can indicate responsiveness to the active agent.

Also disclosed are methods of determining responsiveness of an oncogenic condition in a subject to treatment with an active agent comprising administering the active agent to the subject, determining the expression level of one or more Demethylase System Components in a sample and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the expression level of dnmt1. In the disclosed methods, an increase in the expression level of dnmt1 can indicate responsiveness to the active agent.

Disclosed herein are methods of determining responsiveness of an oncogenic condition in a subject to treatment with an active agent comprising administering the active agent to the subject, determining the expression level of one or more Demethylase System Components in a sample and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the expression level of APC. In the disclosed methods, an increase in the expression level of APC can indicates responsiveness to the active agent.

Disclosed herein is a method of determining responsiveness of an oncogenic condition in a subject to treatment with an active agent comprising administering the active agent to the subject, determining the expression level of one or more Demethylase System Components in a sample and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the expression levels of LEF1 and Groucho2/TLE3. In the disclosed methods, a decrease in the expression levels of LEF1 and Groucho2/TLE3 can indicate responsiveness to the active agent.

Disclosed herein are methods of determining responsiveness of an oncogenic condition in a subject to treatment with an active agent comprising administering the active agent to the subject, determining the expression level of one or more Demethylase System Components in a sample, and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the expression level of LSD1 or Corest. In the disclosed methods, a decrease in the expression level of LSD1 or Corest expression can indicate responsiveness to the active agent.

The term “active agent” refers to various types of compositions, techniques, and devices, that can be used to treat an oncogenic event or condition or to ameliorate one or more symptoms associated with and oncogenic event or condition. For example, an active agent can be a metal, a chemical, a pharmaceutical agent, or combinations thereof, which can be administered to a subject to treat an oncogenic event or oncogenic condition. For example, an active agent can be radiation, chemotherapy, or an anti-cancer drug, or a combination of these. An active agent can also be surgery. In the disclosed methods, an active agent can be delivered or exercised alone or can be delivered exercised in combination with one or more active agents. An active agent can be repeatedly or continuously delivered.

As used herein, the term “in combination” refers to the use of more than one active agents. The use of the term “in combination” does not restrict the order in which active agents are administered to a subject with an oncogenic event or oncogenic condition, e.g., hyperproliferative cell disorder, especially cancer. A first active agent can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 25 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second active agent to a subject which had, has, or is susceptible to an oncogenic event or oncogenic condition. The active agents are administered to a subject in a sequence and within a time interval such that the active agent can act together with the other active agent to provide an increased benefit than if they were administered otherwise. Any additional active agent can be administered in any order with the other additional active agents.

Numerous anti-cancer drugs are available for combination with the present method and compositions. Example of antineoplastic drugs include but are not limited to the following: Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

Other anti-neoplastic compounds include but are not limited to the following: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; atrsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+ myobacterium cell wall sk; mopidamol; multiple drug resistance genie inhibitor; multiple tumor suppressor 1-based therapy; mustard anticancer agent; mycaperoxide B; mycobacterial cell wall extract; myriaporone; N-acetyldinaline; N-substituted benzamides; nafarelino; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sd±1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfmosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

Active agents can also be radiosensitizers. Examples of known radiosensitizers include gemcitabine, 5-fluorouracil, pentoxifylline, and vinorelbine.

Active agents can also be chemotherapeutic drugs. The majority of chemotherapeutic drugs can be divided in to alkylating agents, antimetabolites, anthracyclines, plant alkaloids, topoisomerase inhibitors, monoclonal antibodies, and other antitumour agents. All of these drugs affect cell division or DNA synthesis. Some newer agents do not directly interfere with DNA. These include the new tyrosine kinase inhibitor imatinib mesylate (Gleevec® or Glivec®), which directly targets a molecular abnormality in certain types of cancer (chronic myelogenous leukemia, gastrointestinal stromal tumors). In addition, some drugs can be used which modulate tumor cell behaviour without directly attacking those cells. Hormone treatments fall into this category of adjuvant therapies.

Active agents can also be an alkylating agent. Alkylating agents are so named because of their ability to add alkyl groups to many electronegative groups under conditions present in cells. Cisplatin and carboplatin, as well as oxaliplatin are alkylating agents. Other agents are mechlorethamine, cyclophosphamide, chlorambucil. They work by chemically modifying a cell's DNA.

Active agents can also be an anti-metabolite. Anti-metabolites masquerade as purine ((azathioprine, mercaptopurine)) or pyrimidine—which become the building blocks of DNA. They prevent these substances becoming incorporated in to DNA during the ‘S’ phase (of the cell cycle), stopping normal development and division. They also affect RNA synthesis. Due to their efficiency, these drugs are the most widely used cytostatics.

Active agents can be plant alkaloids or terpenoids. These alkaloids are derived from plants and block cell division by preventing microtubule function. Microtubules are vital for cell division and without them it can not occur. The main examples are vinca alkaloids and taxanes. Active agents can be a vinca alkaloid. Vinca alkaloids bind to specific sites on tubulin, inhibiting the assembly of tubulin into microtubules (M phase of the cell cycle). They are derived from the Madagascar periwinkle, Catharanthus roseus (formerly known as Vinca rosea). The vinca alkaloids include: Vincristine, Vinblastine, Vinorelbine, Vindesine, and Podophyllotoxin. Podophyllotoxin is a plant-derived compound used to produce two other cytostatic drugs, etoposide and teniposide. They prevent the cell from entering the G1 phase (the start of DNA replication) and the replication of DNA (the S phase). The exact mechanism of its action still has to be elucidated. The substance has been primarily obtained from the American Mayapple (Podophyllum peltatum). Recently it has been discovered that a rare Himalayan Mayapple (Podophyllum hexandrum) contains it in a much greater quantity, but as the plant is endangered, its supply is limited. Studies have been conducted to isolate the genes involved in the substance's production, so that it could be obtained recombinantively.

Active agents can be a taxane. The prototype taxane is the natural product paclitaxel, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

Active agents can be a topoisomerase inhibitor. Topoisomerases are essential enzymes that maintain the topology of DNA Inhibition of type I or type II topoisomerases interferes with both transcription and replication of DNA by upsetting proper DNA supercoiling. Some type I topoisomerase inhibitors include the camptothecins irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Active agents can be an antitumour antibiotic (Antineoplastics). The chemotherapeutic of the disclosed method can be an (monoclonal) antibody. Monoclonal antibodies work by targeting tumour specific antigens, thus enhancing the host's immune response to tumour cells to which the agent attaches itself. Examples are trastuzumab (Herceptin), cetuximab, and rituximab (Rituxan or Mabthera). Bevacizumab is a monoclonal antibody that does not directly attack tumor cells but instead blocks the formation of new tumor vessels.

Active agents can be a hormonal therapy. Several malignancies respond to hormonal therapy. Strictly speaking, this is not chemotherapy. Cancer arising from certain tissues, including the mammary and prostate glands, may be inhibited or stimulated by appropriate changes in hormone balance. Steroids (often dexamethasone) can inhibit tumour growth or the associated edema (tissue swelling), and may cause regression of lymph node malignancies. Prostate cancer is often sensitive to finasteride, an agent that blocks the peripheral conversion of testosterone to dihydrotestosterone. Breast cancer cells often highly express the estrogen and/or progesterone receptor Inhibiting the production (with aromatase inhibitors) or action (with tamoxifen) of these hormones can often be used as an adjunct to therapy. Gonadotropin-releasing hormone agonists (GnRH), such as goserelin possess a paradoxic negative feedback effect followed by inhibition of the release of FSH (follicle-stimulating hormone) and LH (luteinizing hormone), when given continuously. Some other tumours are also hormone dependent, although the specific mechanism is still unclear.

Active agents can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the active agent into the nose and nasal passages through one or both of the nares and can comprise delivery by a spraying mechanism or droplet mechanism, or through aerosolization of the nucleic acid or vector. Administration of the active agent by inhalant can be through the nose or mouth via delivery by a spraying or droplet mechanism. Delivery can also be directly to any area of the respiratory system (e.g., lungs) via intubation. The exact amount of the active agent required will vary from subject to subject, depending on the species, age, weight and general condition of the subject, the severity of the inflammatory disorder being treated, the particular nucleic acid or vector used, its mode of administration and the like. Thus, it is not possible to specify an exact amount for every active agent. However, an appropriate amount can be determined by one of ordinary skill in the art using only routine experimentation given the teachings herein.

The active agent may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)), all of which are herein incorporated by reference in their entirety for their teaching of the same. As described herein, the active agents disclosed herein can be administered in a pharmaceutically acceptable carrier and can be delivered to the subject's cells in vivo or ex vivo by a variety of mechanisms well known in the art (e.g., uptake of naked DNA, liposome fusion, intramuscular injection of DNA via a gene gun, endocytosis and the like). Effective dosages and schedules for administering the active agent may be determined using the methods described herein.

By an “effective amount” of a compound as provided herein is meant a sufficient amount of the compound to provide the desired effect. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” can be determined by one of ordinary skill in the art using only routine experimentation.

Also disclosed are methods of determining responsiveness of an oncogenic condition in a subject to treatment with an active agent, said method comprising, wherein the subject expresses one or more Demethylase System thymine glycosylases at a level below the normal expression level of the same Demethylase System thymine glycosylases, administering the active agent to the subject, determining the expression level of the one or more Demethylase System thymine glycosylases after administering the active agent to the subject, and comparing the expression levels to the expression levels of the subject prior to administering the active agent, wherein an increase in the expression levels of the one or more of the Demethylase System thymine glycosylases in the subject after administering the active agent to the subject can indicate responsiveness to the active agent.

Disclosed herein are methods of determining responsiveness of an oncogenic condition in a subject to treatment with an active agent, said method comprising, wherein the subject expresses one or more Demethylase System thymine glycosylases at a level below the normal expression level of the same Demethylase System thymine glycosylases, administering the active agent to the subject, determining the expression level of the one or more Demethylase System thymine glycosylases after administering the active agent to the subject, and comparing the expression levels to the expression levels of the subject prior to administering the active agent, wherein the methods can further comprise determining the expression level of one or more Demethylase System cytidine deaminases prior to administering the active agent to the subject, administering the active agent to the subject, and determining the expression level of the one or more Demethylase System cytidine deaminases after administering the active agent to the subject, wherein an increase in the expression levels of the one or more of the Demethylase System thymine glycosylases in the subject after administering the active agent to the subject can indicate responsiveness to the active agent and a decrease in the expression level of the one or more Demethylase System cytidine deaminases in the subject after administering the active agent to the subject can indicate responsiveness to the active agent.

Also disclosed are methods of determining responsiveness of an oncogenic condition in a subject to treatment with an active agent, said method comprising, wherein the subject expresses one or more Demethylase System thymine glycosylases at a level below the normal expression level of the same Demethylase System thymine glycosylases, administering the active agent to the subject, determining the expression level of the one or more Demethylase System thymine glycosylases after administering the active agent to the subject, and comparing the expression levels to the expression levels of the subject prior to administering the active agent, wherein the methods can further comprise determining the expression level of one or more Demethylase System cofactors prior to administering the active agent to the subject, administering the active agent to the subject, and determining the expression level of the one or more Demethylase System cofactors after administering the active agent to the subject, wherein an increase in the expression levels of the one or more of the Demethylase System thymine glycosylases in the subject after administering the active agent to the subject can indicate responsiveness to the active agent and a decrease in the expression level of the one or more Demethylase System cofactors in the subject after administering the active agent to the subject can indicate responsiveness to the active agent.

Disclosed herein are methods of determining responsiveness of an oncogenic condition in a subject to treatment with an active agent, said method comprising, wherein the subject expresses one or more Demethylase System thymine glycosylases at a level below the normal expression level of the same Demethylase System thymine glycosylases, administering the active agent to the subject, determining the expression level of the one or more Demethylase System thymine glycosylases after administering the active agent to the subject, and comparing the expression levels to the expression levels of the subject prior to administering the active agent, wherein the methods can further comprise determining the expression level of one or more Demethylase System cytidine deaminases and one or more Demethylase System cofactors prior to administering the active agent to the subject, administering the active agent to the subject, and determining the expression level of the one or more Demethylase System cytidine deaminases and one or more Demethylase System cofactors after administering the active agent to the subject, wherein an increase in the expression levels of the one or more of the Demethylase System thymine glycosylases in the subject after administering the active agent to the subject indicates responsiveness to the active agent and a decrease in the expression levels of the one or more Demethylase System cytidine deaminases and one or more Demethylase System cofactors in the subject after administering the active agent to the subject can indicate responsiveness to the active agent

Also disclosed herein are methods of screening the efficacy of an active agent for the ability to treat an oncogenic condition, said method comprising, wherein the subject expresses one or more Demethylase System thymine glycosylases at a level below the normal expression level of the same Demethylase System thymine glycosylases, administering the active agent to the subject, determining the expression level of the one or more Demethylase System thymine glycosylases after administering the active agent to the subject, and comparing the expression levels to the expression levels of the subject prior to administering the active agent, wherein an increase in the expression levels of the one or more of the Demethylase System thymine glycosylases in the subject after administering the active agent to the subject can indicate efficacy of the active agent.

Also disclosed herein are methods of screening the efficacy of an active agent for the ability to treat an oncogenic condition, said method comprising, wherein the subject expresses one or more Demethylase System thymine glycosylases at a level below the normal expression level of the same Demethylase System thymine glycosylases, administering the active agent to the subject, determining the expression level of the one or more Demethylase System thymine glycosylases after administering the active agent to the subject, and comparing the expression levels to the expression levels of the subject prior to administering the active agent, wherein the methods can further comprise determining the expression level of one or more Demethylase System cytidine deaminases prior to administering the active agent to the subject, administering the active agent to the subject, and determining the expression level of the one or more Demethylase System cytidine deaminases after administering the active agent to the subject, wherein an increase in the expression levels of the one or more of the Demethylase System thymine glycosylase in the subject after administering the active agent to the subject and a decrease in the expression level of the one or more Demethylase System cytidine deaminases in the subject after administering the active agent to the subject can indicate efficacy of the active agent.

Also disclosed herein are methods of screening the efficacy of an active agent for the ability to treat an oncogenic condition, said method comprising, wherein the subject expresses one or more Demethylase System thymine glycosylases at a level below the normal expression level of the same Demethylase System thymine glycosylases, administering the active agent to the subject, determining the expression level of the one or more Demethylase System thymine glycosylases after administering the active agent to the subject, and comparing the expression levels to the expression levels of the subject prior to administering the active agent, wherein the methods can further comprise determining the expression level of one or more Demethylase System cofactors prior to administering the active agent to the subject, administering the active agent to the subject, and determining the expression level of the one or more Demethylase System cofactors after administering the active agent to the subject, wherein an increase in the expression levels of the one or more of the Demethylase System thymine glycosylases and a decrease in the expression level of the one or more Demethylase System cofactors in the subject after administering the active agent to the subject can indicate efficacy of the active agent.

Also disclosed herein are methods of screening the efficacy of an active agent for the ability to treat an oncogenic condition, said method comprising, wherein the subject expresses one or more Demethylase System thymine glycosylases at a level below the normal expression level of the same Demethylase System thymine glycosylases, administering the active agent to the subject, determining the expression level of the one or more Demethylase System thymine glycosylases after administering the active agent to the subject, and comparing the expression levels to the expression levels of the subject prior to administering the active agent, wherein the methods can further comprise determining the expression level of one or more Demethylase System cytidine deaminases and one or more Demethylase System cofactors prior to administering the active agent to the subject, administering the active agent to the subject, and determining the expression level of the one or more Demethylase System cytidine deaminases and one or more Demethylase System cofactors after administering the active agent to the subject, wherein an increase in the expression levels of the one or more of the Demethylase System thymine glycosylases and a decrease in the expression levels of the one or more Demethylase System cytidine deaminases and one or more Demethylase System cofactors in the subject after administering the active agent to the subject can indicate efficacy of the active agent.

Also disclosed are methods of screening the efficacy of an active agent for the ability to ameliorate one or more symptoms associated with an oncogenic condition, said method comprising, wherein the subject expresses one or more Demethylase System thymine glycosylases at a level below the normal expression level of the same Demethylase System thymine glycosylases, administering the active agent to the subject, determining the expression level of the one or more Demethylase System thymine glycosylases after administering the active agent to the subject, and comparing the expression levels to the expression levels of the subject prior to administering the active agent, wherein an increase in the expression levels of the one or more of the Demethylase System thymine glycosylase in the subject after administering the active agent to the subject can indicate efficacy of the active agent.

Also disclosed herein are methods of screening the efficacy of an active agent for the ability to ameliorate one or more symptoms associated with an oncogenic condition, said method comprising, wherein the subject expresses one or more Demethylase System thymine glycosylases at a level below the normal expression level of the same Demethylase System thymine glycosylases, administering the active agent to the subject, determining the expression level of the one or more Demethylase System thymine glycosylases after administering the active agent to the subject, and comparing the expression levels to the expression levels of the subject prior to administering the active agent, wherein the methods can further comprise determining the expression level of one or more Demethylase System cytidine deaminases prior to administering the active agent to the subject, administering the active agent to the subject, and determining the expression level of the one or more Demethylase System cytidine deaminases after administering the active agent to the subject, wherein an increase in the expression levels of the one or more of the Demethylase System thymine glycosylases and a decrease in the expression level of the one or more Demethylase System cytidine deaminases in the subject after administering the active agent to the subject can indicate efficacy of the active agent.

Also disclosed herein are methods of screening the efficacy of an active agent for the ability to ameliorate one or more symptoms associated with an oncogenic condition, said method comprising wherein the subject expresses one or more Demethylase System thymine glycosylases at a level below the normal expression level of the same Demethylase System thymine glycosylases, administering the active agent to the subject, determining the expression level of the one or more Demethylase System thymine glycosylases after administering the active agent to the subject, and comparing the expression levels to the expression levels of the subject prior to administering the active agent, wherein the methods can further comprise determining the expression level of one or more Demethylase System cofactors prior to administering the active agent to the subject, administering the active agent to the subject, and determining the expression level of the one or more Demethylase System cofactors after administering the active agent to the subject, wherein an increase in the expression levels of the one or more of the Demethylase System thymine glycosylases and a decrease in the expression level of the one or more Demethylase System cofactors in the subject after administering the active agent to the subject can indicate s efficacy of the active agent.

Also disclosed herein are methods of screening the efficacy of an active agent for the ability to ameliorate one or more symptoms associated with an oncogenic condition, said method comprising, wherein the subject expresses one or more Demethylase System thymine glycosylases at a level below the normal expression level of the same Demethylase System thymine glycosylases, administering the active agent to the subject, determining the expression level of the one or more Demethylase System thymine glycosylases after administering the active agent to the subject, and comparing the expression levels to the expression levels of the subject prior to administering the active agent, wherein the methods can further comprise determining the expression level of one or more Demethylase System cytidine deaminases and one or more Demethylase System cofactors prior to administering the active agent to the subject, administering the active agent to the subject, and determining the expression level of the one or more Demethylase System cytidine deaminases and one or more Demethylase System cofactors after administering the active agent to the subject, wherein an increase in the expression levels of the one or more of the Demethylase System thymine glycosylases and a decrease in the expression levels of the one or more Demethylase System cytidine deaminases and one or more Demethylase System cofactors in the subject after administering the active agent to the subject can indicate efficacy of the active agent.

The one or more Demethylase System Components can include at least one Demethylase System cytidine deaminase. In addition, the one or more Demethylase System Components can include at least one Demethylase System thymine glycosylase. Also, the one or more Demethylase System Components can include at least one Demethylase System cofactor.

The disclosed methods can further comprise determining the level of methylated DNA in the sample. The level of methylated DNA can be determined, for example, by assessing susceptibility to the restriction enzyme HpaII, which is methylation-inhibited. In the disclosed method, an increase in the level of methylated DNA indicates responsiveness to the active agent.

Disclosed herein are methods of determining responsiveness of an oncogenic condition in a subject to treatment with an active agent comprising administering the active agent to the subject, determining the expression level of one or more Demethylase System Components in a sample, and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the level of DNA methylation of one or more of the promoters selected from the group consisting of aldh1a2, hox13a, evx1, pitx2, cyclind1, hoxd13a, junb1, frizzled8a, cdx4, sox9b, cyclinb2, sox4, Fabp2, Raldh2, pcna, and cyclinD1. In the disclosed methods, an increase in the level of DNA methylation of the one or more of the promoters selected from the group consisting of aldh1a2, hox13a, evx1, pitx2, cyclind1, hoxd13a, junb1, frizzled8a, cdx4, sox9b, cyclinb2, sox4, Fabp2, Raldh2, pcna, or cyclinD1 can indicate responsiveness to the active agent.

Disclosed herein are methods of determining responsiveness of an oncogenic condition in a subject to treatment with an active agent comprising administering the active agent to the subject, determining the expression level of one or more Demethylase System Components in a sample, and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the presence of a G:T intermediate. The presence of the G:T intermediate can be determined, for example, using PCR. PCR can be conducted using a forward primer with a 3′-terminal adenosine that is complementary to the thymine base derived from the deamination of 5-meC and using a reverse primer that is complementary to a downstream region of the target. In the disclosed methods, the absence or reduction of a G:T intermediate can indicate responsiveness to an active agent.

Disclosed herein are methods of determining responsiveness of an oncogenic condition in a subject to treatment with an active agent comprising administering the active agent to the subject, determining the expression level of one or more Demethylase System Components in a sample and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the expression level of retinoic acid in the sample. In the disclosed method, an increase in the expression level of retinoic acid in the sample can indicate responsiveness to the active agent.

Disclosed herein are methods of determining responsiveness of an oncogenic condition in a subject to treatment with an active agent comprising administering the active agent to the subject, determining the expression level of one or more Demethylase System Components in a sample and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the expression level of Cebpβ or Pou5f1. In the disclosed methods, a decrease in the expression level of Cebpβ or Pou5f1 can indicate responsiveness to the active agent.

Disclosed herein are methods of determining responsiveness of an oncogenic condition in a subject to treatment with an active agent comprising administering the active agent to the subject, determining the expression level of one or more Demethylase System Components in a sample and comparing those expression levels to the expression levels of the subject prior to administering the active agent, wherein a decrease in the expression level of the one or more Demethylase System Components in the subject after administering the active agent to the subject indicates responsiveness to the active agent, wherein the method can further comprises determining expression level of a retinol dehydrogenase or alcohol dehydrogenase. In the disclosed method, an increase in the expression level of the retinol dehydrogenase or alcohol dehydrogenase can indicate responsiveness to the active agent. The disclosed methods can further comprise determining the expression level of ALDH1, wherein a decrease in the expression level of ALDH1 can indicate responsiveness to the active agent.

Disclosed herein are methods of determining responsiveness of an oncogenic condition in a subject to treatment with an active agent comprising administering the active agent to the subject, determining the expression level of one or more Demethylase System Components in a sample and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining expression levels of a retinol dehydrogenase or alcohol dehydrogenase and the expression level of retinol. In the disclosed methods, an increase in the expression level of retinol can indicate a defect in the absorption process. In the disclosed methods, an increase in the expression level of the retinol dehydrogenase or the alcohol dehydrogenase and a decrease in the expression level of retinol can indicate responsiveness to the active agent. Also disclosed are methods of determining responsiveness of an oncogenic condition in a subject to treatment with an active agent comprising administering the active agent to the subject, determining the expression level of one or more Demethylase System Components in a sample and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the expression level of dnmt1. In the disclosed methods, an increase in the expression level of dnmt1 can indicate responsiveness to the active agent.

Disclosed herein are methods of determining responsiveness of an oncogenic condition in a subject to treatment with an active agent comprising administering the active agent to the subject, determining the expression level of one or more Demethylase System Components in a sample and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the expression level of APC. In the disclosed methods, an increase in the expression level of APC can indicates responsiveness to the active agent.

Disclosed herein is a method of determining responsiveness of an oncogenic condition in a subject to treatment with an active agent comprising administering the active agent to the subject, determining the expression level of one or more Demethylase System Components in a sample and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the expression levels of LEF1 and Groucho2/TLE3. In the disclosed methods, a decrease in the expression levels of LEF1 and Groucho2/TLE3 can indicate responsiveness to the active agent.

Disclosed herein are methods of determining responsiveness of an oncogenic condition in a subject to treatment with an active agent comprising administering the active agent to the subject, determining the expression level of one or more Demethylase System Components in a sample, and comparing those expression levels to the expression levels of the subject prior to administering the active agent, wherein a decrease in the expression level of the one or more Demethylase System Components in the subject after administering the active agent to the subject can indicate responsiveness to the active agent, wherein the methods can further comprise determining the expression level of LSD1 or Corest. In the disclosed methods, a decrease in the expression level of LSD1 or Corest expression can indicate responsiveness to the active agent.

The methods can further further comprise determining the level of DNA methylation of one or more of the promoters selected from the group consisting of: aldh1a2, hox13a, evx1, pitx2, cyclind1, hoxd13a, junb1, frizzled8a, cdx4, sox9b, cyclinb2, sox4, wherein an increase in the level of DNA methylation of the one or more of the promoters selected from the group consisting of: aldh1a2, hox13a, evx1, pitx2, cyclind1, hoxd13a, junb1, frizzled8a, cdx4, sox9b, cyclinb2, and sox4. The methods can further further comprise determining the level of DNA methylation of one or more of the genes or promoter of the genes selected from the group listed in the Examples below.

Disclosed herein are methods for treating a subject with an oncogenic disorder, comprising administering to a subject in need thereof an effective amount of an activator or an inhibitor that affects one or more Demethylase System Components in an amount sufficient to ameliorate one or more symptoms of said oncogenic disorder. The term “activator” refers to various types of compositions, techniques, devices, pharmaceuticals, and treatments that can modulate the one or more Demethylase System Components by acting as an agonist, stimulating, enhancing, or increasing the expression level or activity of the one or more Demethylase System Components. An activator can include, but is not limited to, agonists, antagonists, peptidomimetics, lipids, and nucleic acids. The term “modulate”, as used herein, is meant to alter, by increasing or decreasing. Modulate can refer to an alteration in the biological activity of a gene or peptide. Modulation may be an increase or a decrease in peptide activity, a change in binding characteristics, or any other change in the biological, functional, or immunological properties of the peptide.

The term “agonist”, as used herein, refers to a molecule or a combination of molecules, which modulates directly or indirectly a biological activity of an endogenous gene or peptide. Agonists may include proteins, nucleic acids, aptamers, carbohydrates, or any other molecules, which display the aforementioned properties. The term “inhibitor” is a composition that modulates by decreasing or suppressing the expression or activity of its target. The term “inhibitor” can refer to various types of compositions, techniques, devices, pharmaceuticals, and treatments that can modulate the one or more Demethylase System Components by antagonizing, suppressing, repressing, or silencing the expression level of the one or more Demethylase System Components Inhibitors can include, but are not limited to, antagonists, functional nucleic acids, peptidomimetics, polynucleotides that contain peptide nucleic acids, and antibodies. The term “antagonist” as used herein, refer to a molecule or a combination of molecules, which modulates or blocks directly or indirectly a biological activity of a gene or peptide. Antagonists may include proteins, nucleic acids, aptamers, carbohydrates, or any other molecules which display the aforementioned properties.

Disclosed herein are functional nucleic acids that can interact with the disclosed Demethylase System Components. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of polynucleotide sequences disclosed herein or the genomic DNA of the polynucleotide sequences disclosed herein or they can interact with the polypeptide encoded by the polynucleotide sequences disclosed herein. Often functional nucleic acids are designed to interact with other nucleic acids based on sequence homology between the target molecule (for example the receptor nucleic acids) and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

For example, disclosed herein are functional nucleic acids that interact with the disclosed Demethylase System Components and could thus inhibit the expression of the Demethylase System Components. Functional nucleic acids can include antisense molecules, small interfering RNA, ribozymes, triplex forming functional nucleic acid molecules, external guide sequences that form a complex with the disclosed Demethylase System Components.

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules can be found in the following non-limiting list of U.S. Pat. Nos. 5,168,053, 5,624,824, 5,683,873, 5,728,521, 5,869,248, and 5,877,162.

Also disclosed are polynucleotides that contain peptide nucleic acids (PNAs) compositions that interact with the disclosed Demethylase System Components and could thus inhibit the expression of the receptors. PNA is a DNA mimic in which the nucleobases are attached to a pseudopeptide backbone (Good and Nielsen, Antisense Nucleic Acid Drug Dev. 1997; 7(4) 431-37). PNA is able to be utilized in a number of methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (Trends Biotechnol 1997 June; 15(6):224-9). As such, in certain embodiments, one may prepare PNA sequences that are complementary to one or more portions of an mRNA sequence based on the disclosed polynucleotides, and such PNA compositions may be used to regulate, alter, decrease, or reduce the translation of the disclosed polynucleotides transcribed mRNA, and thereby alter the level of the disclosed polynucleotide's activity in a host cell to which such PNA compositions have been administered.

PNA monomers or ready-made oligomers are commercially available from PerSeptive Biosystems (Framingham, Mass.). PNA syntheses by either Boc or Fmoc protocols are straightforward using manual or automated protocols (Norton et al., Bioorg Med. Chem. 1995 April; 3(4):437-45). The manual protocol lends itself to the production of chemically modified PNAs or the simultaneous synthesis of families of closely related PNAs. As used herein, “small interfering RNA” or “siRNA” (also called short interfering RNA or silencing RNA) refers to a class of double-stranded RNA molecules that can play a variety of roles in biology. Most notably, siRNA can be involved in the RNA interference (RNAi) pathway, where it can interfere with the expression of a specific gene. In addition to the role in the RNAi pathway, siRNAs can act in RNAi-related pathways, e.g., as an antiviral mechanism or in shaping the chromatin structure of a genome. Generally, siRNAs have a well-defined structure—a short (˜20-25 nucleotides) double strand of RNA with 2-nucleotide overhang on either end. Each strand has a 5′ phosphate group and a 3′ hydroxyl group. Essentially any gene with a known sequence can be targeted based on sequence complementarity with an appropriately tailored siRNA.

As used herein, “peptidomimetic” means a mimetic of a peptide which includes some alteration of the normal peptide chemistry. Peptidomimetics typically enhance some property of the original peptide, such as increase stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not. In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Homologs in any desired species, preferably mammalian, such as human, are provided or can readily be obtained by screening a human library, genomic or cDNA, with a probe comprising sequences of the nucleic acids set forth in the sequence listing herein, or fragments thereof, and isolating genes specifically hybridizing with the probe under preferably relatively high stringency hybridization conditions. For example, high salt conditions (e.g., in 6×SSC or 6×SSPE) and/or high temperatures of hybridization can be used. For example, the stringency of hybridization is typically about 5° C. to 20° C. below the Tₘ (the melting temperature at which half of the molecules dissociate from its partner) for the given chain length. As is known in the art, the nucleotide composition of the hybridizing region factors in determining the melting temperature of the hybrid. For 20mer probes, for example, the recommended hybridization temperature is typically about 55-58° C. Additionally, the rat sequence can be utilized to devise a probe for a homolog in any specific animal by determining the amino acid sequence for a portion of the rat protein, and selecting a probe with optimized codon usage to encode the amino acid sequence of the homolog in that particular animal. Any isolated gene can be confirmed as the targeted gene by sequencing the gene to determine it contains the nucleotide sequence listed herein as comprising the gene. Any homolog can be confirmed as a homolog by its functionality. Homologs can also be obtained by comparing the rat sequences to sequences from other species on readily available databases. For example, the rat sequence can be compared with mouse sequences to obtain a homolog. The homolog obtained from this comparison can then be compared to human sequences or other mammalian sequences to obtain additional homologs. The rat sequences can also be compared directly with human sequences or any other mammalian sequences on databases.

As used herein, the term “antibody” or “antibodies” can also refer to a human antibody or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response. The term “antibodies” is used herein in a broad sense and includes both polyclonal and monoclonal antibodies. In addition to intact immunoglobulin molecules, disclosed are antibody fragments or polymers of those immunoglobulin molecules, and human or humanized versions of immunoglobulin molecules or fragments thereof, as long as they are chosen for their ability to interact with the polypeptides disclosed herein.

“Antibody fragments” are portions of a complete antibody. A complete antibody refers to an antibody having two complete light chains and two complete heavy chains. An antibody fragment lacks all or a portion of one or more of the chains. Examples of antibody fragments include, but are not limited to, half antibodies and fragments of half antibodies. A half antibody is composed of a single light chain and a single heavy chain. Half antibodies and half antibody fragments can be produced by reducing an antibody or antibody fragment having two light chains and two heavy chains. Such antibody fragments are referred to as reduced antibodies. Reduced antibodies have exposed and reactive sulfhydryl groups. These sulfhydryl groups can be used as reactive chemical groups or coupling of biomolecules to the antibody fragment. A preferred half antibody fragment is a F(ab). The hinge region of an antibody or antibody fragment is the region where the light chain ends and the heavy chain goes on. The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The antibodies disclosed herein can also be administered to a subject. Nucleic acid approaches for antibody delivery also exist. The broadly neutralizing antibodies to the polypeptides disclosed herein and antibody fragments can also be administered to subjects or subjects as a nucleic acid preparation (e.g., DNA or RNA) that encodes the antibody or antibody fragment, such that the subject's own cells take up the nucleic acid and produce and secrete the encoded antibody or antibody fragment.

Disclosed herein are methods for treating a subject with an oncogenic disorder, comprising administering to a subject in need thereof an effective amount an activator or an inhibitor that affects one or more Demethylase System Components in an amount sufficient to prevent one or more symptoms of said oncogenic disorder. Also disclosed are methods of ameliorating one or more symptoms associated with an oncogenic disorder comprising administering to a subject in need thereof an effective amount of an activator or an inhibitor that affects one or more Demethylase System Components in an amount sufficient to ameliorate one or more symptoms of said oncogenic disorder.

Also disclosed are methods of preventing one or more symptoms associated with an oncogenic disorder comprising administering to a subject in need thereof an effective amount of an activator or an inhibitor that affects one or more Demethylase System Components in an amount sufficient to prevent one or more symptoms of said oncogenic disorder. Also disclosed are methods of treating an oncogenic disorder comprising administering to a subject in need thereof an effective amount of an activator or an inhibitor that affects one or more Demethylase System Components in an amount sufficient to ameliorate one or more symptoms of said oncogenic disorder.

Disclosed herein are methods of preventing an oncogenic disorder comprising administering to a subject in need thereof an effective amount of an activator or an inhibitor that affects one or more Demethylase System Components in an amount sufficient to prevent one or more symptoms of said oncogenic disorder. Disclosed herein are methods of preventing an oncogenic event comprising administering to a subject in need thereof an effective amount of an activator or an inhibitor that affects one or more Demethylase System Components in an amount sufficient to prevent one or more of said oncogenic events. Disclosed herein are methods of treating cancer comprising administering to a subject in need thereof an effective amount of one or more Demethylase System Components in an amount to induce cell differentiation. The disclosed methods of treating, ameliorating and preventing can further comprise, wherein the inhibitor affects the one or more Demethylase System Components by antagonizing, suppressing, repressing, or silencing the expression level of one or more Demethylase System Components. For example, the inhibitor can be a small interfering RNA (siRNA).

The disclosed methods of treating, ameliorating and preventing can further comprise, wherein the inhibitor affects the expression level of Cebpβ or Pou5f1. In the disclosed methods, Cebpβ and Pou5f1 can activate other Demethylase System Components. For example, some Demethylase System Components, such as Gadd45α and aid contain Cebpβ and Pou5f1 sites in their promoters. In the disclosed methods, an increase in the expression level of Cebpβ and Pou5f1DNA can indicate an oncogenic event. For example, in the disclosed methods, the inhibitor is retinoic acid. Retinoic acid can antagonize and suppress the expression level of Cebpβ and Pou5f1. By antagonizing and suppressing the expression level of Cebpβ and Pou5f1 in the disclosed methods, retinoic acid can directly represses or downregulates other Demethylase System Components, thereby promoting DNA methylation of key genes and helping progenitors commit to differentiation.

The disclosed methods of treating, ameliorating and preventing can further comprise, wherein the inhibitor affects ALDH1. ALDH1 is a commonly used marker of stem cells and cancer stems including those derived from human colon and colon carcinoma. In the disclosed methods, there can be an increase in the expression level of ALDH1 following apc mutation. In the disclosed methods, a decrease in the expression level of ALDH1 can be paralleled by increased cellular differentiation.

The disclosed methods of treating, ameliorating and preventing can further comprise, wherein the inhibitor affects LEF1 and Groucho2/TLE3. Due to the transcriptional repression of retinol dehydrogenase via a complex that includes Lef1, Ctbp1, Lsd1, and Corest, all of which associate with the retinol dehydrogenase promoter, APC mutants lack retinoic acid. APC mutants demonstrate high expression levels of LEF and Groucho2/TLE3 and demonstrate suppressed intestinal differentiation. Therefore, in the disclosed methods, an increase in the expression level of LEF and Groucho2/TLE3 can indicate s an oncogenic event.

The disclosed methods of treating, ameliorating and preventing can further comprise, wherein the inhibitor affects LSD1, Corest, or CrBP1. Lef1, Groucho2/TLE3, CtBP1, LSD1 and Corest can work together to repress the production of retinoic acid by direct binding to, and repression of, the rdh1 promoter. The activity of LSD1 can be required for maintaining repression of the RDH promoter in the presence of APC mutation. In the disclosed methods, an increase in expression or LSD1, Corest, or CrBP1 can indicate an oncogenic event.

The disclosed methods of treating, ameliorating and preventing can further comprise, wherein the activator affects the one or more Demethylase System Components by acting as an agonist, stimulating, enhancing, or increasing the expression level of one or more Demethylase System Components.

The disclosed methods of treating, ameliorating and preventing can further comprise, wherein the activator affects retinol dehydrogenase. Production of retinoic acid first requires converting dietary retinol (vitamin A) into retinoic acid, a process that occurs via two enzymatic step: the conversion of retinol into retinal by alcohol dehydrogenases (ADH) and short chain dehydrogenases (SDR), followed by the conversion of retinal into retinoic acid via aldehyde dehydrogenases (ALDH). In the disclosed methods, APC mutation can lead to the loss of retinol dehydrogenase, the enzyme that catalyzes the conversion of dietary retinol into retinaldehyde. The repression of retinoic production in APC mutants can be mediated by multiple factors including LEF1, Groucho2/TLE3, LSD1, Corest and CtBP1, all of which associate with the retinol dehydrogenase promoter. In the disclosed methods, a decrease in the expression level of retinol dehydrogenase can impair the production of retinoic acid, which in turn, can indicate an oncogenic event.

The disclosed methods of treating, ameliorating and preventing can further comprise, wherein the activator affects DNA methyltransferase (dnmt). Dnmt is an enzyme that can generates 5-methylcytosine (5-meC) in vertebrates. The inability of DNA methyltransferase to maintain normal patterns in highly proliferative cells has been implicated in the genome-wide hypomethylation that can occur during tumorigenesis. A loss of dnmt1 can occur in parallel with upregulation of various demethylase system components in apc mutants. In the disclosed methods, Dnmt-mediated loss of methylation can result in oncogenic events such as chromosomal instability and oncogenesis.

The disclosed methods of treating, ameliorating and preventing can further comprise, wherein the activator affects adenomatous polyposis coli (APC). The loss or mutation of APC can be an initiating event in a series of genetic and epigenetic events that lead to oncogenic events. For example, APC is reported to control retinoic acid production, which relies in part upon the transcriptional regulator CtBP1. In the disclosed methods, a loss of APC can upregulate Demethylase System Components and can impair the proper differentiation of intestinal cells, and indicates an oncogenic event.

Also disclosed herein are methods of inducing cell differentiation comprising introducing to a cell one or more an active agent that affects one or more Demethylase System Components. Also disclosed herein are methods of preventing cell differentiation comprising introducing to a cell an active agent that affects one or more Demethylase System Components. The disclosed methods of preventing or inducing cell differentiation can further comprise, wherein the inhibitor affects the one or more Demethylase System Components by antagonizing, suppressing, repressing, or silencing the expression level of one or more Demethylase System Components. For example, the inhibitor can be a small interfering RNA (siRNA).

The disclosed methods of preventing or inducing cell differentiation can further comprise, wherein the inhibitor affects the expression level of Cebpβ or Pou5f1. In the disclosed methods, Cebpβ and Pou5f1 can activate other Demethylase System Components. For example, some Demethylase System Components, such as Gadd45α and aid contain Cebpβ and Pou5f1 sites in their promoters. In the disclosed methods, an increase in the expression level of Cebpβ and Pou5f1DNA can indicate an oncogenic event. For example, in the disclosed methods, the inhibitor is retinoic acid. Retinoic acid can antagonize and suppress the expression level of Cebpβ and Pou5f1. By antagonizing and suppressing the expression level of Cebpβ and Pou5f1 in the disclosed methods, retinoic acid can directly represses or downregulates other Demethylase System Components, thereby promoting DNA methylation of key genes and helping progenitors commit to differentiation.

The disclosed methods of preventing or inducing cell differentiation can further comprise, wherein the inhibitor affects ALDH1. ALDH1 is a commonly used marker of stem cells and cancer stems including those derived from human colon and colon carcinoma. In the disclosed methods, there can be an increase in the expression level of ALDH1 following apc mutation. In the disclosed methods, a decrease in the expression level of ALDH1 can be paralleled by increased cellular differentiation.

The disclosed methods of preventing or inducing cell differentiation can further comprise, wherein the inhibitor affects LEF1 and Groucho2/TLE3. Due to the transcriptional repression of retinol dehydrogenase via a complex that includes Lef1, Ctbp1, Lsd1, and Corest, all of which associate with the retinol dehydrogenase promoter, APC mutants lack retinoic acid. APC mutants demonstrate high expression levels of LEF and Groucho2/TLE3 and demonstrate suppressed intestinal differentiation. Therefore, in the disclosed methods, an increase in the expression level of LEF and Groucho2/TLE3 can indicates an oncogenic event.

The disclosed methods of preventing or inducing cell differentiation can further comprise, wherein the inhibitor affects LSD1, Corest, or CrBP1. Lef1, Groucho2/TLE3, CtBP1, LSD1 and Corest can work together to repress the production of retinoic acid by direct binding to, and repression of, the rdh1 promoter. The activity of LSD1 can be required for maintaining repression of the RDH promoter in the presence of APC mutation. In the disclosed methods, an increase in expression or LSD1, Corest, or CrBP1 can indicate an oncogenic event. The disclosed methods of preventing or inducing cell differentiation can further comprise, wherein the activator affects the one or more Demethylase System Components by acting as an agonist, stimulating, enhancing, or increasing the expression level of one or more Demethylase System Components.

The disclosed methods of preventing or inducing cell differentiation can further comprise, wherein the activator affects retinol dehydrogenase. Production of retinoic acid first requires converting dietary retinol (vitamin A) into retinoic acid, a process that occurs via two enzymatic step: the conversion of retinol into retinal by alcohol dehydrogenases (ADH) and short chain dehydrogenases (SDR), followed by the conversion of retinal into retinoic acid via aldehyde dehydrogenases (ALDH). In the disclosed methods, APC mutation can lead to the loss of retinol dehydrogenase, the enzyme that catalyzes the conversion of dietary retinol into retinaldehyde. The repression of retinoic production in APC mutants can be mediated by multiple factors including LEF1, Groucho2/TLE3, LSD1, Corest and CtBP1, all of which associate with the retinol dehydrogenase promoter. In the disclosed methods, a decrease in the expression level of retinol dehydrogenase can impair the production of retinoic acid, which in turn, can indicate an oncogenic event.

The disclosed methods of preventing or inducing cell differentiation can further comprise, wherein the activator affects DNA methyltransferase (dnmt). Dnmt is an enzyme that can generates 5-methylcytosine (5-meC) in vertebrates. The inability of DNA methyltransferase to maintain normal patterns in highly proliferative cells has been implicated in the genome-wide hypomethylation that can occur during tumorigenesis. A loss of dnmt1 can occur in parallel with upregulation of various demethylase system components in apc mutants. In the disclosed methods, Dnmt-mediated loss of methylation can result in oncogenic events such as chromosomal instability and oncogenesis.

The disclosed methods of preventing or inducing cell differentiation can further comprise, wherein the activator affects adenomatous polyposis coli (APC). The loss or mutation of APC can be an initiating event in a series of genetic and epigenetic events that lead to oncogenic events. For example, APC is reported to control retinoic acid production, which relies in part upon the transcriptional regulator CtBP1. In the disclosed methods, a loss of APC can upregulate Demethylase System Components and can impair the proper differentiation of intestinal cells, and indicates an oncogenic event.

### EXAMPLES

### Example 1

### DNA Demethylation in Zebrafish Involves the Coupling of a Deaminase, a Glycosylase and Gadd45

### Genomic DNA Preparation, Restriction Digestion, and Southern Hybridization

Embryos were harvested at the designated time points and the genomic DNA was harvested using Puregene DNA isolation kit (QIAGEN/Gentra) according to the manufacturer's instructions. Total genomic DNA was digested with HpaII (100 units) or Msp1 (100 units) for 16 hr at 37° C. Uncut, HpaII cut, and MspI cut DNA were then separated on a 1% agarose gel. For Southern blotting, the gel was first incubated in denaturing solution (0.4 M NaOH and 1 M NaCl) twice for 15 mins, transferred to nylon membrane (Amersham), dried, crosslinked, and prehybridized in a buffer containing 6×SSC, 5×Denhardt's solution, 0.5% SDS, and 100 mg/ml salmon sperm DNA. Hybridization was carried out in the same pre-hyb solution with a probe (M-DNA) prepared using Rediprime kit (Amersham). Following hybridization, the membrane was washed twice in buffer 1 (1×SSC, 0.1% SDS) and buffer 2 (0.5×SSC, 0.1% SDS), and exposed to a phosphoimager (Amersham).

Morpholino, Plasmid, and mRNA Injections

Wild-type zebrafish (Tuebingen strain) were maintained in a 14 hr:10 hr light:dark cycle in a Z-Mod at 28.5° C. Embryos were injected at single-cell stage and then grown in a 28.5° C. incubator. Morpholinos were obtained from Gene-tools LLC Ltd. For these experiments, morpholino sequences were designed against the following:

AID
(SEQ ID No. 1)
(5′-GTTTTGTTTTCGCTTACCTGTCCAG-3′);
Apobec2a
(SEQ ID No. 2)
(5′-GCTGCTGCCCTTTCTATCGGCCATC-3′);
Apobec2b
(SEQ ID No. 3)
(5′-CTTGCTGTCCTTTTTGTCTGCCATG-3′);
Gadd45α
(SEQ ID No. 4)
(5′-ATATAAAAACATACCTTTCCGTTGC-3′);
Gadd45αlike
(SEQ ID No. 5)
(5′-CACCGAGTCCATCCTGGAAAACCAC-3′);
Gadd45β
(SEQ ID No. 6)
(5′-AAAGGAACTTACTTTGTATCAGTAA-3′);
Gadd45γ
(SEQ ID No. 7)
(5′-TCCGGCAGTCTGCATTCTGGAGAAA-3′);
zMbd4
(SEQ ID No. 8)
(5′-AGAGAGAAACACACCTGCTCGCTGC-3′);
AID Scr Mo
(SEQ ID No. 9)
(5′-CTGACTTCCCGTTGTTATCTGTGTT-3′);
Mbd4 Scr Mo
(SEQ ID No. 10)
(5′-CACACACGGTGGCAAATACGCGTCA-3′);
Apobec2a mismatch Mo
(SEQ ID No. 11)
(5′-GCTCCTCCCCTTTGTATCCGCGATC-3′);
Apobec2b mismatch Mo
(SEQ ID No. 12)
(5′-CTTGgTGTCgTTTTaGTgTGCgATG-3′);
and
Control
(SEQ ID No. 13)
(5′-CCTCTTACCTCAG TACAATTTATA-3′).

For plasmid injections and transfections, full length cDNAs were cloned as follows:
(1) zAID (Ensemb1 gene ID: ENSDARG00000015734) in pcDNA-nV5-DEST (Invitrogen);
(2) Apobec2a (GenBank Accession No: FJ469677; Ensemb1 gene ID: ENSDARG00000018881; GenBank accession no: NM_{—}001013314) in pDEST-40 (Invitrogen),
(3) Apobec2b (GenBank Accession No: FJ469678; Ensemb1 gene ID: ENSDARG00000034604; GenBank Accession no: XM_{—}690335) in pCMV-3×HA (modified to add two extra HA tags in pCMV-HA from Stratagene);
(4) Gadd45α (Ensemb1 gene ID: ENSDARG00000034604) in pDEST-26 (Invitrogen) or pCMV-3×HA; and
(5) MBD4 (GenBank Accession no: NM_{—}003925) in pCMV-3XMyc (modified to add two extra Myc tags in pCMV-Myc from Stratagene) and pCMV-3×HA.

For mRNA injections, cDNAs for zAID, zApobec2a, zApobec2b, zGadd45α (Ensemb1 gene ID: ENSDARG00000069991), zGaddd45αlike (Ensemble gene ID: ENSDARG00000043581), and zGadd45β (GenBank accession number: AY714220) were cloned in pCRII-TOPO vector (Invitrogen). The partially sequenced zebrafish Mbd4 clones are an assembly of three ESTs present in GenBank database: CA473601, EG576141, AL921290, and Mbd4 morpholino is located in the first exon-intron boundary of the first EST. AID, Apobec2a/2b, MBD4, and Gadd45α mRNAs need to be made fresh in order to see efficient demethylation.

### Chromatin Immunoprecipitation

After injections with plasmids as indicated, embryos were harvested at 12 hpf and crosslinked in 2.2% paraformaldehyde. Crosslinking was stopped using 0.125 M glycine, and after washing in PBS nuclei were isolated by breaking embryos in cell lysis buffer (10 mM Tris-Cl, [pH 8.0], 10 mM NaCl, 0.5% NP-40, and protease inhibitors). Nuclei were then precipitated and broken in nuclei lysis buffer (50 mM Tris-Cl, [pH 8.0], 10 mM EDTA, 1% SDS, protease inhibitors, and phosphatase inhibitors). Extracts were then frozen at −80° C. Extracts were subsequently sonicated to produce DNA fragments between 300 bp-600 bp. After sonication and dilution in IP dilution buffer (16.7 mM Tris-Cl, [pH 8.0], 167 mM NaCl, 1.2 mM EDTA, 1.1% Triton X-100, and 0.01% SDS, protease inhibitors), extracts were precleared using sheep anti-rabbit dynabeads (Invitrogen) and then incubated with the respective antibodies overnight Immunocomplexes were collected by sheep anti-rabbit dynabeads which were then washed twice each in dialysis buffer (50 mM Tris-Cl, [pH 8.0], 2 mM EDTA, 0.2% Sarkosyl) and wash buffer (100 mM Tris-Cl, [pH 9.0], 500 mM LiCl, 1% NP-40, and 1% deoxycholic acid). Finally, DNA was eluted off the beads in elution buffer (50 mM NaHCO₃ and 1% SDS) and eluate incubated in 0.3 M NaCl and 100 ng RNaseA at 50° C. overnight. DNA was then purified using PCR purification kit (QIAGEN).

### RT-PCR

Embryos were harvested at the designated time points and total RNA was isolated using Trizol reagent (Invitrogen) according to manufacturer's instructions. Complimentary DNA library was prepared from 2 μg of total RNA using Superscript III reverse transcriptase enzyme (Invitrogen) according to the manufacturer's instructions.

### LC-MS

LC-MS (liquid chromatography-mass spectrometry) analysis of genomic 5mdC levels was performed as described previously in Song et al., 2005. Briefly, purified genomic DNA (500 ng) was denatured and hydrolysed through sequential digestion by 51 nuclease (Fermentas), venom phosphodiesterase I (Sigma), and alkaline phosphatase (Fermentas). A volume equivalent to 80 ng of the original DNA sample was then subjected to HPLC (high pressure liquid chromatography) (Agilent; model G1322A) first with a guard column (providing background reduction) and followed by an Atlantis DC18 silica column (Waters, # 186001301). Mass Spectrometry (MS) determinations were performed using an Applied Biosystems MDS Sciex API 3000 triple quadrupole mass spectrometer coupled to the LC system through a TurbolonSproay ion source interface (Song et al., 2005).

### Bisulfite Sequencing

Total genomic DNA was isolated from wild-type or injected embryos using Puregene DNA isolation kit (Gentra/QIAGEN). Genomic DNA (2 mg) was heat denatured in the presence of NaOH and bisulfite converted using 3 M sodium metabisulfite, (pH 5.0) (Sigma) and 0.5 mM hydroquinone (Sigma) overnight. The reaction mixture was then desalted using a DNeasy spin column (QIAGEN) and desulphonated in 0.3 M NaOH. Finally, DNA was recovered by ethanol precipitation.

### Whole Mount In Situ Hybridization

Whole mount in situ hybridization was performed as described earlier in Rai et al., 2006 with digoxigenin (Roche) labeled probes against neurogenin-1 and sox-2.

### Co-Immunoprecipitation and Western Blotting

Human colorectal cancer RKO cells were transfected at ˜90-95% confluency with the plasmids as designated and whole cell extracts were prepared within 24 hrs post transfection using IPH buffer (50 mM Tris, pH 8.0, 150 mM NaCl, 5 mM EDTA, 0.5% NP-40, Protease inhibitors, phosphatase inhibitors). Cells were harvested using PBS, pelleted, resuspended in IPH buffer containing protease inhibitors and phosphatase inhibitors, sonicated and spun to pellet the debris. Supernatant was and pre-cleared using washed sheep-anti rabbit dynabeads (Invitrogen). Proteins were then incubated with the designated antibody overnight at 4° C. Immunocomplexes were then pelleted using the rabbit dynabeads, boiled in LDS samples buffer (Invitrogen) and then separated on a 4-12% denaturing polyacrylamide gel. Proteins were then transferred on a PVDF membrane which was then immunoblotted in the designated antibodies.

### Methylated DNA Immunoprecipitation (Me-DIP)

Genomic DNA was prepared using Puregene DNA isolation kit (Gentra), sonicated to 300 bp-1000 bp length and purified using PCR purification kit (Qiagen). Four micrograms (4 μg) of this DNA was incubated with 10 μg of 5-Methylcytosine antibody (Eurogentech) in IP buffer (20 mM Tris (pH7.5), 140 mM NaCl, 0.05% Triton X-100) for 4 hrs at 4° C. DNA-antibody complexes were then pulled down using BSA and poly dAdT saturated sheep-anti-mouse dynabeads (Invitrogen), beads washed three times in IP buffer and then eluted by proteinase K digestion for 3 hrs at 50° C. and subsequent purification using PCR purification kit (Qiagen). Eluted DNA was then subjected to PCR for target identification.

### Statistical Analysis

Error bars represent plus or minus one standard deviation. P values were calculated using an unpaired t test.

### DNA Demethylation/Remethylation Activity in Zebrafish Embryos

Previous work in zebrafish embryos revealed the in vivo demethylation of an in vitro-methylated DNA (M-DNA) fragment (or plasmid) occurring during a particular window of embryo development (Collas, 1998), which indicated the presence of regulated DNA demethylation activity. In that study, and in subsequent assays, the steady-state methylation status of a methylated DNA fragment (M-DNA, 736 bp, injected at the single-cell stage) was assessed by susceptibility to the restriction enzyme HpaII (which is methylation-inhibited). Four HpaII/MspI sites (CCGG) are present, with HpaII or MspI digestion of the unmethylated (U) 736 bp DNA fragment generated five smaller fragments: two co-migrating fragments of 250 bp and 240 bp, one fragment of 176 bp, and two fragments that are too small for detection (32 bp and 38 bp). Based on mapping of HpaII sites, HpaII-resistant (methylated C^{me}CGG) and HpaII-cleaved species (unmethylated) run at 750 bp and 250 bp, respectively. MspI digestion (which is methylation insensitive) generates this spectrum from either unmethylated or fully methylated M-DNA. Full methylation of the DNA fragment by HpaII methylase (which methylates the internal cytosine of an HpaII/MspI restriction enzyme site, C^{me}CGG) and the digestion behavior of the substrate was verified in vitro, with cutting observed on unmethylated but not methylated DNA. MspI-digested and uncut DNA served as controls. Fragments were detected by Southern blot analysis probed with full-length M-DNA probe.

Following M-DNA injection into single-cell fertilized embryos, M-DNA was reisolated from the embryo at different developmental time points, treated with HpaII or MspI, and the cleavage was assessed via Southern analysis. The cleaved products (176 bp-250 bp) transfer much more efficiently to the membrane that does the intact 736 bp M-DNA substrate, providing greater signal). M-DNA remained largely methylated at ˜4 hr post-fertilization (hpf), was slightly demethylated at ˜8 hpf (75% epiboly), became clearly demethylated at 13 hpf (early somite stage), and then became largely remethylated by 28 hpf (prim stage).

This temporal pattern of demethylation/remethylation was also observed with an injected methylated closed circular plasmid. Full methylation was verified by HpaII resistance and MspI susceptibility. Unmethylated (U) and methylated (Me) plasmids were subjected to southern blotting using a 736 bp probe against the luciferase gene cDNA coded on the plasmid (same as M-DNA). In addition, a threshold level (50-100 pg) of M-DNA was required to elicit demethylation. M-DNA injection (or a methylated plasmid, >100 pg) caused the demethylation of 20%-40% of the bulk genome at 13 hpf, as determined by mass spectrometric analysis of 5-meC content and HpaII sensitivity of the bulk genome. The injection of the unmethylated 736 bp DNA fragment also elicited this genome-wide demethylation, though not to the same extent as M-DNA. However, bisulphite sequencing revealed the methylation of >50% of the CpGs on this fragment by 6 hpf, which is consistent with previous observations that injected DNA acquires methylation in early zebrafish embryos (Collas, 1998). Remethylation of the DNA fragment and the bulk genome subsequently occurred by 28 hpf, showing that DNA methylation systems remain functional. Taken together, genome-wide demethylation can be induced in zebrafish embryos by the injection of methylated DNA in a time and concentration dependent manner.

Upregulation of AID/Apobec2a/b Proteins Coincident with Demethylation

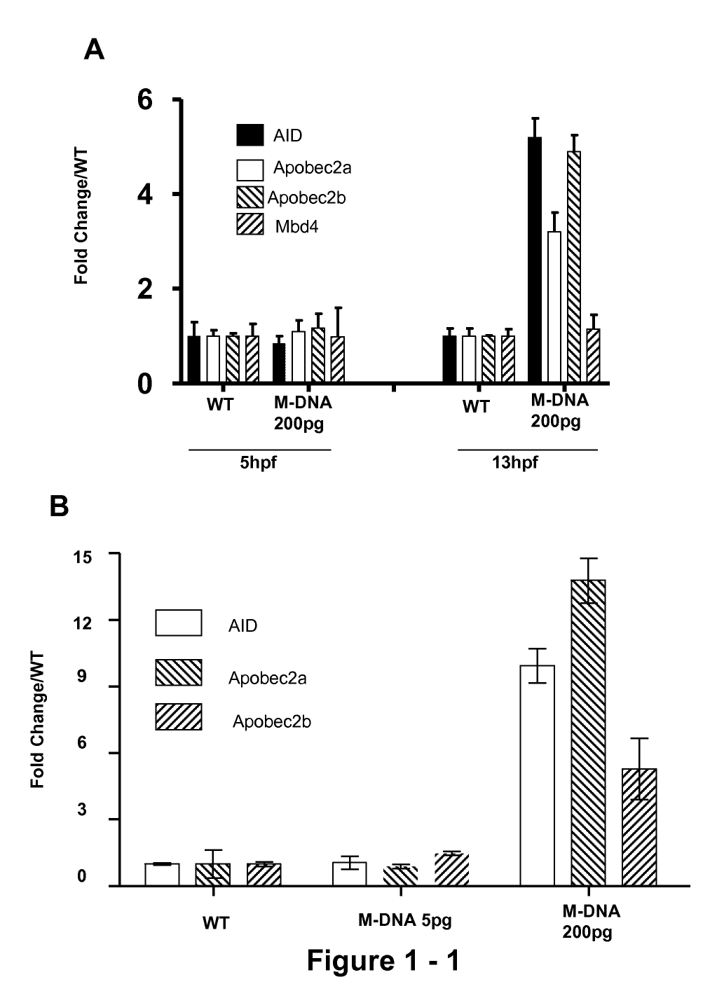

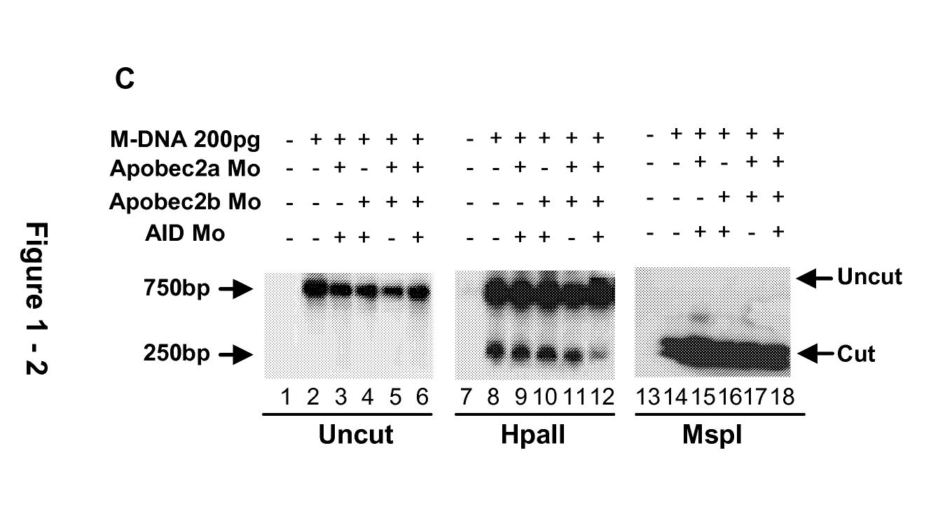

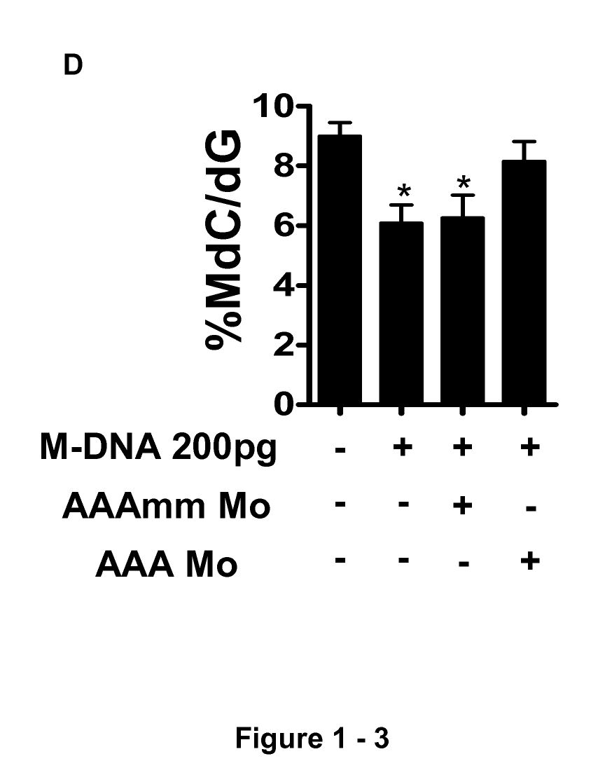

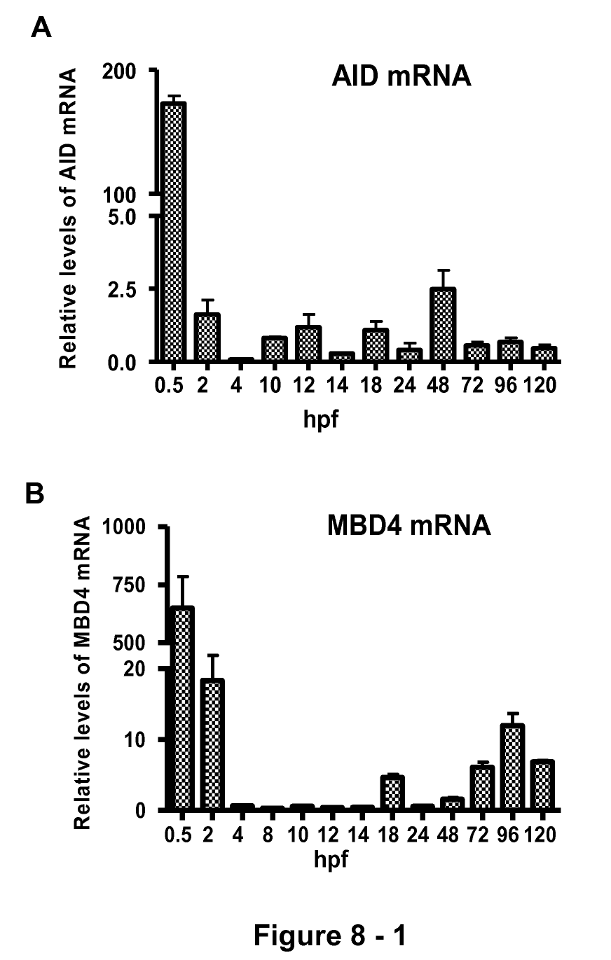

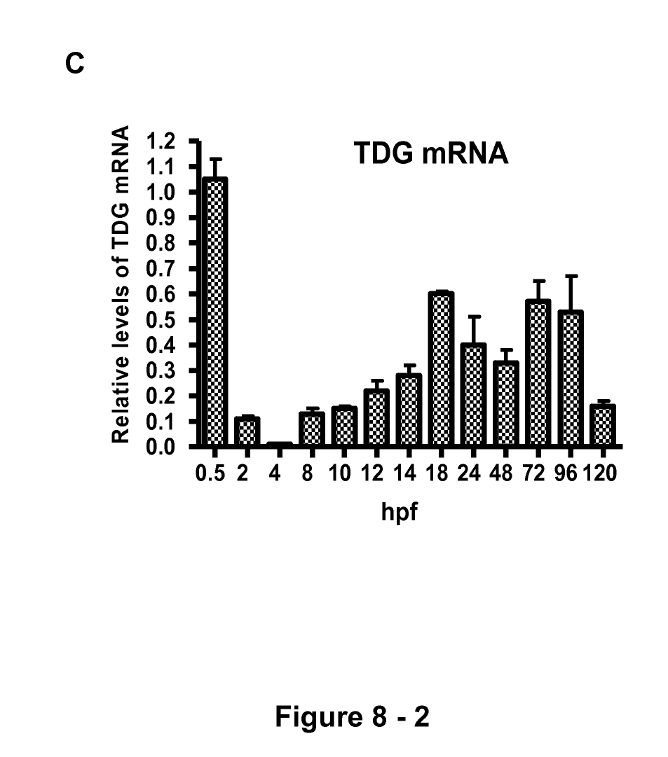

Whether particular candidate enzymes were transcriptionally upregulated during or just prior to the peak of M-DNA demethylation was tested. Although a lack of upregulation to does not exclude their involvement, the upregulation of the following candidates was tested: (1) the three annotated members of the AID/Apobec deaminase family (AID, Apobec2a, and Apobec2b), (2) all six annotated members of the AlkB dioxygenase family, (3) four of the six Gadd45-family proteins, and (4) the sole zebrafish homolog of human Mbd4, zMbd4. Zebra-fish lack Apobec 1/3 families as they are restricted to placental mammals. As assessed by qRT-PCR determinations, M-DNA injection upregulated the three AID/Apobec members (at 13 hpf), but not AlkB-related factors or zMbd4 (FIG. 1A), in a time and concentration-dependent manner (FIG. 1B). Finally, RT-PCR analysis revealed exceptionally high levels of AID and zMbd4, and very low levels of zTDG, in the single-cell-stage embryo (30 min postfertilization, FIG. 8-(A) AID, (B) zMbd4, and (C) TDG), where bulk DNA demethylation has most clearly been demonstrated in other studies (Mhanni and McGowan, 2004). Therefore, these studies focused on AID/Apobec enzymes and Mbd4.

Involvement of AID/Apobec enzymes in demethylation was assessed by knockdown experiments using antisense morpholino-modified oligonucleotides (hereafter referred as morpholinos). The AID knockdown was only partial, whereas Apobec2a/b knockdowns (4 pg and 2 pg, respectively) were efficient with Apobec knockdowns verified by immunoblot analysis using antisera that was raised against zebrafish Apobec2a or 2b. Knockdown of all three AID/Apobec members (but not each separately) attenuated demethylation of injected M-DNA and demethylation of the bulk genome caused by M-DNA injection at 13 hpf (FIGS. 1C and 1D, respectively; in FIG. 1C, lanes 1, 7, and 13 correspond to wild-type sample).

To test whether AID/Apobec enzymes affect the methylation status of the genome during normal development, the global methylation levels in embryos injected with morpholinos against all three deaminase members (AID, Apobec2a and Apobec2b; termed AAAmo) was measured in the absence of M-DNA injection. AAAmm refers to a set of three control morpholinos against AID (4 pg), Apobec2a (4 pg), and Apobec2b (2 pg) (AAA), which each contain five mismatched (mm) bases (of 25 total to prevent binding) relative to the efficacious morpholino (same amount as controls). For HpaII/MspI susceptibility, one representative of at least three biological repeats is shown. At 24 hpf, AAAmo embryos harbor 12% more methylation than wild-type embryos (WT, 8.55+/−0.04%, AAAmo=9.61+/−0.28%). Together, these results indicate that AID/Apobec enzymes normally reduce steady-state methylation levels.

### Coexpression of AID/Apobec and Mbd4 Causes Widespread DNA Demethylation

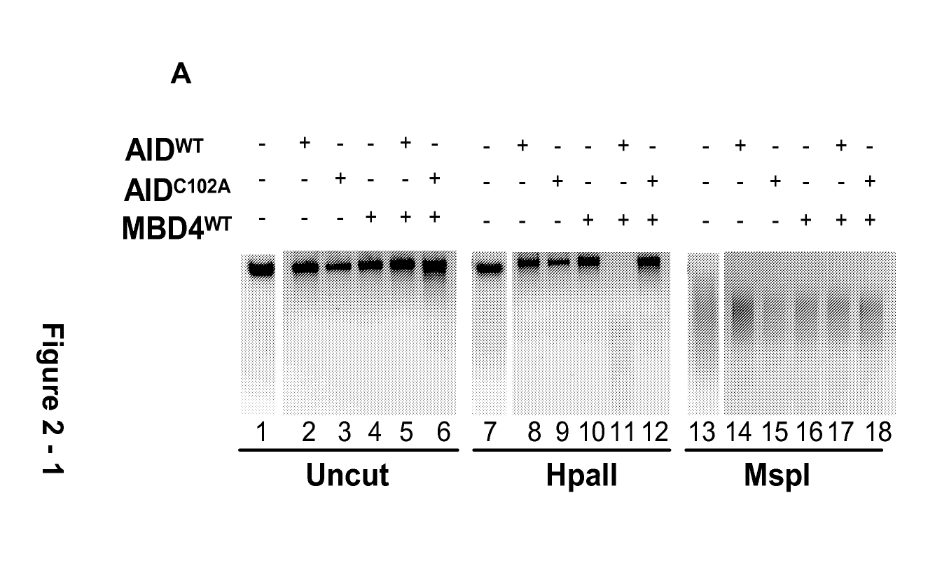

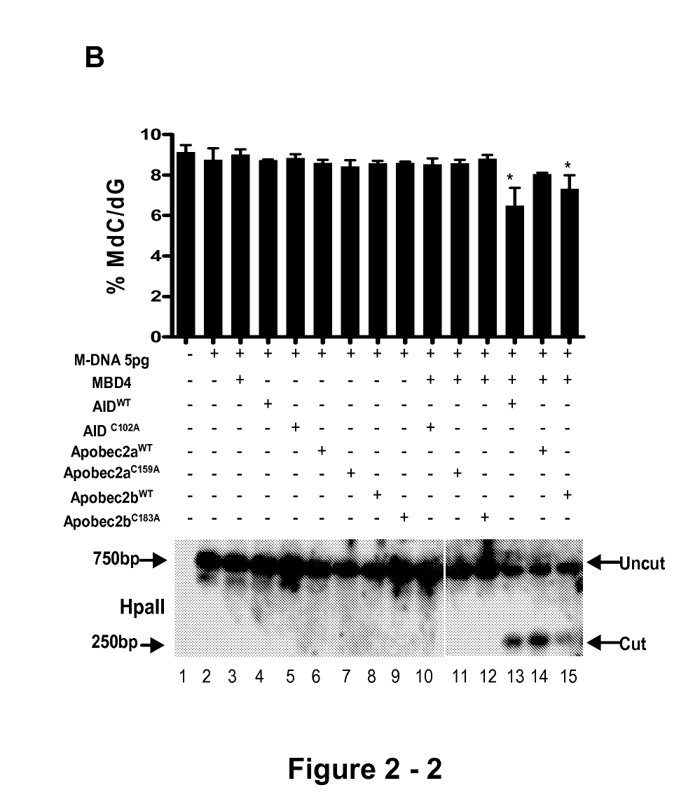

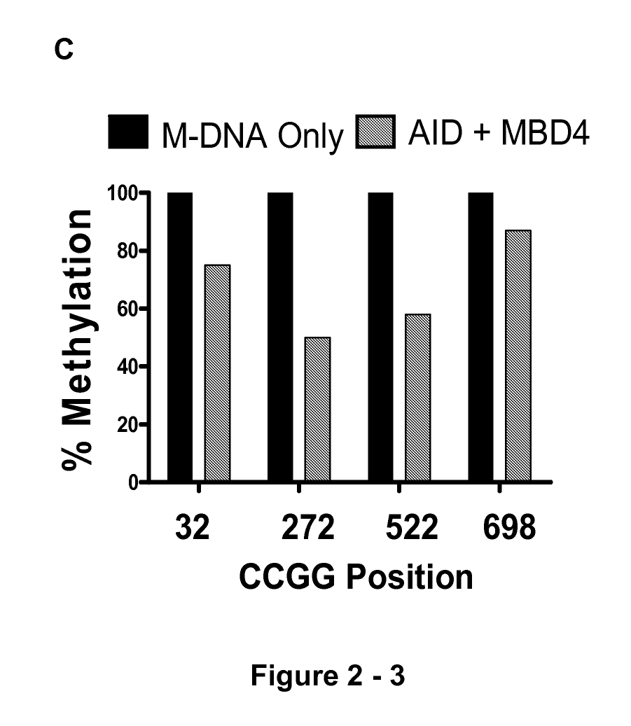

It was determined that AID or Apobec2a/b overexpression (by RNA injection at the single-cell stage) had little or no impact (at 13 hpf) on HpaII cleavage of the bulk genome or the injected M-DNA fragment, or on 5-meC levels assessed by mass spectrometry. This indicates that overexpressed AID/Apobec enzymes were either not targeted, or not activated, or both. Furthermore, the predicted product of successful deamination (a G:T mismatch) is not cleavable by HpaII. The restoration of cleavage requires thymine removal (possibly by base excision) and cytosine reincorporation by repair or replication. Next, whether coupling the deaminase with a glycosylase caused demethylation was tested. Because the current zebrafish genome-build remains incomplete, and lacks the 5 end of zMbd4, these overexpression studies instead employed full-length human Mbd4. The relevance of this approach is supported by the common observation that human orthologs effectively complement their zebrafish orthologs in knockdown/complementation studies (Lan et al., 2007; Rai et al., 2006), and expression of hMbd4 effectively complemented zMbd4 knock-down for phenotypic defects. As with AID/Apobec enzymes, overexpression of hMbd4 alone had little or no effect on methylation status (FIGS. 2A and 2B).

Coexpression of AID or Apobec2a/b along with hMbd4 provided clear DNA demethylation at 13 hpf as assessed by several methods and at multiple loci. FIG. 2 shows the HpaII cleavage of genomic DNA (FIG. 2A, lane 11), the mass spectrometric analysis of the bulk genome (FIG. 2B-upper panel, lanes 13-15), the HpaII cleavage of M-DNA (coinjected at 5 pg (subthreshold), FIG. 2B-lower panel, lanes 13-15), and the bisulphite sequencing of M-DNA (FIG. 2C), where demethylation was pronounced. In FIG. 2C, twenty clones were subjected to bisulphite sequencing, and the methylation status of each HpaII/MspI (CCGG) site reported as a percentage of total sites tested.

Although it is known that AID has an ability to deaminate 5me-C in single-stranded DNA in vitro; the efficacy of Apobec2a/b (especially Apobec2b) was unexpected, as substrates have heretofore been elusive. Furthermore, demethylation worked with the catalytic activity of each AID/Apobec enzyme (FIGS. 2A and 2B), and mutation of the catalytic residue did not affect enzyme abundance. The combination of AID/Apobec and hMbd4 overexpression, but not catalytic mutants, caused lethality by 24 hpf. This is consistent with observations in mice and zebrafish that even moderate changes in DNA methylation are highly detrimental or lethal (Ooi and Bestor, 2008; Rai et al., 2006). Finally, AID or Apobec expression alone did not prevent MspI cleavage of M-DNA or the bulk genome. Here, deamination of 5-meC (if it occurred) prevented MspI cleavage due to creation of G:T mismatches, but was not observed. These results indicate that deamination activity by AID/Apobec can be enhanced if Mbd4 (and possibly other factors) are present and/or activated.

These overexpression experiments showed reductions in bulk methylation which were validated by the definitive bisulphite sequencing method. To this end, the methylation status of two repetitive elements was examined. Here, LINE-1 elements showed a difference in methylation pattern but showed little change in total methylation levels. KenoDr1 showed moderate demethylation. For each experiment, one representative of at least three biological repeats is shown except in LC-MS measurement. As these loci are highly and constitutively methylated, these loci were not expected to be normal targets of the demethylase system in somatic cells. However, the overexpression and the presence of an MBD on Mbd4 could cause the targeting of these loci.

Evidence for Demethylation via a G:T intermediate

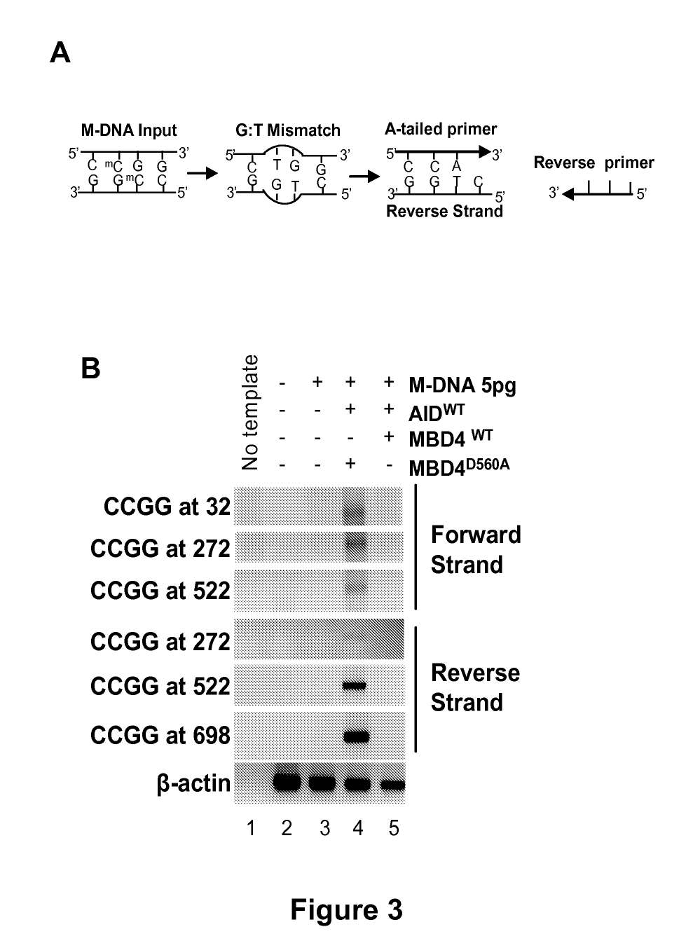

FIG. 3A shows a schematic of the PCR reaction for thymine (C^{me}CGG>CTGG) detection at M-DNA HpaII/MspI sites using an A-tailed primer (only 3 of the 22 bases shown) with an adenosine at the 3′ end. To test whether the demethylation reaction following AID and MBD4 coexpression proceeded through a G:T intermediate, two assays were performed on M-DNA (5 pg, coinjected with AID and Mbd4) reisolated from 13 hpf embryos. First, PCR analysis of all four HpaII/MspI sites on M-DNA for the presence of a G:T base pair was conducted. The technique uses a ‘forward’ primer with a 3-terminal adenosine complementary to the thymine base derived from the deamination of 5-meC at the initial G:5meC base pair in M-DNA (FIG. 3A). The “reverse” primer is perfectly complimentary to a downstream region of M-DNA. Here, a PCR product is generated only when a G:T intermediate is formed.

Second, the isolation and sequencing of the M-DNA fragment for the frequency of 5-meC>T transitions (48 clones) was completed. Following deamination by AID, the thymine base of the mutagenic G:T intermediate can be rapidly removed by Mbd4 glycosylase activity. To stabilize the putative G:T intermediate, and prevent rapid thymine removal, a catalytically inactive hMbd4 derivative (D560A) was co-expressed with wild-type AID. Using a particular PCR strategy (FIG. 3A), the diagnostic PCR product of the G:T intermediate (FIG. 3B, lane 4) at all four initial HpaII sites was detected, and on both DNA strands. In addition, sequencing of the recovered M-DNA fragment revealed a small number (2/48) of C>T transitions at the internal cytosine of the HpaII/MspI site, but no other mutations. When wild-type AID and wild-type hMbd4 were coinjected neither assay yielded evidence for the proposed G:T intermediate (FIG. 3B, lane 5). Furthermore, the catalytically inactive Mbd4 derivative (D560A) was expressed at levels equivalent to active hMbd4. Together, these results provide evidence that the G:T intermediate is created by AID deaminase activity and removed by Mbd4 glycosylase activity, and also indicate the physical coupling of the deaminase and the glycosylase ensures rapid thymine removal.

### Gadd45 Proteins Promote DNA Demethylation in Zebrafish Embryos

The regulation of the DNA demethylation activity and the coupling of deamination and thymine base excision by a glycosylase were addressed. Gadd45α (and Gadd45α-like), a gene activated by DNA damage (Hollander and Fornace, 2002) and implicated previously in DNA demethylation (Barreto et al., 2007), was upregulated prior to and during the demethylation window elicited by M-DNA injection (200 pg, which is sufficient to elicit demethylation).

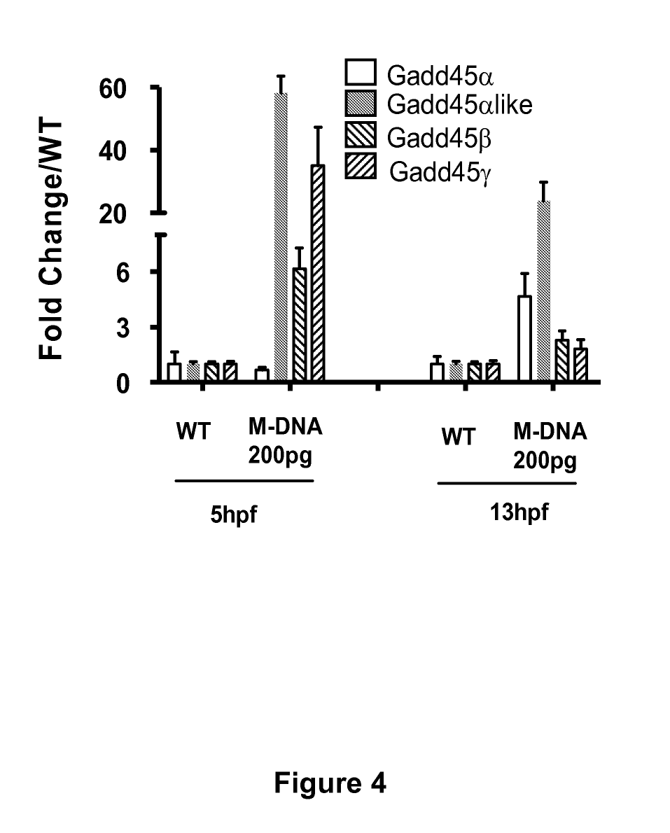

For example, FIG. 4 shows that Gadd45 family members are upregulated by M-DNA, as assessed by RT-PCR. However, zebrafish contain six Gadd45-family members (α/β/γ and α/β/γ-like, all highly similar to their human counterparts) making a comprehensive analysis of the entire family untenable. Therefore, these studies focused on the family member best associated with DNA damage signaling, Gadd45α, and were extended to other family members when appropriate. Zebrafish Gadd45α overexpression elicited moderate demethylation of the M-DNA fragment (injected at 5 pg, below the threshold level for eliciting demethylation on its own, and of the bulk genome (causing a 15% (or 9%) reduction by MS analysis: WT, 9.31±0.17%; Gadd45α, 7.91±0.2% (15% reduction); Gadd45αlike, 8.5±0.27% (9% reduction)). These results are consistent with the demethylation of genome-wide demethylation caused by M-DNA injection at 200 pg, indicating the involvement of Gadd45 members, and their partially redundant roles in M-DNA demethylation.

### Synergy Among Gadd45α, AID, and Mbd4

These data raised the possibility that Gadd45 could cooperate with AID/Apobec and Mbd4. As an initial test for cooperativity, AID, hMbd4 and Gadd45α injections were titrated to derive levels that individually (or as an AID/hMbd4 combination) did not cause demethylation. Injection of all three at these subthreshold levels elicited DNA demethylation of M-DNA and also the bulk genome, assessed by both HpaII cutting and mass spectrometric analysis.

### Gadd45 Proteins Upregulate Specific AID/Apobec Proteins

Whether Gadd45α overexpression upregulated AID/Apobec expression was next tested, which upregulation reflected cooperativity at the transcriptional level. Overexpression of Gadd45α greatly enhanced AID and Apobec2b expression at 13 hpf. In counter distinction, overexpression of Gadd45β strongly stimulated only Apobec2a expression. These observations indicate specific transcriptional relationships between Gadd45- and AID/Apobec-family members that help coordinate the functional interactions among Gadd45 members and particular AID/Apobec enzymes. Moreover, the upregulation of all three AID/Apobec members at 13 hpf was greatly attenuated by knocking down four Gadd45 family members. Whether Gadd45 might further influence demethylation by promoting the coupling of deaminase and glycosylase function was also evaluated.

AID and MBD4 Occupy Methylated DNA Loci that Undergo Demethylation

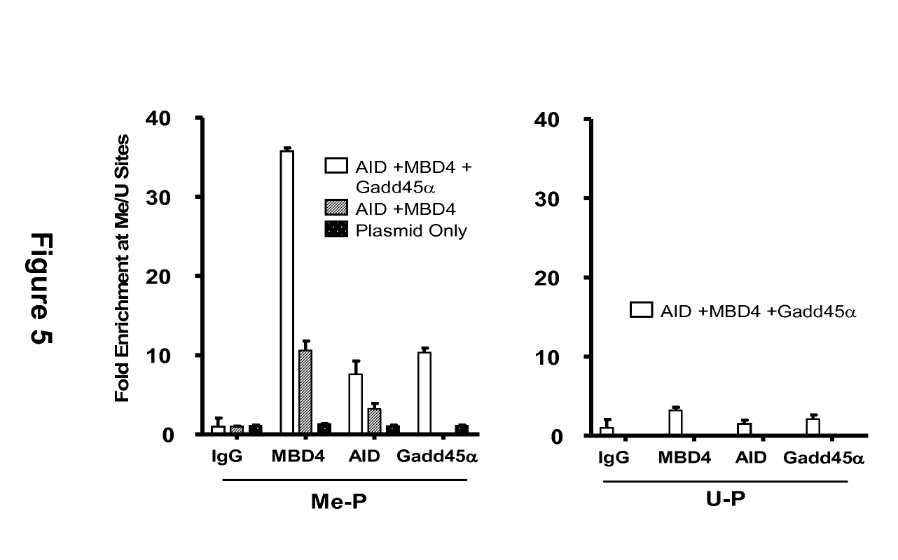

Whether AID and Mbd4 interact directly with a methylated DNA substrate in vivo, and whether this interaction was influenced by Gadd45α, were both tested. Here, a plasmid that has a region dense with HpaII sites and regions lacking HpaII sites was utilized. The plasmid was methylated in vitro with HpaII methylase. This provides a methylated region, and an unmethylated region on the same plasmid which can be compared for factor occupancy. This plasmid was also used for in vivo demethylation. The plasmid was injected into single-cell embryos along with DNA constructs encoding epitope-tagged derivatives of AID or hMbd4, and DNA binding was tested by chromatin immunoprecipitation (ChIP) in zebrafish embryos at 12 hpf. In the absence of Gadd45, binding of hMbd4 and AID was detectable on the methylated region of the methylated plasmid (Me-P) compared to the unmethylated region (FIG. 5, gray bars). Coexpression of Gadd45 enhanced the interaction of hMbd4 and AID proteins with the methylated region on Me-P (FIG. 5, left panel, red bars), but had little effect on the unmethylated plasmid (U-P, FIG. 5, right panel, red bars). This suggests that Gadd45 activity or abundance can affect AID or hMbd4 targeting. In FIG. 5, the y-axis values represent the ratio of enrichment on a DNA segment containing in vitro methylated C^{me}CGG sites to enrichment on a site (also on pCMV-Luc) containing no CCGG elements. Me-P and U-P on the x-axis depict methylated and unmethylated plasmid, respectively. FIG. 5 shows one representative experiment of three biological repeats.

### Gadd45α Promotes Deaminase/Glycosylase Interactions in Human Cells

The physical interactions between AID or Apobec enzymes and Mbd4 were also examined, including whether this was influenced by Gadd45 induction. Here, zebrafish embryos proved intractable for this assay due to low endogenous levels of AID/Apobec and Gadd45 and difficulties in deriving extracts from early embryos. Therefore, extracts derived from transfected human RKO cells to test for interactions by coimmunoprecipitation were used. Zebrafish AID or Apobec2a enzymes displayed a weak but detectable interaction with hMbd4, whereas a more robust interaction between hMbd4 and Apobec2b was detected. Coexpression of Gadd45α moderately enhanced the interaction of AID and Apobec2a enzymes with Mbd4. Furthermore, Gadd45 coprecipitated well with hMbd4, AID, Apobec2a, and Apobec2b, supporting a possibility that Gadd45 is capable of bridging the enzymes. Thus, in this heterologous system, physical interactions can occur among these proteins, though the modest IP efficiencies argue against a highly stable ternary complex.

As described above, Western blots showed the co-immunoprecipitation of V5-AID and Myc-hMbd4) in, V5-Apobec2a and Myc-hMbd4, and HA-Apobec2b and Myc-MBD4 when overexpressed in RKO cells in the absence or presence of Gadd45α. The interaction between Myc-hMbd4 and HA-tagged Gadd45α was also assessed, V5-AID and HA-Gadd45α, V5-Apobec2a and HA-Gadd45α, and HA-Apobec2b and His-Gadd45α were detected in RKO cell extracts overexpressing the two proteins, suggesting that Gadd45α can individually interact with deaminases or hMbd4.

AID, zMbd4, and Gadd45α Morphants Display Hypermethylation at neurod2

To address whether AID/Apobec enzymes, Gadd45, or zebrafish Mbd4 have a role in the control of DNA methylation during normal zebrafish development, a subset of the enzyme family members were knocked down (by morpholino injection) and their impact on development was examined. Either AID, Gadd45α, or Mbd4 knockdown caused the loss of neurons at 24 hpf, shown by the absence of pro-neuronal markers such as neurogenin-1 or sox-2. Specificity was demonstrated by rescue of neuronal markers by coinjecting (along with the morpholino) a spliced RNA refractory to the morpholino. As neurons form in mice lacking Mbd4 (or AID), zebrafish may rely more on these proteins for neurogenesis than do mice, or alternatively, neurogenesis in zebrafish may be more sensitive to misregulation of DNA methylation levels. Table 1 shows the statistics of the morpholino phenotypes. The percentage listed depicts embryos showing positive staining for the marker indicated.

TABLE 1
Statistics of Morpholino Phenotypes

| | Neurogenin-1 | Sox-2 |
|---|---|---|
| Control Mo | 100% (n = 42) | 97% (n = 39) |
| AID Mo | 12% (n = 50) | 13% (n = 38) |
| AID Mo + zAID RNA | 86% (n = 64) | 83% (n = 124) |
| Gadd45α Mo | 11% (n = 52) | 33% (n = 48) |
| Gadd45α Mo + zGadd45α RNA | 77% (n = 61) | 78% (n = 50) |
| Mbd4 Mo | 14% (n = 43) | 15% (n = 53) |
| Mbd4 Mo + hMbd4 RNA | 91% (n = 56) | 87% (n = 64) |

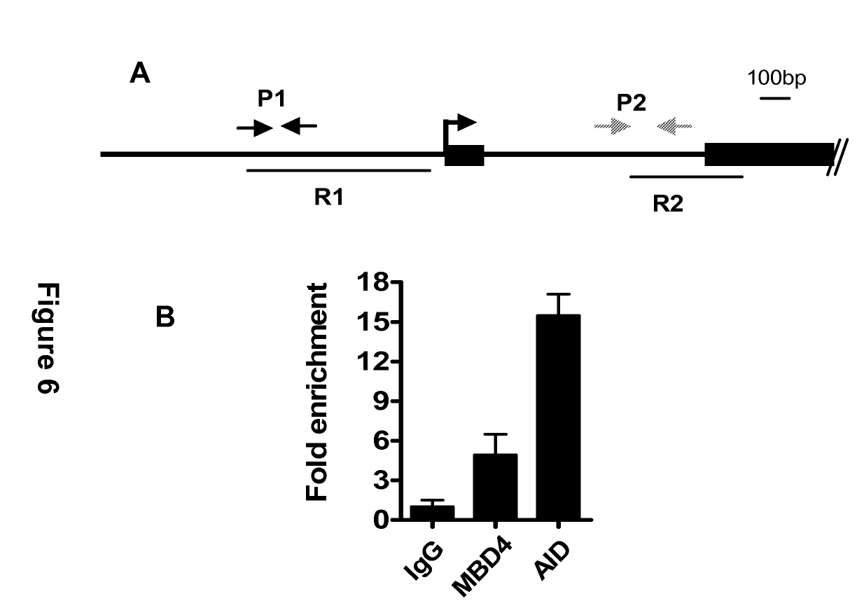

FIG. 6A shows a schematic of the neurod2 promoter and start site region. R1 and R2 show regions of bisulfite sequencing and P1 and P2 depict the amplicons used for ChIP determinations. The differences in methylation status at neurod2 (FIG. 6A) and at sox2, two transcription factors involved in neurogenesis, in both AID or zMbd4 morphants was tested and compared to control morphants at 80% epiboly—this is the latest time point in development where AID and MBD4 morphant embryos are indistinguishable from wild-type embryos (or control morphants), providing an appropriate examination point for methylation differences that might impact future phenotypes. A pronounced increase in CpG methylation near the neurod2 transcription start site was observed, but not 1.6 kb downstream, nor at the sox2 promoter. For these tests, bisulphate sequencing of the R1 region of the neurod 2 promoter, Scr Mo is a control morpholino in which the base composition is maintained, but the order is scrambled were used. To address whether the demethylation near the neurod2 TSS was direct, physical association of tagged exogenous AID and hMbd4 at the TSS of neurod2 by ChIP was examined, which revealed occupancy relative to the downstream control locus. FIG. 6B shows the enrichment of AID and hMbd4 at neurod2 (P1 versus P2). These results show occupancy of a gene involved in the morphant phenotype (neurogenesis) by the candidate enzymes, and provide a correlation between the loss of demethylase candidates and an increase in neurod2 promoter methylation.

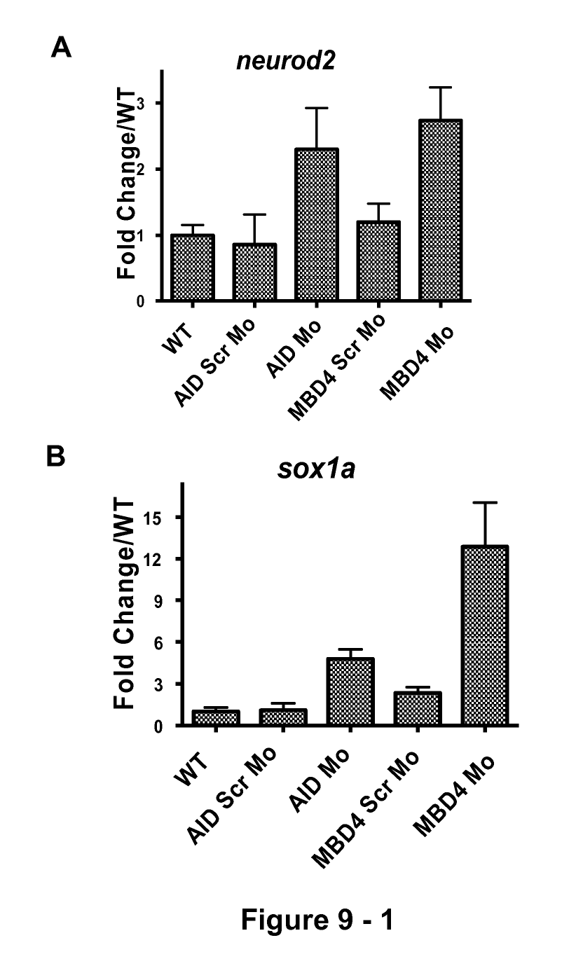

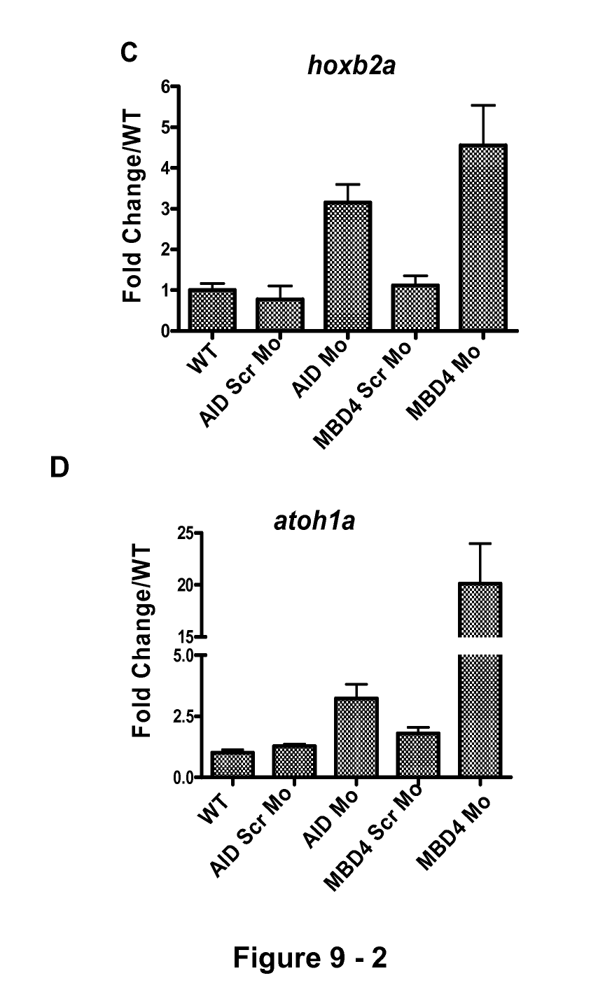

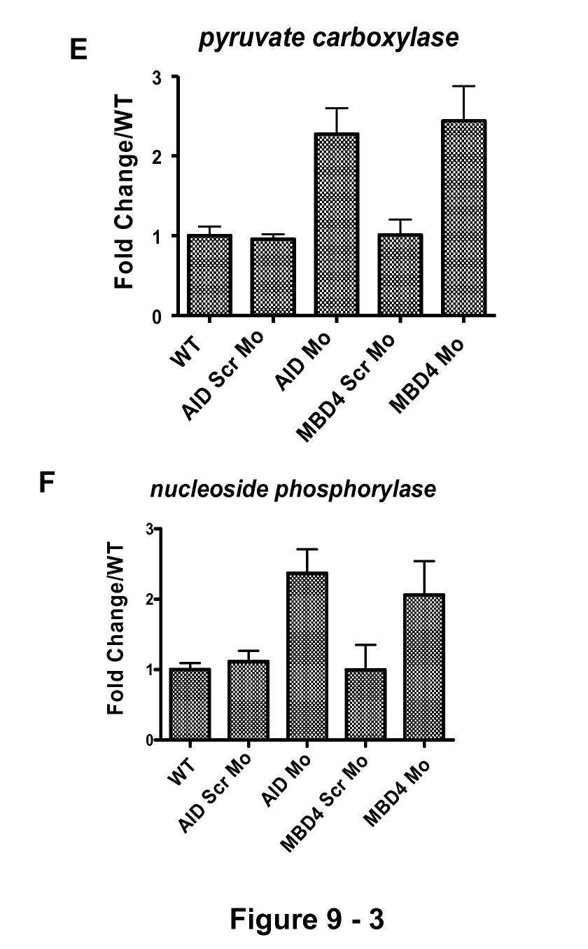

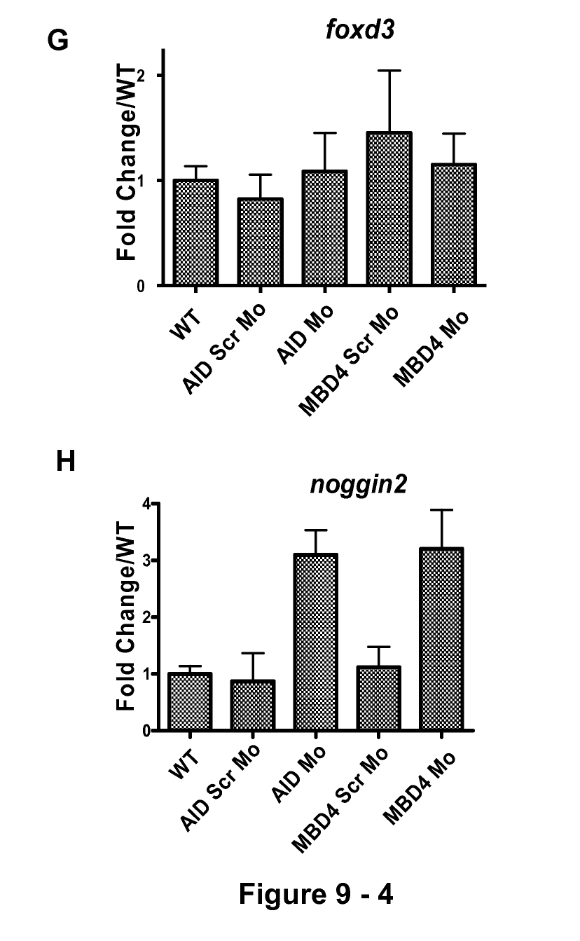

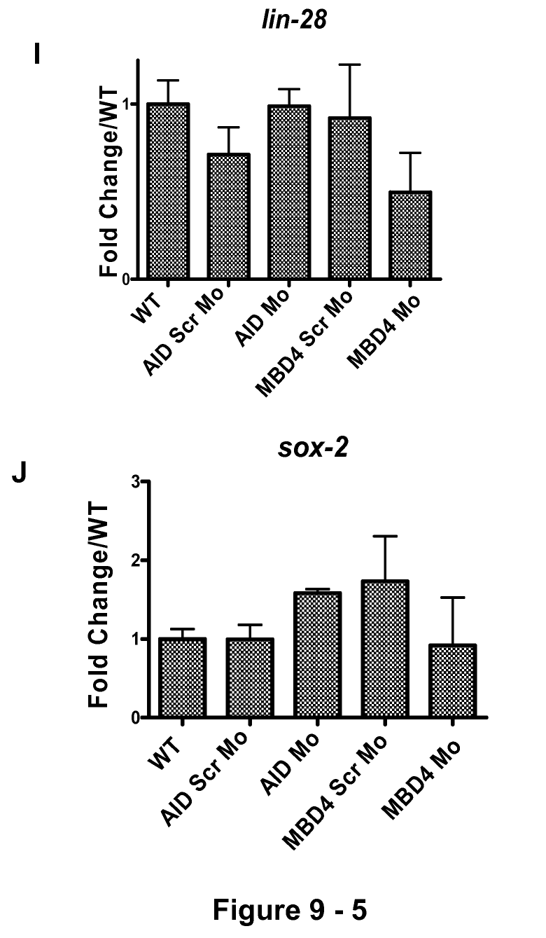

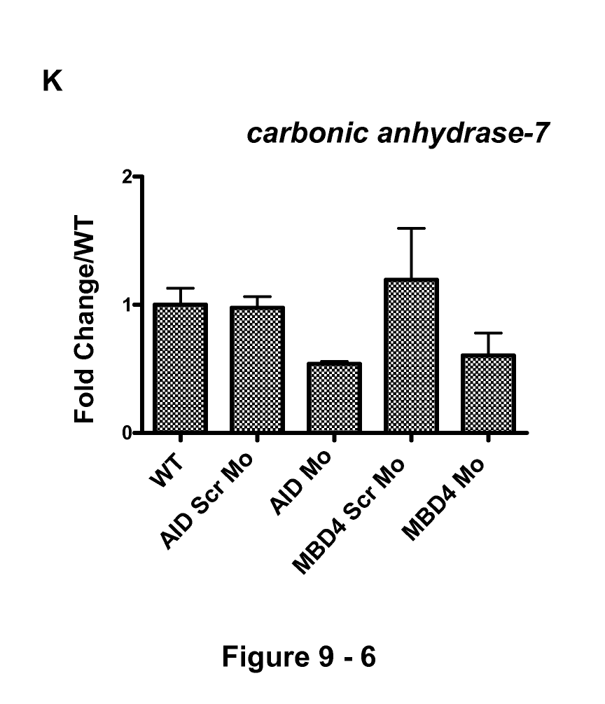

To reveal additional candidate gene targets for this demethylation system, a pilot genomic Me-DIP experiment on AID morphants at 24 hpf was performed. This data set provided candidate affected and unaffected loci, a small portion of which were examined further for methylation differences by qPCR and bisulphite sequencing. These candidates were tested at 80% epiboly, the latest time where AID morphants and control morphants are indistinguishable. These approaches confirmed an additional transcription factor important for neurogenesis (Sox1 a), as well as additional transcription factors and housekeeping genes (FIG. 9). Loci initially identified as highly impacted by the AID morpholino were also highly affected by the Mbd4 morpholino, but not by a control morpholino. Taken together, evidence for a demethylase system that functions at multiple genes during development is provided.

For example, FIG. 9 shows the identification of gene targets of AID and MBD4 by methylated DNA immunoprecipitation (Me-DIP). Genomic DNA was prepared from wild type embryos or those injected (at 80% epiboly) with AID scr mo (sequence scrambled control morpholino; 2 pg), AID morpholino (2 pg), MBD4 scr mo (sequence scrambled control morpholino; 2 pg), or MBD4 morpholino (2 pg). The DNA was immunoprecipitated using an antibody directed against 5-methylcytosine and subsequently PCR analysis was performed for multiple genes including (A) neurod2 (˜200 bp upstream of TSS), (B) sox1a (˜450 bp downstream of TSS), (C) hoxb2a (˜3700 bp upstream of TSS), (D) atoh1a (˜350 bp upstream of TSS), (E) pyruvate carboxylase (˜4800 bp upstream of TSS), (F) nucleoside phosphorylase (˜300 bp upstream of TSS), (G) noggin2 (˜500 bp downstream of TSS), (H) foxd3 (˜10 bp upstream of TSS), (I) sox2 (˜3350 bp upstream of TSS), (J) lin-28 (˜400 bp upstream of TSS), and (K) carbonic anhydrase 7 (˜50 bp upstream of TSS). The Y-axis shows enrichment at these loci relative to a control neurod2 locus. Of the loci tested neurod2, sox1a, hoxb2a, atoh1a, pyruvate carboxylase, nucleoside phosphorylase, and noggin2 promoters/genes showed selective enrichment, whereas foxd3, sox2, lin-28 and carbonic anhydrase 7 did not. Several of these loci were verified by bisulphite sequencing. Graph shows one representative biological experiment (three biological repeats), with the average of two technical replicates shown.

Genomic DNA prepared from wild type embryos or those injected (at 80% epiboly) with AID Scr Mo (sequence scrambled control morpholino; 2 pg) or AID morpholino (2 pg) were subjected to bisulphite conversion and subsequent PCR amplification, cloning and sequencing. Corresponding to the Me-DIP data (FIG. 9), sox1a, hoxb2a, atoh1a, and pyruvate carboxylase showed hypermethylation in AID morphants compared to wild type or control morpholino injected embryos, whereas foxd3, sox2, lin-28 and carbonic anhydrase 7 remained unmethylated. Primer information for the PCR is provided in Table 2.

TABLE 2
Primer Sequence Information.

| | Forward Primer (5′ to 3′) | Reverse Primer (5′ to 3′) |
|---|---|---|
| RT-PCR | | |
| AID | agtgtgctcatgacccaga | cacaacgcacccaagtga |
| | (SEQ ID NO. 31) | (SEQ ID NO. 32) |
| Apobec2a | atggccgatagaaagggc | cctgtgatggtctcgaatg |
| | (SEQ ID NO. 33) | (SEQ ID NO. 34) |
| Apobec2b | atggcagacaaaaaggacag | Cggtctcctacaatgatctc |
| | (SEQ ID NO. 35) | (SEQ ID NO. 36) |
| MBD4 | aagagacgctcttccatga | ttatgatcgtctggagtgac |
| | (SEQ ID NO. 37) | (SEQ ID NO. 38) |
| TDG | Atggatgaaaggctgtatggatc | tcctctggatgtacaggcat |
| | (SEQ ID NO. 39) | (SEQ ID NO. 40) |
| Gadd45α | atgacttttgaagaaccgtgtgg | gatctggagggccacat |
| | (SEQ ID NO. 41) | (SEQ ID NO. 42) |
| Gadd45αlike | tccgttctggattttacatgc | acccgattttgggtttcagt |
| | (SEQ ID NO. 43) | (SEQ ID NO. 44) |
| Gadd45β | Tctcacagtcggcgtttatg | cggctctcctcacagtaggt |
| | (SEQ ID NO. 45) | (SEQ ID NO. 46) |
| Gadd45γ | caacgacatcaacatcgttcg | tcagcgttcaggcagagtaa |
| | (SEQ ID NO. 47) | (SEQ ID NO. 48) |
| M-DNA | atggaagacgccaaaaacataa | cgtgatggaatggaacaaca |
| | (SEQ ID NO. 49) | (SEQ ID NO. 50) |
| Bisulfite | | |
| Line-1 | tttaattagatggtagtttttatatt | aacaaaccaacctaaaatataaa |
| | (SEQ ID NO. 51) | (SEQ ID NO. 52) |
| | ggttggagatggtttatttt | ttcctaaaccacaaatataaacat |
| | (SEQ ID NO. 53) | (SEQ ID NO. 54) |
| M-DNA | atggaagatgttaaaaatataaa | taataaaataaaacaacacttaaaat |
| | (SEQ ID NO. 55) | (SEQ ID NO. 56) |
| | gatgttaaaaatataaagaaaggt | aaacaacacttaaaatcacaatat |
| | (nested) (SEQ ID NO. 57) | (nested) (SEQ ID NO. 58) |
| KenoDr1 | Gtttgtattgaattattggt | aaaatcattttccttaaaaatcaa |
| | (SEQ ID NO. 59) | (SEQ ID NO. 60) |
| | gagaagggataaatggattat | aaatcaactctaatccctcta |
| | (nested) (SEQ ID NO. 61) | (nested) (SEQ ID NO. 62) |
| Neurod2 | gtttttaaataggtataggt | caaacaaaattacataccta |
| | (SEQ ID NO. 63) | (SEQ ID NO. 64) |
| | aggtataggttaggttatgt | catacctactcttataccaaa |
| | (nested) (SEQ ID NO. 65) | (nested) (SEQ ID NO. 66) |
| Sox1 a | ttttgtttaaaaaggataagtat | aatacatactaatcatctct |
| | (SEQ ID NO. 67) | (SEQ ID NO. 68) |
| | gttagaggttggagagttt | taatcatctctctcaaatctc |
| | (nested) (SEQ ID NO. 69) | (nested) (SEQ ID NO. 70) |
| Hoxb2a | ggataagtttttttatggttagt | aaacttcctataaaaaaaaaccc |
| | (SEQ ID NO. 71) | (SEQ ID NO. 72) |
| | tggttagttttattagtggga | ccctaataataaaccaatacct |
| | (nested) (SEQ ID NO. 73) | (nested) (SEQ ID NO. 74) |
| Atoh1 a | aaagattgtttggggtaat | aattattatacccaaactcta |
| | (SEQ ID NO. 75) | (SEQ ID NO. 76) |
| | gtaattaaaaggtttttgtgtgt | aactctaccttttttccataata |
| | (nested) (SEQ ID NO. 77) | (nested) (SEQ ID NO. 78) |
| Pyruvate Carboxylase | ttggataaagtaaggaaagg | tcttaccaaatcaatattatcct |
| | (SEQ ID NO. 79) | (SEQ ID NO. 80) |
| | aaggaaagggttggttttgt | caatattatcctctctttccttc |
| | (nested) (SEQ ID NO. 81) | (nested) (SEQ ID NO. 82) |
| Foxd3 | gattttattttgggttgttttagt | ttttctcctcctcaaaaaac |
| | (SEQ ID NO. 83) | (SEQ ID NO. 84) |
| | tttgggttgttttagtagtaaag | ctcctcctcaaaaaactac |
| | (nested) (SEQ ID NO. 85) | (nested) (SEQ ID NO. 86) |
| Sox2 | ggaggataagaattataatt | tattcaacttcctataaaaaaa |
| | (SEQ ID NO. 87) | (SEQ ID NO. 88) |
| | aagagtggttgtggatttt | acctattactctacaaatatcta |
| | (nested) (SEQ ID NO. 89) | (nested) (SEQ ID NO. 90) |
| Lin-28 | gtttttgatggatagaaataa | gatggatagaaataatttgaattat |
| | (SEQ ID NO. 91) | (SEQ ID NO. 92) |
| | atataatccaaacaaaaccaa | ctcttcatataataatcaaacata |
| | (nested) (SEQ ID NO. 93) | (nested) (SEQ ID NO. 94) |
| Carbonic Anhydrase | atgagtagatttaatgatgt | caaaataccacatacaaaaa |
| | (SEQ ID NO. 95) | (SEQ ID NO. 96) |
| | ggattttattaggtgaaaaaaat | cacatacaaaaattcaccaa |
| | (nested) (SEQ ID NO. 97) | (nested) (SEQ ID NO. 98) |
| ChIP | | |
| pCMV-Luc^{pos} (w/CCGG) | agatcgtggattacgtcgc | cttggcctttatgaggatctc |
| | (SEQ ID NO. 99) | (SEQ ID NO. 100) |
| pCMV-Luc^{neg} (w/o CCGG) | cattgacgtcaatgggagtttg | ttagccagagagctctgc |
| | (SEQ ID NO. 101) | (SEQ ID NO. 102) |
| Neurod2^{pos} (P1) | atacatttgtggctgctgtgt | acatcctgacgatatatggaga |
| | (SEQ ID NO. 103) | (SEQ ID NO. 104) |
| Neurod2^{neg} (P2) | ggttgaaaatggacattctgc | gctaaacagtgctgaattagg |
| | (SEQ ID NO. 105) | (SEQ ID NO. 106) |
| MeDIP | | |
| Neurod2 | atacatttgtggctgctgtgt | acatcctgacgatatatggaga |
| | (SEQ ID NO. 107) | (SEQ ID NO. 108) |
| Sox1 a | gcatgatgatggaaacggac | ttccactcagcaccgagt |
| | (SEQ ID NO. 109) | (SEQ ID NO. 110) |
| Hoxb2a | ggaagaccttcaatcagcgt | caatgacaagaacacagcgtc |
| | (SEQ ID NO. 111) | (SEQ ID NO. 112) |
| Atoh1 a | gtgtgaagacggctgaatac | tcctcacgccatgttttgga |
| | (SEQ ID NO. 113) | (SEQ ID NO. 114) |
| Pyruvate Carboxylase | tgaatcgagtggctcgaag | catccgctctcaaggtttac |
| | (SEQ ID NO. 115) | (SEQ ID NO. 116) |
| Nucleoside Phosphorylase | ctcacatgcctttgtgtacc | attcctgaagcgtcgctct |
| | (SEQ ID NO. 117) | (SEQ ID NO. 118) |
| Noggin2 | tgtgaccaagaccttcctg | cgtgggtaaacagtgcaatc |
| | (SEQ ID NO. 119) | (SEQ ID NO. 120) |
| Foxd3 | aagccttcggactggagaa | gaccacgtcgatatccacat |
| | (SEQ ID NO. 121) | (SEQ ID NO. 122) |
| Sox-2 | tttgcacctgtacctccgaa | gaaatccacagccactcttg |
| | (SEQ ID NO. 123) | (SEQ ID NO. 124) |
| Lin-28 | gacaacctaccatattagcttgc | tcaagcatgtgcttcaacgg |
| | (SEQ ID NO. 125) | (SEQ ID NO. 126) |
| Carbonic Anhydrase 7 | cagtggatttcaccaggtga | aggtgaaggaagagcgatga |
| | (SEQ ID NO. 127) | (SEQ ID NO. 128) |
| Negative Control 1 | ggttgaaaatggacattctgc | gctaaacagtgctgaattagg |
| (neurod2^{neg} region) | (SEQ ID NO. 129) | (SEQ ID NO. 130) |
| Negative Control 2 (1 Kb | aacagagtggcaggcatcattta | acagtcatatttcttggaagacccc |
| region in Chr 1-3 CpGs) | (SEQ ID NO. 131) | (SEQ ID NO. 132) |
| Negative Control 3 (1 Kb | ctgaactttcatcaaattggtttcc | gtgatggcaaacttaaagtccttca |
| region in Chr 14-no CpGs) | (SEQ ID NO. 133) | (SEQ ID NO. 134) |
| C to T detection | | |
| (CCGG position) | | |
| For. Strand 32 | ccaaaaacataaagaaaggccca | atatccttgcctgatacctg |
| | (SEQ ID NO. 135) | (SEQ ID NO. 136) |
| For. Strand 272 | aaactctcttcaattctttatgcca | atatccttgcctgatacctg |
| | (SEQ ID NO. 137) | (SEQ ID NO. 138) |
| For. Strand 522 | acatctcatctacctccca | atatccttgcctgatacctg |
| | (SEQ ID NO. 139) | (SEQ ID NO. 140) |
| Rev. Strand 272 | atggaagacgccaaaaacataa | aaataacgcgcccaacacca |
| | (SEQ ID NO. 141) | (SEQ ID NO. 142) |
| Rev. Strand 522 | atggaagacgccaaaaacataa | tggcacaaaatcgtattcattaaaacca |
| | (SEQ ID NO. 143) | (SEQ ID NO. 144) |
| Rev. Strand 698 | atggaagacgccaaaaacataa | caacacttaaaatcgcagtatcca |
| | (SEQ ID NO. 145) | (SEQ ID NO. 146) |

Evidence is accumulating for an active DNA demethylation process in vertebrates, including studies on the activation of the interleukin-2 locus (Bruniquel and Schwartz, 2003), active demethylation following fertilization (Mayer et al., 2000; Oswald et al., 2000; Reik, 2007), and recent evidence for cyclical DNA demethylation at ERo targets (Kangaspeska et al., 2008; Metivier et al., 2008). However, candidate enzymes for active demethylation have remained elusive or controversial.

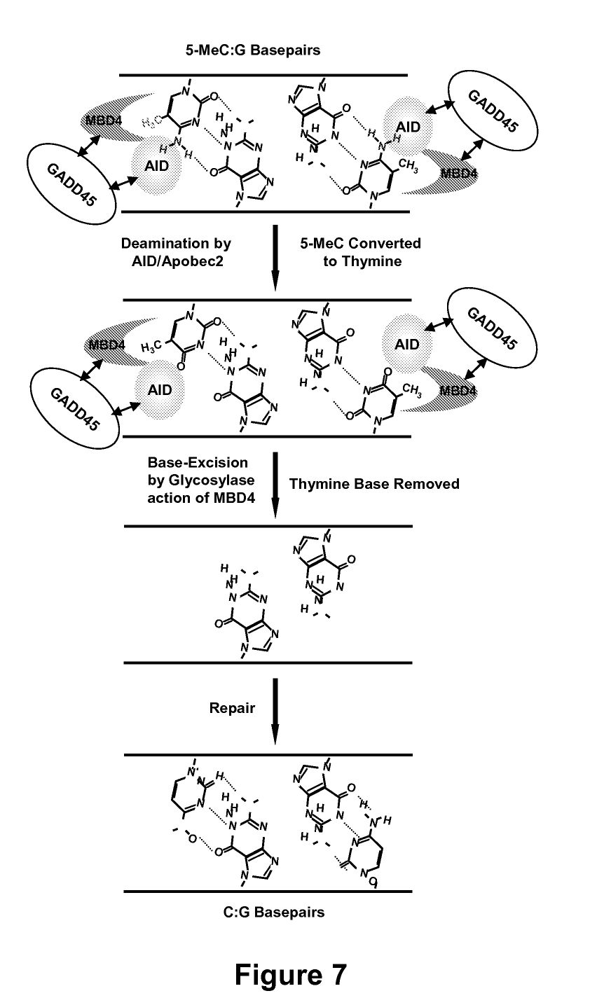

Here, this identification of demethylase candidates was enabled by a versatile zebrafish embryo assay system, which allowed for eliciting and monitoring demethylation, and in which overexpression or knockdown allowed for the evaluation of candidates. This work provides multiple lines of evidence that demethylation involves a coordinated system involving at least one or more of three factors: an AID/Apobec deaminase, an Mbd4-related G:T glycosylase, and a Gadd45 family member (for example, see a proposed mechanism in FIG. 7). First, the demethylation of an injected DNA fragment (or plasmid), along with 20%-40% of the 5meC nucleotides in the zebrafish genome, was temporally correlated with the upregulation of Gadd45 and AID/Apobec members. Morpholino knockdown studies showed a reliance on these factors for demethylation. Although zMbd4 was not upregulated by M-DNA injection, there are high levels of the RNA encoding Mbd4 in the early embryo (FIG. 8), and Mbd4 protein can be activated following M-DNA injection in a posttranscriptional manner. Evidence that these enzymes conduct demethylation in the normal developing embryo was provided by examining the AAAmo (which lowers AID and eliminates Apobec members), which caused 12% increase in bulk methylation levels, a result comparable to the observation in Arabidopsis mutants lacking all three major 5-meC glycosylases, which likewise show only a modest upregulation of bulk 5-meC levels (Lister et al., 2008; Penterman et al., 2007). Furthermore, AID and hMbd4 were localized to methylated regions on a plasmid (FIG. 5), and also to a gene (neurod2) that is related to a phenotype (loss of neurons) observed in AID and Mbd4 morphants (FIG. 6B). These are findings that indicate roles in vivo at genes. The list of gene targets by examining candidates revealed in a pilot genomic Me-DIP study in AID morphants was expanded, which revealed transcription factors and housekeeping genes that relied on AID, and on Mbd4, for their demethylated status (at 80% epiboly).

Overexpression of AID/Apobec along with hMbd4, but not either alone, caused significant demethylation of the bulk genome (FIG. 2). The magnitude of this reduction (20%-40%) is pronounced, as studies in zebrafish and mice indicate that perturbations beyond these levels are not compatible with viability (Ooi and Bestor, 2008; Rai et al., 2006). AID/Apobec expression alone did not prevent MspI cleavage, indicating that AID/Apobec activity is promoted by Mbd4 and/or another cofactor (such as Gadd45) at 5-meC sites. Here, physical association of AID/Apobec with Mbd4 helps prevent the persistence of mutagenic G:T intermediates, as Mbd4 rapidly removed the thymine. Indeed, the ability to detect a G:T intermediate (using a PCR priming strategy) only in hMbd4 catalytic mutants supports the reaction mechanism described herein, while also underscoring the importance for proper regulation (FIG. 3). Coexpression of a catalytically inactive version of hMbd4 (along with AID) elicited a small number of 5-meC>T transitions in the injected M-DNA fragment, but not other mutations. By analogy, misregulation of demethylation might underlie the preponderance of C>T transitions in certain genes linked to cancer in mammals. Although mice lacking Mbd4 are viable, they show a higher frequency of mutations at CpG sites (Millar et al., 2002; Wong et al., 2002).

Two recent studies clearly showed cyclical demethylation (2 hr cycles of methylation and demethylation) at ERo targets (Kangaspeska et al., 2008; Metivier et al., 2008), and one study (Metivier et al., 2008) suggested a related stepwise deamination/glycosylase mechanism for demethylation that utilized different enzymes and deamination chemistry than the present mechanism. One counterintuitive aspect of this work was the proposed use of DNMT3 both to methylate cytosine and to deaminate 5-meC, relying on an inefficient deaminase activity (displayed in vitro) that deaminates 5-meC only under conditions of low S-adenosylmethionine (SAM). Although plausible, it is unclear how SAM levels are held low in vivo, or how the low turnover number accounts for the demethylation kinetics. In counter distinction, the mechanism described here employs a dedicated deaminase family (AID/Apobec).

FIG. 7 shows that demethylation can occur through a two-step coupled enzymatic process, promoted by Gadd45. The first enzymatic step involves deamination of 5-meC by AID (amine group removed, in blue), generating a thymine product and a G:T mismatch. The second step involves thymine base removal by Mdb4, generating an abasic site. As the transient G:T intermediate is not detected in cells with active Mbd4, but is with catalytically inactive Mbd4, the thymine is likely rapidly removed, suggesting a coupling between deaminase and glycosylase activity. Gadd45 promotes functional or physical interactions between AID and Mbd4 at the site of demethylation. Mbd4 couples with a lyase to help promote base replacement through base excision repair (neither shown nor addressed). The recognition of the 5-meCpG (methyl group in red), or through another mechanisms, promotes the targeting of AID/Mbd4.

Here, new roles are indicated for Gadd45 family members in regulating DNA demethylation. First, as observed in human cells, Gadd45α (and α-like) overexpression promotes moderate DNA demethylation (FIG. 4). Furthermore, Gadd45 family members appeared important in, but also redundant for, promoting DNA demethylation elicited by M-DNA injection. Particular Gadd45 members upregulated the transcription of specific AID/Apobec enzymes, suggesting possible partnerships. Physical interactions between Gadd45α and both AID/Apobec and Mbd4 occurred, raising the possibility that Gadd45 might couple these two enzymes physically and/or activate them functionally, either alone or through known interactions with kinases downstream of Gadd45. Taken together, these results argue for a regulated demethylase system that involves enzyme and regulator families functioning in a coordinated manner.

### Example 2

### DNA Demethylase Activity Maintains Zebrafish Intestinal Cells in a Progenitor-Like State Following Loss of APC

### Zebrafish Care and Injections

Wild type (Tu strain) and apc heterozygous adults were maintained in Z-Mods at 28° C. in a 14:10 hour light cycle. For all whole mount in situ experiments embryos were raised in 0.003% phenylthiourea (PTU) treated embryo water to inhibit pigment formation. Morpholinos (Gene-tools), DNA and RNA were injected at 1-cell stage.

### Drug Treatments

Embryos were treated with all-trans retinoic acid (1 μM) or DMSO (vehicle) every day starting at 24 hpf for 45 min. NS-398 treatment was similar except at 10 μM. For pargyline treatment, drug (3 mM or water control) was left in embryo water starting at 75% epiboly and new drug added every day. Cells were treated with pargyline at 3 mM or all-trans retinoic acid (or DMSO) at 5 μM each day for two days and RNA was isolated.

### MeDIP (Methylated DNA Immunoprecipitation), ChIP and Array Hybridization

This procedure was described in Weber et al., 2007. Briefly, Dynabeads (conjugated with sheep anti-mouse secondary antibody, Invitrogen) were incubated with 10 μg of 5MeC antibody (Eurogentec) for 2 hrs, washed, and then incubated with 4 μg of sonicated DNA (300 bp-1000 bp fragments) overnight Immunoprecipitated DNA was amplified using for whole genome amplification kit (Sigma Aldrich). Following amplification, 2 μg of amplified DNA was labeled with Cy5, and 2 μg of input DNA was labeled with Cy3 (Bio labs). Samples were competitively hybridized to 244K oligonucleotide promoter arrays (Agilent Inc).

### Chromatin Immunoprecipitation

ChIP was performed as described in Rai et al., 2008. ChIP for Cebpβ and Pou5f1 were performed in wild type (Tu) embryos. V5-zfCEBPβ (along with pou5f1 morpholino, 80 pg) or V5-zfPou5f1 (both cloned in pcDNA3.1/nV5 DEST; Invitrogen) was injected at one cell stage and embryos collected at 48hpf. ChIP was performed using V5 antibody (Abcam). ChIP for Lef1 was performed using rabbit-anti-zfLef1 antibody in extracts made from apc^{wt} and apc^{mcr} embryos collected at 80 hpf. ChIP in SW-480 cells were performed using antibodies α-CtBP (Santa Cruz; sc-11390), α-LSD-1 (Abcam; ab-17721) and rabbit IgG (control).

### Co-Immunoprecipitation

Constructs expressing tagged proteins were transfected in SW480 cells and protein harvested in 1× IPH buffer 48 hrs after transfection Immunoprecipitation was performed as described in Rai et al., 2008 using rabbit-α-V5 antibody (Abcam) or α-Myc monoclonal antibody. Western blot was probed using mouse-α-V5 (Invitrogen) and mouse-α-GFP (BD Biosciences) or rabbit-α-myc (Santa Cruz) and rabbit-α-V5 (Abcam) antibodies respectively.

### MeDIP-CHIP Data Analysis

Statistical Analysis of the MeDIP-chip data was conducted using the SLAM software (http://statistics.byu.edu/johnson/SLAM/). SLAM (GausSian non-Linear Analysis of MeDIP-chip data) estimates DNA methylation levels from two-color MeDIP-chip assays. The SLAM algorithm combines MeDIP-chip specific normalization, Gaussian dynamic smoothing, finite mixture modeling, and a Probit transformation to provide robust and accurate estimates of methylation levels across the genome. The MeDIP-chip specific normalization method adjusts for biases due to slide and probe effects such as GC and CpG di-nucleotide content. In addition, SLAM uses a Gaussian dynamic linear model to combine to neighboring probes. A two-state (methylated or not) finite normal mixture model followed by transformation using the cumulative distribution of the normal distribution (Probit transformation) is applied to estimate the methylation status of each probe and to provide a accurate estimates for the percentage of methylation in the given region. SLAM compares the methylation level of two samples, and determines the statistical significance of differentially methylated regions.

### Tissue Samples and Immunostaining

Human tissue samples were biopsy samples taken from FAP patients. Tissues were sectioned in 5 micron size by a Leica microtome and stained as described in Nadauld et al., 2004.

### Morpholinos

For these experiments, morpholino sequences were designed against the following:

(SEQ ID NO. 14)
Pou5f 1 Mo: 5′-CGCTCTCTCCGTCATCTTTCCGCTA-3′;
(SEQ ID NO. 15)
Pitx2 Mo: 5′-TTTATCAAACTTACTCGGACTCTGG-3′;
and
(SEQ ID NO. 16)
Tdg Mo: 5′-AGCCCTTCTT TGTTTACCTGTCTGC-3′.

The morpholino sequences for Aid Mo, Apobec2a Mo, Apobec2b Mo, Mbd4 Mo were previously in Rai et al., 2008. Similarly, Cebpβ Mo and CtBP1 Mo are described in Eisinger et al., 2006 and Nadauld et al., 2006, which are hereby incorporated by reference in their entireties into this application. Other morpholinos used are as follows:
Lef 1 morpholino (Li et al., 2006) (hereby incorporated by reference in its entirety into this application);
Cox2 morpholino (Eisinger et al., 2006) (hereby incorporated by reference in its entirety into this application);

Groucho2 morpholino
(SEQ ID NO. 17)
(5′-GCCCTGTGGATACATCTTGAAATGT-3′);
Lsd 1 morpholino
(SEQ ID NO. 18)
(5′-GGTCTGACTTCTTATTGGACAACAT-3′);
Corest morpholino
(SEQ ID NO. 19)
(5′-CCTTCTCTAACATTGCGGGCATCTT-3′);
and
Control morpholino
(SEQ ID NO. 20)
(5′-CCTCTTACCTCAGTTACAATTTATA-3′).

The Lef1 morpholino, the Cox2 morpholino, the Groucho2 morpholino, the Corest morpholino, and the control morpholino were solubilized in 1× Danieau buffer. Morpholinos were injected at the 1-cell stage using 1 nL of the final concentration for each morpholino. The concentrations used in apc^{mcr} zebrafish embryos were Lef 1 (0.1 mM), Groucho2 (0.25 mM), Cox2 (0.25 mM), Lsd1 (0.25 mM), and Corest (0.75 mM). Control injections were done using control morpholino at similar final concentration. Zebrafish Groucho2 (Accession Number: NM_{—}1 31012) and Groucho3 (Accession Number NM_{—}131780) were cloned in pCRII-TOPO (Invitrogen). For RNA injections, full length Groucho2, and Lef 1 RNA were transcribed from linearized pCRIITOPO/groucho2, pCSMTlef 1, and pCSMT-dominant-negative-Lef 1 construct using mMessage Machine kit (Roche Applied Science) according to manufacturer's protocol. For injections, 50 pg of groucho2 and lef1 mRNA was injected into one-cell stage wild type zebrafish embryos.

### Quantitative RT-PCR

Quantitative RT-PCR primers for amplification of zebrafish rdh1l (Nadauld et al., 2005), human DHRS9 (Jette et al., 2004) 28S rRNA (Eisinger et al., 2006), and h18S rRNA (Delaunay et al., 2000) were described previously, and are (hereby incorporated by reference in its entirety into this application). A template-free negative control was included in each experiment.

Cell Culture and siRNA Transfection

HT29, DLD1, and SW480 cells were cultured as recommended by the American Type Culture Collection. For silencing of TLE3, the sequence of the small interfering RNAs (siRNAs) were as follows:

(SEQ ID NO. 21)
sense, 5′-GCCGAUACGACAGUGAUGGAGACAA-3′;
and
(SEQ ID NO. 22)
antisense, 5′-UUGUCUCCAUCACUGUCGUAUCGGC-3′.

For silencing of LEF1, the sequences of the siRNAs were as follows:

(SEQ ID NO. 23)
sense, 5′-CACCUCAGGUCAAACAGGAdTdT-3′;
and
(SEQ ID NO. 24)
antisense, 5′-UCCUGUUUGACCUGAGGUGdTdT-3′.

For silencing of COREST, the sequence of the small interfering RNAs (siRNA1) was as follows:

(SEQ ID NO. 25)
sense, 5′-GGAAUUGGUUUCAGUCAAAdTdT-3′;
and
(SEQ ID NO. 26)
antisense, 5′-TTTGACTGAAACCAATTCCdTdT-3′;

For silencing of COREST, the sequence of the siRNAs (siRNA2) was as follows:

(SEQ ID NO. 27)
sense, 5′-CGACGCCGCUUCAACAUAGdTdT-3′;
and
(SEQ ID NO. 28)
antisense, 5′-CTATGTTGAAGCGGCGTCGdTdT-3′.

The sequence for the control siRNA was as follows:

(SEQ ID NO. 29)
sense, 5′-AGACAGAAGACAGAAGCdTdT-3′;
and
(SEQ ID NO. 30)
antisense, 5′-GCCUAUCUGUCUCGCUdTdT-3′.

For silencing of LSD1, the sequences of the siRNAs were taken from Shi et al. 2004.

Transfection experiments were performed using Lipofectamine 2000 (Invitrogen) according to the manufacturer's recommendations. Following transfection with 100 nM or 200 nM siRNA, cells were incubated for 72 hrs and then harvested for Western blotting analysis or RT-PCR.

### Antibodies

The following primary antibodies were used: rabbit-anti-Tle3 (Santa Cruz), rabbit-anti-lef 1 for zebrafish (a generous gift from Richard Dorsky), mouse-anti-3-actin (Novus Biologicals), mouse-anti-vinculin (Sigma), mouse-anti-GFP (BD Biosciences), rabbit-anti-Groucho2 (Sigma) and mouse anti-V5 antibody (Invitrogen). Lsd-1 for zebrafish was detected using an antibody that was generated against S. pombe LSD1.

### Whole Mount In Situ Hybridization

Whole mount in situ hybridizations were performed as described in Nadauld et al., 2004. Riboprobes for the indicated genes were made using digoxigenin labeled UTPs (Roche). Genes were cloned in pCR-II-TOPO vector (Invitrogen).

### Colon Cancer Cell Line Culture and Transfection

SW480, DLD1 and HT29 cells were purchased from ATCC and grown as per guidelines of manufacturer. Transfection experiments were performed using Lipofectamine 2000 (Invitrogen) according to the manufacturer's recommendations. Following transfection, cells were incubated for 72 hrs (siRNA) or 24 hrs (DNA) and then harvested for Western blotting analysis or RT-PCR.

### RT-PCR

RNA from embryos or human adenoma tissues was isolated using Trizol (Invitrogen). cDNA library was prepared using Superscript III kit (Invitrogen). RT-PCR was performed on a Roche light cycler.

### Western Blotting

Protein extracts were made and western blotting performed as described in Rai et al., 2008.

Loss of APC Upregulates Demethylase Components and Correlates with Demethylation of Key Target Genes

As provided above in Example I, a mechanism for active DNA demethylation in zebrafish involving the cooperative actions of proteins from the cytidine deaminase family (activation induced deaminase (Aid), and Apobec2), the G:T mismatch specific glycosylase family (MBD4), and a DNA repair protein family (Gadd45) was described. To build upon the teachings of Example I, a partially redundant role in demethylation for the Mbd4 paralog Tdg (thymine glycosylase) was observed. Given the potential of DNA hypomethylation as an early event in adenoma initiation following loss of APC, the demethylase components in zebrafish embryos harboring homozygous apc mutations (apc^{mcr}) were examined. Whole mount in situ hybridization revealed the robust upregulation of components of the DNA demethylase system including aid, apobec2a, apobec2b, mbd4, gadd45α, gadd45αlike, gadd45β, and gadd45γ, and a slight upregulation of tdg in apc mutants (apc^{mcr}) and siblings (apc^{wt}) zebrafish embryos at 72 hpf).

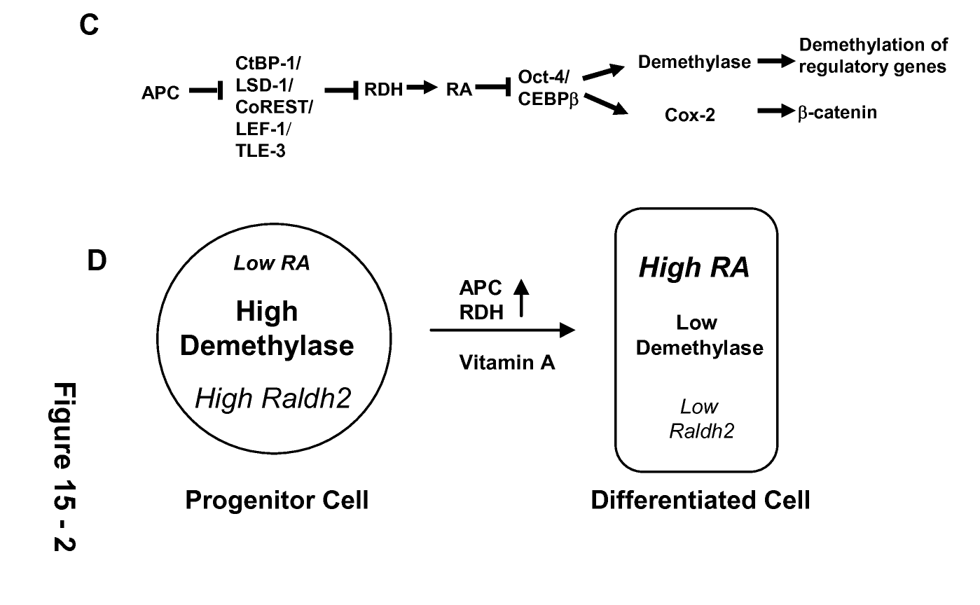

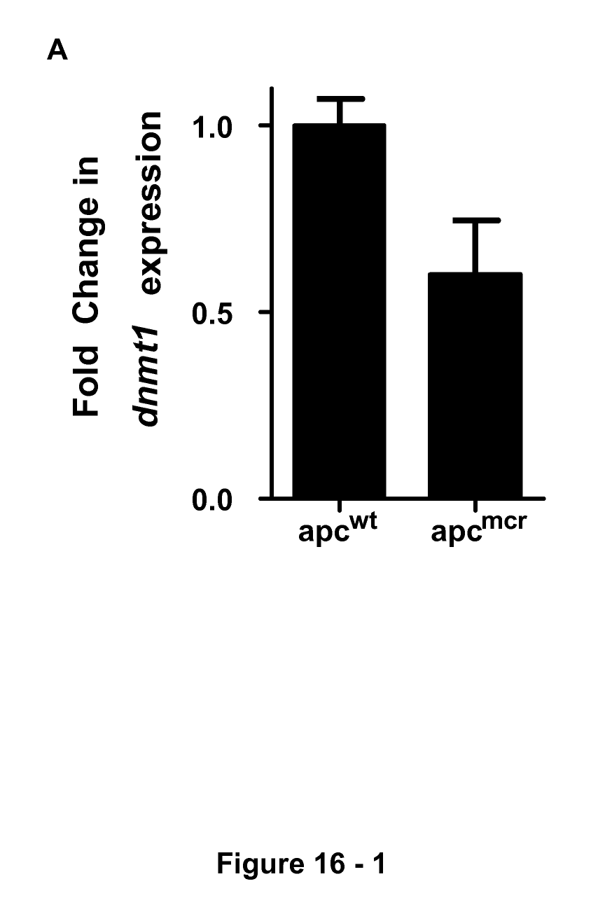

Table 3 shows the expression of demethylase genes and Dnmt1 in APC mutants and siblings. At 72 hpf, there was increased expression in the eyes, brain and intestine relative to control wild type sibling embryos (apc^{wt}). In addition, homozygous apc mutant embryos show greatly reduced dnmt 1 mRNA expression (FIG. 16A). Whole mount in situ expression analysis for aid, mbd4 and gadd45α in apc mutants (apc^{mcr}) and siblings (apc^{wt}) embryos at 72 hpf. The dorsal view is shown and the arrows refer to intestine. FIG. 16A shows RT-PCR measuring dnmt1 levels in apc mutants (apc^{mcr}) and siblings (apc^{wt}). The Y-axis shows fold change normalized to 28S levels first and then to dnmt1/28S ratio from apc^{wt}, valued as 1. Western blots showed Dnmt1 levels in apc mutants (apc^{mcr}) and siblings (apc^{wt}) injected with V5-Dnmt1 plasmid (0.1 pg). Therefore, examination of both Dnmt1 and the demethylase system on the impact on cell fating and DNA methylation status was undertaken.

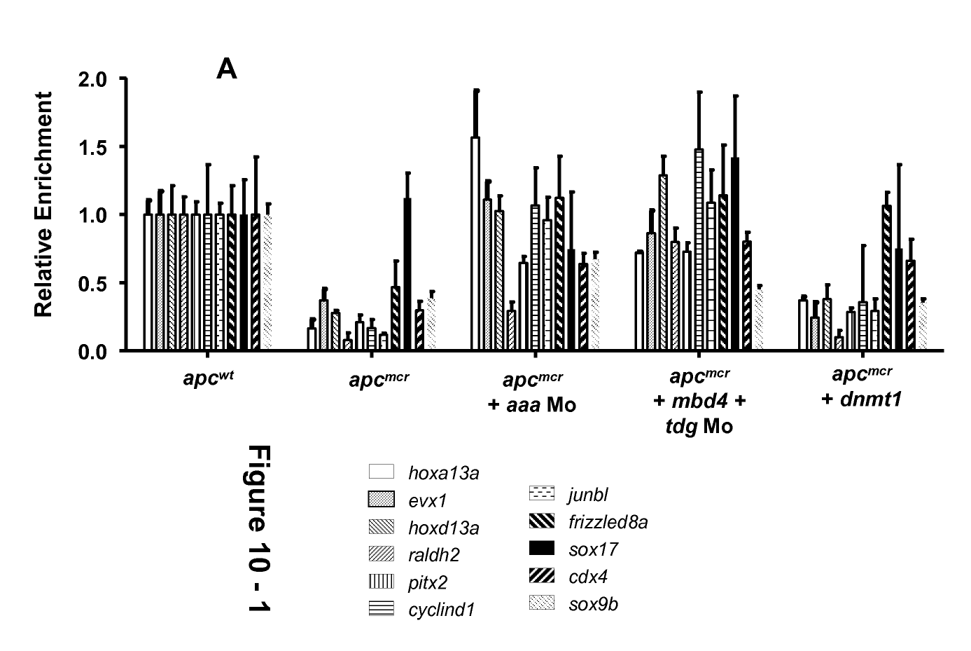

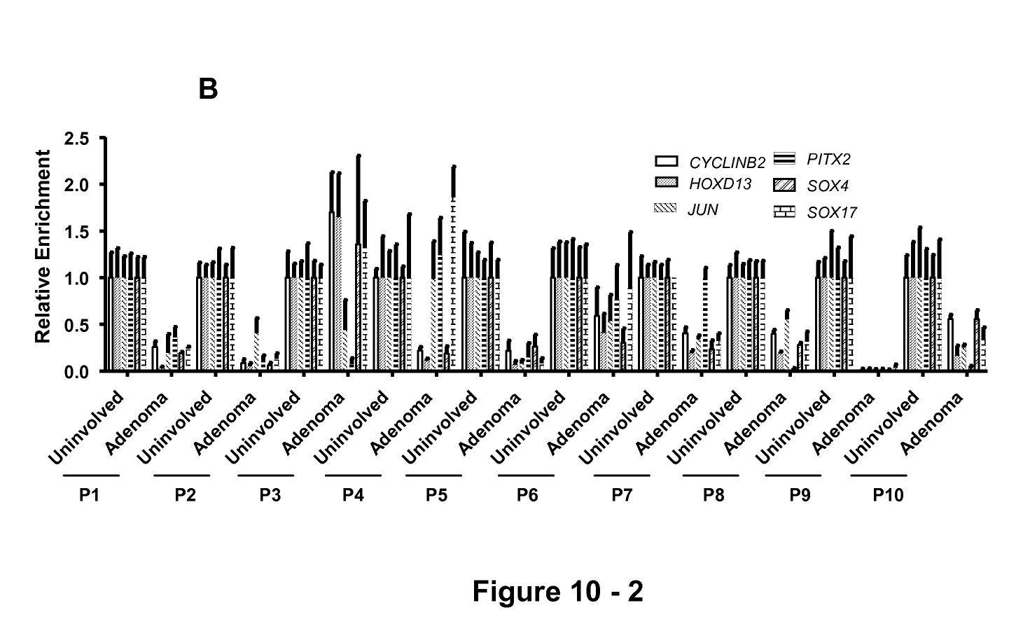

Given the strong induction of the demethylase components in apc mutant zebrafish embryos, bulk levels of DNA methylation were tested. Significant changes were not observed. However, as bulk determinations monitor repetitive heterochromatin and transposons, genome-wide changes in DNA methylation at promoters was surveyed instead by methylated DNA immunoprecipitation (MeDIP, (Weber et al., 2007)), which uses an antibody directed against 5-methyl-cytosine (5meC), followed by hybridization to Agilent 244K oligonucleotide arrays that tile the majority of zebrafish gene promoters (−9 to +2 kb). This analysis uncovered hypomethylation on a number of genes that have been implicated in intestinal fating and development of colorectal cancer including aldh1a2, hoxa13a, evx1, pitx2, cyclind1, hoxd13a, junb1, frizzled8a, cdx4, sox9b, cyclinb2, and sox4. Verification of methylation status for a subset was conducted by qPCR of MeDIP samples (FIG. 10A) or bisulfite sequencing.

TABLE 3
Expression of Demethylase Genes and Dnmt1 in APC Mutants and Siblings

| | apc^{wt} | apc^{mcr} |
|---|---|---|
| aid | 90% | 4% |
| | (n = 40) | (n = 51) |
| apobec2a | 99% | 7% |
| | (n = 80) | (n = 38) |
| apobec2b | 98% | 9% |
| | (n = 86) | (n = 42) |
| gadd45α | 96% | 4% |
| | (n = 73) | (n = 49) |
| gadd45β | 97% | 6% |
| | (n = 72) | (n = 36) |
| gaddγ | 98% | 6% |
| | (n = 62) | (n = 34) |
| gadd45α like | 96% | 6% |
| | (n = 70) | (n = 35) |
| mbd4 | 95% | 4% |
| | (n = 79) | (n = 47) |
| tdg | 96% | 12% |
| | (n = 68) | (n = 25) |
| dnmt1 | 96% | 7% |
| | (n = 130) | (n = 47) |

To test whether the demethylation of these promoters resulted from the upregulation of the demethylase, the demethylase components were knocked down using morpholino antisense oligonucleotides. In FIG. 10A, the morpholinos were either aaa Mo (combination of aid, apobec2a and apobec2b morpholinos; 0.5 ng each), or mbd4 and tdg morpholinos together (1 ng each), or V5-Dnmt1 expressing plasmid (1 pg). Following injection, hypomethylation was reversed for most of the genes tested (FIG. 10A). Overexpression of dnmt1 (via mRNA injection at the 1-cell stage, protein abundance) largely failed to restore promoter methylation (FIG. 10A). In both FIGS. 10A and 10B, the Y-axis shows values for each promoter region normalized to a negative control region lacking CpGs, and then normalized to the values from wild type or uninvolved, valued at 1.

The conservation of the above findings in human colon adenomas obtained from FAP patients was examined. Consistent with the studies in zebrafish embryos, AID, MBD4 and GADD45α were significantly upregulated in at least six out of ten adenoma samples obtained from FAP patients which harbor germline APC mutations.

Next, genome-wide changes in DNA methylation were determined in adenoma samples from two different FAP patients by comparing the adenoma sample to normal appearing colonic tissue using MeDIP and hybridization to Agilent 244K oligonucleotide human promoter arrays. This analysis revealed that many promoters with altered methylation in apc mutant zebrafish also showed changes in FAP adenoma tissues.

A subset of these regions were tested and verified by qPCR from MeDIP performed in FAP adenoma tissue samples from 10 different patients. The promoter regions of CYCLINB2, HOXD13, JUN, PITX2, SOX4 and SOX17 showed demethylation (FIG. 10B; P1-P10 refers to ten different patients), and showed demethylation of a key factor for subsequent experiments in zebrafish. PITX2 promoter hypomethylation was verified by bisulfite sequencing in adenoma samples from five different patients. Changes in methylation were seen in the following genes: AAMP, ABCD2, ABHD11, ACAA1, ACAD11, ACAP3, ACD, ACHE, ACTR1A, ACYP1, ADAMTS4, ADAT2, ADCK4, ADCY4, ADHFE1, ADPRHL2, AGER, AGFG1, AGPAT1, AGRP, AHI1, AIF1, AK3L1, AK5, AKAP3, AKR7A2, ALDH6A1, ALDOC, ALG2, ALG3, AMIGO3, ANAPC10, ANAPC13, ANKHD1-EIF4EBP3, ANKMY1, ANKMY2, ANKRA2, ANKRD16, ANO8, AP4B1, APBB3, APOA2, APTX, AQP10, AQP12B, AQP4, ARAP1, ARF1, ARFRP1, ARHGAP26, ARHGAP30, ARHGEF11, ARHGEF4, ARHGEF7, ARID1A, ARID1B, ARID3C, ARID4A, ARID4B, ARL13B, ARL3, ARL4A, ARL6, ARL61P6, ARMC8, ARMCX2, ARMCX3, ARMCX6, ARMET, ARPC4, ARPM1, ARPP-21, ARSB, ART3, ARTN, ASAH1, ASAH2, ASAP2, ASB10, ASB13, ASB17, ASB3, ASB6, ASB7, ASCC3, ASGR2, ASH1L, ASPH, ASS1, ASTE1, ASTL, ASTN², ATE1, ATF3, ATG10, ATG16L1, ATG4C, ATG7, ATG9A, ATL2, ATP13A2, ATP1A1, ATP1B1, ATP1B4, ATP2A1, ATP2A2, ATP2B4, ATP2C1, ATP5C1, ATP5D, ATP5G2, ATP5G3, ATP5I, ATP5J, ATP5J2, ATP5SL, ATP6VOA4, ATP6VOB, ATP6VOE2, ATP6V1C2, ATP6V1G2, ATP6V1G3, ATP6V1H, ATP7B, ATP8A1, ATP8B2, ATPAF1, ATPAF2, ATPBD4, ATRIP, ATXN1, ATXN7, ATXN7L1, AUP1, AURKA, AURKC, AZI2, AZIN1, B3GALNT1, B3GALT5, B3GAT1, B4GALT2, B4GALT4, BAALC, BAAT, BACE2, BACH1, BAG1, BAIAP2, BANF1, BANK1, BAP1, BAT1, BAT3, BAT4, BAX, BAZ1A, BBS7, BBX, BCAN, BCAP29, BCClP, BCKDHB, BCL11A, BCL11B, BCL2, BCL2L11, BCL6, BCLAF1, BCR, BCS1L, BDH1, BDNF, BEGAIN, BEND4, BEND7, BEX5, BHLHB9, BICD2, BID, BIN1, BLOClS2, BMF, BMI1, BMP1, BMP2K, BMP4, BMX, BNIP1, BNIPL, BOD1, BOLA3, BOLL, BOP1, BPGM, BPHL, BPY2B, BPY2C, BRAP, BRD2, BRD4, BRD8, BRD9, BRE, BR13, BRMS1, BRP44, BRPF1, BRUNOL4, BSCL2, BSDC1, BSG, BTBD7, BTF3, BTF3L4, BTLA, BTN2A1, BTN2A2, BTN3A1, BTN3A3, BTNL3, BUD13, BZRAP1, BZRPL1, BZW1L1, BZW2, C10orf108, C10orf125, C10orf25, C10orf32, C10orf53, C10orf71, C10orf72, C10orf78, C11orf10, C11orf17, C11orf21, C11orf35, C11orf57, C11orf63, C11orf68, C11orf71, C11orf92, C12orf11, C12orf4, C12orf44, C12orf47, C12orf62, C12orf72, C13orf31, C13orf37, C14orf1, C14orf100, C14orf102, C14orf118, C14orf133, C14orf148, C14orf149, C14orf181, C14orf33, C14orf48, C14orf53, C15orf23, C15orf40, C15orf44, C15orf57, C16orf10, C16orf42, C16orf53, C16orf65, C16orf86, C16orf93, C17orf49, C17orf58, C17orf65, C17orf69, C17orf81, C17orf91, C18orf1, C18orf25, C18orf56, C19orf2, C19orf23, C19orf26, C19orf36, C19orf42, C19orf43, C19orf48, C19orf60, C19orf63, C1D, C1orf102, C1orf104, C1orf106, C1orf109, C1orf111, C1orf116, C1orf122, C1orf124, C1orf125, C1orf126, C1orf131, C1orf156, C1orf174, C1orf175, C1orf177, C1orf183, C1orf198, C1orf2, C1orf200, C1orf213, C1orf226, C1orf25, C1orf26, C1orf35, C1orf38, C1orf43, C1orf59, C1orf66, C1orf71, C1orf9, C1orf91, C1QC, C1QTNF3, C1QTNF5, C20orf12, C20orf132, C20orf134, C20orf173, C20orf199, C20orf200, C20orf201, C20orf7, C21orf57, C21orf58, C21orf90, C22orf23, C22orf26, C2CD3, C2orf21, C2orf24, C2orf28, C2orf30, C2orf44, C2orf56, C2orf64, C2orf65, C2orf68, C2orf7, C2orf76, C2orf77, C2orf81, C2orf86, C2orf88, C3orf17, C3orf18, C3orf23, C3orf37, C3orf42, C3orf71, C3orf74, C4B, C4BPB, C4orf16, C4orf30, C4orf33, C4orf38, C4orf41, C4orf44, C4orf46, C5orf13, C5orf20, C5orf24, C5orf26, C5orf33, C5orf37, C5orf39, C5orf44, C5orf45, C5orf55, C6orf1, C6orf105, C6orf106, C6orf108, C6orf127, C6orf130, C6orf136, C6orf146, C6orf15, C6orf154, C6orf162, C6orf165, C6orf25, C6orf47, C6orf52, C7orf13, C7orf16, C1orf25, C7orf27, C7orf46, C7orf53, C7orf58, C7orf68, C8orf40, C8orf41, C8orf45, C8orf51, C8orf56, C8orf58, C8orf59, C80RFK29, C9orf116, C9orf127, C9orf128, C9orf130, C9orf131, C9orf23, C9orf24, C9orf25, C9orf66, C9orf68, C9orf7, C9orf72, C9orf75, C9orf78, C9orf86, C9orf95, C9orf98, CA10, CAB39, CACNA1D, CACNA1F, CACNA1G, CACNA11, CACNA2D2, CACNB1, CACNB2, CACNB4, CADM1, CADM3, CADPS, CADPS2, CAGE1, CALB2, CALCOCO1, CALD1, CALHM2, CALU, CAMK1D, CAMK2A, CAMK2B, CAMK2D, CAMK2G, CANX, CAP1, CAPN10, CAPN3, CAPN9, CARD9, CARHSP1, CASK, CASK1N2, CASP10, CASP2, CASP3, CASP6, CASP8, CASP9, CAST, CAV2, CAV3, CBFB, CBLN3, CBX2, CBX3, CBX5, CCBL1, CCBL2, CCDC102A, CCDC108, CCDC114, CCDC115, CCDC12, CCDC121, CCDC122, CCDC123, CCDC127, CCDC132, CCDC142, CCDC148, CCDC34, CCDC36, CCDC47, CCDC50, CCDC51, CCDC58, CCDC59, CCDC66, CCDC7, CCDC71, CCDC80, CCDC82, CCDC87, CCDC88A, CCDC96, CCHCR1, CCL14, CCL20, CCL23, CCNA1, CCNB3, CCNC, CCND3, CCNG1, CCNT2, CCNYL1, CCR10, CCR3, CCR9, CCT3, CCT6A, CCT6B, CCT7, CD109, CD14, CD164, CD1E, CD200, CD200R1, CD247, CD276, CD2BP2, CD33, CD34, CD36, CD3D, CD44, CD46, CD47, CD53, CD55, CD58, CD68, CD83, CD8A, CD96, CD99L2, CDC14A, CDC2, CDC20B, CDC25A, CDC25C, CDC26, CDC27, CDC2L5, CDC42, CDC42SE1, CDCA4, CDCA5, CDCA7, CDCA7L, CDCP1, CDH23, CDH29, CDH7, CDK10, CDK2, CDK4, CDK5, CDK5RAP2, CDK6, CDKL3, CDKN1A, CDKN2C, CDKN2D, CDSN, CDV3, CDY2A, CEACAM20, CEACAM21, CEBPZ, CENPA, CENPK, CENPT, CEP170, CEP290, CEP55, CEP63, CEPT1, CERKL, CES1, CES2, CES7, CETN2, CFC1, CFH, CFLAR, CGB7, CGGBP1, CGREF1, CHAC1, CHAT, CHCHD4, CHCHD7, CHCHD8, CHDH, CHI3L2, CHM, CHMP2A, CHN1, CHPF, CHRAC1, CHRM2, CHRNA1, CIDEA, CIDECP, CILP2, CIR, CIRBP, CISH, CKAP2, CKMT2, CKS1B, CLASP1, CLCN2, CLCN3, CLCN6, CLCNKA, CLDN14, CLDN19, CLDN6, CLDN9, CLDND1, CLDND2, CLEC10A, CLEC4C, CLIC1, CLIC3, CLIP2, CLK2, CLP1, CLRN1, CLSTN1, CLTA, CLTB, CLU, CMAH, CMPK1, CMTM4, CMTM5, CMTM7, CNBP, CNGA1, CNGB1, CNKSR1, CNOT4, CNOT7, CNR1, CNRIP1, CNTFR, CNTROB, COBRA1, COG2, COG4, COL11A1, COL11A2, COL13A1, COL25A1, COL2A1, COL4A3, COL4A3BP, COL4A4, COL4A5, COL4A6, COL6A3, COL8A1, COLEC11, COLQ, COMMD3, COMMD6, COPA, COPB2, COPE, COPS8, COX11, CPA5, CPA6, CPEB2, CPEB3, CPSF1, CPSF3L, CPSF4, CPT1C, CPVL, CPZ, CRAT, CRCT1, CRELD1, CREM, CRISP2, CRMP1, CROP, CROT, CRTC1, CRYBB1, CRYZ, CSAD, CSAG1, CSF1, CSGALNACT1, CSK, CSMD2, CSMD3, CSNK1A1, CSNK1G3, CSNK2B, CSRP1, CST11, CSTF1, CTAGE5, CTBP1, CTDSP1, CTDSPL, CTH, CTLA4, CTNNB1, CTSA, CTSB, CTSE, CTSL1, CUGBP2, CUL7, CUTA, CUX1, CXCL12, CXCL9, CXCR4, CXorf1, CXorf48, CXorf65, CYB561, CYB561D1, CYB5R1, CYBRD1, CYC1, CYFIP2, CYGB, CYHR1, CYP11B1, CYP24A1, CYP26A1, CYP39A1, CYP4B1, CYTSB, D4S234E, DACH2, DALRD3, DARC, DAXX, DAZ4, DAZAP1, DAZAP2, DBI, DBN1, DBNDD2, DBP, DCDC2, DCDC2B, DCHS2, DCLK2, DCT, DCTD, DCTN1, DCTN3, DCTN4, DCUN1D2, DCUN1D3, DCUN1D4, DDAH1, DDAH2, DDB1, DDC, DDIT3, DDO, DDR1, DDR2, DDT, DDX17, DDX28, DDX31, DDX4, DDX41, DDX42, DDX47, DEDD, DEF8, DEFA1, DEFA3, DEK, DENND1B, DEPDC1B, DERL1, DEXI, DFNA5, DFNB31, DGKA, DGKB, DGKG, DHDDS, DHFR, DHFRL1, DHRS1, DHRS12, DHRS2, DHRS9, DHX29, DHX36, DIAPH2, DIDO1, DIO1, DIO2, DISC1, DKFZP686I15217, DLC1, DLG1, DLK2, DLX1, DMAP1, DMBT1, DMBX1, DMGDH, DMKN, DMRT2, DMTF1, DNAH12, DNAJA3, DNAJB12, DNAJB14, DNAJB2, DNAJB5, DNAJB6, DNAJC12, DNAJC17, DNAJC30, DNAJC7, DNHD1, DNM1, DNM3, DNMT3A, DOK2, DOK3, DOLK, DOLPP1, DOM3Z, DPH2, DPH3, DPH5, DPM3, DPYD, DQX1, DRD3, DSC1, DSC2, DSC3, DSCR4, DSCR8, DSP, DST, DSTYK, DTNA, DTNB, DTNBP1, DUOX1, DUOX2, DUOXA1, DUSP10, DUSP11, DUSP15, DUSP19, DUSP2, DUSP3, DUSP6, DUSP7, DUT, DYNC2L11, DYNLT1, DYRK2, DYRK3, DYSF, DZIP1, E2F5, EARS2, EBAG9, EBNA1BP2, ECE2, ECHDC1, ECM1, ECSIT, EDARADD, EDEM2, EDF1, EDNRB, EEF1B2, EEF1D, EEF1E1, EFCAB1, EFCAB2, EFCAB4A, EFCAB6, EFEMP1, EFNA4, EFS, EFTUD2, EGFL7, EGFLAM, EGFR, EGLN2, EHBP1, EHMT2, EIF1AD, EIF2B4, EIF2C3, EIF3B, EIF4A1, EIF4A2, EIF4E, EIF4E3, EIF4H, ELAVL3, ELAVL4, ELF3, ELF5, ELK4, ELMO1, ELMO2, ELMOD1, ELMOD3, ELN, ELOVL6, EMCN, EML4, ENG, ENO3, ENOSF1, ENPP2, EPB41, EPB41L2, EPB41L4A, EPB41L4B, EPB49, EPHA3, EPHA8, EPHB2, EPM2A, EPM2AIP1, EPN1, EPS8L3, ERAP1, ERBB21P, ERBB3, ERCC1, ERCC8, ERG, ERGIC3, ERLIN2, ERMAP, ERMN, ERP44, ESF1, ESM1, ESR1, ESRRG, ETV3, EVC2, EVI2A, EVX2, EXD1, EXD3, EXO1, EXOC1, EXOC4, EXOC5, EXOC8, EXOG, EXOSC1, EXOSC10, EXOSC3, EXOSC4, EXOSC7, EXOSC9, EXTL2, EYA1, EYA4, EZR, FABP7, FAIM3, FAM107A, FAM110A, FAM111A, FAM111B, FAM114A2, FAM118A, FAM119A, FAM119B, FAM120AOS, FAM124B, FAM125B, FAM126B, FAM131B, FAM13B, FAM158A, FAM160A2, FAM164C, FAM166A, FAM167B, FAM173B, FAM186B, FAM19A3, FAM19A4, FAM3C, FAM40B, FAM45B, FAM53C, FAM54A, FAM54B, FAM55C, FAM58A, FAM63A, FAM63B, FAM70A, FAM71E1, FAM83A, FAM89B, FAM96B, FANCA, FANCD2, FANCG, FASTK, FASTKD2, FASTKD3, FBLIM1, FBLN1, FBLN2, FBLN7, FBP1, FBXL12, FBXL13, FBXL6, FBXO11, FBXO15, FBXO16, FBXO22, FBXO24, FBXO28, FBXO34, FBXO4, FBXO43, FBXO48, FBXO5, FBXO7, FBXO8, FBXO9, FBXW11, FBXW12, FBXW5, FCAMR, FCGR2A, FCGR3A, FCGRT, FCHO2, FCN2, FCN3, FDPS, FDXR, FERMT3, FEZ2, FEZF1, FGA, FGF1, FGF11, FGF5, FGFR1, FGFR10P, FGFR3, FGFR4, FGG, FGGY, FGL1, FGR, FHL1, FHL2, FIBCD1, FIBP, FIGNL1, FILIP1L, FIP1L1, FKBP1B, FKBP3, FKBP5, FKBP6, FKBP7, FKTN, FLAD1, FLCN, FLII, FLJ10404, FLJ11506, FLJ12825, FLJ13197, FLJ25328, FLJ33630, FLJ35024, FLJ37201, FLJ39653, FLJ40852, FLJ42709, FLJ42875, FLJ45983, FLJ90757, FLNA, FLNC, FLOT1, FLRT3, FLT1, FMO3, FN1, FNBP1L, FNDC3B, FNDC4, FNTA, FOSB, FOXI1, FOXL2, FOXO3, FOXP1, FOXP2, FOXP3, FOXP4, FPGS, FPR2, FRS3, FSD1L, FST, FSTL5, FTSJ1, FTSJ2, FUT7, FUZ, FXN, FXR1, FXYD2, FYB, FYTTD1, FZR1, G3BP1, G3BP2, G6PC2, GAA, GABBR1, GABRA1, GABRA2, GABRB2, GABRG2, GAD1, GAD2, GAK, GALNS, GALNT4, GALR2, GAP43, GAR1, GAS5, GATA2, GATA3, GBA, GBA2, GBA3, GBP5, GCC1, GCC2, GCDH, GCET2, GCK, GCM2, GCNT1, GCNT7, GCOM1, GDAP1, GDAP2, GDE1, GDF9, GDI2, GDNF, GEFT, GEMIN7, GEN1, GFAP, GFI1, GFM2, GFOD1, GFRA4, GGA1, GGCX, GGPS1, GGT6, GHRHR, GHRL, GHRLOS, GHSR, GIT2, GJB3, GJB4, GJB5, GJC2, GK, GLA, GLB1L, GLE1, GLI1, GLMN, GLOD4, GLRA1, GLRA3, GLRX, GLT8D1, GLUL, GLYCTK, GMEB1, GMPPB, GMPR2, GNAI3, GNAS, GNASAS, GNAT1, GNAT2, GNG4, GNG5, GNL3, GNLY, GNRH1, GNRHR2, GOLGA2, GOLGA7, GOLM1, GOPC, GORAB, GORASP1, GP2, GPA33, GPAA1, GPATCH2, GPATCH4, GPBAR1, GPBP1, GPBP1L1, GPC2, GPD1, GPD2, GPER, GPLD1, GPM6A, GPN1, GPNMB, GPR1, GPR107, GPR110, GPR113, GPR152, GPR175, GPR177, GPR18, GPR63, GPR85, GPS1, GPT2, GPX1, GPX5, GRAMD3, GRB2, GREB1, GRHL3, GRIA1, GRIA2, GRIA3, GRIK2, GRIN1, GRIN2A, GRINA, GRIPAP1, GRK4, GRK6, GRM1, GRM2, GRM7, GRM8, GRSF1, GSG1, GSK3B, GSN, GSTCD, GSTK1, GSTM1, GSTM2, GSTM4, GSTZ1, GTF2A1, GTF2A1L, GTF2H1, GTF2H4, GTF2I, GTF21RD1, GTF3C1, GTF3C2, GTPBP10, GTPBP2, GTPBP3, GTPBP8, GTSF1L, GUK1, GYPC, H1F0, H1FX, H2AFJ, H2AFV, H2AFY, H2AFZ, H3F3A, HACL1, HAND2, HARB11, HARS, HAS2, HAUS8, HAVCR1, HAX1, HBS1L, HCCS, HCFC1R1, HCG18, HCST, HDAC11, HDAC3, HDAC7, HDAC9, HDDC3, HDGF, HDGF2, HDHD1A, HDLBP, HEBP1, HECTD2, HECTD3, HELQ, HEPACAM2, HEPH, HES4, HESS, HES6, HEY1, HFE, HFE2, HGF, HHAT, HIBCH, HIGD1A, HILS1, HINT1, HINT2, HIPK1, HIRA, HIRIP3, HIST1H1A, HIST1H1B, HIST1H1D, HIST1H1E, HIST1H1T, HIST1H2AA, HIST1H2AD, HIST1H2AH, HIST1H2AK, HIST1H2AM, HIST1H2BB, HIST1H2BC, HIST1H2BD, HIST1H2BG, HIST1H2BJ, HIST1H2BK, HIST1H2BL, HIST1H3A, HIST1H3D, HIST1H3F, HIST1H3G, HIST1H3H, HIST1H3I, HIST1H4A, HIST1H4B, HIST1H4D, HIST1H4I, HIST1H4K, HIST2H2AA3, HIST2H2AA4, HIST2H2AB, HIST2H2BE, HIST2H3A, HIST2H3C, HIST3H2A, HIST4H4, HK1, HLA-DPA1, HLA-F, HLTF, HM13, HMGA1, HMGB2, HMGB4, HMGCLL1, HMGCR, HMGN3, HMMR, HNF4A, HNMT, HNRNPA1, HNRNPA2B1, HNRNPC, HNRNPD, HNRNPF, HNRNPH2, HNRNPK, HNRNPR, HNRNPU, HNRPA1L-2, HNRPDL, HNRPLL, HOPX, HOTAIR, HOXA1, HOXA10, HOXA11, HOXA2, HOXA3, HOXA4, HOXA6, HOXB13, HOXB3, HOXC5, HOXC9, HOXD12, HPCAL1, HPGD, HPS4, HPS5, HPSE, HR, HRAS, HRASLS, HRC, HRSP12, HS2ST1, HS3ST3B1, HS6ST2, HSD17B8, HSDL1, HSF2, HSPA1L, HSPB2, HSPB6, HSPB7, HSPBAP1, HSPD1, HTATIP2, HTR3A, HTR3D, HTR4, HTRA2, HVCN1, HYAL1, HYAL2, HYAL3, HYOU1, IARS, ICA1, ICA1L, ICK, ICMT, IDH3B, IDS, IER3, IFFO1, IF16, IFIT3, IFLTD1, IFNA10, IFT122, IFT172, IFT20, IFT74, IFT88, IGF2, IGF2BP2, IGFBP3, IGSF11, IGSF9, IK, IKBKAP, IL10RA, IL11RA, IL12RB1, IL15, IL15RA, IL17RC, IL17RE, IL18BP, IL1F10, IL1F5, IL1F7, IL1RAP, IL1RN, IL21R, IL22RA2, IL24, IL25, IL28RA, IL4, IL5RA, IL6R, IL9R, IMMT, IMPA1, IMPDH1, ING3, INPP1, INPP4A, INPP5D, INPP5F, INPP5K, INSIG1, INSRR, INTS12, INTS7, INVS, IP6K1, IP6K3, IPP, IQCB1, IQCC, IQCE, IQCG, IQCH, IQWD1, IRAK4, IRF2BP2, IRF5, IRF7, IRX2, ISG20L2, ISLR, ITGA1, ITGA7, ITGAV, ITGB1, ITGB1BP1, ITGB3BP, ITGB4, ITIH4, ITIH5, ITM2C, ITPK1, ITPR1, ITPRIPL1, IWS1, JDP2, JMJD4, JMJD5, JMJD7, JMJD7-PLA2G4B, JOSD1, JPH4, JTB, KALRN, KAT, KAT2A, KBTBD3, KBTBD4, KCNAB2, KCNAB3, KCNC1, KCNC4, KCNG3, KCNH1, KCNH2, KCNH6, KCNH7, KCNIP1, KCNIP2, KCNJ1, KCNJ16, KCNK16, KCNK17, KCNMB3, KCNQ4, KCNQ5, KCP, KCTD10, KCTD15, KCTD21, KCTD6, KDELR2, KDM1, KDM2B, KDM3A, KDM4C, KEAP1, KHK, KIAA0090, KIAA0141, KIAA0196, KIAA0226, KIAA0232, KIAA0319L, KIAA0406, KIAA0430, KIAA0562, KIAA0652, KIAA0895, KIAA0922, KIAA1026, KIAA1143, KIAA1161, KIAA1217, KIAA1407, KIAA1429, KIAA1524, KIAA1712, KIAA1875, KIAA1908, KIAA1949, KIAA1967, KIAA1984, KIF13A, KIF17, KIF1B, KIF23, KIF24, KIF25, KIF9, KIN, KIT, KITLG, KLC4, KLF1, KLF6, KLHDC9, KLHL23, KLHL4, KLHL5, KLHL7, KLK10, KLK11, KLK12, KLK3, KLK8, KLRAQ1, KLRC1, KLRD1, KNG1, KPNA1, KRAS, KRIT1, KRT10, KRT72, KRT80, KRTAP10-6, KRTAP6-2, KRTCAP2, KTELC1, KTN1, KYNU, L3 MBTL3, LACTB2, LAGE3, LAIR1, LAMA2, LAMA4, LAMB3, LANCL1, LARGE, LARP2, LARP6, LASS2, LAT, LAT2, LAX1, LBR, LBX2, LCA5, LCE1C, LCE5A, LCLAT1, LCMT1, LDB2, LDB3, LEF1, LEFTY1, LENEP, LEPR, LEPRE1, LEPREL1, LEPROT, LEPROTL1, LETMD1, LGALS12, LGALS8, LGALS9, LGI3, LGSN, LHX6, LHX9, LIAS, LILRA2, LILRB4, LIMCH1, LIMK2, LIN54, LINS1, LIPT1, LIX1L, LMAN2L, LMBRD2, LMLN, LMNA, LMOD1, LMX1A, LOC100128003, LOC100128164, LOC100128191, LOC100128788, LOC100130093, LOC100130426, LOC100130430, LOC100130557, LOC100130691, LOC100131193, LOC100132215, LOC100190938, LOC100191040, LOC100216545, LOC100268168, LOC100272217, LOC145783, LOC158381, LOC158572, LOC199800, LOC220930, LOC26010, LOC285456, LOC285548, LOC285696, LOC285847, LOC286260, LOC339524, LOC388387, LOC389813, LOC401093, LOC401431, LOC401463, LOC440926, LOC441476, LOC492303, L00541471, LOC643923, LOC646799, LOC653566, LOC678655, LOC729338, LOC730101, LOC91149, LONP1, LOXL3, LPAR1, LPO, LQK1, LRCH4, LRDD, LRIT1, LRP8, LRRC15, LRRC17, LRRC2, LRRC20, LRRC24, LRRC29, LRRC32, LRRC40, LRRC41, LRRC61, LRRC8A, LRRCC1, LRRFIP1, LRRFIP2, LRRIQ1, LRRIQ3, LRRN2, LRRTM4, LRSAM1, LRTOMT, LSM1, LSM5, LSM7, LST1, LTA, LTB, LUZP1, LY6G5B, LY6G6C, LY6G6E, LY6G6F, LY6H, LY6K, LY9, LYAR, LYN, LYNX1, LYPD1, LYPD4, LYRM1, LYRM4, LYSMD1, LYSMD2, LZIC, MAD2L2, MADD, MAGEA10, MAGEAl2, MAGEB1, MAGEB4, MAGED2, MAGI1, MAGI3, MAL, MALT1, MAMDC4, MAMSTR, MANBAL, MANEAL, MAP2, MAP3K12, MAP3K4, MAP3K6, MAP3K7, MAP4, MAP4K1, MAPK10, MAPK14, MAPK3, MAPK7, MAPK8, MAPK81P2, MAPKAPK2, MAPKAPK5, MAPKSP1, MARCH2, MARCH8, MARK2, MARVELD2, MASP1, MASP2, MAT2B, MATN2, MAX, MB, MBLAC2, MBNL1, MBNL2, MBP, MBTD1, MCART1, MCEE, MCHR2, MCM10, MCM3AP, MCM8, MCTP2, MDC1, MDH1B, MDM1, ME3, MEA1, MECR, MED15, MED20, MED22, MED23, MEDT, MEDS, MEF2A, MEST, MESTIT1, MET, METT11D1, METTL1, METTL13, METTL5, METTL6, MFAP2, MFAP3, MFGE8, MFI2, MFN2, MFSD10, MFSD2, MFSD3, MFSD8, MGAT4B, MGC12982, MGC15885, MGC16275, MGC16703, MGC23284, MGC24975, MGC2889, MGC29506, MGC70857, MGEA5, MGLL, MICAL1, MIER1, MINA, MITD1, MITF, MKNK1, MKNK2, MKRN1, MLC1, MLF1, MLL5, MLLT10, MLN, MLPH, MLXIPL, MME, MMP1, MMP16, MMP28, MOBKL2A, MOBKL2B, MOBKL2C, MOBP, MOCS1, MOCS2, MOG, MOGS, MON1A, MORC2, MORN1, MORN4, MOSPD3, MPDU1, MPZ, MPZL1, MPZL2, MRAS, MRE11A, MRI1, MRPL10, MRPL11, MRPL13, MRPL14, MRPL2, MRPL21, MRPL24, MRPL30, MRPL33, MRPL35, MRPL36, MRPL4, MRPL43, MRPL46, MRPL47, MRPL50, MRPL55, MRPS11, MRPS21, MRPS27, MRPS33, MRRF, MRVI1, MS4A1, MS4A14, MS4A3, MS4A4A, MS4A6A, MSH4, MSH5, MSL2, MSL3, MSMB, MSMP, MSR1, MSRA, MT1G, MT1M, MTERFD1, MTFR1, MTMR11, MTMR14, MTO1, MTP18, MTPN, MTRF1L, MTRR, MTUS1, MTX2, MUC1, MUC15, MUC4, MUDENG, MUT, MUTYH, MVD, MYBPC1, MYEOV2, MYH2, MYL12B, MYL4, MYL6, MYLK, MYO18A, MYO19, MYO1C, MYO9B, MYOCD, MYOT, MYOZ3, MYST1, MYST3, N4BP2L1, NAAA, NAIF1, NAP1L3, NAPEPLD, NARF, NARG1L, NASP, NAT1, NAT10, NAT5, NAT6, NAT8, NBEAL1, NBL1, NCAM1, NCBP2, NCDN, NCF2, NCF4, NCKAP1, NCKIPSD, NCOA1, NCOA3, NCOA4, NCR1, NCR3, NCRNA00095, NCRNA00105, NCRNA00120, NCRNA00176, NDE1, NDOR1, NDRG1, NDRG2, NDRG4, NDUFA2, NDUFA8, NDUFAF3, NDUFS1, NECAB3, NECAP2, NEDD9, NEFM, NEIL2, NEK11, NEK4, NEK6, NELF, NEURL2, NEURL4, NF1, NFE2, NFE2L2, NFIA, NFKB2, NFKBIB, NFKBID, NFKBIE, NFKBILl, NFRKB, NFYA, NFYC, NGDN, NGLY1, NHEDC1, NHLH2, NICN1, NIF3L1, NKAIN4, NKIRAS1, NKIRAS2, NKPD1, NKX3-2, NLRP11, NLRP3, NLRX1, NME7, NNAT, NNT, NOC2L, NOL6, NOL8, NONO, NOP16, NOP56, NOSTRIN, N-PAC, NPAT, NPC1L1, NPHP1, NPHP3, NPL2, NPSR1, NPW, NQO1, NR1D2, NR1H3, NR112, NR3C1, NR4A1, NR4A3, NR5A2, NR6A1, NRAP, NRCAM, NRG1, NRG2, NRGN, NRM, NRP2, NRXN3, NSD1, NSDHL, NSFL1C, NSL1, NSMAF, NSUN2, NSUN5, NT5C1B, NT5C2, NT5DC2, NTF4, NTRK1, NTRK2, NTRK3, NUDCD1, NUDT1, NUDT2, NUDT5, NUDT6, NUDT9, NUF2, NUP155, NUP37, NUP62CL, NUTF2, NXF1, OAS1, OAS2, OASL, OBSCN, OBSL1, OCIAD2, ODF2L, OGDH, OGDHL, OGFR, OGG1, OGT, OLA1, OLAH, OLFM1, OPA1, OPN4, OPN5, OPRL1, OPRM1, OPTN, OR1J2, OR2H2, OR51D1, OR51E1, OR51G2, OR6B3, OR6Q1, OR6T1, OR8U1, ORAI2, ORC1L, ORC3L, ORC4L, ORC5L, ORM2, ORMDL3, OS9, OSBPL10, OSBPL9, OSGIN2, OSR2, OTOF, OTUB1, OTUD4, OTUD5, OXSM, P2RX6, P4HA2, P4HTM, PABPC4, PACRG, PACRGL, PAEP, PAF1, PAICS, PAIP2, PAK3, PALB2, PALM2-AKAP2, PAM, PAMR1, PAN2, PANK1, PANK4, PAP2D, PAPD4, PAPPA2, PAQR6, PARD6A, PARK2, PARK7, PARL, PARP10, PARP3, PARP9, PARVG, PASK, PAX3, PAX6, PAX7, PAX8, PBRM1, PBX2, PBX3, PCBP3, PCBP4, PCDH1, PCDH10, PCDH7, PCDHA11, PCDHA13, PCDHA3, PCDHA4, PCDHA5, PCDHA6, PCDHAC1, PCDHAC2, PCDHB12, PCDHB13, PCDHB9, PCDHGA10, PCDHGA11, PCDHGA2, PCDHGA3, PCDHGB7, PCDHGB8P, PCDHGC4, PCDHGC5, PCGF1, PCGF2, PCP2, PCSK7, PCTK1, PCTK3, PDAP1, PDCD2, PDE10A, PDE11A, PDE1A, PDE4DIP, PDE6B, PDE6D, PDE8A, PDIA2, PDIK1L, PDILT, PDLIM2, PDLIM5, PDLIM7, PDPN, PDS5A, PDZD11, PDZD7, PECI, PEG10, PEG3, PELO, PEMT, PENK, PEX1, PEX10, PEX19, PFDN2, PFKFB1, PFKFB2, PFKFB4, PFN1, PFN2, PGLYRP4, PGM3, PHACTR3, PHB2, PHF1, PHF10, PHF12, PHF17, PHF19, PHF20L1, PHF6, PHF7, PHKA1, PHPT1, PHTF2, PHYHD1, PHYHIP, PI4KA, PIAS2, PICK1, PID1, PIGA, PIGC, P1GF, PIGG, PIGN, PIGO, PIGS, PIGY, PIH1D1, PIK3R1, PIK3R3, PILRA, PIP5K1A, PIP5KL1, PIR, PITX2, PITX3, PIWIL2, PJA1, PKD1L2, PKD1L3, PKIB, PKLR, PKP1, PKP2, PKP4, PLA2G4C, PLACE, PLAGL1, PLAT, PLAU, PLCB4, PLCD1, PLCD3, PLCG1, PLCXD2, PLCZ1, PLD1, PLEC1, PLEKHB1, PLEKHB2, PLEKHG5, PLEKHH3, PLEKHN1, PLK3, PLOD3, PLP1, PLSCR4, PLUNC, PLXNB1, PMFBP1, PMP22, PMS1, PMS2, PMS2L3, PNKD, PNPLA4, PODXL, POGZ, POLDIP2, POLDIP3, POLE3, POLL, POLR1A, POLR1B, POLR1C, POLR2I, POLR3C, POLR3GL, POMC, POMGNT1, POMT1, PON2, POP1, POPS, POPDC3, POSTN, POTEA, POU1F1, PPAP2A, PPAP2B, PPAP2C, PPAPDC1B, PPARA, PPARD, PPARG, PPEF1, PPFIA1, PPFIBP1, PPIL6, PPM1B, PPM1G, PPM1J, PPM1M, PPDX, PPP1CB, PPP1R10, PPP1R11, PPP1R14B, PPP1R1B, PPP1R3D, PPP1R8, PPP2CB, PPP2R2B, PPP2R2D, PPP2R4, PPP2R5D, PPP6C, PPPDE2, PPT2, PQBP1, PQLC1, PQLC2, PRAC, PRAF2, PRCC, PRCP, PRDM10, PRDM15, PRDM16, PRDM2, PRDX1, PRDX2, PREB, PREX2, PRG4, PRH2, PRKAA1, PRKAB2, PRKACB, PRKAG1, PRKCB, PRKCD, PRKRA, PRM1, PRM2, PRM3, PRMT3, PRMT5, PRNT, PROK2, PROM1, PRPF40A, PRR21, PRRT1, PRRX1, PSAT1, PSD, PSEN2, PSIP1, PSMA2, PSMA4, PSMA8, PSMB1, PSMB10, PSMB8, PSMB9, PSMD5, PSME1, PSME3, PSMG1, PSPH, PSPN, PSRC1, PTCD1, PTCH1, PTER, PTGDS, PTGER3, PTGES2, PTGR1, PTGS1, PTK2, PTK2B, PTK7, PTMA, PTP4A3, PTPN12, PTPN13, PTPN18, PTPN22, PTPN5, PTPN6, PTPN7, PTPRA, PTPRF, PTPRJ, PTPRK, PTPRU, PTRH1, PUF60, PUM1, PURG, PVRL2, PXMP3, PYDC1, PYGO2, PYHIN1, QDPR, QRICH1, QSOX1, RAB11FIP1, RAB13, RAB1A, RAB23, RAB24, RAB27A, RAB28, RAB34, RAB41, RAB7L1, RABGGTB, RABL3, RABL5, RAD1, RAD17, RAD51AP1, RAD51L1, RAG1AP1, RAI14, RANBP3, RAP1A, RAP1B, RAP1GAP, RAP1GDS1, RAPH1, RARB, RARRES1, RARS2, RASGRP2, RASSF1, RASSF5, RASSF6, RASSF7, RB1CC1, RBBP4, RBCK1, RBM12B, RBM18, RBM19, RBM24, RBM38, RBMS3, RBMY1A1, RBMY1D, RBMY1E, RBMY1F, RBP1, RBP5, RBPJ, RBPMS, RC3H2, RCAN1, RCC2, RCHY1, RCOR3, RDBP, RDM1, REC8, RECQL, RECQL4, REG3A, REG4, RELA, RELL1, RELL2, RELN, REPIN1, REPS1, REPS2, RERE, RET, RETN, REV1, REXO4, RFC2, RFC4, RFESD, RFK, RFNG, RFWD2, RFX2, RFX5, RG9MTD2, RGAG4, RGR, RGS12, RGS13, RGS19, RGS4, RGS8, RHBDD2, RHBDF2, RHBG, RHCE, RHOA, RHOBTB1, RHOBTB2, RHOC, RIBC1, R10K2, RIPPLY2, RLN2, RNASE1, RNASE11, RNASE13, RNASE4, RNASE9, RNASEN, RNF113A, RNF121, RNF133, RNF138, RNF138P1, RNF14, RNF148, RNF167, RNF170, RNF180, RNF19B, RNF208, RNF214, RNF216, RNF25, RNF38, RNF39, RNF5P1, RNF7, RNH1, RNLS, ROBLD3, ROBO1, ROR1, RORA, RORC, RPE, RPH3A, RPL18A, RPL18AP3, RPL28, RPL31, RPL32, RPL32P3, RPL34, RPL36, RPL37, RPL38, RPL6, RPL7, RPL8, RPL9, RPN2, RPP14, RPP30, RPS14, RPS18, RPS20, RPS24, RPS3, RPS6KC1, RPSA, RPUSD3, RRBP1, RRP9, RRS1, RSPO4, RSU1, RTEL1, RTKN, RTN2, RTN4, RTN41P1, RUFY1, RUFY2, RUNDC3B, RUSC1, RWDD1, RWDD3, RWDD4A, RXRG, RYK, S100A1, S100A13, S100A4, S100A5, S100A6, S100PBP, SAMD10, SAMD12, SAP130, SAP30L, SARDH, SARNP, SASS6, SATB2, SBDS, SC4MOL, SC65, SCARA3, SCLT1, SCN2A, SCN3A, SCN4B, SCN5A, SCNN1D, SCO1, SCOC, SCP2, SCRN1, SCRN2, SCYL3, SDC1, SDCBP, SDCCAG3, SDF4, SDHC, SEBOX, SEC13, SEC22C, SEC24B, SEC61A2, SEC61G, SEMA3B, SEMA4D, SEMA7A, SENP6, SEP15, SEPP1, SEPSECS, SEPT10, SEPT12, SEPT2, SEPT4, SEPT7, SERBP1, SERINC1, SERINC4, SERP1, SERPINA1, SERPINA10, SERPINA9, SERPINB2, SERPINE2, SERPING1, SERPINI1, SESN1, SETD6, SETMAR, SEZ6L2, SF1, SF3A1, SF3B14, SF3B4, SFMBT1, SFRS121P1, SFRS15, SFRS18, SFRS2, SFTA2, SFTPB, SGCD, SGCE, SGK1, SGK3, SGMS2, SGOL1, SGOL2, SGSM2, SGTB, SH2D2A, SH2D3C, SH2D4B, SH3BP2, SH3D19, SH3GL1, SH3YL1, SHANK1, SHARPIN, SHBG, SHC1, SHOX2, SHPRH, SIGLEC7, SIGMAR1, SIKE, SILL SIM2, SIN3A, SIRPG, SIRT2, SIRTS, SIVA1, SKAP1, SKIL, SKP1, SKP2, SLA2, SLAMF9, SLC10A7, SLC12A3, SLC14A1, SLC15A2, SLC17A3, SLC17A8, SLC18A1, SLC18A3, SLC1A4, SLC1A5, SLC22A1, SLC22A6, SLC22A7, SLC23A1, SLC23A3, SLC24A4, SLC25A11, SLC25A13, SLC25A14, SLC25A25, SLC25A32, SLC25A36, SLC25A40, SLC25A44, SLC26A1, SLC26A10, SLC26A5, SLC26A6, SLC26A7, SLC26A9, SLC27A2, SLC27A6, SLC28A1, SLC29A1, SLC2A11, SLC2A5, SLC2A6, SLC30A2, SLC30A7, SLC37A3, SLC38A4, SLC39A1, SLC39A10, SLC39A12, SLC39A14, SLC39A4, SLC39A6, SLC39A7, SLC39A8, SLC41A3, SLC44A3, SLC44A5, SLC45A2, SLC47A2, SLC5A3, SLC5A6, SLC5A9, SLC6A12, SLC6A20, SLC6A6, SLC6A9, SLC7A2, SLC7A9, SLC8A1, SLC9A3R2, SLCO2B1, SLCO5A1, SLITRK2, SLTM, SLUT, SMAD1, SMAD2, SMAD5, SMAD6, SMARCA2, SMARCAD1, SMARCAL1, SMARCD2, SMARCD3, SMC1A, SMC2, SMC6, SMCR7, SMEK2, SMG5, SMG7, SMOX, SMPD1, SMPD4, SMPDL3B, SMTN, SMURF1, SNAP23, SNAP47, SNAP91, SNCA, SNCB, SNHG3-RCC1, SNIP1, SNORA13, SNORA38, SNORA39, SNORA48, SNORA6, SNORA60, SNORA63, SNORA67, SNORA7A, SNORA7B, SNORA81, SNORA84, SNORA9, SNORD100, SNORD101, SNORD110, SNORD116-21, SNORD116-22, SNORD116-23, SNORD116-24, SNORD116-25, SNORD12C, SNORD15B, SNORD2, SNORD24, SNORD36B, SNORD37, SNORD38A, SNORD42A, SNORD42B, SNORD45A, SNORD45C, SNORD46, SNORD47, SNORD51, SNORD55, SNORD61, SNORD62A, SNORD62B, SNORD66, SNORD72, SNORD74, SNORD75, SNORD82, SNORD88C, SNORD95, SNRNP40, SNRPB2, SNRPD2, SNRPN, SNX11, SNX14, SNX16, SNX18, SNX7, SOCS5, SOD2, SOHLH1, SORBS1, SORBS2, SOX15, SOX30, SP100, SP140, SP8, SPAG16, SPAG4L, SPAG6, SPAG8, SPARCL1, SPAST, SPATA2, SPATC1, SPDYA, SPEF2, SPHKAP, SPIN3, SPINLW1, SPNS1, SPOCK2, SPOCK3, SPON2, SPP1, SPPL2B, SPRR3, SPRY1, SPRY4, SPSB2, SPTAN1, SPTB, SPTLC1, SQSTM1, SRA1, SREBF1, SRGAP3, SR1, SRP14P1, SRP68, SRP9, SRPR, SRRT, SS18, SSBP3, SSFA2, SSRP1, SSX5, ST3GAL1, ST3GAL3, ST5, ST6GAL1, ST6GAL2, ST6GALNAC3, ST6GALNAC4, ST7, ST7L, ST70T4, ST8SIA4, STAMBP, STAR, STARD8, STAT1, STATH, STEAP2, STEAP3, STIL, STK16, STK19, STK31, STMN1, STOM, STOX1, STRA6, STX2, STXBP2, STXBP5, SULF1, SULT1C2, SUPT3H, SUPT5H, SUPT7L, SURF1, SYN2, SYNCRIP, SYNE1, SYNGR1, SYNPO, SYNPO2, SYPL1, SYT3, SYTL2, TAAR2, TACC2, TACR1, TAF11, TAF12, TAF15, TAF5L, TAF9, TAGAP, TAGLN, TAGLN3, TANC1, TANK, TAP1, TAP2, TAPT1, TARBP2, TAS1R1, TAS2R8, TATDN1, TATDN3, TAX1BP1, TBC1D14, TBC1D15, TBC1D24, TBC1D5, TBC1D7, TBCCD1, TBL1X, TBL1Y, TBRG1, TBRG4, TBX20, TBX5, TBXAS1, TCEAL1, TCEAL8, TCERG1, TCF12, TCF19, TCF20, TCF21, TCF3, TCF4, TCF7, TCIRG1, TCL1B, TCL6, TCOF1, TCP1, TCP11, TCP11L1, TCTE3, TCTEX1D4, TDRKH, TENC1, TEPP, TERC, TERF1, TERT, TET2, TEX11, TEX264, TEX28, TFAP2A, TFB2M, TFEC, TFG, TFPI, TFPT, TFRC, TGFA, TGFB2, TGFBR2, TGFBRAP1, TGM5, TH, THADA, THAP1, THAP11, THOC6, THOC7, THPO, THRB, THTPA, THYN1, TIA1, TIFAB, TIGD1, TIGD6, TIGD7, TIMD4, TIMM8B, TIPRL, TJAP1, TJP1, TKT, TKTL1, TLE3, TLN1, TLR10, TLR4, TMCO6, TMED5, TMED8, TMEM100, TMEM106B, TMEM107, TMEM108, TMEM127, TMEM129, TMEM130, TMEM132A, TMEM139, TMEM14A, TMEM14B, TMEM150, TMEM155, TMEM169, TMEM176B, TMEM183A, TMEM194A, TMEM2, TMEM20, TMEM201, TMEM209, TMEM214, TMEM223, TMEM25, TMEM30A, TMEM39A, TMEM51, TMEM52, TMEM53, TMEM59, TMEM60, TMEM64, TMEM67, TMEM68, TMEM71, TMEM77, TMEM79, TMEM8, TMOD2, TMPO, TMPRSS3, TMUB2, TNF, TNFRSF10B, TNFRSF12A, TNFRSF25, TNFSF13B, TNNC1, TNNI3K, TNNT2, TNPO3, TOMM40, TOMM5, TOMM6, TOP3A, TOR1AlP2, TOR2A, TP5313, TP531NP1, TP63, TPD52, TPD52L3, TPI1, TPK1, TPM2, TPM3, TPO, TPR, TRAF31P2, TRAF5, TRAK2, TRAPPC2P1, TRAPPC6B, TRAPPC9, TRDMT1, TREX1, TRIB2, TRIM10, TRIM14, TRIM17, TRIM24, TRIMS, TRIM32, TRIM33, TRIM34, TRIM35, TRIM36, TRIM39, TRIM41, TRIM45, TRIM50, TRIM54, TRIM55, TRIM69, TRIM7, TRIP12, TRMT5, TRMT6, TRPM3, TRPV1, TRPV5, TRUB2, TSC1, TSC22D3, TSC22D4, TSKS, TSLP, TSPAN17, TSPAN19, TSPAN3, TSPYL1, TSR1, TSSC1, TTC13, TTC14, TTC16, TTC21A, TTC22, TTC37, TTC8, TTF1, TTLL10, TTLL11, TTN, TTRAP, TUBA4A, TUBA4B, TUBD1, TUBE1, TUFT1, TULP4, TUSC3, TUSC4, TXNDC9, TXNL4B, TXNRD1, TYMP, TYROBP, UBA1, UBA3, UBA5, UBA6, UBAC2, UBAP2L, UBE2A, UBE2CBP, UBE2D2, UBE2D3, UBE2E1, UBE2E3, UBE2H, UBE2I, UBE2J2, UBE2K, UBE2Q2, UBE2V1, UBE3B, UBL5, UBN1, UBP1, UBQLN1, UBQLN3, UBQLN4, UBTF, UBXN11, UCK1, UCKL1, UCN2, UCP3, UEVLD, UFD1L, UGCGL1, UGP2, UGT1A6, UGT2A3, UHRF1BP1L, ULK2, UNC119, UNC45A, UNC5CL, UNC93A, UOX, UPF3A, UPK3B, URG4, URM1, UROC1, USF1, USH1C, USH2A, USMG5, USP1, USP20, USP21, USP33, USP37, USP4, USP46, USP48, UTS2, UTS2D, UXT, VAMPS, VAMP7, VAMPS, VARS2, VASH2, VAV2, VCAM1, VCAN, VCL, VDAC3, VDR, VEGFA, VGLL2, VHL, VHLL, VIP, VLDLR, VNN2, VNN3, VPS13A, VPS13B, VPS26A, VPS28, VPS35, VPS36, VPS37A, VPS41, VPS52, VPS54, VTN, VWA5A, WARS, WASF1, WBP1, WBP11, WBP5, WDFY3, WDR1, WDR12, WDR17, WDR21A, WDR23, WDR26, WDR27, WDR31, WDR35, WDR46, WDR5, WDR51B, WDR53, WDR67, WDR77, WDR8, WDR92, WDSUB1, WEE1, WFDC10B, WFDC3, WFS1, WHSC1L1, WIBG, WIPI2, WISP1, WISP3, WNK3, WNT2, WSB1, WTAP, WWOX, XAF1, XCR1, XPA, XPC, XPO5, XPO7, XRCC4, XRN1, YARS, YIF1B, YIPF3, YME1L1, YOD1, YPEL5, YRDC, YTHDC1, YWHAE, YWHAZ, ZAK, ZAN, ZBP1, ZBTB12, ZBTB16, ZBTB37, ZBTB40, ZBTB43, ZC3HAV1, ZCCHC11, ZCCHC9, ZCWPW1, ZDHHC15, ZDHHC16, ZDHHC3, ZDHHC4, ZEB1, ZFAND5, ZFP112, ZFP36, ZFP36L2, ZFYVE27, ZFYVE9, ZGPAT, ZHX1, ZIC4, ZIM2, ZKSCAN5, ZMAT3, ZMAT4, ZMYM5, ZMYND10, ZMYND11, ZNF133, ZNF142, ZNF155, ZNF160, ZNF167, ZNF187, ZNF189, ZNF197, ZNF2, ZNF219, ZNF222, ZNF226, ZNF238, ZNF239, ZNF250, ZNF271, ZNF295, ZNF3, ZNF30, ZNF32, ZNF323, ZNF326, ZNF34, ZNF37A, ZNF384, ZNF385A, ZNF394, ZNF3970S, ZNF502, ZNF562, A2BP1, A2M, A4GNT, AAAS, AACS, AADAC, AADACL1, AADACL2, AADACL4, AADAT, AAK1, AANAT, AASDH, AASDHPPT, ABCA1, ABCA12, ABCA13, ABCA4, ABCB1, ABCB10, ABCB11, ABCB4, ABCB5, ABCB6, ABCB7, ABCB9, ABCC10, ABCC11, ABCD3, ABCE1, ABCF1, ABCF3, ABHD1, ABHD10, ABHD14A, ABHD2, ABHD5, ABI1, ABI2, ABI3BP, ABL1, ABLIM1, ABLIM2, ABLIM3, ABO, ABP1, ABRA, ABT1, ABTB1, ACAD10, ACAD8, ACAD9, ACADM, ACADVL, ACAP1, ACAP2, ACAT2, ACBD3, ACBD4, ACBD5, ACBD7, ACCN1, ACCN3, ACCN4, ACCN5, ACE, ACER1, ACMSD, ACN9, ACO1, ACOT11, ACOT12, ACOT13, ACOT2, ACOT7, ACOX2, ACOX3, ACP1, ACPL2, ACPP, ACR, ACRC, ACSBG1, ACSBG2, ACSF2, ACSL1, ACSL3, ACSL4, ACSL6, ACSM1, ACSM5, ACSS1, ACTA1, ACTB, ACTBL2, ACTG1, ACTG2, ACTL6A, ACTL6B, ACTL7A, ACTL7B, ACTL8, ACTN2, ACTN4, ACTR2, ACTR3, ACTR3B, ACTR8, ACTRT2, ACVR1, ACVR1B, ACVR1C, ACVR2A, ACVR2B, ACY1, ACYP2, ADAD1, ADAM10, ADAM15, ADAM17, ADAM19, ADAM2, ADAM21, ADAM22, ADAM23, ADAM29, ADAM30, ADAM32, ADAM5P, ADAM9, ADAMDEC1, ADAMTS1, ADAMTS13, ADAMTS15, ADAMTS16, ADAMTS17, ADAMTS19, ADAMTS2, ADAMTS8, ADAMTS9, ADAMTSL1, ADAMTSL2, ADAMTSL3, ADAMTSL4, ADAP1, ADAP2, ADAR, ADARB2, ADAT1, ADC, ADCK1, ADCK2, ADCK5, ADCY1, ADCY10, ADCY2, ADCY3, ADCY5, ADCY7, ADCY8, ADCYAP1, ADCYAP1R1, ADD1, ADD3, ADFP, ADH1A, ADH1B, ADH1C, ADH4, ADH5, ADH6, ADH7, ADI1, ADIG, ADIPOQ, ADIPOR1, ADM, ADORA1, ADORA2B, ADORA3, ADPGK, ADPRH, ADPRHL1, ADRA1A, ADRA1B, ADRA2B, ADRB2, ADSS, AEBP1, AEN, AFAP1L1, AFAP1L2, AFF3, AFF4, AFM, AFMID, AFTPH, AGA, AGAP1, AGAP3, AGBL3, AGBL5, AGGF1, AGK, AGL, AGMAT, AGPAT2, AGPAT4, AGPAT5, AGPAT6, AGPS, AGR2, AGR3, AGT, AGTPBP1, AGTR1, AGTR2, AGTRAP, AGXT, AGXT2, AGXT2L2, AHCYL1, AHR, AHRR, AHSA1, AHSG, AIF1L, AIFM3, AIG1, AIM1, AIM2, AIMP1, AIMP2, AJAP1, AK1, AK2, AK3, AK7, AKAP1, AKAP12, AKAP13, AKAP7, AKAP9, AKIRIN1, AKIRIN2, AKNA, AKR1A1, AKR1B10, AKR1C2, AKR1C3, AKR1C4, AKR1CL2, AKR1D1, AKR7L, AKT3, AKTIP, ALAD, ALAS1, ALB, ALCAM, ALDH16A1, ALDH18A1, ALDH1A1, ALDH1A2, ALDH1A3, ALDH1L1, ALDH3B2, ALDH4A1, ALDH5A1, ALDH8A1, ALDOB, ALG10B, ALG13, ALG9, ALK, ALKBH3, ALKBH5, ALKBH6, ALKBH7, ALLC, ALMS1, ALMS1P, ALOX5, ALOX5AP, ALPI, ALPL, ALS2, ALS2CL, ALS2CR11, ALS2CR12, ALS2CR8, ALX1, ALX3, ALX4, AMBP, AMD1, AMICA1, AMIGO1, AMIGO2, AMOTL2, AMPD1, AMPD2, AMPH, AMT, AMTN, AMY2B, ANAPC4, ANAPC5, ANAPC7, ANG, ANGEL2, ANGPT1, ANGPT2, ANGPT4, ANGPTL1, ANGPTL2, ANGPTL4, ANGPTL7, ANK1, ANK3, ANKFY1, ANKHD1, ANKIB1, ANKLE2, ANKRD1, ANKRD11, ANKRD13A, ANKRD13D, ANKRD17, ANKRD23, ANKRD26, ANKRD26L1, ANKRD34A, ANKRD37, ANKRD44, ANKRD46, ANKRD49, ANKRD50, ANKRD56, ANKRD57, ANKRD7, ANKRD9, ANKS1A, ANKS1B, ANKZF1, ANLN, ANO1, ANO4, ANO5, ANP32B, ANP32C, ANP32E, ANTXR1, ANTXR2, ANUBL1, ANXA1, ANXA13, ANXA2, ANXA3, ANXA5, ANXA6, ANXA7, ANXA9, AOAH, AOC3, AP1G2, AP1S3, AP2A1, AP2A2, AP2M1, AP3M2, AP3S1, APBA1, APBA2, APBBHP, APBB2, APC, APCDD1L, APCS, APEH, APEX1, APEX2, APH1A, APLF, APLNR, APOA1BP, APOA5, APOB, APOBEC1, APOBEC2, APOBEC4, APOL3, APOL6, APOM, APPL1, AQP1, AQP12A, AQP3, AQP9, ARAF, ARAP2, ARAP3, ARC, AREG, ARF5, ARF6, ARFGAP2, ARFGEF1, ARFGEF2, ARG1, ARG2, ARHGAP12, ARHGAP15, ARHGAP18, ARHGAP19, ARHGAP21, ARHGAP22, ARHGAP24, ARHGAP25, ARHGAP29, ARHGAP6, ARHGDIB, ARHGDIG, ARHGEF10, ARHGEF10L, ARHGEF16, ARHGEF19, ARHGEF2, ARHGEF3, ARID5A, ARIH2, ARL1, ARL14, ARL15, ARL2BP, ARL4C, ARL4D, ARL61P5, ARL8A, ARL8B, ARL9, ARMC10, ARMC2, ARMC3, ARMC4, ARMC9, ARMCX1, ARMCX5, ARPC5L, ARRDC1, ARRDC2, ARRDC4, ARSE, ARSI, ARSJ, ARSK, ART4, ARV1, ASAM, ASAP1, ASB1, ASB12, ASB14, ASB15, ASB2, ASB5, ASCL2, ASF1A, ASNA1, ASNSD1, ASPHD1, ASPN, ASPRV1, ASXL1, ASXL2, ATAD1, ATAD2, ATAD3A, ATCAY, ATF2, ATF6, ATG9B, ATIC, ATM, ATN1, ATOH1, ATOH7, ATOH8, ATP10D, ATP11B, ATP13A4, ATP1A2, ATP1A4, ATP4A, ATP4B, ATP5F1, ATP50, ATP6AP1, ATP6AP1L, ATP6AP2, ATP6V0D1, ATP6V0D2, ATP6V1B1, ATP6V1B2, ATP6V1C1, ATP6V1E2, ATP6V1G1, ATP8B4, ATP9A, ATP9B, ATR, ATRNL1, ATXN7L2, AUH, AVEN, AVIL, AVL9, AVP, AVPR1B, B3GALNT2, B3GALT1, B3GALT4, B3GALT6, B3GALTL, B3GAT2, B3GNT1, B3GNT2, B3GNT5, B4GALNT3, B4GALT1, B4GALT3, B4GALT7, BACH2, BAG2, BAG3, BAG4, BAHD1, BAIL BAI2, BAI3, BAIAP2L1, BAK1, BAMBI, BARD1, BARHL1, BARHL2, BARX1, BARX2, BASE, BASP1, BAT2, BAT2D1, BATF2, BATF3, BAZ1B, BBOX1, BBS12, BBS5, BCAR3, BCAS1, BCAS2, BCDIN3D, BCHE, BCL10, BCL3, BCL6B, BCL9, BDH2, BDP1, BEND2, BENDS, BEND6, BEST3, BEST4, BET1, BFSP2, BGLAP, BGN, BHLHA15, BHLHE22, BHLHE40, BHMT, BHMT2, BIN3, BIRC2, BIRC6, BLID, BLK, BLMH, BLOC1S1, BLZF1, BMP10, BMP3, BMP5, BMP6, BMP8B, BMPR1B, BMPR2, BMS1, BNIP3L, BOC, BPESC1, BPIL1, BPIL2, BPY2, BRCA2, BRD1, BRD3, BREA2, BRF1, BRF2, BRP44L, BRPF3, BRS3, BRSK1, BSN, BSND, BST1, BTBD10, BTC, BTD, BTG2, BTN1A1, BTN2A3, BTN3A2, BTNL2, BTNL9, BUB1, BUB1B, BUD31, BXDC1, BXDC2, BXDC5, BYSL, BZW1, C10orf10, C10orf107, C10orf118, C10orf120, C10orf137, C10orf140, C10orf2, C10orf46, C10orf47, C10orf54, C10orf62, C10orf67, C10orf82, C10orf90, C10orf97, C11orf16, C11orf31, C11orf34, C11orf40, C11orf42, C11orf45, C11orf46, C11orf51, C11orf52, C11orf53, C11orf61, C11orf64, C11orf66, C11orf82, C11orf87, C12orf26, C12orf34, C12orf36, C12orf39, C12orf40, C12orf41, C12orf43, C12orf48, C12orf54, C12orf60, C12orf68, C12orf76, C13orf16, C13orf26, C13orf27, C13orf34, C13orf35, C13orf36, C14orf105, C14orf139, C14orf145, C14orf165, C14orf166, C14orf166B, C14orf174, C14orf176, C14orf177, C14orf21, C14orf43, C14orf49, C14orf70, C15orf21, C15orf32, C15orf5, C15orf52, C15orf53, C15orf54, C16orf45, C16orf5, C16orf52, C16orf58, C16orf72, C16orf78, C16orf82, C16orf91, C17orf28, C17orf39, C17orf44, C17orf47, C17orf48, C17orf50, C17orf59, C17orf64, C17orf93, C18orf16, C18orf21, C18orf55, C19orf10, C19orf15, C19orf18, C19orf33, C19orf39, C19orf40, C19orf45, C19orf55, C19orf59, C19orf61, C19orf66, C19orf69, C1orf100, C1orf105, C1orf107, C1orf112, C1orf113, C1orf115, C1orf123, C1orf127, C1orf128, C1orf135, C1orf14, C1orf149, C1orf150, C1orf151, C1orf157, C1orf158, C1orf159, C1orf161, C1orf162, C1orf163, C1orf168, C1orf172, C1orf182, C1orf186, C1orf187, C1orf189, C1orf190, C1orf192, C1orf201, C1orf21, C1orf210, C1orf212, C1orf216, C1orf220, C1orf223, C1orf228, C1orf229, C1orf230, C1orf27, C1orf49, C1orf50, C1orf51, C1orf55, C1orf56, C1orf57, C1orf64, C1orf65, C1orf69, C1orf74, C1orf77, C1orf83, C1orf84, C1orf85, C1orf86, C1orf87, C1orf88, C1orf92, C1orf93, C1orf94, C1orf96, C1orf97, C1QA, C1QB, C1QBP, C1QL2, C1QL3, C1QTNF2, C1QTNF4, C1QTNF7, C1QTNF9, C1R, C1RL, C20orf106, C20orf11, C20orf112, C20orf114, C20orf118, C20orf141, C20orf144, C20orf152, C20orf166, C20orf177, C20orf186, C20orf197, C20orf20, C20orf29, C20orf39, C20orf4, C20orf43, C20orf70, C20orf79, C21orf84, C22orf28, C22orf33, C22orf43, C2orf16, C2orf18, C2orf29, C2orf39, C2orf40, C2orf42, C2orf48, C2orf49, C2orf50, C2orf51, C2orf52, C2orf53, C2orf55, C2orf57, C2orf58, C2orf61, C2orf67, C2orf69, C2orf70, C2orf71, C2orf73, C2orf78, C2orf83, C2orf84, C3orf1, C3orf14, C3orf15, C3orf19, C3orf20, C3orf21, C3orf22, C3orf24, C3orf25, C3orf26, C3orf27, C3orf30, C3orf31, C3orf32, C3orf33, C3orf36, C3orf38, C3orf44, C3orf47, C3orf48, C3orf49, C3orf52, C3orf54, C3orf57, C3orf58, C3orf59, C3orf63, C3orf64, C3orf67, C3orf70, C3orf72, C3orf75, C4A, C4BPA, C4orf10, C4orf12, C4orf17, C4orf19, C4orf22, C4orf23, C4orf26, C4orf29, C4orf3, C4orf31, C4orf32, C4orf34, C4orf35, C4orf37, C4orf39, C4orf40, C4orf42, C4orf43, C4orf49, C4orf50, C4orf6, C4orf7, C5, C5AR1, C5orf15, C5orf22, C5orf23, C5orf27, C5orf32, C5orf38, C5orf4, C5orf40, C5orf41, C5orf46, C5orf48, C5orf53, C5orf54, C5orf56, C6, C6orf10, C6orf114, C6orf118, C6orf120, C6orf122, C6orf125, C6orf126, C6orf129, C6orf134, C6orf138, C6orf141, C6orf142, C6orf145, C6orf153, C6orf167, C6orf168, C6orf173, C6orf174, C6orf182, C6orf201, C6orf208, C6orf217, C6orf222, C6orf223, C6orf225, C6orf27, C6orf58, C6orf64, C6orf70, C6orf81, C6orf89, C7, C7orf20, C7orf23, C7orf26, C7orf28B, C7orf29, C7orf30, C7orf31, C7orf33, C7orf34, C7orf36, C7orf38, C7orf42, C7orf43, C7orf44, C7orf45, C7orf47, C7orf51, C7orf52, C7orf54, C7orf60, C7orf64, C7orf67, CBA, C8B, CBG, C8orf22, C8orf34, C8orf37, C8orf39, C8orf4, C8orf42, C8orf47, C8orf48, C8orf76, C8orf79, C8orf84, C8orf86, C9, C9orf100, C9orf102, C9orf103, C9orf106, C9orf11, C9orf114, C9orf117, C9orf119, C9orf123, C9orf125, C9orf139, C9orf140, C9orf142, C9orf150, C9orf16, C9orf167, C9orf169, C9orf171, C9orf4, C9orf43, C9orf45, C9orf46, C9orf6, C9orf64, C9orf70, C9orf71, C9orf79, C9orf89, C9orf9, C9orf91, C9orf93, CA13, CA14, CA2, CA3, CA5B, CA6, CA8, CA9, CABC1, CABLES1, CABP4, CACNA1B, CACNA1E, CACNA2D1, CACNA2D3, CACNA2D4, CACNG2, CACNG3, CACNG8, CADM2, CALB1, CALCR, CALCRL, CALM2, CALML3, CALML5, CALML6, CALN1, CAMK1, CAMK1G, CAMK2N1, CAMK2N2, CAMKV, CAMLG, CAMP, CAMSAP1L1, CAMTA1, CAMTA2, CAP2, CAPG, CAPN11, CAPN2, CAPN6, CAPNS2, CAPSL, CAPZA1, CAPZA2, CAPZA3, CARD11, CARD14, CARD6, CASD1, CASP14, CASQ1, CASQ2, CATSPER3, CATSPER4, CATSPERB, CAV1, CBLB, CBLN1, CBR1, CBR4, CBS, CBX4, CBX8, CC2D1B, CCBE1, CCDC103, CCDC104, CCDC111, CCDC112, CCDC126, CCDC129, CCDC136, CCDC140, CCDC141, CCDC147, CCDC149, CCDC157, CCDC158, CCDC17, CCDC18, CCDC19, CCDC21, CCDC22, CCDC24, CCDC28A, CCDC28B, CCDC3, CCDC33, CCDC37, CCDC46, CCDC52, CCDC53, CCDC54, CCDC6, CCDC63, CCDC64, CCDC67, CCDC69, CCDC70, CCDC72, CCDC76, CCDC84, CCDC89, CCDC9, CCDC90A, CCDC90B, CCDC97, CCIN, CCK, CCKAR, CCL1, CCL13, CCL16, CCL17, CCL18, CCL19, CCL2, CCL21, CCL22, CCL26, CCL27, CCL28, CCL5, CCL7, CCM2, CCNA2, CCNB1, CCNB2, CCND2, CCNE2, CCNG2, CCNH, CCNI, CCNJL, CCNL1, CCNO, CCNY, CCR1, CCR4, CCR6, CCR8, CCRL1, CCRN4L, CCS, CCT4, CCT5, CD160, CD163L1, CD164L2, CD180, CD1A, CD1C, CD1D, CD2, CD207, CD22, CD226, CD244, CD27, CD2AP, CD300C, CD300LB, CD300LF, CD302, CD320, CD38, CD3E, CD3G, CD4, CD40LG, CD48, CD52, CD6, CD7, CD70, CD72, CD80, CD84, CD86, CD93, CDA, CDC123, CDC20, CDC2L6, CDC37L1, CDC40, CDC42EP3, CDC45L, CDC5L, CDC6, CDC73, CDCA8, CDCP2, CDGAP, CDH1, CDH10, CDH12, CDH17, CDH20, CDH22, CDH26, CDH5, CDH6, CDH9, CDK3, CDK5R2, CDK5RAP3, CDK9, CDKAL1, CDKL5, CDKN2AIP, CDO1, CDR1, CDR2, CDS1, CDX4, CDY2B, CDYL, CDYL2, CEACAM3, CEACAM4, CEACAM5, CEACAM6, CEACAM7, CEACAM8, CEBPB, CEBPD, CECR1, CECR6, CEL, CELA1, CELA2A, CELA2B, CELSR2, CELSR3, CENPC1, CENPE, CENPI, CENPP, CENPQ, CENPV, CEP135, CEP152, CEP350, CEP70, CEP72, CEP97, CER1, CERCAM, CERK, CETN1, CETN3, CETP, CFB, CFC1B, CFHR3, CFHR4, CFP, CFTR, CGA, CGN, CGRRF1, CHAC2, CHAF1A, CHCHD1, CHCHD2, CHCHD5, CHCHD6, CHD1L, CHD4, CHD5, CHD6, CHERP, CHGA, CHI3L1, CHIC2, CHIT1, CHL1, CHMP1B, CHMP2B, CHMP4C, CHMP5, CHN2, CHP, CHRD, CHRNA2, CHRNA3, CHRNA6, CHRNA9, CHRNB1, CHRNB2, CHRNB3, CHRNB4, CHRND, CHRNG, CHST1, CHST10, CHST12, CHST13, CHST4, CHST6, CHST9, CIAO1, CIB3, CIB4, CIC, CIDEC, CIITA, CILP, CISD1, CISD2, CITED2, CKAP2L, CKS2, CLASP2, CLCA2, CLCA3P, CLCN1, CLCN4, CLCNKB, CLDN1, CLDN11, CLDN12, CLDN16, CLDN17, CLDN18, CLDN2, CLDN20, CLDN23, CLDN3, CLDN4, CLDN8, CLEC11A, CLEC14A, CLEC16A, CLEC2A, CLEC3A, CLEC3B, CLEC4D, CLEC4E, CLEC4F, CLEC5A, CLIC4, CLIC5, CLIP3, CLIP4, CLK1, CLK3, CLK4, CLN5, CLN8, CLNK, CLOCK, CLPB, CLPS, CLPTM1L, CLRN10S, CLRN2, CLSPN, CLSTN2, CLSTN3, CLTC, CMA1, CMBL, CMC1, CMPK2, CMTM6, CMTM8, CNBD1, CNDP1, CNDP2, CNGA2, CNGA4, CNGB3, CNIH3, CNIH4, CNKSR2, CNKSR3, CNN3, CNNM4, CNOT10, CNOT3, CNOT6, CNOT8, CNP, CNPY2, CNR2, CNTF, CNTN2, CNTN4, CNTN6, CNTNAP1, CNTNAP2, CNTNAP5, COBL, COBLL1, COG3, COL10A1, COL16A1, COL17A1, COL1A1, COL1A2, COL21A1, COL22A1, COL23A1, COL24A1, COL27A1, COL3A1, COL5A1, COL5A2, COL6A1, COL7A1, COL8A2, COL9A1, COL9A2, COL9A3, COLEC10, COLEC12, column, COMMD10, COMMD2, COMMD8, COMTD1, COPG, COPG2, COPS3, COPS4, COPS6, COPS7B, COPZ1, COPZ2, COQ10B, COQ2, COQ4, CORIN, CORO1A, CORO2A, COX10, COX16, COX18, COX19, COX5B, COX6B2, COX6C, CP, CP110, CPA1, CPA2, CPA3, CPA4, CPD, CPE, CPEB4, CPLX1, CPLX2, CPLX4, CPN1, CPNE4, CPNE5, CPNE9, CPO, CPDX, CPS1, CPSF3, CPT2, CRABP2, CRADD, CRB1, CRB2, CRBN, CREB3, CREB3L2, CREB3L4, CREB5, CREG1, CRH, CRHBP, CRHR2, CRIM1, CRIPAK, CRIPT, CRISP3, CRISPLD2, CRNN, CRP, CRTAP, CRTC2, CRX, CRYBA4, CRYBB2, CRYBB3, CRYGB, CRYGC, CRYGN, CRYGS, CRYL1, CRYM, CSDC2, CSF1R, CSF2, CSF2RB, CSN2, CSN3, CSNK1A1L, CSPG5, CSRNP1, CSRNP3, CST3, CST4, CST5, CST7, CST8, CST9, CSTA, CSTF2, CSTL1, CTAGE1, CTBS, CTGF, CTHRC1, CTNNA1, CTNNA2, CTNNAL1, CTNNBL1, CTNND2, CTPS, CTRC, CTRL, CTSG, CTSK, CTSL2, CTSS, CTTNBP2NL, CUBN, CUL1, CUL3, CUL4B, CUL9, CUX2, CX3CR1, CXCL1, CXCL11, CXCL13, CXCL14, CXCL17, CXCL2, CXCL3, CXCL6, CXCR6, CXCR7, CXorf22, CXorf23, CXorf41, CXorf42, CXorf57, CXXC4, CXXC5, CYB561D2, CYB5B, CYB5R1, CYB5R4, CYBA, CYBB, CYorf16, CYP11A1, CYP17A1, CYP1A2, CYP1B1, CYP20A1, CYP26B1, CYP27B1, CYP27C1, CYP2C9, CYP2E1, CYP2U1, CYP3A4, CYP3A7, CYP46A1, CYP4A11, CYP4F12, CYP4V2, CYP4×1, CYP7B1, CYP8B1, CYR61, CYSLTR2, CYTIP, CYTL1, DAAM2, DAB2, DAB21P, DACT2, DAD1, DAG1, DAGLA, DAK, DAND5, DAP, DAPL1, DAPP1, DARS, DAZ1, DAZL, DBC1, DBF4, DBH, DBT, DBX1, DCBLD1, DCBLD2, DCC, DCD, DCLRE1B, DCP1A, DCP1B, DCP2, A5732AAMP, DCTN5, DCTN6, DCUN1D1, DDB2, DDHD2, DDI2, DDIT4L, DDOST, DDX1, DDX18, DDX20, DDX23, DDX25, DDX27, DDX3X, DDX46, DDX49, DDX50, DDX53, DDX56, DDX58, DDX59, DDX6, DECR1, DEF6, DEFA4, DEFA5, DEFA6, DEFB1, DEFB118, DEFB123, DENND1C, DENND2C, DENND2D, DENND3, DENND4C, DEPDC6, DES, DFFB, DFNB59, DGAT2L6, DGCR2, DGCR6, DGCR6L, DGCR8, DGKD, DGKE, DGKI, DHCR24, DHODH, DHRS4L2, DHTKD1, DHX15, DHX16, DHX34, DHX38, DHX8, DIP2C, DIRAS1, DIRAS2, DIRC1, DIRC2, DIS3L2, DIXDC1, DKFZP56400823, DKK2, DKK4, DLD, DLEU1, DLG4, DLGAP1, DLGAP2, DLL1, DLL4, DLX2, DLX3, DLX5, DLX6, DMC1, DMRT1, DMRT3, DMRTA1, DMRTC2, DMXL1, DNAH1, DNAH11, DNAH5, DNAH6, DNAH7, DNAH8, DNAH9, DNAI1, DNAJA1, DNAJB11, DNAJB13, DNAJB3, DNAJB4, DNAJB8, DNAJC10, DNAJC16, DNAJC18, DNAJC19, DNAJC22, DNAJC5, DNAJC5B, DNAJC5G, DNAJC6, DNAL1, DNALI1, DNASE1L2, DNASE1L3, DNASE2, DNASE2B, DND1, DNER, DNPEP, DNTTIP1, DOCK2, DOCK4, DOCK6, DOCK7, DOCKS, DOK1, DOK4, DOK7, DONSON, DOPEY1, DPAGT1, DPCR1, DPEP2, DPF1, DPM2, DPP10, DPP4, DPP8, DPPA2, DPPA4, DPT, DPY30, DPYS, DPYSL2, DPYSL3, DPYSL5, DR1, DRAP1, DRD1, DRG2, DSCC1, DSCR10, DSCR3, DSE, DSG1, DTL, DTWD2, DTX3L, DUOXA2, DUPD1, DUS1L, DUS2L, DUSP14, DUSP18, DUSP22, DUSP23, DUSP26, DUSP27, DUSP28, DUSP4, DUSP9, DVL1, DVL3, DYM, DYNC1L12, DYNLRB1, DYRK1A, DZIP1L, DZIP3, E2F2, E2F3, E2F8, EAF1, EAF2, EBF1, EBI3, EBP, ECEL1, ECHDC2, ECHDC3, ECM2, ECOP, EDA2R, EDAR, EDC4, EDEM1, EDEM3, EDIL3, EDN1, EDN2, EDNRA, EEF1A1, EEF1G, EEF2, EEFSEC, EEPD1, EFCAB3, EFCAB7, EFHA2, EFHB, EFHC1, EFHD1, EFHD2, EFNA3, EFNA5, EFNB1, EGF, EGFL8, EGLN1, EGLN3, EGR1, EGR3, EGR4, EHD1, EHD3, EHD4, EHHADH, EIF1B, EIF2A, EIF2AK3, EIF2B2, EIF2B5, EIF2C1, EIF2C2, EIF3D, EIF3H, EIF3I, EIF3K, EIF4E1B, E1F4E2, EIF4EBP1, EIF4EBP2, EIF4EBP3, EIF4G1, EIF5A2, EIF5B, ELAVL2, ELF2, ELL2, ELOVL1, ELOVL5, ELP2, ELP3, ELTD1, EMB, EMD, EMG1, EMID1, EMID2, EMILIN1, EMILIN3, EMP1, EMP2, EMP3, EMX1, EN1, EN2, ENC1, ENDOD1, ENDOG, ENHO, ENO2, ENOPH1, ENPEP, ENPP1, ENPP4, ENPP5, ENPP6, ENTPD1, ENTPD3, ENY2, EOMES, EPB41L5, EPC1, EPC2, EPGN, EPHA1, EPHA2, EPHA4, EPHA7, EPHB1, EPHB3, EPHB4, EPHB6, EPHX1, EPO, EPR1, EPRS, EPS15, EPX, ERAL1, ERC2, ERCC3, ERCC6, EREG, ERGIC1, ERICH1, ERMP1, ERN2, ERO1L, ERP27, ERRF11, ESAM, ESCO1, ESD, ESPN, ESPNL, ESR2, ESRP2, ESSPL, ETAA1, ETFDH, ETNK2, ETV1, ETV3L, ETV5, ETV6, ETV7, EVC, EVI5L, EVX1, EXOC2, EXOC3, EXOC6, EXT1, EXTL1, EXTL3, EYA3, F11, F11R, F12, F13A1, F2R, F2RL1, F2RL2, F3, F5, F9, FA2H, FAAH2, FABP1, FABP3, FABP4, FABP5, FABP6, FADS6, FAF1, FAF2, FAHD2A, FAIM2, FAM102A, FAM102B, FAM105A, FAM105B, FAM107B, FAM114A1, FAM115A, FAM117B, FAM120A, FAM120B, FAM122C, FAM126A, FAM128B, FAM129A, FAM12B, FAM131A, FAM131C, FAM132A, FAM133A, FAM134A, FAM134B, FAM136A, FAM13A, FAM150B, FAM151A, FAM154A, FAM159A, FAM160B2, FAM162A, FAM163A, FAM164A, FAM167A, FAM168B, FAM170A, FAM171A1, FAM171B, FAM174A, FAM176A, FAM176B, FAM178B, FAM180A, FAM181A, FAM184A, FAM187B, FAM19A2, FAM20B, FAM20C, FAM26D, FAM26E, FAM26F, FAM27L, FAM36A, FAM38B, FAM3D, FAM40A, FAM43A, FAM43B, FAM45A, FAM46A, FAM46B, FAM46C, FAM47A, FAM49A, FAM50B, FAM53A, FAM53B, FAM55A, FAM59A, FAM5B, FAM5C, FAM62B, FAM64A, FAM65B, FAM69A, FAM71A, FAM71B, FAM71C, FAM71F1, FAM73B, FAM78A, FAM78B, FAM82A1, FAM82A2, FAM82B, FAM83C, FAM83H, FAM84A, FAM84B, FAM89A, FAM8A1, FAM91A1, FAM92A1, FAM98A, FAM98C, FANCC, FANCE, FANCM, FAP, FARP2, FARS2, FASLG, FAT1, FAT2, FATE1, FBN2, FBP2, FBXL15, FBXL17, FBXL19, FBXL22, FBXL3, FBXL7, FBXO18, FBXO30, FBXO32, FBXO33, FBXO36, FBXO40, FBXO42, FBXO44, FBXO45, FBXO6, FCER1A, FCER1G, FCGR1A, FCGR2C, FCHO1, FCHSD1, FCHSD2, FCN1, FCRL2, FCRL3, FCRL4, FCRL5, FCRL6, FCRLB, FDFT1, FEM1B, FEN1, FER, FERD3L, FERMT1, FETUB, FEY, FGB, FGD2, FGD3, FGD5, FGD6, FGF10, FGF12, FGF13, FGF17, FGF18, FGF2, FGF23, FGF3, FGF6, FGF9, FGFBP1, FGFBP2, FGFR10P2, FGL2, FH, FHDC1, FHIT, FHL3, FHOD3, HULA, FILIP1, FIS1, FJX1, FKBP10, FKBP15, FKBP2, FKBP8, FKBP9, FLJ10213, FLJ11235, FLJ16779, FLJ1120184, FLJ20674, FLJ23834, FLJ32063, FLJ34503, FLJ35220, FLJ40125, FLJ42393, FLJ43980, FLJ46321, FLNB, FLOT2, FLVCR1, FLVCR2, FMNL2, FMO1, FMO2, FMO4, FMO6P, FMO9P, FMOD, FNBP4, FNDC1, FNDC5, FOS, FOSL2, FOXA1, FOXB1, FOXC1, FOXD2, FOXD3, FOXE3, FOXF2, FOXH1, FOXJ1, FOXJ3, FOXN3, FOXN4, FOXO1, FOXQ1, FOXR2, FOXRED1, FPGT, FPR1, FRAP1, FRAS1, FRAT2, FREQ, FRK, FRMD1, FRMD3, FRMD4A, FRMD6, FRMPD1, FRMPD4, FRZB, FSCB, FSCN1, FSCN3, FSIP1, FSTL1, FSTL4, FTMT, FTSJD2, FUBP1, FUBP3, FUCA1, FUCA2, FURIN, FUT10, FUT11, FUT4, FUT9, FXYD4, FXYD6, FXYD7, FYN, FZD1, FZD3, FZD5, FZD6, FZD7, FZD8, FZD9, GOS2, G6PC, GAB2, GABBR2, GABPA, GABPB2, GABRA4, GABRA6, GABRB1, GABRD, GABRG1, GABRP, GABRQ, GABRR1, GABRR2, GADD45A, GADD45G, GAL, GAL3ST2, GAL3ST3, GAL3ST4, GALK2, GALM, GALNT10, GALNT11, GALNT12, GALNT14, GALNT3, GALNT5, GALNT7, GALNTL2, GALNTL5, GALR1, GALR3, GALT, GAN, GAPT, GARNL3, GAS1, GAS2L2, GAS7, GATA4, GATA5, GATAD1, GATS, GBAS, GBF1, GBGT1, GBP1, GBP3, GBP4, GBP6, GBP7, GBX2, GC, GCA, GCAT, GCG, GCKR, GCLC, GCLM, GCM1, GCNT2, GCNT4, GDA, GDEP, GDF10, GDF11, GDF2, GDF5, GDF6, GDF7, GDPD4, GEMIN6, GFM1, GFPT1, GFPT2, GFRA3, GFRAL, GGA2, GGH, GGT1, GHR, GIGYF1, GIMAP1, GIMAP2, GIMAP5, GIMAP7, GIMAP8, GIN1, GINS4, GIPC2, GJA1, GJA10, GJA4, GJA5, GJA8, GJB1, GJB7, GJC3, GJD2, GJD4, GK2, GK5, GKN1, GLCCI1, GLIPR2, GLIS1, GLIS3, GLP1R, GLP2R, GLRB, GLS, GLT25D1, GLTP, GLTPD1, GM2A, GMCL1, GMCL1L, GMDS, GMIP, GML, GMPR, GNA11, GNA13, GNA15, GNAI1, GNAI2, GNAL, GNAQ, GNB1, GNB2, GNB2L1, GNB3, GNG10, GNG11, GNG3, GNG7, GNGT1, GNL2, GNPAT, GNPDA1, GNPTG, GNS, GOLGA1, GOLGA4, GOLGA5, GOLGB1, GOLIM4, GOLPH3, GOLPH3L, GOLT1A, GOLT1B, GORASP2, GP1BA, GPS, GP9, GPATCH3, GPC1, GPC3, GPC4, GPC5, GPC6, GPD1L, GPHB5, GPR101, GPR111, GPR114, GPR116, GPR119, GPR12, GPR124, GPR125, GPR128, GPR133, GPR141, GPR146, GPR148, GPR15, GPR151, GPR153, GPR156, GPR157, GPR160, GPR17, GPR171, GPR172A, GPR174, GPR182, GPR21, GPR22, GPR25, GPR27, GPR3, GPR31, GPR35, GPR37, GPR37L1, GPR39, GPR45, GPR52, GPR55, GPR61, GPR62, GPR65, GPR75, GPR78, GPR81, GPR82, GPR83, GPR84, GPR87, GPRC5A, GPRC5B, GPRC6A, GPRIN1, GPRIN2, GPRIN3, GPS2, GPSM2, GPSM3, GPT, GPX2, GPX3, GPX6, GPX7, GPX8, GRAMD1C, GRAMD2, GRB10, GREM2, GRHL2, GRHPR, GRID2, GRIK3, GRIN3A, GRK1, GRK5, GRK7, GRM3, GRM4, GRM6, GRPEL1, GRPEL2, GRPR, GRRP1, GRWD1, GSC, GSDMA, GSDMB, GSDMD, GSG2, GSPT2, GSS, GSTA1, GSTA3, GSTA5, GSTM5, GSTO1, GSTP1, GSTT2, GSTTP1, GSTTP2, GSX1, GSX2, GTDC1, GTF2B, GTF2E1, GTF2E2, GTF3C3, GTF3C4, GTPBP1, GUCA1A, GUCA1B, GUCA1C, GUCA2A, GUCA2B, GUCY1B2, GUCY2F, GULP1, GUSB, GYG1, GYPB, GZMA, GZMB, H6PD, HAAO, HABP4, HACE1, HADH, HAL, HAMP, HAND1, HAPLN1, HAPLN2, HARS2, HAS2AS, HAUS3, HAUS6, HAVCR2, HBB, HBEGF, HBM, HBP1, HBXIP, HBZ, HCG4, HCG9, HCLS1, HCN1, HCN3, HCP5, HCRTR1, HCRTR2, HDAC2, HDAC4, HDAC8, HDC, HDDC2, HDGFL1, HDHD2, HDHD3, HDX, HEATR1, HEATR4, HEBP2, HECA, HECW2, HELT, HEMK1, HEPACAM, HERC3, HERC5, HERPUD2, HES1, HES3, HEST, HEY2, HEYL, HHATL, HHIP, HHLA2, HIATL2, HIBADH, HIGD1B, HIGD2A, HINTS, HISPPD1, HIST1H1C, HIST1H2AB, HIST1H2AC, HIST1H2AE, HIST1H2AG, HIST1H2AI, HIST1H2AL, HIST1H2BA, HIST1H2BE, HIST1H2BF, HIST1H2BH, HIST1H2BI, HIST1H2BN, HIST1H2BO, HIST1H3C, HIST1H3E, HIST1H4C, HIST1H4E, HIST1H4F, HIST1H4G, HIST1H4J, HIST1H4L, HIST2H2AC, HIST3H2BB, HIST3H3, HIVEP1, HJURP, HK2, HK3, HKDC1, HLA-A, HLA-C, HLA-DMA, HLA-DMB, HLA-DOA, HLA-DOB, HLA-DPB1, HLA-DPB2, HLA-DQA1, HLA-DQA2, HLA-DQB2, HLA-DRB5, HLA-G, HLCS, HLF, HMCN1, HMGCL, HMGCS2, HMGN1, HMGN2, HMGN4, HMGXB3, HMP19, HNRNPA3, HNRNPH1, HOMER1, HOOK3, HORMAD1, HOXA11AS, HOXA13, HOXA5, HOXA7, HOXA9, HOXB1, HOXB4, HOXB9, HOXC10, HOXC11, HOXC12, HOXC4, HOXC6, HOXC8, HOXD1, HOXD11, HOXD13, HOXD3, HOXD4, HOXD8, HOXD9, HP1BP3, HPCA, HPCAL4, HPDL, HPS3, HRG, HRH1, HRH2, HS1BP3, HS3ST1, HS3ST2, HS3ST5, HS6ST1, HSD11B1, HSD11B2, HSD17B11, HSD17B14, HSD17B2, HSD17B3, HSD17B4, HSD3B1, HSD3B2, HSDL2, HSF1, HSP90AB1, HSPA1A, HSPA1B, HSPA2, HSPA4, HSPA5, HSPA6, HSPA9, HSPB3, HSPB8, HSPB9, HSPE1, HTATSF1, HTN3, HTR1A, HTR1D, HTR1E, HTR2A, HTR3B, HTR3C, HTR3E, HTR5A, HTR7P, HTRA4, HTT, HUNK, HUS1, HUS1B, HYPK, IBSP, ICOS, ID2, ID3, ID4, IDE, IDH2, IDO1, IDO2, IDUA, IER5, IER5L, IF127L2, IFI30, IF144, IF144L, IFIT1, IFIT2, IFITM3, IFNA1, IFNA2, IFNA5, IFNA6, IFNA7, IFNA8, IFNB1, IFNE, IFNK, IFNW1, IFRD1, IFRD2, IFRG15, IGBP1, IGDCC3, IGDCC4, IGF2AS, IGF2BP3, IGF2R, IGFBP1, IGFBP2, IGFBP5, IGFBPL1, IGFL1, IGFL3, IGFN1, IGHMBP2, IGSF10, IGSF2, IGSF21, IGSF6, IGSF8, 1HH, IKBKB, IKBKE, IKZF1, IL10, IL12A, IL12B, IL12RB2, IL13, IL13RA2, IL17A, IL17B, IL17F, IL17RB, IL17RD, IL17REL, IL18, IL18RAP, IL19, IL1A, IL1B, 1L1F6, IL1F9, IL1R1, IL1R2, IL1RL1, IL1RL2, IL20, IL20RA, IL20RB, IL21, IL22RA1, IL23A, IL23R, IL29, IL2RA, IL2RB, IL2RG, IL3, IL31RA, IL33, IL34, IL6, IL7, IL7R, IL8, IL8RA, IL8RB, IL9, ILDR1, ILDR2, ILKAP, IMMP2L, IMP4, IMPA2, IMPAD1, IMPDH2, IMPG1, IMPG2, INA, INADL, INE1, ING2, INHA, INHBA, INHBB, INHBE, INMT, IN080B, IN080D, IN080E, INPP5B, INPP5E, INSL3, INSL4, INSL5, INSL6, INSM1, INTS10, INTS3, INTS8, INTU, IPO11, IPO13, IPO4, IPO8, IPO9, IPPK, IQCF2, IQGAP2, IQGAP3, IQSEC1, IRAK1BP1, IRF1, IRF2, IRF4, IRF6, IRF9, IRS1, IRS2, IRS4, IRX1, IRX4, IRX5, ISCA1, ISG15, ISG20, ISL1, ISOC1, ISX, ISY1, ITGA10, ITGA2, ITGA4, ITGA5, ITGA8, ITGA9, ITGAX, ITGB1BP2, ITGB2, ITGB5, ITGB6, ITGB7, ITGB8, ITIH1, ITIH2, ITIH3, ITK, ITLN1, ITLN2, ITM2A, ITPKA, ITPKC, ITPR3, IVL, IVNS1ABP, IYD, JAG1, JAGN1, JAKMIP2, JARID2, JAZF1, JPH3, JRKL, JUN, KAAG1, KAL1, KANK1, KANK4, KAT2B, KATNA1, KATNAL2, KATNB1, KAZALD1, KBTBD11, KBTBD2, KBTBD5, KBTBD8, KCMF1, KCNA10, KCNA2, KCNA3, KCNAB1, KCNB1, KCND1, KCND2, KCNE4, KCNF1, KCNG4, KCNH5, KCNH8, KCNIP3, KCNIP4, KCNJ10, KCNJ11, KCNJ14, KCNJ15, KCNJ3, KCNJ6, KCNJ9, KCNK1, KCNK10, KCNK12, KCNK18, KCNK3, KCNK4, KCNK5, KCNK9, KCNMB1, KCNMB2, KCNN2, KCNN3, KCNQ1, KCNQ3, KCNS1, KCNS2, KCNS3, KCNT1, KCNV1, KCTD12, KCTD17, KCTD20, KCTD3, KCTD4, KCTD7, KCTD8, KDM3B, KDM5B, KEL, KHDC1, KHDRBS2, KHDRBS3, KIAA0020, KIAA0040, KIAA0100, KIAA0319, KIAA0323, KIAA0408, KIAA0415, KIAA0494, KIAA0556, KIAA0649, KIAA0746, KIAA0776, KIAA0802, KIAA1009, KIAA1267, KIAA1274, KIAA1383, KIAA1409, KIAA1468, KIAA1529, KIAA1539, KIAA1586, KIAA1680, KIAA1715, KIAA1804, KIAA1919, KIAA1958, KIAA2013, KIDINS220, KIF12, KIF13B, KIF15, KIF1A, KIF20A, KIF20B, KIF27, KIF2B, KIF2C, KIF3A, KIF4A, KIF5B, KIF6, KIFAP3, KIFC1, KIFC2, KIR3DX1, KIRREL, KISS1, KLF11, KLF15, KLF17, KLF3, KLF4, KLF7, KLF9, KLHDC10, KLHDC2, KLHDC3, KLHDC7A, KLHDC7B, KLHDC8B, KLHL17, KLHL18, KLHL20, KLHL21, KLHL24, KLHL3, KLHL30, KLHL32, KLHL36, KLHL6, KLHL8, KLHL9, KLK14, KLKB1, KLKBL4, KLRA1, KLRG2, KNCN, KPNA5, KPNB1, KRBA1, KRCC1, KR11, KRT1, KRT14, KRT19, KRT2, KRT222P, KRT26, KRT3, KRT32, KRT33A, KRT33B, KRT35, KRT36, KRT37, KRT5, KRT6A, KRT6B, KRT6C, KRT75, KRT76, KRT77, KRT78, KRT79, KRT85, KRT9, KRTAP10-10, KRTAP10-12, KRTAP10-7, KRTAP11-1, KRTAP13-2, KRTAP15-1, KRTAP21-1, KRTAP22-1, KRTAP26-1, KRTAP5-5, KRTAP8-1, KRTCAP3, KRTDAP, KTI12, KY, L1TD1, L3 MBTL4, LACE1, LACRT, LAD1, LAG3, LALBA, LAMB1, LAMB2, LAMB4, LAMC1, LAMC3, LANCL2, LANCL3, LAP3, LAPTM4A, LAPTM4B, LAPTM5, LARP1, LARP5, LARS2, LAS1L, LASS4, LASS5, LASS6, LATS1, LAYN, LBH, LBXCOR1, LCAT, LCE1A, LCE1B, LCE1D, LCE1E, LCE1F, LCE2A, LCE2B, LCE2C, LCE2D, LCE3A, LCE3B, LCE3C, LCE3D, LCE3E, LCE4A, LCK, LCN1, LCN12, LCN15, LCN2, LCN8, LCNL1, LCORL, LCP1, LCP2, LDHAL6B, LDLR, LDOC1L, LEAP2, LECT2, LEFTY2, LEKR1, LEMD1, LENG1, LETM2, LGALS1, LGALS13, LGALS3, LGALS3BP, LGALS4, LGI1, LGI2, LGR6, LGTN, LHCGR, LHFPL3, LHFPL4, LHFPL5, LHPP, LHX1, LHX2, LHX4, LHX8, LIFR, LILRA1, LILRA4, LILRP2, LIMD1, LIMK1, LIMS1, LIN28, LIN28B, LIN37, LIN52, LINGO1, LINGO4, LIPC, LIPF, LIPH, LIPJ, LIX1, LLGL1, LMAN2, LMCD1, LMF1, LMNB1, LMO1, LMO2, LMO4, LMOD3, LMTK2, LMX1B, LNPEP, LNX1, LOC100127888, LOC100129726, LOC100132111, LOC100133612, LOC100133957, LOC100134368, LOC100189589, LOC100192379, LOC120376, LOC143188, LOC144571, LOC149134, LOC149478, LOC150381, LOC151534, LOC152217, LOC153328, LOC153684, LOC157627, LOC202051, LOC253039, LOC256880, LOC257358, LOC284009, LOC284067, LOC286016, LOC339483, LOC347376, LOC348840, LOC388965, LOC389791, LOC400696, LOC402573, LOC440093, LOC440173, LOC440957, LOC441046, LOC441931, L00550112, LOC642502, LOC642587, LOC643837, LOC644672, LOC645323, LOC645676, LOC646227, LOC648740, LOC728358, LOC84856, LOC84931, LOC91431, LOH3CR2A, LOR, LOX, LOXHD1, LOXL1, LOXL2, LOXL4, LPAR2, LPCAT1, LPCAT2, LPGAT1, LPHN2, LPIN1, LPL, LPPR4, LRAT, LRBA, LRCH3, LRFN5, LRGUK, LRIG1, LRIG2, LRIT3, LRMP, LRP1, LRP11, LRP1B, LRP2, LRP2BP, LRP5L, LRPPRC, LRRC1, LRRC14, LRRC16A, LRRC18, LRRC19, LRRC26, LRRC33, LRRC37A, LRRC3B, LRRC4, LRRC43, LRRC46, LRRC50, LRRC52, LRRC55, LRRC56, LRRC6, LRRC66, LRRC67, LRRC7, LRRC70, LRRC8C, LRRN1, LRRN4, LRRTM1, LRRTM2, LRRTM3, LRTM1, LSAMP, LSG1, LSM11, LSM12, LSM14B, LSM2, LSM3, LSM4, LSM8, LTB4R2, LTC4S, LTF, LUC7L2, LUZP4, LUZP6, LVRN, LXN, LY6D, LY6G5C, LY6G6D, LY75, LY86, LY96, LYG1, LYG2, LYL1, LYPD2, LYPD3, LYPD6, LYPLA2, LYPLAL1, LYRM2, LYRM7, LYSMD3, LYST, LYVE1, LYZL1, LYZL4, LYZL6, LZTFL1, LZTS1, LZTS2, M6PR, M6PRBP1, MAB21L1, MAB21L2, MACC1, MACF1, MAD2L1, MAD2L1BP, MAF1, MAFA, MAFB, MAGEA1, MAGEA11, MAGEA5, MAGEB18, MAGEB2, MAGEB3, MAGEC2, MAGEC3, MAGEE2, MAGEF1, MAGEH1, MAGI2, MAGOH, MAGOHB, MAK, MAK10, MAK16, MALL, MAMDC2, MAML1, MAMLD1, MAN1A1, MAN1A2, MAN1B1, MAN1C1, MAN2A1, MANBA, MANEA, MANSC1, MAP1B, MAP1D, MAP1LC3C, MAP2K1, MAP2K3, MAP2K6, MAP2K7, MAP3K13, MAP3K14, MAP3K2, MAP3K5, MAP3K7IP2, MAP3K8, MAP4K3, MAP6D1, MAP7, MAPK13, MAPK11P1L, MAPK81P1, MAPKAPK3, MAPRE1, MARCKS, MARCKSL1, MARCO, MARK1, MARS2, MAS1, MAS1L, MAST2, MAST4, MASTL, MAT1A, MAT2A, MATN1, MATR3, MBD3L1, MBD6, MBL2, MBLAC1, MBOAT1, MC2R, MC3R, MC4R, MC5R, MCC, MCCC2, MCCD1, MCFD2, MCM2, MCM3, MCM6, MCM7, MCOLN3, MCTP1, MDFI, MDFIC, MDH1, MDS1, ME1, MED10, MED12L, MED16, MED25, MED26, MED27, MED29, MED30, MEF2C, MEF2D, MEFV, MEGF10, MEIS1, MEIS2, MELK, MEOX2, MEP1A, MEPCE, METTL14, MFAP3L, MFHAS1, MFN1, MFRP, MFSD1, MFSD11, MFSD4, MFSD6L, MFSD7, MFSD9, MGAM, MGAT4A, MGAT5, MGC12916, MGC16121, MGC23270, MGC26647, MGC42105, MGC45800, MGC72080, MGMT, MGP, MGST2, MGST3, MICA, MICAL2, MID1, MIDN, MIF4GD, MIIP, MIMT1, MIOS, MIXL1, MKLN1, MKRN3, MKX, MLANA, MLH1, MLL2, MLLT1, MLLT11, MLNR, MMADHC, MMEL1, MMP12, MMP15, MMP19, MMP21, MMRN1, MN1, MNAT1, MND1, MNDA, MNT, MNX1, MOBKL1A, MOBKL1B, MOGAT1, MOGAT2, MORC1, MORNS, MOS, MOSC1, MOSC2, MOXD1, MPDZ, MPHOSPH10, MPHOSPH8, MPI, MPL, MPP4, MPP6, MPV17, MRAP2, MRFAP1L1, MRGPRX1, MRGPRX2, MRGPRX4, MRM1, MRPL1, MRPL15, MRPL17, MRPL18, MRPL3, MRPL32, MRPL37, MRPL40, MRPL48, MRPL53, MRPS10, MRPS14, MRPS15, MRPS18B, MRPS18C, MRPS2, MRPS22, MRPS24, MRPS25, MRPS28, MRPS30, MRPS6, MRPS9, MRS2, MRTO4, MS4A5, MSC, MSH2, MSH3, MSH6, MSRB2, MST1R, MSTN, MSTO1, MSX1, MSX2, MT1A, MT1B, MT1H, MT2A, MT4, MTA1, MTA2, MTA3, MTAP, MTBP, MTCH1, MTDH, MTERF, MTF1, MTHFD1L, MTHFD2L, MTM1, MTMR8, MTMR9, MTNR1B, MTSS1, MTSS1L, MTTP, MTX3, MUCL1, MUL1, MUM1L1, MURC, MUSTN1, MUTED, MX2, MXD4, MXI1, MYBL2, MYBPC2, MYBPH, MYBPHL, MYC, MYCBP, MYCN, MYCT1, MYD88, MYEOV, MYF5, MYF6, MYH1, MYH13, MYH3, MYH4, MYH6, MYH7, MYH7B, MYL1, MYL10, MYL3, MYL5, MYL6B, MYLIP, MYLK2, MYLK3, MYLPF, MYNN, MY010, MYO15A, MYO1D, MYO1G, MYO3A, MYO6, MYO7B, MYOC, MYOG, MYOM2, MYOM3, MYOZ1, MYOZ2, MYRIP, MYST2, MYT1L, N4BP3, NAALAD2, NAALADL1, NAALADL2, NAB1, NAB2, NACC2, NADK, NAGPA, NAGS, NAMPT, NANOG, NANS, NAPB, ZNF41, ZNF414, ZNF436, ZNF451, ZNF512, ZNF514, ZNF547, ZNF593, ZNF597, ZNF668, ZNF721, ZNF747, ZRANB3, ZW10.

### Regulation of the Demethylation System by Retinoic Acid

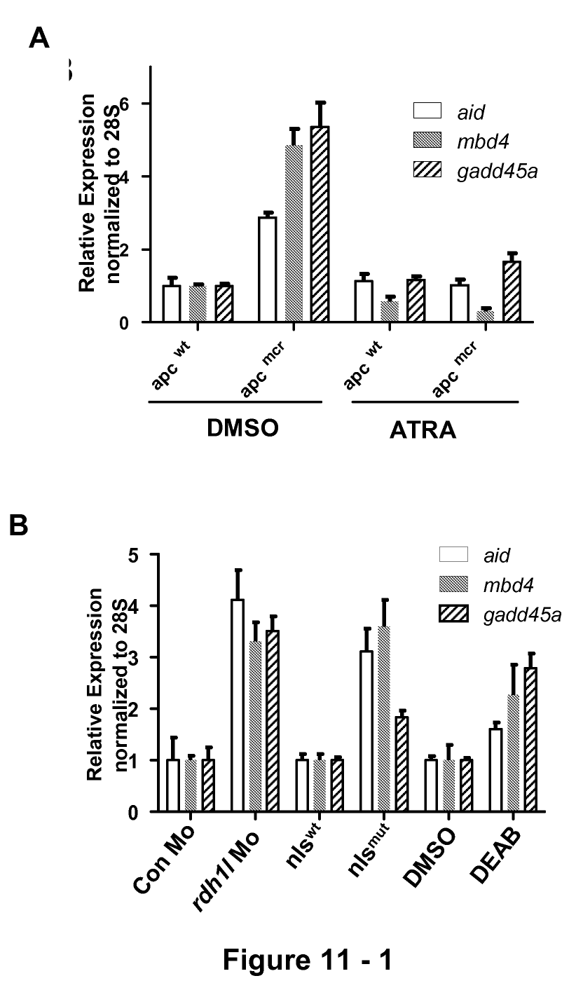

APC has been indicated in the control of both Wnt signaling and retinoic acid (RA) biosynthesis (Phelps et al., 2009a), providing two potential signaling mechanisms for demethylase regulation. To determine whether aberrant demethylase expression resulted from misregulated Wnt signaling or loss of RA production in apc mutant zebrafish, embryos were treated with either all-trans retinoic acid or knocked down levels of β-catenin by blocking Cox-2 activity using the pharmacological inhibitor (NS-398) (Eisinger et al., 2007). Treatment of apc mutants with retinoic acid greatly reduced the expression of aid, gadd45α and mbd4 whereas treatment with NS-398 had little effect (FIG. 11A). FIGS. 11A-B show the quantitative RT-PCR for aid, mbd4 and gadd45α in apc^{mcr} and apc^{wt} treated with DMSO or all-trans retinoic acid (ATRA) (A) or in different RA deficiency models in zebrafish (B). Expression of genes are first normalized to 28S, and then to the control embryo mRNA/28S ratio, valued as 1. Table 4 shows the expression of demethylase genes in apc mutants with different treatments.

However, RA treatment did not restore dnmt1 expression. Three additional models of RA deficiency support a role for RA in downregulating the demethylase: rdh11 morphants (Nadauld et al., 2005), nls mutants (Begemann et al., 2001) and DEAB (4-(diethylamino) benzaldehyde; an inhibitor of aldehyde dehydrogenases) treated wild type embryos all showed increased expression of the demethylase genes (FIG. 11B). Moreover, exogenous all-trans retinoic acid (ATRA) suppressed expression of the demethylase genes in human colon cancer cell lines treated with RA (FIG. 11C; DMSO is vehicle).

TABLE 4
Expression of Demethylase Genese in APC mutants with
Different Treatments

| | aid | gadd45α | mbd4 | dnmt1 |
|---|---|---|---|---|
| apc^{wt} + | 94% | 94% | 95% | 97% |
| DMSO | (n = 96)^{$} | (n = 105) | (n = 87) | (n = 69) |
| apc^{mcr} + | 17% | 9% | 3% | 11% |
| DMSO | (n = 42) | (n = 43) | (n = 39) | (n = 28) |
| apc^{mcr} + | 85% | 91% | 65% | 89% |
| ATRA | (n = 55) | (n = 53) | (n = 52) | (n = 37) |
| apc^{mcr} + | 20% | 8% | 9%% | n/d |
| Cox-2 inh | (n = 45) | (n = 9) | (n = 47) | |
| apc^{wt} + | 95% | 93% | 93% | n/d |
| Control mo | (n = 98) | (n = 103) | (n = 110) | |
| apc^{mcr} + | 14% | 14% | 9% | n/d |
| Control Mo | (n = 35) | (n = 37) | (n = 43) | |
| apc^{mcr} + | 81% | 83% | 67% | n/d |
| Pou5f1 Mo | (n = 53) | (n = 48) | (n = 55) | |
| apc^{mcr} + | 84% | 83% | 65% | n/d |
| Cebpb Mo | (n = 49) | (n = 47) | (n = 60) | |
| apc^{mcr} + | 91% | 89% | 73% | n/d |
| CtBP-1 Mo | (n = 45) | (n = 47) | (n = 62) | |
| Control | 94% | 96% | 92% | n/d |
| | (n = 63) | (n = 59) | (n = 63) | |
| Oct4 | 11% | 9% | 13% | n/d |
| overexp | (n = 62) | (n = 69) | (n = 69) | |
| Cebpb | 13% | 10% | 13% | n/d |
| overexp | (n = 72) | (n = 59) | (n = 68) | |

Similar to the results following morpholino knock down of demethylase components, treatment of apc mutant embryos with RA reversed the loss of DNA methylation observed on the panel of genes implicated in intestinal differentiation and proliferation (FIG. 11D). Taken together, these studies provide multiple lines of evidence for robust downregulation of demethylase components by retinoic acid.

Cebpβ and pou5f1 Mediate Regulation of the Demethylase Downstream of Retinoic Acid

Retinoic acid production involves two steps: (1) conversion of dietary retinol into retinaldehyde by retinol dehydrogenases (RDHs), and (2) conversion of retinaldehyde into retinoic acid by aldehyde dehydrogenases (ALDHs or RALDHs). Previous studies have demonstrated that apc mutation leads to loss of RDH expression (Jette et al., 2004; Nadauld et al., 2004; Nadauld et al., 2005). To better understand the status of RA biosynthesis following apc mutation, the expression of aldh1a2, which is closely related to Aldh1a1, was examined. Zebrafish lack aldh1a1. ALDH1 is a commonly use marker of stems cells and cancer stems including those derived from human colon and colon carcinomas (Huang et al., 2009). Whole mount in situ hybridization revealed upregulation of aldh1a2 in apc mutants beginning at 36 hpf which remained elevated through 72 hpf. This was paralleled by increased aldh1a2 protein levels). The upregulation of aldh1a2 in apc^{mcr} embryos combined with an absence of rdh1 and rdh1l suggested poising of the intestinal progenitor cells for retinoic acid biosynthesis upon production of the required substrate. Indeed, treatment of apc^{mcr} embryos with exogenous RAL partially restored intestinal differentiation marked by fabp2 expression a result also observed in rdh1l morphant embryos. Consistent with studies indicating that aldh1a2 expression can be repressed by RA (Dobbs-McAuliffe et al., 2004) (Niederreither et al., 1997), the treatment of apc^{mcr} mutant, rdh1l morphant or nls mutant zebrafish with RA decreased the expression of aldh1a2

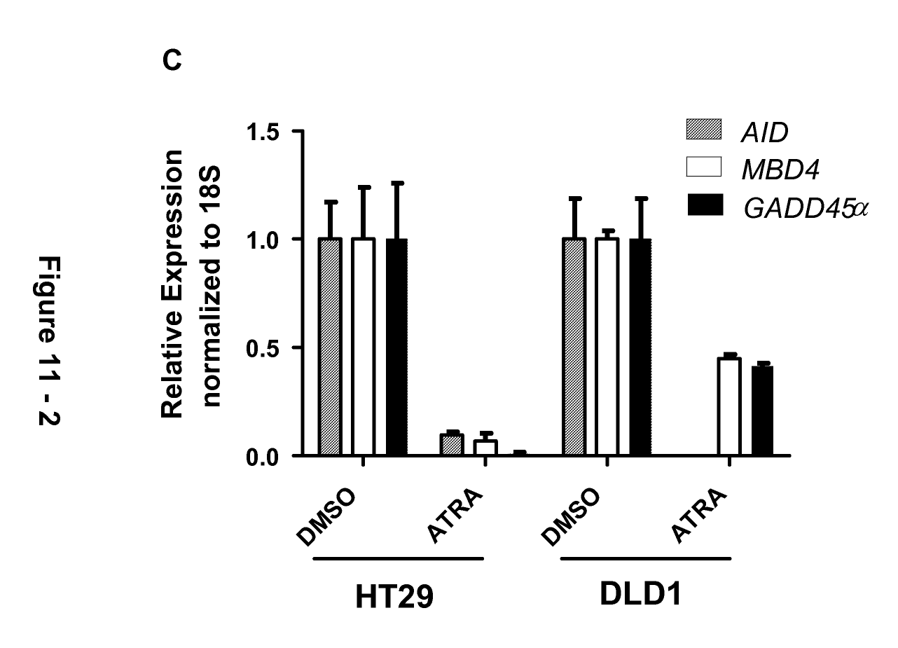

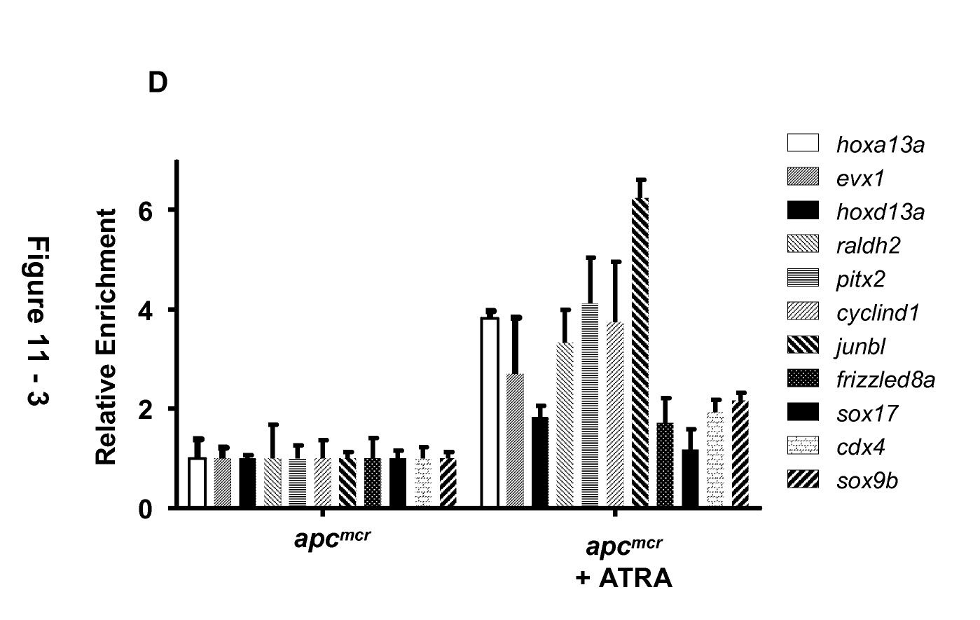

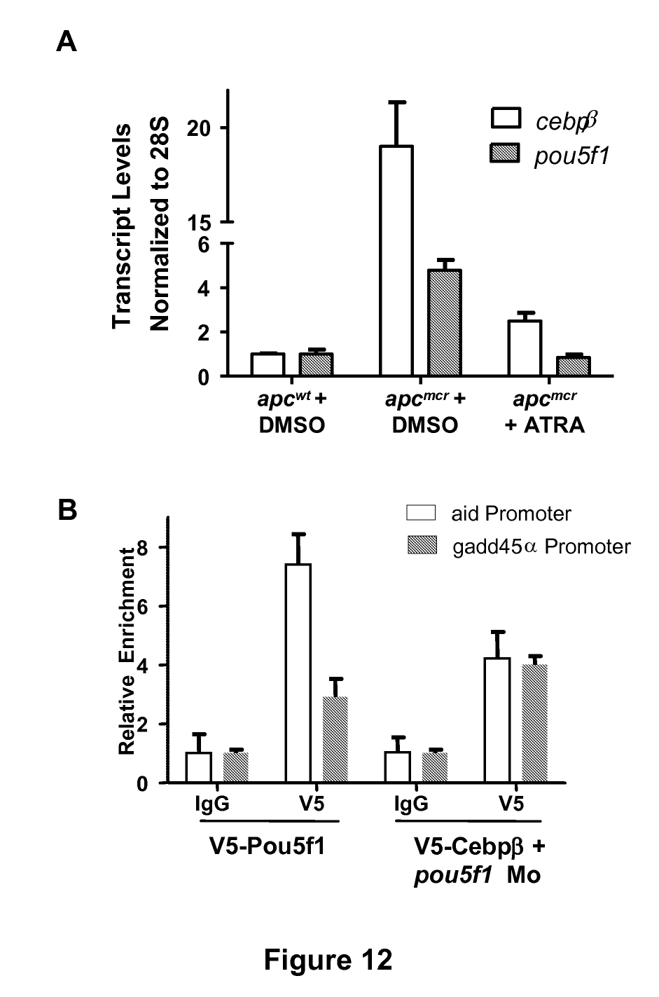

As RA can directly repress transcription of pou5f1 in the zebrafish by binding to an RARE in the pou5f1 promoter (Parvin et al., 2008), and since a related you family transcription factor activates expression of aldh1a1 (Guimond et al., 2002), the expression of pou5f1 in apc^{mcr} mutant embryos was examined. Expression level was elevated as assessed by RT-PCR (FIG. 12A). Furthermore, treatment apc^{mcr} mutant embryos with RA reduced the expression of pou5f1 (FIG. 12A). In FIG. 12A the Y-axis shows fold induction normalized to 28S and wild type DMSO treated sample. Cebpβ is another transcription factor that is upregulated in APC mutants and is down regulated by RA treatment (FIG. 12A) (Eisinger et al., 2006). Since components of the demethylase complex contain OCT and CEBP sites in their promoter, Cebpβ and Pou5f1 levels in apc mutants were suppressed by injection of morpholinos. Reduced expression of the demethylase was observed to an extent similar to that elicited by treatment with RA (Table 4). To test if Pou5f1 and Cebpβ were sufficient to upregulate these genes, Pou5f1 and Cebpβ were overexpressed. With overexpression, robust upregulation of all demethylase components in multiple tissues including the intestine was observed. Knockdown of pou5f1 also caused a significant reduction in the aldh1a2 expression in apc mutants as measured by whole mount in situ and quantitative RT-PCR.

Gadd45α and aid promoters both contain multiple OCT and CEBP binding sites. Gadd45α and aid promoters contain adjacent and partially overlapping OCT and CEBP recognition sequences at +400 and −1300 position from their respective TSS. To assess binding, chromatin immunoprecipitation (ChIP) following the expression of tagged derivatives in 56 hpf embryos was performed. Here, Pou5f1 enrichment was readily detected when overexpressed, but not Cebpβ (FIG. 12B). However, significant Cebpβ enrichment was detected when Cebpβ was overexpressed along with a small amount of pou5f1 morpholino (FIG. 12B). FIG. 12B shows the fold enrichment near the aid or gadd45α TSS (a region which contains overlapping Oct and Cebp binding sites) for Cebpfi to and Pou5f1 in embryos injected with V5-Cebpfi (along with Pou5f1 mo, 80 pg) or V5-Pou5f1 expressing plasmids. ChIP was performed with antibodies against the tags. Normalization control primers are located 3 kb upstream (a region without Cebpfi sites) of TSS of Gadd45α gene. This finding indicates that endogenous Pou5f1 might block the binding of exogenous Cebpβ, The blocking of the binding could be through steric occlusion. These data indicate that both Pou5f1 and Cebpβ, whose levels are regulated by retinoic acid, are direct positive regulators of demethylase genes.

### The Demethylase System Helps Maintain Intestinal Epithelial Cells in a Progenitor-Like State Following Loss of Apc

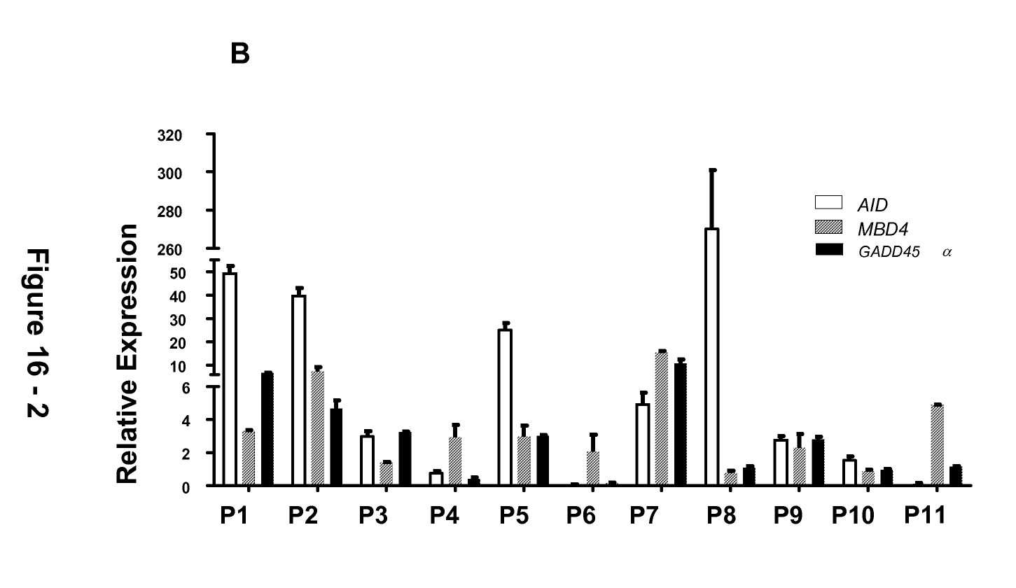

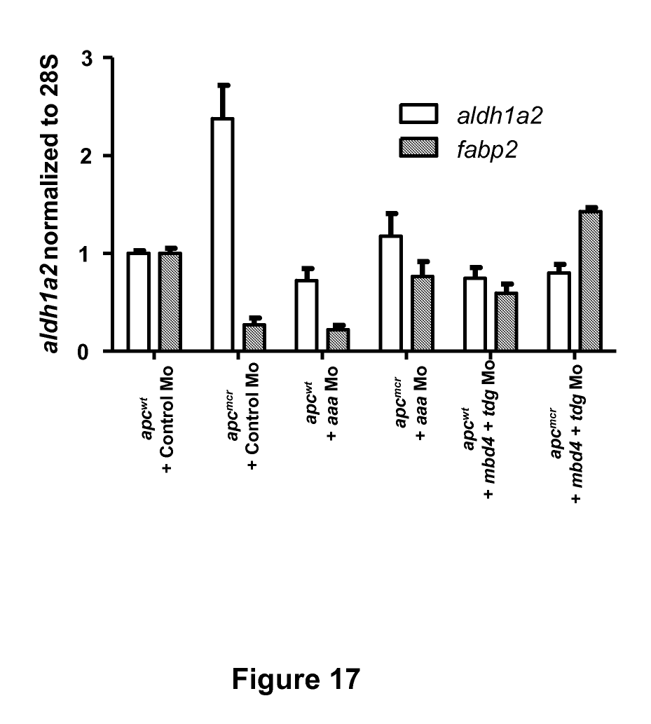

Recent studies indicate that APC mutation leads to increased numbers of improperly fated progenitor-like cells (Huang et al., 2009). ALDH1 is a marker of both normal and colonic stem cells and is upregulated in APC mutant human tissues (Huang et al., 2009). In agreement with this, apc mutant zebrafish embryos expressed high levels of aldh1a2 was hypomethylated in apc mutant embryos and this hypomethylation relied on demethylase components. Whether demethylase components were involved in maintaining an undifferentiated progenitor population in apc mutants was evaluated. The knock down of demethylase components in apc mutant embryos reduced expression of aldh1a2 as determined both by whole mount in situ hybridization or RT-PCR (FIG. 17 (injected with control Mo/aaa Mo/mbd4+tdg Mo) and Table 5). Whole mount in situ for aldh1a2, fabp2 and hoxa13a in apc^{mcr} and apc^{wt} injected with control Mo, or aaa Mo (0.5 ng each of aid, apobec2a and apobec2b Mo), or combined mbd4 and tdg Mo (1 ng each), or overexpression of dnmt1 (0.1 pg, an amount that rescues Dnmt1 morphants with 97% knockdown of Dnmt1 levels was conducted). Loss of this progenitor cell marker was paralleled by increased intestinal differentiation (ifabp/fabp2) (FIG. 17). Table 5 shows the statistics for APC mutant phenotypes upon demethylase knock down.

TABLE 5
Statistics for APC Mutant Phenotypes Upon
Demethylase Knock Down

| | Fabp2 | Hoxa13a | Raldh2 | Pcna | cyclinD1 | Pitx2 |
|---|---|---|---|---|---|---|
| apc^{wt} + | 99% | 96% | 98% | 97% | 96% | 94% |
| Control | (n = 95) | (n = 75) | (n = 105 | (n = 103) | (n = 109) | (n = 112) |
| Mo | | | | | | |
| Apc^{mer} + | 3% | 3% | 3% | 10% | 17% | 10% |
| Control | (n = 38) | (n = 33) | (n = 39 | (n = 41) | (n = 40) | (n = 40) |
| Mo | | | | | | |
| Apc^{mer} + | 71% | 95% | 73% | 63% | 58% | 71% |
| AAA | (n = 49) | (n = 39) | (n = 49) | (n = 48 | (n = 50) | (n = 35) |
| Mo | | | | | | |
| Apc^{mer} + | 75% | 86% | 70% | 64% | 60% | 73% |
| MBD4 + | (n = 52) | (n = 35) | (n = 53) | (n = 55) | (n = 42) | (n = 37) |
| TDG | | | | | | |
| Mo | | | | | | |
| apc^{mer} + | 4% | 6% | 6% | 12% | 10% | 15% |
| Dnmt1 | (n = 45) | (n = 35) | (n = 35) | (n = 52) | (n = 42) | (n = 33) |
| apc^{mer} + | n/d | n/d | n/d | 85% | 90% | n/d |
| Pitx2 | | | | (n = 33) | (n = 30) | |
| Mo | | | | | | |
| apc^{mer} + | 59% | n/d | n/d | n/d | n/d | n/d |
| RAL | (n = 46) | | | | | |
| Control | 100% | n/d | n/d | n/d | n/d | n/d |
| Mo | (n = ) | | | | | |
| rdh1l | 41% | n/d | n/d | n/d | n/d | n/d |
| Mo | (n = ) | | | | | |
| rdh1l | 75% | n/d | n/d | n/d | n/d | n/d |
| Mo + | (n = ) | | | | | |
| RAL | | | | | | |

There is a effect on the expression pattern of hoxa13a; wild type embryos restrict hoxa13a expression to the distal tip of the gut. Expression is observed throughout the gut in apc mutants. Knock down of demethylase components restores proper spatial expression of hoxa13a to apc mutant embryos (Table 5). Furthermore, hoxa13a was hypomethylated in apc mutants in a demethylase-dependent manner. Finally, overexpression of dnmt1 (at the concentrations tested) did not affect expression of these markers, focusing the causal effect on the upregulation of the demethylase rather than the possible downregulation of dnmt1.

In addition, key genes for proliferation (i.e., cyclind1, cyclinb2 and pitx2) were hypomethylated in zebrafish apc mutants in a demethylase-dependent fashion, and Pitx2 directly activates CyclinD1 expression (Baek et al., 2003). High levels (by in situ staining) of pcna, cyclind1 and pitx2 expression within brain of apc mutants was observed. The high level of expression was greatly attenuated by knock down of demethylase components (Table 5), but not through ectopic dnmt1 expression. Furthermore, increased pcna and cyclind1 expression depended on Pitx2 (. These data indicate a role for hypomethylation (and the demethylase) in governing proliferation following loss of Apc, and this relationship is observed in the developing brain.

Lef1 and Groucho2 Directly Repress rdh1l and Suppress Intestinal Differentiation

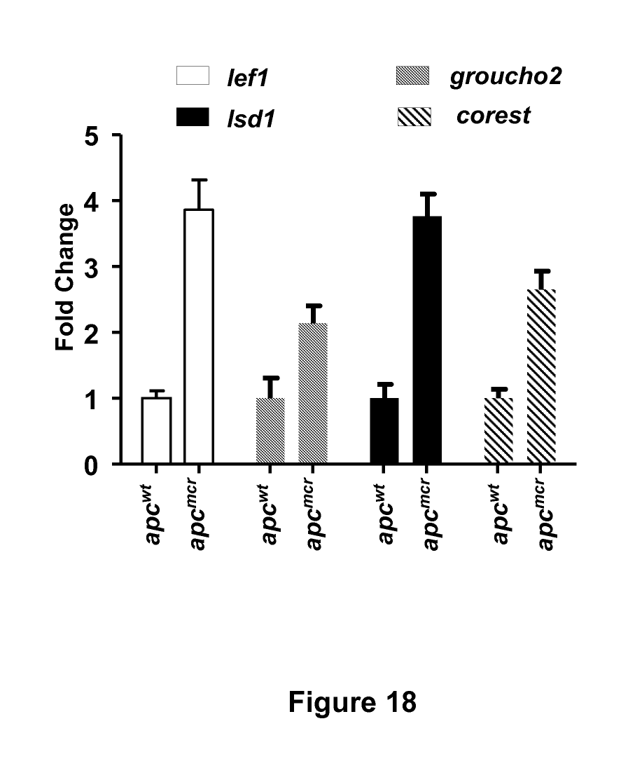

Apc^{mcr} zebrafish lack retinoic acid production due to loss of retinol dehydrogenase (RDH) expression and display upregulation of Ctbp1 (Nadauld et al., 2006). To better understand how APC regulates retinoic acid and demethylase expression, whether CtBP1 in conjunction with other transcriptional repressors directly represses RDH was assessed. The RDH promoter in both humans and zebrafish contains numerous TCF/LEF binding sites, and apc^{mcr} zebrafish expressed aberrantly high levels of lef1 and its co-regulator groucho2, but not tcf4 or groucho3 (FIG. 18—quantitative RT-PCR showing fold upregulation of lef1, groucho2, lsd1 and corest in apc^{mcr} zebrafish compared to wild type siblings (ape). Transcript levels normalized to 28S rRNA transcripts and then to wild type values).

Knock down of Lef1 in apc^{mcr} zebrafish embryos improved many of the overt morphological defects present in apc^{mcr} embryos including intestinal and pancreatic differentiation, but not improve eye differentiation defects (Table 6). Knockdown of either Lef1 or Groucho2 was accompanied by the induction of rdh1l expression within the intestine (Table 6).

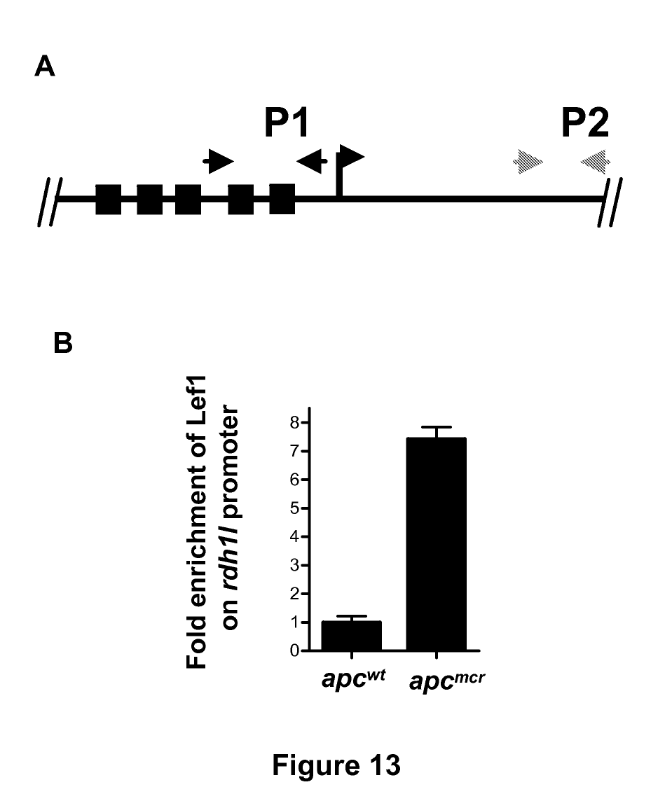

Lef1 directly regulates rdh1l expression, as ChIP experiments revealed a 7.4-fold enrichment of Lef1 on the rdh1l promoter (second and the third Lef1 binding sites upstream of the TSS in apc^{mcr} zebrafish embryos compared to their wild type siblings (FIG. 13). In FIG. 13, the Y-axis shows fold enrichment on a region containing Lef1 sites compared to an internal control region (without Lef1 binding site, P2) on the rdh1l promoter. Values obtained from Lef1 antibody were normalized to ones obtained using a non specific antibody and then expressed as fold enrichment compared to apc^{wt}. Co-injection of both lef1 and groucho2 mRNA, but not either alone, reduced expression of fabp2 (a marker of intestinal differentiation) in the intestine of 80 hpf wild type zebrafish embryos (Table 6). The expression of gata6, a marker of primordial gut, was unaffected by injection of lef1 and groucho2 mRNA, thereby eliminating the possibility that the intestine failed to develop. Also, as stated above, Table 6 shows a summary of lef1 and groucho2 morphant phenotypes.

TABLE 6
Summary of Lef1 and Groucho2 Morphant Phenotypes

| | fabp2 | rdh1l | trypsin | irbp |
|---|---|---|---|---|
| apc^{sib} | 100% | 100% | 100% | 96% |
| | (n = 48) | (n = 42) | (n = 38) | (n = 48) |
| apc^{mcr} | 0% | 0% | 0% | 0% |
| | (n = 52) | (n = 41) | (n = 35) | (n = 52) |
| | p < 0.0001 | p < 0.0001 | p < 0.0001 | p < 0.0001 |
| apc^{mcr} + | 87% | 88% | 86% | 5% |
| Lef 1 mo | (n = 42) | (n = 41) | (n = 44) | (n = 42) |
| | p < 0.0001 | p < 0.0001 | p < 0.0001 | p = ns |
| apc^{mcr} + | 8% | 9% | 10% | n/d |
| Tcf4 mo | (n = 39) | (n = 35) | (n = 20) | |
| | p = ns | p = ns | p = ns | |
| apc^{mcr} + | 95% | 83% | 86% | 5% |
| Groucho2 mo | (n = 61) | (n = 46) | (n = 36) | (n = 61) |
| | p < 0.0001 | p < 0.0001 | p < 0.0001 | p = ns |
| apc^{mcr} + | 83% | n/d | n/d | 8% |
| Lsd1 mo | (n = 65) | | | (n = 65) |
| | p <0.0001 | | | p = ns |
| apc^{mcr} + | 79% | n/d | n/d | 11% |
| Corest mo | (n = 62) | | | (n = 62) |
| | p< 0.0001 | | | p = ns |
| WT | 100% | 100% | n/a | n/d |
| | (n = 21) | (n =18) | | |
| | p = ns | p = ns | | |
| full length | 87.2% | 87.9% | n/a | n/d |
| lef1 RNA | (n = 42) | (n = 46) | | |
| | p < 0.0001 | p = ns | | |
| dom-ve | 100% | 87.9% | n/a | n/d |
| lef1 RNA | (n = 21) | (n = 46) | | |
| | p = ns | p = ns | | |
| groucho2 | 90% | 86% | n/a | n/d |
| RNA | (n = 30) | (n = 42) | | |
| | p = ns | p = ns | | |
| full length | 47% | 24.9% | n/a | n/d |
| lef1 + | (n = 349) | (n = 40) | | |
| groucho2 | p < 0.0001 | p < 0.0001 | | |
| RNA | | | | |
| dom-ve lef1 + | 57.1% | n/a | n/a | n/d |
| groucho2 | (n = 91) | | | |
| RNA | p < 0.0001 | | | |

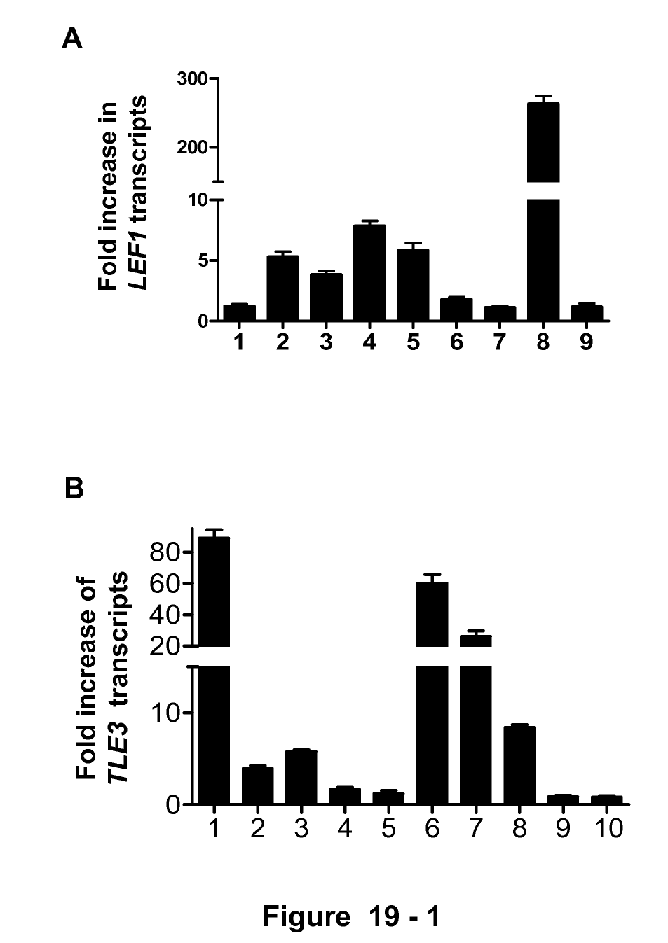

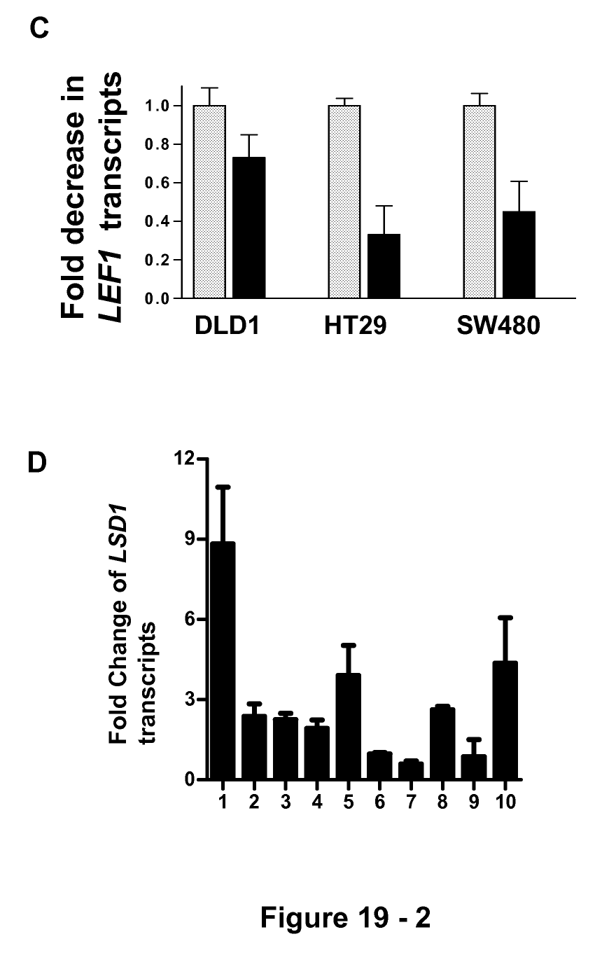

Human LEF1 physically interacts with Groucho/TLE family members in the absence of WNT signaling. Human LEF1 and TLE3 (a homolog of Groucho2) showed robust interaction by co-immunoprecipitation, and similarly zebrafish Lef1 and Groucho2 interacted significantly in SW-480 cells which harbor APC mutation. A role for LEF1 and TLE3 overexpression could extend to human samples, as LEF1 and TLE3 were each significantly upregulated in 50-60% of the ten adenoma samples tested (FIG. 19A-B—quantitative RT-PCR analyses; Y-axis values are fold changes in expression of indicated genes in adenomas normalized first to 28s levels and then mRNA/28S ratio from matching to uninvolved tissue, valued at 1; X-axis numbers refer to patients sample number).

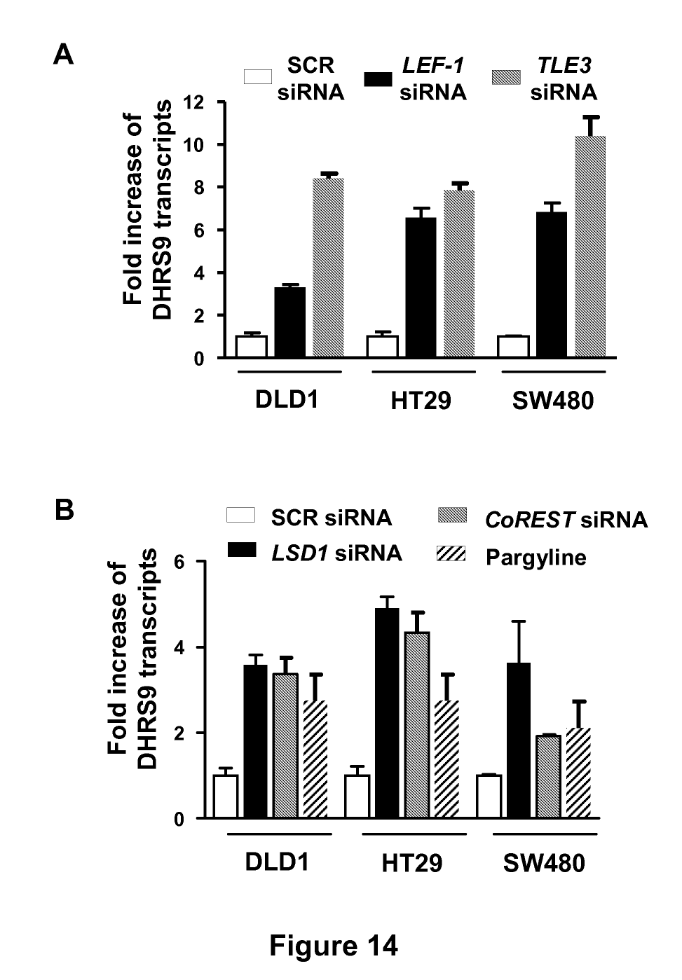

Next, the roles of LEF1 and Groucho2/TLE3 in repressing retinol dehydrogenase expression in three colon cancer cell lines harboring APC mutations was analyzed—DLD1, HT29 and SW480. LEF1 and TLE3 were knocked down using short interfering RNAs (siRNAs), which significantly reduced expression of their respective genes (FIG. 19C (quantitative RT-PCR) and S5D (immunoblot)) and restored RDHL (DHRS9) expression in all three cell lines (FIG. 14A). For example, FIG. 14A shows the quantitative PCR measuring DHRS9 (RDHL) expression in DLD1, SW480, and HT29 cells, which were transfected with either a Scrambled (Scr) siRNA or a specific siRNA against LEF1 or TLE3 (B) or siRNAs against LSD1. The Y-axis values represent fold change in DHRS9 expression. Normalization for DHRS9 absolute values was done first to 18S rRNA values and then to DHRS9/18S ratio from Scr siRNA. Error bars indicate standard deviation. These data support roles of LEF1 and TLE3 together in repressing RDH expression in zebrafish and humans.

LEF1/TLE3 Represses Retinol Dehydrogenase through Recruitment of the CtBP1/LSD1 Corepressor Complex

CtBP1 exists in a multi-protein repressor complex, which includes the histone demethylase LSD1 and the scaffold CoREST (Shi et al., 2003) Immunoprecipitation of TLE3 from SW480 cells efficiently co-precipitated CtBP1, and TLE3 co-precipitated LSD1. Furthermore, LEF1 co-precipitated LSD1, indicating that CtBP1/LSD1 complex can interact with both LEF1 and TLE3. Next, whether these interacting proteins can occupy the DHRS9 (previously known as RDHL) promoter was tested. To test this, ChIP of CtBP1 and LSD1 on the DHRS9 promoter in SW480 cells was performed. ChIP revealed significant enrichment of CtBP1 and LSD1 on the DHRS9 promoter region containing LEF1 sites.

Also, knockdown of LSD1 by siRNA was sufficient to restore DHRS9 levels in DLD1, HT29 and SW480 cells (FIG. 14B). Furthermore, inhibition of LSD1 enzymatic activity by treatment with pargyline (Shi et al., 2004) also upregulated DHRS9 expression in all three cell lines (FIG. 14B). Finally, knockdown of CoREST also restored DHRS9 expression in the same three cell lines (FIG. 14B). Taken together, these data indicate that a LEF1/TLE3 module binds to the retinol dehydrogenase promoter and recruits the CtBP1/LSD1/CoREST complex.

### LSD1 and CoREST are Upregulated in APC Mutant Tissues and Repress DHRS9

CtBP1 protein levels are elevated in APC mutant cells (Nadauld et al., 2006), as they lack APC- and proteasome-mediated CtBP1 destruction. However, immunostaining on cross sections of FAP colonic adenomas showed upregulation of both LSD1 and CtBP1 compared to matched normal mucosa samples Western blot further confirmed upregulation of LSD1 protein in FAP polyps compared to matched normal tissue. Here, unlike CtBP1, and similar to LEF1 and TLE3, LSD1 transcript levels were upregulated in multiple adenoma tissues by RT-PCR analysis (FIG. 19D).

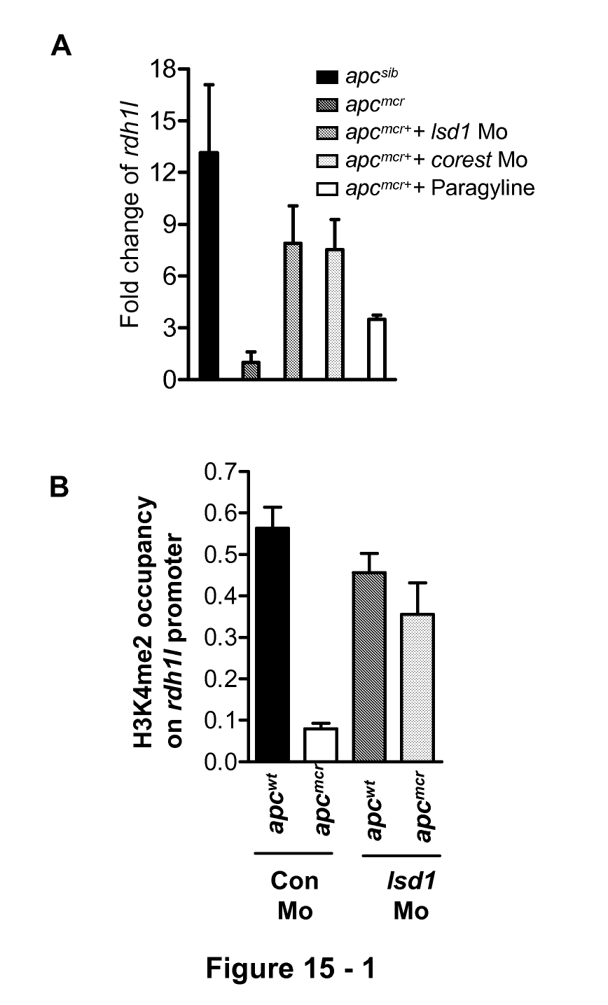

Next, whether LSD1 and CoREST function in intestinal differentiation and retinol dehydrogenase regulation was tested using zebrafish. Immunoblot analysis revealed Lsd1 and Ctbp1 protein upregulation in apc^{mcr} zebrafish. Similar to adenomas, the transcript levels of Lsd1 and Corest are also upregulated in apc^{mcr} zebrafish embryos at 80 hpf as examined by whole mount in situ analysis and verified by semi-quantitative RT-PCR (FIG. 18). Knockdown of Lsd1 and Corest rescued the expression of intestinal rdh1l in apc^{mcr} zebrafish embryos (FIG. 15A). FIG. 15A shows the RT-PCR results for rdh1l levels compared to 28S levels in apc^{wt} and apc^{mcr} embryos injected with control/lsd1/corest morpholinos or treated with pargyline (to inhibit Lsd1 activity). Furthermore, rescue of rdh1l expression was accompanied by restoration of intestinal differentiation (fabp2). Treatment of apc^{mcr} zebrafish embryos with pargyline also rescued expression of rdh1l (FIG. 15A) and fabp2, indicating that the catalytic activity of Lsd1 is present in such regulation in zebrafish. These data indicate that Lsd1 and Corest are involved to maintain the repression of rdh1l and resulting undifferentiated status of the intestines of apc^{mcr} zebrafish embryos.

LSD1 Demethylates H3K4 on the rdh1l Promoter to Cause Repression

In addition, the role of Lsd1 in the repression of rdh1l transcription by demethylating H3K4me2 was examined (Shi et al., 2004)). To test this, H3K4me2 on the rdh1l promoter was monitored by ChIP in apc^{mcr} zebrafish injected with lsd1 or control morpholino. Consistent with higher rdh1l expression, apc^{wt} zebrafish have higher H3K4me2 compared to apc^{mcr} zebrafish (FIG. 15B). FIG. 15B shows the ChIP analysis for H3K4me2 marks on the rdh1l promoter performed in apc^{wt} and apc^{mcr} zebrafish embryos injected with either control or lsd1 morpholino. Knockdown of Lsd1 did not appreciably alter the H3K4me2 levels on rdh1l promoter in apc^{wt} zebrafish embryos. However, knockdown of Lsd1 in apc^{mcr} zebrafish embryos increased H3K4me2 on the rdh1l promoter, indicating that Lsd1 upregulation (in Apc mutant conditions) leads to H3K4me2 demethylation and rdh1l repression (FIG. 15B). This is consistent with the rescue of rdh1l expression by pargyline treatment in human colon cancer cell lines (FIG. 14B) and apc^{mcr} zebrafish (FIG. 15B).

Genomic hypomethylation was proposed by Holliday as an oncogenic mechanism nearly 25 years ago (Holliday and Jeggo, 1985). Since then, many studies have focused on DNA methyltransferases and the paradoxical hypermethylation of tumor suppressor genes that occurs in the background of genome wide hypomethylation. Mechanisms explaining the underlying hypomethylation, its contribution to tumor initiation and progression and its relationship to genetic events are largely unknown. The model disclosed herein links APC loss, as a key genetic determinant of colon adenoma development, including the misregulation of DNA methylation dynamics through activation of a DNA demethylase system was presented. This dynamic regulation appears to be involved jn the proper fating of intestinal progenitor cells in zebrafish and targets key regulators of progenitor cell maintenance and differentiation in both zebrafish and human tissues. Mechanistically, loss of APC results in suppression of retinoic acid biosynthesis, which causes activation of the demethylase components via the transcriptional regulators Pou5f1 and Cebpβ. Retinoic acid biosynthesis is impaired due to repression of RDH by aberrant upregulation of a transcriptional complex including LEF1, Groucho2/TLE3 CtBP1, LSD1 and Corest.

The model described offers a new mechanistic explanation for previous studies implicating DNA hypomethylation in cancer development (Goelz et al., 1985). Other models have considered the possibility that genome-wide demethylation during tumorigenesis results from inability of DNA methyltransferases to maintain normal DNA methylation patterns in highly proliferative cells. A loss of dnmt1 was observed in parallel with upregulation of the demethylase system in apc mutant zebrafish embryos. However, unlike knock down of the demethylase components, injection of dnmt1 into apc mutants failed to restore intestinal differentiation and methylation on specific genes. This finding indicates a major role for the misregulation of the demethylase, rather than Dnmt1 loss, in maintaining the progenitor-like state of intestinal cell present in Apc mutants. Consistent with this model knockout of Apobec-1, a mammalian 5meC deaminase highly related to AID, reduced adenoma formation in the ApcMin mouse model (Kunal Morgan add REF) (Blanc et al., 2007).

This work also provides insight into an apparent contradiction brought about by to previous studies indicating that genetic loss or pharmacologic inhibition of DNMT1 appears protective in some circumstances (Eads et al., 2002; Laird et al., 1995). The demethylase system can work through a process that first involves deamination of 5meC to thymidine, a process that can enhance C to T transitions. Loss of methylation due to DNMT1 inhibition could lower the yield of deamination products (5meC>T transitions) by removing the substrate, 5meC. In turn, this can slow adenoma progression by limiting this mode of mutation. Laird et al. speculated reduced mutation rates in the ApcMin mice treated with 5-Aza-cytidine as this treatment effectively prevented adenoma initiation but failed to affect established polyps (Laird et al., 1995).

Although the transcriptional effects of retinoic acid in controlling cell patterning, fate and differentiation are well documented (Blaner WS, 1999; Vogel S, 1999), the regulation of RA production as a determinant of cell fate decisions mechanism is less well understood. Production of retinoic acid first requires converting dietary retinol (vitamin A) into retinoic acid, a process that occurs via two enzymatic steps (Duester, 2000): (1) the conversion of retinol into retinal by alcohol dehydrogenases (ADH) and short chain dehydrogenases (SDR), followed by (2) the conversion of retinal into retinoic acid via aldehyde dehydrogenases (ALDH) (Duester, 2000). Studies in mice and chickens indicated that RALDHs (Duester et al., 2003), rather than RDHs, represent the primary point for regulating tissue-specific production of retinoic acid. Recent studies in zebrafish, however, have demonstrated that Rdh1 and Rdh1l are essential for intestinal differentiation, and the development of the pectoral fin, jaw, eye, and exocrine pancreas (Nadauld et al., 2004; Nadauld et al., 2005). Furthermore, mice carrying a mutation in Rdh10 died during midgestation and displayed craniofacial, limb, and organ abnormalities due to an inability to oxidize retinol to retinal (Sandell et al., 2007). These findings provide a model indicating a dynamic inverse relationship between RDHs and ALDHs in regulating RA production in intestinal cells. In this model, high levels of ALDH appear to poise cells for RA production upon generation of RAL by RDH. This, in turn, initiates a program of differentiation that relies in part on the suppression of the demethylase system (FIG. 15C-D).

Multiple transcriptional repressors, namely LEF1, Groucho2/TLE3, CtBP1, LSD1 and CoREST work together to repress the production of retinoic acid by direct binding to, and repression of, the rdh1 promoter. Further, the activity of LSD1 maintains repression of the RDH promoter in the presence of APC mutation. Consistent with the model, knockdown of these proteins restored rdh1l expression and intestinal differentiation to apc mutants. Furthermore, these data indicate that APC regulation of RDHs and the demethylase system occurs independently β-catenin, a finding consistent with previous studies that indicate 3-catenin independent loss of intestinal cell fating in apc mutant zebrafish (Jette et al., 2004; Nadauld et al., 2006; Phelps et al., 2009a; Phelps et al., 2009b). These findings also reveal an oncogenic role for LEF1, which is known to be upregulated in sporadic colon carcinomas (Hovanes et al., 2001), but thought previously to act primarily through an interaction with β-catenin. These data indicate that upregulation of multiple corepressors (LEF1, Groucho2/TLE3, CtBP1, LSD1 and CoREST) is an immediate consequence of APC loss and precedes dysregulation of Wnt signaling. Consistent with this idea, human FAP adenoma tissues also showed increased levels of all of these factors. These new findings indicate an initiating role for these corepressors in the formation of colon adenomas. Taken together, these data support the model disclosed herein wherein mutations in APC cause upregulation of LEF1, Groucho2/TLE3, CtBP1, LSD1 and CoREST and that this complex serves to repress intestinal differentiation by directly targeting RDHs.

These results indicate a mechanism for a rapid and robust change in DNA methylation dynamics during development, mediated by retinoic acid. A marked upregulation of demethylase components in RA deficient embryos was observed, as well as a marked downregulation of demethylase components following treatment with retinoic acid. These studies demonstrate that retinoic acid antagonizes two key direct activators of demethylase components, Pou5f1 and Cebpβ, leading to demethylase downregulation. These results indicate that part of the basis for the developmental plasticity and maintenance of progenitor cells is the DNA methylation dynamics provided by the demethylase system. Following exposure to retinoic acid and differentiation, this dynamic is lost. The loss of this dynamic can favor the imposition of DNA methylation patterns at the promoters of key developmental regulators, which can help commit cells to differentiation and help maintain that commitment.

In summary, DNA cytosine demethylases are upregulated in APC mutant human and zebrafish tissue, and are responsible for demethylation of some key cancer genes which are important for maintaining a progenitor cell population in APC mutant tissues. These poised progenitor cells can be stimulated to differentiate. Further, APC controls demethylase expression by retinoic acid in a manner which is mediated by Pou5f1 and Cebpβ. These findings offer a mechanistic model implicating active DNA demethylation in contributing to cell fating defects following loss of APC. In addition, for the first time, these to studies show that certain cancer genes are demethylated early during genome-wide hypomethylation, possibly poised for activation. Additional events can signal other activators for these genes to become transcriptionally active. Disclosed herein is a model wherein APC control of RA production contributes methylation machinery to promote differentiation and in the absence of APC these demethylases are activated, demethylating critical fate regulators which lead to the poising of cells in a progenitor like cells that can be induced to proliferate by other (growth factor/Ras-like) signals.

### Example 3

### Expression of Demethylase System Components in Cancer Stem Cells

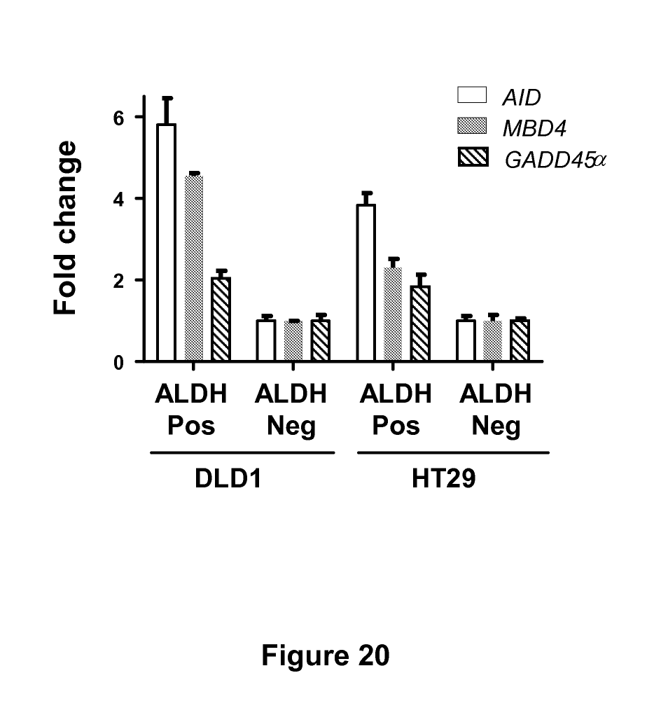

DLD-1 and HT-29 cells were grown in DMEM culture medium. 1×10⁷ cells were subjected to aldefluour assay according to manufacturer's instructions (Stem Cell Technologies) and then ALDH positive cells were sorted in FACScan Vantage machine (BD Biosciences). ALDH combined with DEAB was used as negative control for sorting. ALDH positive and negative cells were then subjected to RNA isolation. mRNA was isolated and rt-PCR performed to examine the levels of the respective demethylase components. The results show increased expression of various Demethylase System Components in human Cancer Stem Cells (See FIG. 20).

What is claimed is:

1. A method of detecting an oncogenic event in a sample comprising determining the expression level of one or more Demethylase System Components in a sample and comparing those expression levels to the expression levels of a normal sample, wherein an increase in the expression of the one or more Demethylase System Components compared to the expression levels of a normal sample indicates an oncogenic event.

2. The method of claim 1, wherein the one or more Demethylase System Components includes at least one Demethylase System cytidine deaminase.

3. The method of claim 1, wherein the one or more Demethylase System Components includes at least one Demethylase System thymine glycosylase.

4. The method of claim 1, wherein the one or more Demethylase System Components includes at least one Demethylase System cofactor.

5. The method of claim 1, further comprising, determining the level of methylated DNA in the sample, wherein a decrease in the level of methylated DNA indicates an oncogenic event.

6. The method of claim 1, further comprising, determining the level of DNA methylation of one or more of the promoters selected from the group consisting of: aldh1a2, hox13a, evx1, pitx2, cyclind1, hoxd13a, junb1, frizzled8a, cdx4, sox9b, cyclinb2, sox4, Fabp2, Raldh2, pcna, and cyclinD1, wherein a decrease in the level of DNA methylation of the one or more of the promoters selected from the group consisting of: aldh1a2, hox13a, evx1, pitx2, cyclind1, hoxd13a, junb1, frizzled8a, cdx4, sox9b, cyclinb2, sox4, Fabp2, Raldh2, pcna, or cyclinD1 indicates an oncogenic event.

7. The method of claim 1, further comprising, determining the presence of a G:T intermediate, wherein the presence of a G:T intermediate indicates an oncogenic event.

8. The method of claim 1, further comprising, determining the level of retinoic acid in the sample, wherein a decrease in the level of retinoic acid in the sample indicates an oncogenic event.

9. The method of claim 1, further comprising determining the level of expression of Cebpβ or Pou5f1, wherein an increase in the level of expression of Cebpβ or Pou5f1 indicates an oncogenic event.

10. The method of claim 1, further comprising determining the presence or absence of a mutation in the adenomatous polyposis coli tumor suppressor gene, wherein a mutation in the adenomatous polyposis coli tumor suppressor gene indicates an oncogenic event.

11. The method of claim 12, further comprising determining level of a retinol dehydrogenase or alcohol dehydrogenase, wherein a mutation in the adenomatous polyposis coli tumor suppressor gene and a decrease in the level of the retinol dehydrogenases or alcohol dehydrogenase indicates an oncogenic event.

12. The method of claim 1, further comprising determining levels of a retinol dehydrogenase or alcohol dehydrogenase and retinol, wherein a decrease in the level of the retinol dehydrogenase or alcohol dehydrogenase and an increase in the level of retinol indicates an oncogenic event.

13. The method of claim 12, further comprising determining the level of ALDH1, wherein an increase in ALDH1 and the presence of a mutation in APC indicates an oncogenic event.

14. The method of claim 1, further comprising determining the level of dnmt1, wherein a decrease in the level of dnmt1 indicates an oncogenic event.

15. The method of claim 1, further comprising determining the level of adenomatous polyposis coli tumor suppressor gene expression, wherein a decrease in the level of adenomatous polyposis coli tumor suppressor gene expression indicates an oncogenic event.

16. The method of claim 1, further comprising determining the level of LEF1 and Grouch2/TLE3 expression, wherein an increase in the level of LEF1 and Grouch2/TLE3 expression indicates an oncogenic event.

17. The method of claim 1, further comprising determining the level of LSD1, CoREST or CrBP1, wherein an increase in the level of LSD1, CoREST or CrBP1 expression indicates an oncogenic event.


SEQUENCE LISTING (146 sequences)

SEQ ID NO: 1 - DNA, 25 bp, Artificial Sequence
  Description of Artificial Sequence Note = Synthetic Construct
gttttgttttcgcttacctgtccag

SEQ ID NO: 2 - DNA, 25 bp, Artificial Sequence
  Description of Artificial Sequence Note = Synthetic Construct
gctgctgccctttctatcggccatc

SEQ ID NO: 3 - DNA, 25 bp, Artificial Sequence
  Description of Artificial Sequence Note = Synthetic Construct
cttgctgtcctttttgtctgccatg

SEQ ID NO: 4 - DNA, 25 bp, Artificial Sequence
  Description of Artificial Sequence Note = Synthetic Construct
atataaaaacatacctttccgttgc

SEQ ID NO: 5 - DNA, 25 bp, Artificial Sequence
  Description of Artificial Sequence Note = Synthetic Construct
caccgagtccatcctggaaaaccac

SEQ ID NO: 6 - DNA, 25 bp, Artificial Sequence
  Description of Artificial Sequence Note = Synthetic Construct
aaaggaacttactttgtatcagtaa

SEQ ID NO: 7 - DNA, 25 bp, Artificial Sequence
  Description of Artificial Sequence Note = Synthetic Construct
tccggcagtctgcattctggagaaa

SEQ ID NO: 8 - DNA, 25 bp, Artificial Sequence
  Description of Artificial Sequence Note = Synthetic Construct
agagagaaacacacctgctcgctgc

SEQ ID NO: 9 - DNA, 25 bp, Artificial Sequence
  Description of Artificial Sequence Note = Synthetic Construct
ctgacttcccgttgttatctgtgtt

SEQ ID NO: 10 - DNA, 25 bp, Artificial Sequence
  Description of Artificial Sequence Note = Synthetic Construct
cacacacggtggcaaatacgcgtca

SEQ ID NO: 11 - DNA, 25 bp, Artificial Sequence
  Description of Artificial Sequence Note = Synthetic Construct
gctcctcccctttgtatccgcgatc

SEQ ID NO: 12 - DNA, 25 bp, Artificial Sequence
  Description of Artificial Sequence Note = Synthetic Construct
cttggtgtcgttttagtgtgcgatg

SEQ ID NO: 13 - DNA, 24 bp, Artificial Sequence
  Description of Artificial Sequence Note = Synthetic Construct
cctcttacctcagtacaatttata

SEQ ID NO: 14 - DNA, 25 bp, Artificial Sequence
  Description of Artificial Sequence Note = Synthetic Construct
cgctctctccgtcatctttccgcta

SEQ ID NO: 15 - DNA, 25 bp, Artificial Sequence
  Description of Artificial Sequence Note = Synthetic Construct
tttatcaaacttactcggactctgg

SEQ ID NO: 16 - DNA, 25 bp, Artificial Sequence
  Description of Artificial Sequence Note = Synthetic Construct
agcccttctttgtttacctgtctgc

SEQ ID NO: 17 - DNA, 25 bp, Artificial Sequence
  Description of Artificial Sequence Note = Synthetic Construct
gccctgtggatacatcttgaaatgt

SEQ ID NO: 18 - DNA, 25 bp, Artificial Sequence
  Description of Artificial Sequence Note = Synthetic Construct
ggtctgacttcttattggacaacat

SEQ ID NO: 19 - DNA, 25 bp, Artificial Sequence
  Description of Artificial Sequence Note = Synthetic Construct
ccttctctaacattgcgggcatctt

SEQ ID NO: 20 - DNA, 25 bp, Artificial Sequence
  Description of Artificial Sequence Note = Synthetic Construct
cctcttacctcagttacaatttata

SEQ ID NO: 21 - RNA, 25 nt, Artificial Sequence
  Description of Artificial Sequence Note = Synthetic Construct
gccgauacgacagugauggagacaa

SEQ ID NO: 22 - RNA, 25 nt, Artificial Sequence
  Description of Artificial Sequence Note = Synthetic Construct
uugucuccaucacugucguaucggc

SEQ ID NO: 23 - DNA, 23 bp, Artificial Sequence
  Description of Artificial Sequence Note = Synthetic Construct
caccucaggucaaacaggadtdt

SEQ ID NO: 24 - DNA, 23 bp, Artificial Sequence
  Description of Artificial Sequence Note = Synthetic Construct
uccuguuugaccugaggugdtdt

SEQ ID NO: 25 - DNA, 23 bp, Artificial Sequence
  Description of Artificial Sequence Note = Synthetic Construct
ggaauugguuucagucaaadtdt

SEQ ID NO: 26 - DNA, 23 bp, Artificial Sequence
  Description of Artificial Sequence Note = Synthetic Construct
tttgactgaaaccaattccdtdt

SEQ ID NO: 27 - DNA, 23 bp, Artificial Sequence
  Description of Artificial Sequence Note = Synthetic Construct
cgacgccgcuucaacauagdtdt

SEQ ID NO: 28 - DNA, 23 bp, Artificial Sequence
  Description of Artificial Sequence Note = Synthetic Construct
ctatgttgaagcggcgtcgdtdt

SEQ ID NO: 29 - DNA, 21 bp, Artificial Sequence
  Description of Artificial Sequence Note = Synthetic Construct
agacagaagacagaagcdtdt

SEQ ID NO: 30 - DNA, 20 bp, Artificial Sequence
  Description of Artificial Sequence Note = Synthetic Construct
gccuaucugucucgcudtdt

SEQ ID NO: 31 - DNA, 19 bp, Artificial Sequence
  Description of Artificial Sequence Note = Synthetic Construct
agtgtgctcatgacccaga

SEQ ID NO: 32 - DNA, 18 bp, Artificial Sequence
  Description of Artificial Sequence Note = Synthetic Construct
cacaacgcacccaagtga

SEQ ID NO: 33 - DNA, 18 bp, Artificial Sequence
  Description of Artificial Sequence Note = Synthetic Construct
atggccgatagaaagggc

SEQ ID NO: 34 - DNA, 19 bp, Artificial Sequence
  Description of Artificial Sequence Note = Synthetic Construct
cctgtgatggtctcgaatg

SEQ ID NO: 35 - DNA, 20 bp, Artificial Sequence
  Description of Artificial Sequence Note = Synthetic Construct
atggcagacaaaaaggacag

SEQ ID NO: 36 - DNA, 20 bp, Artificial Sequence
  Description of Artificial Sequence Note = Synthetic Construct
cggtctcctacaatgatctc

SEQ ID NO: 37 - DNA, 19 bp, Artificial Sequence
  Description of Artificial Sequence Note = Synthetic Construct
aagagacgctcttccatga

SEQ ID NO: 38 - DNA, 20 bp, Artificial Sequence
  Description of Artificial Sequence Note = Synthetic Construct
ttatgatcgtctggagtgac

SEQ ID NO: 39 - DNA, 23 bp, Artificial Sequence
  Description of Artifical Sequence Note = Synthetic Construct
atggatgaaaggctgtatggatc

SEQ ID NO: 40 - DNA, 20 bp, Artificial Sequence
  Description of Artificial Sequence Note = Synthetic Construct
tcctctggatgtacaggcat

SEQ ID NO: 41 - DNA, 23 bp, Artificial Sequence
  Description of Artificial Sequence Note = Synthetic Construct
atgacttttgaagaaccgtgtgg

SEQ ID NO: 42 - DNA, 17 bp, Artificial Sequence
  Description of Artificial Sequence Note = Synthetic Construct
gatctggagggccacat

SEQ ID NO: 43 - DNA, 21 bp, Artificial Sequence
  Description of Artificial Sequence Note = Synthetic Construct
tccgttctggattttacatgc

SEQ ID NO: 44 - DNA, 20 bp, Artificial Sequence
  Description of Artificial Sequence Note = Synthetic Construct
acccgattttgggtttcagt

SEQ ID NO: 45 - DNA, 20 bp, Artificial Sequence
  Description of Artificial Sequence Note = Synthetic Construct
tctcacagtcggcgtttatg

SEQ ID NO: 46 - DNA, 20 bp, Artificial Sequence
  Description of Artificial Sequence Note = Synthetic Construct
cggctctcctcacagtaggt

SEQ ID NO: 47 - DNA, 21 bp, Artificial Sequence
  Description of Artificial Sequence Note = Synthetic Construct
caacgacatcaacatcgttcg

SEQ ID NO: 48 - DNA, 20 bp, Artificial Sequence
  Description of Artificial Sequence Note = Synthetic Construct
tcagcgttcaggcagagtaa

SEQ ID NO: 49 - DNA, 22 bp, Artificial Sequence
  Description of Artificial Sequence Note = Synthetic Construct
atggaagacgccaaaaacataa

SEQ ID NO: 50 - DNA, 20 bp, Artificial Sequence
  Description of Artificial Sequence Note = Synthetic Construct
cgtgatggaatggaacaaca

SEQ ID NO: 51 - DNA, 26 bp, Artificial Sequence
  Description of Artificial Sequence Note = Synthetic Construct
tttaattagatggtagtttttatatt

SEQ ID NO: 52 - DNA, 23 bp, Artificial Sequence
  Description of Artificial Sequence Note = Synthetic Construct
aacaaaccaacctaaaatataaa

SEQ ID NO: 53 - DNA, 20 bp, Artificial Sequence
  Description of Artificial Sequence Note = Synthetic Construct
ggttggagatggtttatttt

SEQ ID NO: 54 - DNA, 24 bp, Artificial Sequence
  Description of Artificial Sequence Note = Synthetic Construct
ttcctaaaccacaaatataaacat

SEQ ID NO: 55 - DNA, 23 bp, Artificial Sequence
  Description of Artificial Sequence Note = Synthetic Construct
atggaagatgttaaaaatataaa

SEQ ID NO: 56 - DNA, 26 bp, Artificial Sequence
  Description of Artificial Sequence Note = Synthetic Construct
taataaaataaaacaacacttaaaat

SEQ ID NO: 57 - DNA, 24 bp, Artificial Sequence
  Description of Artificial Sequence Note = Synthetic Construct
gatgttaaaaatataaagaaaggt

SEQ ID NO: 58 - DNA, 24 bp, Artificial Sequence
  Description of Artificial Sequence Note = Synthetic Construct
aaacaacacttaaaatcacaatat

SEQ ID NO: 59 - DNA, 20 bp, Artificial Sequence
  Description of Artificial Sequence Note = Synthetic Construct
gtttgtattgaattattggt

SEQ ID NO: 60 - DNA, 24 bp, Artificial Sequence
  Description of Artificial Sequence Note = Synthetic Construct
aaaatcattttccttaaaaatcaa

SEQ ID NO: 61 - DNA, 21 bp, Artificial Sequence
  Description of Artificial Sequence Note = Synthetic Construct
gagaagggataaatggattat

SEQ ID NO: 62 - DNA, 21 bp, Artificial Sequence
  Description of Artificial Sequence Note = Synthetic Construct
aaatcaactctaatccctcta

SEQ ID NO: 63 - DNA, 20 bp, Artificial Sequence
  Description of Artificial Sequence Note = Synthetic Construct
gtttttaaataggtataggt

SEQ ID NO: 64 - DNA, 20 bp, Artificial Sequence
  Description of Artificial Sequence Note = Synthetic Construct
caaacaaaattacataccta

SEQ ID NO: 65 - DNA, 20 bp, Artificial Sequence
  Description of Artificial Sequence Note = Synthetic Construct
aggtataggttaggttatgt

SEQ ID NO: 66 - DNA, 21 bp, Artificial Sequence
  Description of Artificial Sequence Note = Synthetic Construct
catacctactcttataccaaa

SEQ ID NO: 67 - DNA, 23 bp, Artificial Sequence
  Description of Artificial Sequence Note = Synthetic Construct
ttttgtttaaaaaggataagtat

SEQ ID NO: 68 - DNA, 20 bp, Artificial Sequence
  Description of Artificial Sequence Note = Synthetic Construct
aatacatactaatcatctct

SEQ ID NO: 69 - DNA, 19 bp, Artificial Sequence
  Description of Artificial Sequence Note = Synthetic Construct
gttagaggttggagagttt

SEQ ID NO: 70 - DNA, 21 bp, Artificial Sequence
  Description of Artificial Sequence Note = Synthetic Construct
taatcatctctctcaaatctc

SEQ ID NO: 71 - DNA, 23 bp, Artificial Sequence
  Description of Artificial Sequence Note = Synthetic Construct
ggataagtttttttatggttagt

SEQ ID NO: 72 - DNA, 23 bp, Artificial Sequence
  Description of Artificial Sequence Note = Synthetic Construct
aaacttcctataaaaaaaaaccc

SEQ ID NO: 73 - DNA, 21 bp, Artificial Sequence
  Description of Artificial Sequence Note = Synthetic Construct
tggttagttttattagtggga

SEQ ID NO: 74 - DNA, 22 bp, Artificial Sequence
  Description of Artificial Sequence Note = Synthetic Construct
ccctaataataaaccaatacct

SEQ ID NO: 75 - DNA, 19 bp, Artificial Sequence
  Description of Artificial Sequence Note = Synthetic Construct
aaagattgtttggggtaat

SEQ ID NO: 76 - DNA, 21 bp, Artificial Sequence
  Description of Artificial Sequence Note = Synthetic Construct
aattattatacccaaactcta

SEQ ID NO: 77 - DNA, 23 bp, Artificial Sequence
  Description of Artificial Sequence Note = Synthetic Construct
gtaattaaaaggtttttgtgtgt

SEQ ID NO: 78 - DNA, 23 bp, Artificial Sequence
  Description of Artificial Sequence Note = Synthetic Construct
aactctaccttttttccataata

SEQ ID NO: 79 - DNA, 20 bp, Artificial Sequence
  Description of Artificial Sequence Note = Synthetic Construct
ttggataaagtaaggaaagg

SEQ ID NO: 80 - DNA, 23 bp, Artificial Sequence
  Description of Artificial Sequence Note = Synthetic Construct
tcttaccaaatcaatattatcct

SEQ ID NO: 81 - DNA, 20 bp, Artificial Sequence
  Description of Artificial Sequence Note = Synthetic Construct
aaggaaagggttggttttgt

SEQ ID NO: 82 - DNA, 23 bp, Artificial Sequence
  Description of Artificial Sequence Note = Synthetic Construct
caatattatcctctctttccttc

SEQ ID NO: 83 - DNA, 24 bp, Artificial Sequence
  Description of Artificial Sequence Note = Synthetic Construct
gattttattttgggttgttttagt

SEQ ID NO: 84 - DNA, 20 bp, Artificial Sequence
  Description of Artificial Sequence Note = Synthetic Construct
ttttctcctcctcaaaaaac

SEQ ID NO: 85 - DNA, 23 bp, Artificial Sequence
  Description of Artificial Sequence Note = Synthetic Construct
tttgggttgttttagtagtaaag

SEQ ID NO: 86 - DNA, 19 bp, Artificial Sequence
  Description of Artificial Sequence Note = Synthetic Construct
ctcctcctcaaaaaactac

SEQ ID NO: 87 - DNA, 20 bp, Artificial Sequence
  Description of Artificial Sequence Note = Synthetic Construct
ggaggataagaattataatt

SEQ ID NO: 88 - DNA, 22 bp, Artificial Sequence
  Description of Artificial Sequence Note = Synthetic Construct
tattcaacttcctataaaaaaa

SEQ ID NO: 89 - DNA, 19 bp, Artificial Sequence
  Description of Artificial Sequence Note = Synthetic Construct
aagagtggttgtggatttt

SEQ ID NO: 90 - DNA, 23 bp, Artificial Sequence
  Description of Artificial Sequence Note = Synthetic Construct
acctattactctacaaatatcta

SEQ ID NO: 91 - DNA, 21 bp, Artificial Sequence
  Description of Artificial Sequence Note = Synthetic Construct
gtttttgatggatagaaataa

SEQ ID NO: 92 - DNA, 25 bp, Artificial Sequence
  Description of Artificial Sequence Note = Synthetic Construct
gatggatagaaataatttgaattat

SEQ ID NO: 93 - DNA, 21 bp, Artificial Sequence
  Description of Artificial Sequence Note = Synthetic Construct
atataatccaaacaaaaccaa

SEQ ID NO: 94 - DNA, 24 bp, Artificial Sequence
  Description of Artificial Sequence Note = Synthetic Construct
ctcttcatataataatcaaacata

SEQ ID NO: 95 - DNA, 20 bp, Artificial Sequence
  Description of Artificial Sequence Note = Synthetic Construct
atgagtagatttaatgatgt

SEQ ID NO: 96 - DNA, 20 bp, Artificial Sequence
  Description of Artificial Sequence Note = Synthetic Construct
caaaataccacatacaaaaa

SEQ ID NO: 97 - DNA, 23 bp, Artificial Sequence
  Description of Artificial Sequence Note = Synthetic Construct
ggattttattaggtgaaaaaaat

SEQ ID NO: 98 - DNA, 20 bp, Artificial Sequence
  Description of Artificial Sequence Note= Synthetic Construct
cacatacaaaaattcaccaa

SEQ ID NO: 99 - DNA, 19 bp, Artificial Sequence
  Description of Artificial Sequence Note = Synthetic Construct
agatcgtggattacgtcgc

SEQ ID NO: 100 - DNA, 21 bp, Artificial Sequence
  Description of Artifical Sequence Note = Synthetic Construct
cttggcctttatgaggatctc

SEQ ID NO: 101 - DNA, 22 bp, Artificial Sequence
  Description of Artificial Sequence Note = Synthetic Construct
cattgacgtcaatgggagtttg

SEQ ID NO: 102 - DNA, 18 bp, Artificial Sequence
  Description of Artificial Sequence Note = Synthetic Construct
ttagccagagagctctgc

SEQ ID NO: 103 - DNA, 21 bp, Artificial Sequence
  Description of Artificial Sequence Note = Synthetic Construct
atacatttgtggctgctgtgt

SEQ ID NO: 104 - DNA, 22 bp, Artificial Sequence
  Description of Artificial Sequence Note = Synthetic Construct
acatcctgacgatatatggaga

SEQ ID NO: 105 - DNA, 21 bp, Artificial Sequence
  Description of Artificial Sequence Note = Synthetic Construct
ggttgaaaatggacattctgc

SEQ ID NO: 106 - DNA, 21 bp, Artificial Sequence
  Description of Artificial Sequence Note = Synthetic Construct
gctaaacagtgctgaattagg

SEQ ID NO: 107 - DNA, 21 bp, Artificial Sequence
  Description of Artificial Sequence Note = Synthetic Construct
atacatttgtggctgctgtgt

SEQ ID NO: 108 - DNA, 22 bp, Artificial Sequence
  Description of Artificial Sequence Note = Synthetic Construct
acatcctgacgatatatggaga

SEQ ID NO: 109 - DNA, 20 bp, Artificial Sequence
  Description of Artificial Sequence Note = Synthetic Construct
gcatgatgatggaaacggac

SEQ ID NO: 110 - DNA, 18 bp, Artificial Sequence
  Description of Artificial Sequence Note = Synthetic Construct
ttccactcagcaccgagt

SEQ ID NO: 111 - DNA, 20 bp, Artificial Sequence
  Description of Artificial Sequence Note = Synthetic Construct
ggaagaccttcaatcagcgt

SEQ ID NO: 112 - DNA, 21 bp, Artificial Sequence
  Description of Artificial Sequence Note = Synthetic Construct
caatgacaagaacacagcgtc

SEQ ID NO: 113 - DNA, 20 bp, Artificial Sequence
  Description of Artificial Sequence Note = Synthetic Construct
gtgtgaagacggctgaatac

SEQ ID NO: 114 - DNA, 20 bp, Artificial Sequence
  Description of Artificial Sequence Note = Synthetic Construct
tcctcacgccatgttttgga

SEQ ID NO: 115 - DNA, 19 bp, Artificial Sequence
  Description of Artificial Sequence Note = Synthetic Construct
tgaatcgagtggctcgaag

SEQ ID NO: 116 - DNA, 20 bp, Artificial Sequence
  Description of Artificial Sequence Note= Synthetic Construct
catccgctctcaaggtttac

SEQ ID NO: 117 - DNA, 20 bp, Artificial Sequence
  Description of Artificial Sequence Note = Synthetic Construct
ctcacatgcctttgtgtacc

SEQ ID NO: 118 - DNA, 19 bp, Artificial Sequence
  Description of Artificial Sequence Note = Synthetic Construct
attcctgaagcgtcgctct

SEQ ID NO: 119 - DNA, 19 bp, Artificial Sequence
  Description of Artificial Sequence Note = Synthetic Construct
tgtgaccaagaccttcctg

SEQ ID NO: 120 - DNA, 20 bp, Artificial Sequence
  Description of Artificial Sequence Note = Synthetic Construct
cgtgggtaaacagtgcaatc

SEQ ID NO: 121 - DNA, 19 bp, Artificial Sequence
  Description of Artificial Sequence Note = Synthetic Construct
aagccttcggactggagaa

SEQ ID NO: 122 - DNA, 20 bp, Artificial Sequence
  Description of Artificial Sequence Note = Synthetic Construct
gaccacgtcgatatccacat

SEQ ID NO: 123 - DNA, 20 bp, Artificial Sequence
  Description of Artificial Sequence Note = Synthetic Construct
tttgcacctgtacctccgaa

SEQ ID NO: 124 - DNA, 20 bp, Artificial Sequence
  Description of Artificial Sequence Note = Synthetic Construct
gaaatccacagccactcttg

SEQ ID NO: 125 - DNA, 23 bp, Artificial Sequence
  Description of Artificial Sequence Note = Synthetic Construct
gacaacctaccatattagcttgc

SEQ ID NO: 126 - DNA, 20 bp, Artificial Sequence
  Description of Artificial Sequence Note = Synthetic Construct
tcaagcatgtgcttcaacgg

SEQ ID NO: 127 - DNA, 20 bp, Artificial Sequence
  Description  of Artificial Sequence Note = Synthetic Construct
cagtggatttcaccaggtga

SEQ ID NO: 128 - DNA, 20 bp, Artificial Sequence
  Description of Artificial Sequence Note = Synthetic Construct
aggtgaaggaagagcgatga

SEQ ID NO: 129 - DNA, 21 bp, Artificial Sequence
  Description of Artificial Sequence NOte = Synthetic Construct
ggttgaaaatggacattctgc

SEQ ID NO: 130 - DNA, 21 bp, Artificial Sequence
  Description of Artificial Sequence Note = Synthetic Construct
gctaaacagtgctgaattagg

SEQ ID NO: 131 - DNA, 23 bp, Artificial Sequence
  Description of Artificial Sequence Note = Synthetic Construct
aacagagtggcaggcatcattta

SEQ ID NO: 132 - DNA, 25 bp, Artificial Sequence
  Description of Artificial Sequence Note = Synthetic Construct
acagtcatatttcttggaagacccc

SEQ ID NO: 133 - DNA, 25 bp, Artificial Sequence
  Description of Artificial Sequence Note = Synthetic Construct
ctgaactttcatcaaattggtttcc

SEQ ID NO: 134 - DNA, 25 bp, Artificial Sequence
  Description of Artificial Sequence Note = Synthetic Construct
gtgatggcaaacttaaagtccttca

SEQ ID NO: 135 - DNA, 23 bp, Artificial Sequence
  Description of Artificial Sequence Note = Synthetic Construct
ccaaaaacataaagaaaggccca

SEQ ID NO: 136 - DNA, 20 bp, Artificial Sequence
  Description of Artificial Sequence Note = Synthetic Construct
atatccttgcctgatacctg

SEQ ID NO: 137 - DNA, 25 bp, Artificial Sequence
  Description of Artificial Sequence Note = Synthetic Construct
aaactctcttcaattctttatgcca

SEQ ID NO: 138 - DNA, 20 bp, Artificial Sequence
  Description of Artificial Sequence Note = Synthetic Construct
atatccttgcctgatacctg

SEQ ID NO: 139 - DNA, 19 bp, Artificial Sequence
  Description of Artificial Sequence Note = Synthetic Construct
acatctcatctacctccca

SEQ ID NO: 140 - DNA, 20 bp, Artificial Sequence
  Description of Artificial Sequence Note = Synthetic Construct
atatccttgcctgatacctg

SEQ ID NO: 141 - DNA, 22 bp, Artificial Sequence
  Description of Artificial Sequence Note = Synthetic Construct
atggaagacgccaaaaacataa

SEQ ID NO: 142 - DNA, 20 bp, Artificial Sequence
  Description of Artificial Sequence Note = Synthetic Construct
aaataacgcgcccaacacca

SEQ ID NO: 143 - DNA, 22 bp, Artificial Sequence
  Description of Artificial Sequence Note = Synthetic Construct
atggaagacgccaaaaacataa

SEQ ID NO: 144 - DNA, 28 bp, Artificial Sequence
  Description of Artificial Sequence Note = Synthetic Construct
tggcacaaaatcgtattcattaaaacca

SEQ ID NO: 145 - DNA, 22 bp, Artificial Sequence
  Description of Artificial Sequence Note = Synthetic Construct
atggaagacgccaaaaacataa

SEQ ID NO: 146 - DNA, 24 bp, Artificial Sequence
  Description of Artificial Sequence Note = Synthetic Construct
caacacttaaaatcgcagtatcca